SEQUENCE LISTING (288 sequences)

SEQ ID NO: 1 - protein, 29 aa, artificial
  GHRH
YADAIFTAAYRKVLGQLSARKLLQDIXSR

SEQ ID NO: 2 - protein, 9 aa, artificial
  PR1 (T-cell epitope)
VLQELNVTV

SEQ ID NO: 3 - protein, 10 aa, artificial
  Protease-3 peptide (1)
RLFPDFFTRV

SEQ ID NO: 4 - protein, 10 aa, artificial
  Protease-3 peptide (2)
VLQELNVTVV

SEQ ID NO: 5 - protein, 9 aa, artificial
  Protease-3 peptide (3)
NLSASVTSV

SEQ ID NO: 6 - protein, 9 aa, artificial
  Protease-3 peptide (4)
IIQGIDSFV

SEQ ID NO: 7 - protein, 11 aa, artificial
  Protease-3 peptide (5)
VLLALLLISGA

SEQ ID NO: 8 - protein, 9 aa, artificial
  Protease-3 peptide (6)
QLPQQDQPV

SEQ ID NO: 9 - protein, 11 aa, artificial
  Protease-3 peptide (7)
FLNNYDAENKL

SEQ ID NO: 10 - protein, 8 aa, artificial
  Protease-3 peptide (8)
VLQELWTV

SEQ ID NO: 11 - protein, 9 aa, artificial
  Protease-3 peptide (9)
VLQELNVKV

SEQ ID NO: 12 - protein, 8 aa, artificial
  Protease-3 peptide (10)
VLQELWKV

SEQ ID NO: 13 - protein, 8 aa, artificial
  Protease-3 peptide (11)
VMQELWTV

SEQ ID NO: 14 - protein, 6 aa, artificial
  P3, B-CELL EPITOPE
PEQGYN

SEQ ID NO: 15 - protein, 8 aa, artificial
  P3, B-CELL EPITOPE (WITH SPACER)
SGPEQGYN

SEQ ID NO: 16 - protein, 30 aa, artificial
  GLP1
HAEGTFTSDVSSYLEGQAAKEFIAWLVKGR

SEQ ID NO: 17 - protein, 10 aa, artificial
  LHRH
XHWSYXLRPG

SEQ ID NO: 18 - protein, 34 aa, artificial
  PTH
SVSEIQLMHNLGKHLNSMERVEWLRKKLQDVHNF

SEQ ID NO: 19 - protein, 11 aa, artificial
  SUBSTANCE P
RPKPQQFFGLM

SEQ ID NO: 20 - protein, 10 aa, artificial
  NEUROKININ A
HKTDSFVGLM

SEQ ID NO: 21 - protein, 10 aa, artificial
  NEUROKININ B
DMHDFFVGLM

SEQ ID NO: 22 - protein, 14 aa, artificial
  BOMBESIN
XQRLGNQWAVGHLM

SEQ ID NO: 23 - protein, 8 aa, artificial [sequence omitted]

SEQ ID NO: 24 - protein, 5 aa, artificial
  LEUCINE ENKEPHALIN
YGGFL

SEQ ID NO: 25 - protein, 5 aa, artificial
  METHIONINE ENKEPHALIN
YGGFM

SEQ ID NO: 26 - protein, 18 aa, artificial
  [Des Ala20, Gln34] Dermaseptin
ALWKTMLKKGTMALHAGK

SEQ ID NO: 27 - protein, 7 aa, artificial
  Antimicrobial Peptide, Surfactant
DDDDDDD

SEQ ID NO: 28 - protein, 7 aa, artificial
  Antimicrobial Anionic Peptide, Surfactant- associated AP
GADDDDD

SEQ ID NO: 29 - protein, 18 aa, artificial
  Apidaecin IA
GNNRPVYIPQPRPPHPRI

SEQ ID NO: 30 - protein, 18 aa, artificial
  Apidaecin IB
GNNRPVYIPQPRPPHPRL

SEQ ID NO: 31 - protein, 14 aa, artificial
  OV-2
LRRIIRKIIHIIKK

SEQ ID NO: 32 - protein, 20 aa, artificial
  1025, Acetyl-Adhesin Peptide (1025 - 1044) amide
QLKTADLPAGRDETTSFVLV

SEQ ID NO: 33 - protein, 15 aa, artificial
  Theromacin (49 - 63)
QRLPNNKQCRCINAR

SEQ ID NO: 34 - protein, 22 aa, artificial
  Pexiganan (MSI-78)
GIGKFLKKAKKFGKAFVKILKK

SEQ ID NO: 35 - protein, 13 aa, artificial
  Indolicidin
ILPWKWPWWPWRR

SEQ ID NO: 36 - protein, 15 aa, artificial
  Apelin - 15 (63-77)
RRQRPRLSHKGPMPF

SEQ ID NO: 37 - protein, 15 aa, artificial
  CFP10 (71-85)
EISTNIRQAGVQYSR

SEQ ID NO: 38 - protein, 10 aa, artificial
  Lethal Factor (LF) Inhibitor Anthrax related
MKARRKKVYP

SEQ ID NO: 39 - protein, 12 aa, artificial
  Bactenecin
RLCRIVVIRVCR

SEQ ID NO: 40 - protein, 6 aa, artificial
  Hepatitis Virus C NS3 Protease Inhibitor 2
DEXEXC

SEQ ID NO: 41 - protein, 6 aa, artificial
  Hepatitis Virus C NS3 Protease Inhibitor 3
DEXIXC

SEQ ID NO: 42 - protein, 6 aa, artificial
  Hepatitis Virus  NS3 Protease Inhibitor 4
DELEXC

SEQ ID NO: 43 - protein, 6 aa, artificial
  NS4A-NS4B Hepatitis Virus C, NS3 Protease Inhibitor 1
DEMEEC

SEQ ID NO: 44 - protein, 10 aa, artificial
  HIV-1, HIV-2 Protease Substrate
ATLNFPISPW

SEQ ID NO: 45 - protein, 6 aa, artificial
  Anti-Flt1 Peptide
GNQWFI

SEQ ID NO: 46 - protein, 16 aa, artificial
  Bak - BH3
GQVGRQLAIIGDDINR

SEQ ID NO: 47 - protein, 20 aa, artificial
  Bax BH3 peptide (55-74), wild type
STKKLSECLKRIGDELDSNM

SEQ ID NO: 48 - protein, 29 aa, artificial
  Bid BH3 - r8
RRRRRRRRGEDIIRNIARHLAQVGDSMDR

SEQ ID NO: 49 - protein, 10 aa, artificial
  CTT, Gelatinase Inhibitor
CTTHWGFTLC

SEQ ID NO: 50 - protein, 9 aa, artificial
  E75, Her -2/neu (369 - 377)
KIFGSLAFL

SEQ ID NO: 51 - protein, 24 aa, artificial
  GRP78 Binding Chimeric Peptide Motif
WIFPWIQLGGKLAKLAKKLAKLAK

SEQ ID NO: 52 - protein, 10 aa, artificial
  p53(17-26)
ETFSDLWKLL

SEQ ID NO: 53 - protein, 7 aa, artificial
  EGFR2/KDR Antagonist
ATWLPPR

SEQ ID NO: 54 - protein, 26 aa, artificial
  Colivelin
SALLRSIPAPAGASRLLLLTGEIDLP

SEQ ID NO: 55 - protein, 17 aa, artificial
  AGA-(C8R) HNG17, Humanin derivative
PAGASRLLLLTGEIDLP

SEQ ID NO: 56 - protein, 9 aa, artificial
  Activity - Dependent Neurotrophic Factor, ADNF
SALLRRIPA

SEQ ID NO: 57 - protein, 14 aa, artificial
  Beta - Secretase Inhibitor 1
KTEEISEVNXVAEF

SEQ ID NO: 58 - protein, 14 aa, artificial
  Beta - Secretase Inhibitor 2
KTEEISEVNXVAEF

SEQ ID NO: 59 - protein, 15 aa, artificial
  ch - Amyloid (30-16)
CTFVRTHIFCKEHQF

SEQ ID NO: 60 - protein, 24 aa, artificial
  Humanun, HN
MAPRGFSCLLLLTSEIDLPVKRRA

SEQ ID NO: 61 - protein, 24 aa, artificial
  sHNG, [Gly14] - HN, [Gly14]-Humanin
MAPRGFSCLLLLTGEIDLPVKRRA

SEQ ID NO: 62 - protein, 9 aa, artificial
  Angiotensin Converting Enzyme Inhibitor, BPP
XWPRPQIPP

SEQ ID NO: 63 - protein, 9 aa, artificial
  Renin Inhibitor III
RRPFHXIHK

SEQ ID NO: 64 - protein, 11 aa, artificial
  Annexin 1 (ANXA - 1; Ac2-12)
AMVSEFLKQAW

SEQ ID NO: 65 - protein, 9 aa, artificial
  Anti-Inflammatory Peptide 1
MQMKKVLDS

SEQ ID NO: 66 - protein, 9 aa, artificial
  Anti-Inflammatory Peptide 2
HDMNKVLDL

SEQ ID NO: 67 - protein, 12 aa, artificial
  Anti-Inflammatory Apelin 12
RPRLSHKGPMPF

SEQ ID NO: 68 - protein, 14 aa, artificial
  [D - Phe12, Leu14]-Bombesin
XQRLGNQWAVGFLL

SEQ ID NO: 69 - protein, 16 aa, artificial
  Antennapedia Peptide, acid (penetratin)
RQIKIWFQNRRMKWKK

SEQ ID NO: 70 - protein, 17 aa, artificial
  Antennepedia Leader Peptide (CT)
KKWKMRRNQFWVKVQRG

SEQ ID NO: 71 - protein, 14 aa, artificial
  Mastoparan
INLKALAALAKKIL

SEQ ID NO: 72 - protein, 9 aa, artificial
  [Thr28, Nle31]-Cholecystokinin (25-33) sulfated
RNYTGWXDF

SEQ ID NO: 73 - protein, 13 aa, artificial
  Nociceptin (1-13), amide
FGGFTGARKSARK

SEQ ID NO: 74 - protein, 4 aa, artificial
  Fibrinolysis Inhibiting Factor
GPRP

SEQ ID NO: 75 - protein, 19 aa, artificial
  Gamma - Fibrinogen (377-395)
YSMKETTMKIIPFNRLSIG

SEQ ID NO: 76 - protein, 25 aa, artificial
  Xenin
MLTKFETKSARVKGLSFHPKRPWIL

SEQ ID NO: 77 - protein, 23 aa, artificial
  Obestatin, human
FNAPFDVGIKLSGVQYQQHSQAL

SEQ ID NO: 78 - protein, 6 aa, artificial
  [His1, Lys6]-GHRP, GHRP-6
HWAWFK

SEQ ID NO: 79 - protein, 7 aa, artificial
  [Ala5, -Ala8]-Neurokinin A (4-10)
DAFVXLM

SEQ ID NO: 80 - protein, 10 aa, artificial
  Neuromedin B
GNLWATGHFM

SEQ ID NO: 81 - protein, 10 aa, artificial
  Neuromedin C
GNHWAVGHLM

SEQ ID NO: 82 - protein, 6 aa, artificial
  Neuromedin N
KIPYIL

SEQ ID NO: 83 - protein, 14 aa, artificial
  Activity-Dependent Neurotrophic Factor, ADNF-14
VLGGGSALLRSIPA

SEQ ID NO: 84 - protein, 6 aa, artificial
  Acetalin 1, Opioid Receptor Antagonist 1
RFMWMR

SEQ ID NO: 85 - protein, 6 aa, artificial
  Acetalin 2, Opioid Receptor Antagonist 2
RFMWMK

SEQ ID NO: 86 - protein, 6 aa, artificial
  Acetalin 3, Opioid Receptor Antagonist 3
RFMWMT

SEQ ID NO: 87 - protein, 39 aa, artificial
  ACTH (1-39), human
SYSMEHFRWGKPVGKKRRPVKVYPNGAEDESAEAFPLEF

SEQ ID NO: 88 - protein, 32 aa, artificial
  ACTH (7-38), human
FRWGKPVGKKRRPVKVYPNGAEDESAEAFPLE

SEQ ID NO: 89 - protein, 40 aa, artificial
  Sauvagine
XGPPISIDLSLELLRKMIEIEKQEKEKQQAANNRLLLDTI

SEQ ID NO: 90 - protein, 8 aa, artificial
  Adipokinetic Hormone, Locusta Migratoria
XLNFSAGW

SEQ ID NO: 91 - protein, 12 aa, artificial
  Myristoylated ADP-Ribosylation Factor 6, myr-ARF6 (2-13)
GKVLSKIFGNKE

SEQ ID NO: 92 - protein, 20 aa, artificial
  PAMP (1-20); Proadrenomedullin (1-20) human
ARLDVASEFRKKWNKWALSR

SEQ ID NO: 93 - protein, 27 aa, artificial
  AGRP (25-51)
LAPMEGIRRPDQALLPELPGLGLRAPL

SEQ ID NO: 94 - protein, 30 aa, artificial
  Amylin (8-37), human
ATQRLANFLVHSSNNFGAILSSTNVGSNTY

SEQ ID NO: 95 - protein, 10 aa, artificial
  Angiotensin I, human
DRVYIHPFHL

SEQ ID NO: 96 - protein, 8 aa, artificial
  Angiotensin II, human
DRVYIHPF

SEQ ID NO: 97 - protein, 3 aa, artificial
  Apstatin, Aminopeptidase P Inhibitor
PPA

SEQ ID NO: 98 - protein, 24 aa, artificial
  Brevinin - 1
FLPVLAGIAAKVVPALFCKITKKC

SEQ ID NO: 99 - protein, 23 aa, artificial
  Magainin 1
GIGKFLHSAGKFGKAFVGEIMKS

SEQ ID NO: 100 - protein, 37 aa, artificial
  RL-37
RLGNFFRKVKEKIGGGLKKVGQKIKDFLGNLVPRTAS

SEQ ID NO: 101 - protein, 37 aa, artificial
  LL-37, Antimicrobial Peptide, human
LLGDFFRKSKEKIGKEFKRIVQRIKDFLRNLVPRTES

SEQ ID NO: 102 - protein, 37 aa, artificial
  Cecropin A
KWKLFKKIEKVGQNIRDGIIKAGPAVAVVGQATQIAK

SEQ ID NO: 103 - protein, 6 aa, artificial
  Antioxidant peptide A
PHCKRM

SEQ ID NO: 104 - protein, 10 aa, artificial
  Antioxidant peptide B
TRNYYVRAVL

SEQ ID NO: 105 - protein, 2 aa, artificial
  L-Carnosine
XH

SEQ ID NO: 106 - protein, 45 aa, artificial
  Bcl 9-2
GSEGLSKEQLEHRERSLQTLRDIERLLLRSGETEPFLKGPPGGAG

SEQ ID NO: 107 - protein, 8 aa, artificial
  NPVF
VPNLPQRF

SEQ ID NO: 108 - protein, 18 aa, artificial
  Neuropeptide AF (hNPAF), Human
AGEGLNSQFWSLAAPQRF

SEQ ID NO: 109 - protein, 20 aa, artificial
  Bax BH3 peptide (55-74)
STKKLSECLKRIGDELDSNM

SEQ ID NO: 110 - protein, 8 aa, artificial
  bFGF Inhibitory Peptide
APSGHYKG

SEQ ID NO: 111 - protein, 16 aa, artificial
  bFGF inhibitory Peptide II
MWYRPDLDERKQQKRE

SEQ ID NO: 112 - protein, 9 aa, artificial
  Bradykinin
RPPGFSPFR

SEQ ID NO: 113 - protein, 9 aa, artificial
  [Des-Arg10]-HOE I40
RRPXGXSXX

SEQ ID NO: 114 - protein, 4 aa, artificial
  Caspase 1 Inhibitor II
YVAD

SEQ ID NO: 115 - protein, 4 aa, artificial
  Caspase 1 Inhibitor VIII
WEHD

SEQ ID NO: 116 - protein, 7 aa, artificial
  Smac N7 Protein
AVPIAQK

SEQ ID NO: 117 - protein, 13 aa, artificial
  MEK1 Derived Peptide Inhibitor 1
MPKKKPTPIQLNP

SEQ ID NO: 118 - protein, 36 aa, artificial
  hBD-1, -Defensin-1, human
DHYNCVSSGGQCLYSACPIFTKIQGTCYRGKAKCCK

SEQ ID NO: 119 - protein, 45 aa, artificial
  hBD-3, -Defensin-3, human
GIINTLQKYYCRVRGGRCAVLSCLPKEEQIGKCSTRGRKCCRRKK

SEQ ID NO: 120 - protein, 37 aa, artificial
  hBD-4, -Defensin-4, human
ELDRICGYGTARCRKKCRSQEYRIGRCPNTYACCLRK

SEQ ID NO: 121 - protein, 30 aa, artificial
  HNP-1, Defensin Human Neutrophil Peptide 1
ACYCRIPACIAGERRYGTCIYQGRLWAFCC

SEQ ID NO: 122 - protein, 30 aa, artificial; identical to SEQ ID NO: 121
  HNP-2, Defensin Human neutrophil peptide-2

SEQ ID NO: 123 - protein, 4 aa, artificial
  Endomorphin- 1
YPWF

SEQ ID NO: 124 - protein, 31 aa, artificial
  -Endorphin, human porcine
YGGFMTSEKSQTPLVTLFKNAIIKNAYKKGE

SEQ ID NO: 125 - protein, 21 aa, artificial
  Endothelin 2, human
CSCSSWLDKECVYFCHLDIIW

SEQ ID NO: 126 - protein, 12 aa, artificial
  Fibrinogen Binding Inhibitor Peptide
HHLGGAKQAGDV

SEQ ID NO: 127 - protein, 6 aa, artificial
  Cyclo(-GRGDSP)
GRGDSP

SEQ ID NO: 128 - protein, 23 aa, artificial
  TP508, Thrombin-derived Peptide
AGYKPDEGKRGDACEGDSGGPFV

SEQ ID NO: 129 - protein, 30 aa, artificial
  Galanin, human
GWTLNSAGYLLGPHAVGNHRSFSDKNGLTS

SEQ ID NO: 130 - protein, 42 aa, artificial
  GIP, human
YAEGTFISDYSIAMDKIHQQDFVNWLLAQKGKKNDWKHNITQ

SEQ ID NO: 131 - protein, 27 aa, artificial
  Gastrin Releasing Peptide, human
VPLPAGGGTVLTKMYPRGNHWAVGHLM

SEQ ID NO: 132 - protein, 17 aa, artificial
  Gastrin - 1, human
XGPWLEEEEEAYGWMDF

SEQ ID NO: 133 - protein, 28 aa, artificial
  Ghrelin, human
GSXFLSPEHQRVQQRKESKKPPAKLQPR

SEQ ID NO: 134 - protein, 8 aa, artificial
  PDGF-BB peptide
CSRNLIDC

SEQ ID NO: 135 - protein, 6 aa, artificial
  [D-Lys3] - GHRP - 6
HWKWFK

SEQ ID NO: 136 - protein, 20 aa, artificial
  HCV Core Protein (1-20)
MSTNPKPQRKTKRNTNRRPQ

SEQ ID NO: 137 - protein, 17 aa, artificial
  a31 Integrin Peptide Fragment (325), amide
PRHRHMGAVFLLSQEAG

SEQ ID NO: 138 - protein, 5 aa, artificial
  Laminin Pentapeptide, amide
YIGSR

SEQ ID NO: 139 - protein, 4 aa, artificial
  Melanotropin - Potentiating Factor, MPF
KKGE

SEQ ID NO: 140 - protein, 24 aa, artificial
  VA--MSH, Lipotropin - Y, Proopiomelanocortin - derived
VAAEKKDEGPYRMEHFRWGSPPKD

SEQ ID NO: 141 - protein, 28 aa, artificial
  Atrial Natriuretic Peptide (1-28), human
SLRRSSCFGGRMDRIGAQSGLGCNSFRY

SEQ ID NO: 142 - protein, 27 aa, artificial
  Vasonatrin Peptide (1-27)
GLSKGCFGLKLDRIGSMSGLGCNSFRY

SEQ ID NO: 143 - protein, 7 aa, artificial
  [Ala5,  - Ala8]-Neurokinin A (4-10)
DAFVXLM

SEQ ID NO: 144 - protein, 10 aa, artificial
  Neuromedin L; NKA
HKTDSFVGLM

SEQ ID NO: 145 - protein, 13 aa, artificial
  Ac-(Leu28, 31)-Neuropeptide Y (24-26)
LRHYLNLLTRQRY

SEQ ID NO: 146 - protein, 14 aa, artificial
  Alytesin
XGRLGTQWAVGHLM

SEQ ID NO: 147 - protein, 8 aa, artificial
  Brain Neuropeptide II
FLFQPPRF

SEQ ID NO: 148 - protein, 13 aa, artificial
  [D-tyr11]-Neurotensin
XLYENKPRRPYIL

SEQ ID NO: 149 - protein, 28 aa, artificial
  IKKy NEMO Binding Domain (NBD) Inhibitory Peptide
DRQIKIWFQNRRMKWKKTALDWSWLQTE

SEQ ID NO: 150 - protein, 27 aa, artificial
  PTD-p50 (NLS) Inhibitory Peptide
DRQIKIWFQNRRMKWKKVQRKRQKLMP

SEQ ID NO: 151 - protein, 33 aa, artificial
  Orexin A, bovine, human, mouse, rat
XPLPDCCRQKTCSCRLYELLHGAGNHAAGILTL

SEQ ID NO: 152 - protein, 28 aa, artificial
  Orexin B, human
RSGPPGLQGRLQRLLQASGNHAAGILTM

SEQ ID NO: 153 - protein, 14 aa, artificial
  Aquaporin- 2(254-267), human
RQSVELHSPQSLPR

SEQ ID NO: 154 - protein, 36 aa, artificial
  Pancreatic Polypeptide, human
APLEPVYPGDNATPEQMAQYAADLRRYINMLTRPRY

SEQ ID NO: 155 - protein, 39 aa, artificial
  Neuropeptide
SLALADDAAFRERARLLAALERRHWLNSYMHKLLVLDAP

SEQ ID NO: 156 - protein, 34 aa, artificial
  Peptide YY (3-36), human
IKPEAPGEDASPEELNRYYASLRHYLNLVTRQRY

SEQ ID NO: 157 - protein, 7 aa, artificial
  Hydroxymethyl-Phytochelatin 2
XECXECS

SEQ ID NO: 158 - protein, 27 aa, artificial
  PACAP (1-27), amide, human, bovine,rat
HSDGIFTDSYSRYRKQMAVKKYLAAVL

SEQ ID NO: 159 - protein, 31 aa, artificial
  Prolactin Releasing Peptide (1-31), human
SRTHRHSMEIRTPDINPAWYASRGIRPVGRF

SEQ ID NO: 160 - protein, 28 aa, artificial
  Salusin-alpha
SGALPPAPAAPRPALRAQRAGPAGPGAK

SEQ ID NO: 161 - protein, 20 aa, artificial
  Salusin-beta
AIFIFIRWLLKLGHHGRAPP

SEQ ID NO: 162 - protein, 22 aa, artificial
  Saposin C22
CEFLVKEVTKLIDNNKTEKEIL

SEQ ID NO: 163 - protein, 27 aa, artificial
  Secretin, human
HSDGTFTSELSRLREGARLQRLLQGLV

SEQ ID NO: 164 - protein, 12 aa, artificial
  L-Selectin
CQKLDKSFSMIK

SEQ ID NO: 165 - protein, 10 aa, artificial
  Endokinin A/B
GKASQFFGLM

SEQ ID NO: 166 - protein, 14 aa, artificial
  Endokinin C (Human)
KKAYQLEHTFQGLL

SEQ ID NO: 167 - protein, 14 aa, artificial
  Endokinin D (Human)
VGAYQLEHTFQGLL

SEQ ID NO: 168 - protein, 7 aa, artificial
  Thrombin Receptor (42-48) Agonist, human
SFLLRNP

SEQ ID NO: 169 - protein, 4 aa, artificial
  LSKL, Inhibitor of Thrombospondin
LSKL

SEQ ID NO: 170 - protein, 3 aa, artificial
  Thyrotropin Releasing Hormone; TRH
XHP

SEQ ID NO: 171 - protein, 12 aa, artificial
  P55-TNFR Fragment
LPQIENVKGTED

SEQ ID NO: 172 - protein, 11 aa, artificial
  Urotensin II, human
ETPDCFWKYCV

SEQ ID NO: 173 - protein, 28 aa, artificial
  VIP, human, porcine, rat
HSDAVFTDNYTRLRKQMAVKKYLNSILN

SEQ ID NO: 174 - protein, 28 aa, artificial
  VIP Antagonist
KPRRPYTDNYTRLRKQMAVKKYLNSILN

SEQ ID NO: 175 - protein, 35 aa, artificial
  Helodermin
HSDAIFTQQYSKLLAKLALQKYLASILGSRTSPPP

SEQ ID NO: 176 - protein, 39 aa, artificial
  Exenatide
HGEGTFTSDLSKQMEEEAVRLFIEWLKNGGPSSGAPPPS

SEQ ID NO: 177 - protein, 37 aa, artificial
  ZP10, AVE00100
HAEGTFTSDVSSYLEGQAAKEFIAWLVKGRGKKKKKK

SEQ ID NO: 178 - protein, 37 aa, artificial
  Pramlinitide
KCNTATCATQRLANFLVHSSNNFGPILPPTNVGSNTY

SEQ ID NO: 179 - protein, 33 aa, artificial
  AC162352, PYY(3-36)
KPEAPGEDASPEELNRYYASLRHYLNLVTRQRY

SEQ ID NO: 180 - protein, 36 aa, artificial
  PYY
YPIKPEAPGEDASPEELNRYYASLRHYLNLVTRQRY

SEQ ID NO: 181 - protein, 36 aa, artificial
  Obinepitide
APLEPVYPGNKATPEQMAQYAADLRRYINMLTRQRY

SEQ ID NO: 182 - protein, 31 aa, artificial
  Glucagon
HSQGTFTSDYSKYLDSRRAQDFVQWLMNTKR

SEQ ID NO: 183 - protein, 27 aa, artificial
  GRP
VPLPAGGGTVLTKNMYPGNHWAVGHLM

SEQ ID NO: 184 - protein, 6 aa, artificial
  Ghrelin, GHRP6
HWAWFK

SEQ ID NO: 185 - protein, 9 aa, artificial
  Leuprolide
XHWSYLLRP

SEQ ID NO: 186 - protein, 9 aa, artificial
  Histrelin
XHWSYXLRP

SEQ ID NO: 187 - protein, 9 aa, artificial
  Oxytocin
CYIQNCPLG

SEQ ID NO: 188 - protein, 10 aa, artificial
  Atosiban, RWJ22164
XXILTNCPXG

SEQ ID NO: 189 - protein, 29 aa, artificial
  Sermorelin
YADAIFTNSYRKVLGQLSARKLLQDIMSR

SEQ ID NO: 190 - protein, 32 aa, artificial
  Nesiritide
SPKMVQGSGCFGRKMDRISSSSGLGCKVLRRH

SEQ ID NO: 191 - protein, 20 aa, artificial
  bivalirudin, Hirulog
FPRPGGGGNGDFEEIPEEYL

SEQ ID NO: 192 - protein, 9 aa, artificial
  Icatibant
RRPXXSXXR

SEQ ID NO: 193 - protein, 28 aa, artificial
  Aviptadil
HSDAVFTDNYTRLRKQMAVKKYLNSILN

SEQ ID NO: 194 - protein, 6 aa, artificial
  Rotigaptide, ZP123, GAP486
YPXGAG

SEQ ID NO: 195 - protein, 5 aa, artificial
  Cilengitide,EMD-121924, RGD Peptides
RGDFV

SEQ ID NO: 196 - protein, 32 aa, artificial; identical to SEQ ID NO: 190
  AlbuBNP

SEQ ID NO: 197 - protein, 32 aa, artificial; identical to SEQ ID NO: 190
  BN-054

SEQ ID NO: 198 - protein, 8 aa, artificial
  Angiotensin II
DRVYIHPF

SEQ ID NO: 199 - protein, 17 aa, artificial
  MBP-8298
DENPVVHFFKNIVTPRT

SEQ ID NO: 200 - protein, 18 aa, artificial
  Peptide Leucine Arginine
LVRGCWTKSYPPKPCFVR

SEQ ID NO: 201 - protein, 24 aa, artificial
  Ziconotide
CKGKGAKCSRLYDCCTGSCRSGKC

SEQ ID NO: 202 - protein, 8 aa, artificial
  AL-208
NAPVSIPQ

SEQ ID NO: 203 - protein, 8 aa, artificial
  AL-108
NAPVSIPQ

SEQ ID NO: 204 - protein, 9 aa, artificial
  Carbeticon
CYIQNCPLG

SEQ ID NO: 205 - protein, 3 aa, artificial
  Tripeptide
GPA

SEQ ID NO: 206 - protein, 9 aa, artificial
  SAL
SALLRSIPA

SEQ ID NO: 207 - protein, 26 aa, artificial
  Coliven
SALLRSIPAPAGASRLLLLTGEIDLP

SEQ ID NO: 208 - protein, 24 aa, artificial
  Humanin
MAPRGFSCLLLLTSEIDLPVKRRA

SEQ ID NO: 209 - protein, 14 aa, artificial
  ADNF-14
VLGGGSALLRSIPA

SEQ ID NO: 210 - protein, 28 aa, artificial
  VIP (Vasoactive Intestinal Peptide)
HSDAVFTDNYTRLRLQMAVKKYLNSILN

SEQ ID NO: 211 - protein, 28 aa, artificial
  Thymalfasin
SDAAVDTSSEITTKDLKEKKEVVEEAEN

SEQ ID NO: 212 - protein, 7 aa, artificial
  Bacitracin, USP
XIFHDNK

SEQ ID NO: 213 - protein, 15 aa, artificial
  Gramidicin, USP
VGALAVVVWLWLWLW

SEQ ID NO: 214 - protein, 22 aa, artificial
  Pexiganan, MSI-78
GIGKFLKKAKKFGKAFVKILKK

SEQ ID NO: 215 - protein, 12 aa, artificial
  P113
AKRHHGYKRKFH

SEQ ID NO: 216 - protein, 12 aa, artificial
  PAC-113
AKRHHGYKRKFH

SEQ ID NO: 217 - protein, 2 aa, artificial
  SCV-07
XW

SEQ ID NO: 218 - protein, 11 aa, artificial
  HLF1-11, Lactoferrin
GRRRRSVQWCA

SEQ ID NO: 219 - protein, 8 aa, artificial
  DAPTA
ASTTTNYT

SEQ ID NO: 220 - protein, 37 aa, artificial
  TRI-1144
TTWEAWDRAIAEYAAREIALLRALQEQQENEAALREL

SEQ ID NO: 221 - protein, 13 aa, artificial
  Tritrpticin
VRRFPWWWPFLRR

SEQ ID NO: 222 - protein, 9 aa, artificial
  Antiflammin 2
HDMNKVLDL

SEQ ID NO: 223 - protein, 33 aa, artificial
  Gattex, Teduglutide, ALX-0600
HGDGSFSDEMNTILDNLAARDFINWLIQTKITD

SEQ ID NO: 224 - protein, 25 aa, artificial
  Stimuvax, L-BLP25
STAPPAHGVTSAPDTRPAPGSTAPP

SEQ ID NO: 225 - protein, 23 aa, artificial
  Chrysalin, TP508
AGYKPDEGKRGDACEGDSGGPFV

SEQ ID NO: 226 - protein, 7 aa, artificial
  Melanonan II
XDHFRWK

SEQ ID NO: 227 - protein, 8 aa, artificial
  Spantide II
RPKPQQWF

SEQ ID NO: 228 - protein, 7 aa, artificial
  Ceruletide
YTGWXDF

SEQ ID NO: 229 - protein, 10 aa, artificial
  Sincalide
DYMGWMDDFA

SEQ ID NO: 230 - protein, 5 aa, artificial
  Pentagastin
XWMDF

SEQ ID NO: 231 - protein, 27 aa, artificial
  Secretin
HSDGTFTSELSRLRDSARLQRLLQGLV

SEQ ID NO: 232 - protein, 12 aa, artificial
  Endostatin peptide
PSPHRPNRPTEP

SEQ ID NO: 233 - protein, 7 aa, artificial
  E-selectin
IELLQAR

SEQ ID NO: 234 - protein, 12 aa, artificial
  HER-2
YCDGFYACYMDV

SEQ ID NO: 235 - protein, 13 aa, artificial
  IL-6
GGCKLWTIPQCGG

SEQ ID NO: 236 - protein, 6 aa, artificial
  IL-8
RRWWCR

SEQ ID NO: 237 - protein, 9 aa, artificial
  IL-10
AYMTMKIRN

SEQ ID NO: 238 - protein, 19 aa, artificial
  PDGF
YGRPRESGKKRKRKRLKPT

SEQ ID NO: 239 - protein, 9 aa, artificial
  Thrombospondin
XGVXTXIRP

SEQ ID NO: 240 - protein, 10 aa, artificial
  uPA (1)
LNFSGYLWTY

SEQ ID NO: 241 - protein, 6 aa, artificial
  uPA (2)
RRKRRR

SEQ ID NO: 242 - protein, 6 aa, artificial
  VEGF
RRKRRR

SEQ ID NO: 243 - protein, 7 aa, artificial
  VEGF (2)
ATWLPPR

SEQ ID NO: 244 - protein, 5 aa, artificial
  Pentapeptide-3
KTTKS

SEQ ID NO: 245 - protein, 3 aa, artificial
  Glutathione
ECG

SEQ ID NO: 246 - protein, 5 aa, artificial
  XXLRR
XXLRR

SEQ ID NO: 247 - protein, 5 aa, artificial
  Beta - Amyloid Fibrillogenesis
KLVFF

SEQ ID NO: 248 - protein, 4 aa, artificial
  Endomorphin - 2
YPFF

SEQ ID NO: 249 - protein, 39 aa, artificial; identical to SEQ ID NO: 155
  TIP 39, Tuberoinfundibular Neuropeptide

SEQ ID NO: 250 - protein, 38 aa, artificial
  PACAP (1-38), amide, human, bovine, rat
HSDGIFTDSYSRYRKQMAVKKYLAAVLGKRYKQRVKNK

SEQ ID NO: 251 - protein, 4 aa, artificial
  TGF activating peptide
KRFK

SEQ ID NO: 252 - protein, 4 aa, artificial
  Insulin sensitizing factor; ISF402
XHTD

SEQ ID NO: 253 - protein, 8 aa, artificial
  Transforming Growth Factor 1 Peptide, TGF-1(60-66), amide
KVLALYNK

SEQ ID NO: 254 - protein, 10 aa, artificial
  Caerulein Releasing Factor
XQDYTGWMDF

SEQ ID NO: 255 - protein, 7 aa, artificial
  IELLQAR (8-branch MAPS)
IELLQAR

SEQ ID NO: 256 - protein, 14 aa, artificial
  Tigapotide PK3145
EWQTDNCECTCYQT

SEQ ID NO: 257 - protein, 10 aa, artificial
  Goserelin
XHWSYSLRPX

SEQ ID NO: 258 - protein, 10 aa, artificial
  Abarelix
XXXSYNLKPA

SEQ ID NO: 259 - protein, 10 aa, artificial
  Cetrorelix
XXXSYXLRPA

SEQ ID NO: 260 - protein, 9 aa, artificial
  Ganirelix
XXXSYLXPA

SEQ ID NO: 261 - protein, 10 aa, artificial
  Degarelix, Triptorelin
XXXSXXLXPA

SEQ ID NO: 262 - protein, 6 aa, artificial
  Barusiban, FE 200440
WIXNXX

SEQ ID NO: 263 - protein, 6 aa, artificial
  Pralmorelin
AXAWFL

SEQ ID NO: 264 - protein, 8 aa, artificial
  Octreotide
FCFWKTCT

SEQ ID NO: 265 - protein, 6 aa, artificial
  Eptifibatide
XGDWPC

SEQ ID NO: 266 - protein, 5 aa, artificial
  Netamiftide, INN-00835
XXRGW

SEQ ID NO: 267 - protein, 13 aa, artificial
  Daptamycin
WNDTGXDADGSXX

SEQ ID NO: 268 - protein, 11 aa, artificial
  Spantide II (1)
KPXPXNWFWLX

SEQ ID NO: 269 - protein, 10 aa, artificial
  Delmitide, RDP-58
RXXXRXXXGY

SEQ ID NO: 270 - protein, 8 aa, artificial
  AL-209
NAPVSIPQ

SEQ ID NO: 271 - protein, 36 aa, artificial
  Enfuvirtide
YTSLIHSLIEESQNQQEKNEQELLELDKWASLWNWF

SEQ ID NO: 272 - protein, 13 aa, artificial
  IDR-1
LLCRIVPVIPWCK

SEQ ID NO: 273 - protein, 6 aa, artificial
  Hexapeptide-6
FALLKL

SEQ ID NO: 274 - protein, 21 aa, artificial
  Insulin- A chain
GIVEQCCTSICSLYQLENYCN

SEQ ID NO: 275 - protein, 8 aa, artificial
  Lanreotide
XCYWKVCT

SEQ ID NO: 276 - protein, 6 aa, artificial
  Hexapeptide-3
EEMQRR

SEQ ID NO: 277 - protein, 28 aa, artificial
  Insulin B-chain
FVNQHLCGSHLVEZLYLVCGRGFFYPLT

SEQ ID NO: 278 - protein, 21 aa, artificial
  Glargine- A chain
GIVEQCCTSICSLYQLENYCG

SEQ ID NO: 279 - protein, 31 aa, artificial
  Glargine- B chain
FVNQHLCGSHLVEALYLVCGERGFFYPKTRR

SEQ ID NO: 280 - protein, 30 aa, artificial
  Insulin- LisPro B- chain analog
FVNQHLCGSHLVEALYLVCGERGFFYTKPT

SEQ ID NO: 281 - protein, 30 aa, artificial
  Insulin- Aspart B- chain analog
FVNQHLCGSHLVEALYLVCGERGFFYTDPT

SEQ ID NO: 282 - protein, 30 aa, artificial
  Insulin- Glulisine B chain analog
FVKQHLCGSHLVEALYLVCGERGFFYTPET

SEQ ID NO: 283 - protein, 29 aa, artificial
  Insulin- Determir B chain analog
FVNQHLCGSHLVEALYLVCGERGFFYTPK

SEQ ID NO: 284 - protein, 14 aa, artificial
  Somatostatin
AGCKNFFWKTFTSC

SEQ ID NO: 285 - protein, 8 aa, artificial
  Somatostatin Tumor Inhibiting Analog
XCYWKVCT

SEQ ID NO: 286 - protein, 16 aa, artificial
  Pancreastatin 37-52)
EEEEEMAVVPQGLFRG

SEQ ID NO: 287 - protein, 4 aa, artificial
  Vasoactive Intestinal Peptide fragment (KKYL-NH2)
KKYL

SEQ ID NO: 288 - protein, 17 aa, artificial
  Dynorphin A
YGGFLRRIRPKLKWDNQ

(19) United States
(12) Patent Application Publication
(10) Pub. No.: US 2010/0168443 A1
(43) Pub. Date: Jul. 1, 2010

(54) Ethoid-Containing Compounds, Methods for Preparing Ethoid-Containing Compounds, and Methods of Use

(75) Inventor: Hendrik Mario Geysen, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/513,210

(22) PCT Filed: Nov. 2, 2007
(86) PCT No.: PCT/US2007/083502

§ 371 Date: May 1, 2009

Related U.S. Application Data
(60) Provisional application No. 60/856,108, filed on Nov. 2, 2006.

(51) Int. Cl. C07D 207/09

(52) U.S. Cl. 548/568

(57) ABSTRACT

Ethoid-containing compounds comprising one or more ethoid moieties (e.g., a methyleneoxy, Ψ[CH₂O]) as a substitutive, isosteric replacement for an amide moiety of a polyaminoacid are disclosed. Universal, modular approaches for preparing such ethoid-containing compounds are also disclosed. Such ethoid-containing compounds can be polyaminoacid analogs, and are useful as food additives, as cosmetics ingredients, as research reagents, as diagnostic agents, and as therapeutic agents.

### BACKGROUND

Polyaminoacids such as polypeptides and proteins are an important group of compounds and are widely used in numerous applications, including for example as food additives, as cosmetics ingredients, as research reagents, as diagnostic agents, and as therapeutic agents such as drugs. Polypeptides and proteins can be formed from sequential condensation of an amine of one alpha-amino acid, and a carboxylic acid of another alpha-amino acid, with the resulting macromolecule comprising amino acid residues linked by amide bonds. Proteins can also be formed using cell-based expression systems. Polyaminoacids such as proteins and polypeptides possess a number of useful activities and properties, including generally, being selective, being immunilogically acceptable, and in many cases being therapeutically validated and commercially recognized. Nonetheless, polyaminoacid compounds are notoriously impractical to use. It is well-known that the amide bonds in proteins and polypeptides are susceptible to enzymatic digestion, including especially by protease or peptidase enzymes. Such enzymatic instability contributes to the poor bioavailability of these compounds. Consequently, the use of proteins or polypeptides as therapeutics typically requires administration via injection or in some cases as an aerosol (e.g., via deep inhalation or nasal administration). As another consequence of enzymatic instability, therapeutic agents and diagnostic agents based on polypeptides or proteins generally have very short half-lives or active windows after administration. Another general disadvantage of using proteins or polypeptides in foods, cosmetics or as therapeutic agents is that the amide bonds can be susceptible to chemical instability, especially in pH or temperature-dependent applications. The large-scale manufacturing of biologics such as proteins or polypeptides provides further challenges.

Chemical analogs of polyaminoacids such as proteins and polypeptides are known in the art. In particular, a number of approaches are described in the literature for isosteric substitutions of the amide moiety of a polyaminoacid. Within this general area of research, only a relatively small effort has been directed toward identifying suitable polyaminoacid analogs based on methyleneoxy and related isosteres. See for example, Ondetti, et. al., I. Chem. Bio. Pept., Proc. Am. Pept. Symp., 3^{rd} See for example, Ondetti, et. al., I. Chem. Bio. Pept., Proc. Am. Pept. Symp., 3^{rd} 1972, 525-31; Blomberg, et. al. Organic & Biomolecular Chemistry 2006, 4, (3), 416-423; Holm et. al., Bioorganic & Medicinal Chemistry 2006, 14, (17), 5921-5932; Hlavacek, J. et. al., Amino Acids 2004, 27, (1), 19-27; Blomberg, D. et. al., Journal of Organic Chemistry 2004, 69, (10), 3500-508; Ten Brink, R. E., et. al., Journal of Medicinal Chemistry 1988, 31, (3), 671-7; Allerton, C. M. N, et. al. “Preparation of 3-(imidazolyl)-2-alkoxypropanoic acids as selective TAFIa inhibitors for treating thrombotic and other conditions associated with fibrin deposition.” 2003-IB602003061652, 20030110, 2003; and Fitzgerald, et. al., J. Chem. Inf. Model. 2006, 46, 1588-1597. However, such approaches generally suffer from one or more deficiencies. For example, many such approaches suffer from not being able to create polyaminoacid analogs having necessary molecular diversity (e.g, with respect to side chain structure). Further, many such approaches do not maintain the extent of chirality (e.g., as may be necessary for specificity). Many such approaches suffer from isosteric structures which fundamentally alter the spatial geometry of functionally-related side chain groups. Many such isosteric approaches change the electronic characteristics of the amide moities of polyaminoacids, thus potentially impacting the electronic interaction of such a macromolecule with other compounds or with other regions of the same macromolecule.

Thus there remains a need in the art for compounds which can be analogs of polyaminoacids, but which overcome one or more deficiencies, including one or more of the foregoing deficiencies.

### SUMMARY

The present inventions, as described or claimed herein, overcome many of the deficiencies associated with known polyaminoacid analogs having isosteric substitutions for the amide moities and/or for the synthesis protocols for preparing such compounds. Among the advantages realized by the compounds and methods of the present invention are included: polyaminoacid analogs having improved resistance to digesting enzymes, such as proteases or peptidases; methods and compounds which maintain chirality of biologically important carbon centers; methods having universality and modularity for preparing compounds having substantial molecular diversity (e.g, with respect to side chain structure); compounds having spatial geometry (e.g., of functionally-related side chain groups) which conservatively approaches the spatial geometry of polyaminoacids; and compounds which are biologically active and/or have other useful properties of interest. Such advantages, considered alone and in various combinations, evidence a significant advance in the art.

Briefly, therefore, the present invention is directed in various aspects and embodiments to certain compounds, methods for preparing compounds, and methods for using such compounds. The invention is also directed in various aspects and embodiments to sets of compounds, methods for preparing sets of compounds, and methods of using sets of compounds. The invention is also directed in various aspects and embodiments to data sets derived from the compounds or sets of compounds, from the methods for preparing the compound or sets of compounds, or from the methods of using the compounds or sets of the compounds.

### Ethoid-Containing Compounds

The compounds of the invention generally comprise one or more ethoid moieties, preferably —CHR¹⁰O—, each R¹⁰ being independently selected hydrogen, hydrocarbyl or substituted hydrocarbyl, more preferably each R¹⁰ being independently selected from the group consisting of H, C₁-C₈ alkyl and substituted C₁-C₈ alkyl, even more preferably each R¹⁰ being independently selected from the group consisting of H, C₁-C₃ alkyl and substituted C₁-C₃ alkyl, where in each case, R¹⁰ optionally forming one or more ring structures with adjacent atoms or moieties (e.g., in some embodiments with adjacent pendant moieties). In many aspects and embodiments, compounds of the invention comprise one or more ethoid moieties which are an unsubstituted methyleneoxy moiety, —CH₂O—.

The compounds of the invention generally comprise one or more ethoid moieties as a substitutive, isosteric replacement for an amide moiety of a polyaminoacid, such as a polypeptide or a protein (e.g., comprising α-amino acid residues linked by amide moieties, such as are derived from coupling of α-amino acids). In addition to ethoid isosteres, generally represented as ψ[ethoid], and including preferred ethoid isosteres such as ψ[CHR¹⁰O] and ψ[CH₂O], the compounds of the invention can also comprise other isosteres, generally represented as ψ[ ].

Hence, among the compounds of the invention are compounds which comprise an ethoid moiety or a polyethoid moiety, preferably as isosteres. Such compounds can generally comprise a structural moiety of a polyaminoacid having one or more ethoid isosteres at a corresponding one or more sequence positions, each as a substitutive replacement for an amide moiety. In some embodiments, the ethoid-containing compounds of the invention can comprise a polyethoid moiety (e.g., a moiety including two or more ethoid moieties, or in some embodiments three or more ethoid moieties), preferably as isosteres. In some embodiments, the ethoid-containing compounds of the invention can comprise a polyethiodpeptide moiety (e.g., a moiety including two or more ethoid moieties, or in some embodiments three or more ethoid moieties, and in each case additionally comprising one or more amide moieties). For example, polyethoidpeptides of the invention can comprise a structural moiety of a polyaminoacid having one or more ethoid isosteres at a corresponding one or more sequence positions, each as a substitutive replacement for an amide moiety, and additionally one or more amide moieties, each of such ethoid moieties and amide moieties linking amino acid residues within the compound. In some embodiments, the compounds of the invention can comprise a fully-ethoid-substituted moiety (e.g., a moiety including two or more ethoid moieties, or in some embodiments three or more ethoid moieties, and in each case to the exclusion of amide moieties—such amide moieties having been substitutively replaced by the ethoid isosteres, alone or in combination with other isosteres, ψ[ ]. For example, fully-ethoid-substituted polyethiods of the invention can comprise a moiety comprising the structural moiety of a polyaminoacid with only ethoid isosteres as substitutive replacements for each of the amide moieties within the structural moiety of the polyaminoacid. As an alternative example, a fully-ethoid-substituted polyethiods of the invention can comprise a moiety comprising the structural moiety of a polyaminoacid with primarily ethoid isosteres as substitutive replacements for each of the amide moieties within the structural moiety of the polyaminoacid, but allowing for a fewer number of other isosteres, ψ[ ], considered cumulatively relative to the number ethoid moieties.

Although various concepts and features of the invention have been introduced and described in the preceding paragraphs in the context of compounds of the invention, such concepts and features are equally applicable to other aspects and embodiments of the invention, and are expressly contemplated in connection therewith.

In a first aspect, therefore, the invention is directed to a compound comprising an ethoid moiety or a polyethoid moiety.

In a first general embodiment of the first aspect, the invention is directed to a compound comprising a polyethoid moiety having a formula

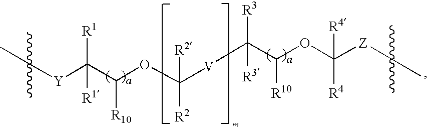

wherein, generally, m is an integer ≧0, and each a is an independently selected integer=1 or =2. Generally, R¹, each R² and R⁴ are each an independently selected side chain moiety comprising hydrocarbyl or substituted hydrocarbyl. Generally, R¹, each R² and R⁴ can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, each R²′, and R⁴′, respectively) or with adjacent side chain moieties (e.g., R¹ with a nearest R²) or with an atom on the backbone of the polyethioid moiety (e.g, R² with an adjacent N atom in an embodiment where a V is an N-substituted methyleneamine isostere. Preferably, R¹, each R² and R⁴ can each be an independently selected side chain moiety having a structure of an amino acid side chain (including natural amino acid side chains, and non-natural amino acid side chains). Generally, R³ is side chain moiety having a structure of an amino acid side chain (including natural amino acid side chains, and non-natural amino acid side chains), with the proviso that R³ does not include a —H or —CH₃ or other side chain moieties of polyethylene glycol (PEG) or polypropyleneglycol (PPG) or known derivatives of PEG or PPG. In preferred embodiments within this first general embodiment, R³ is selected from the group consisting of (a) a side chain moiety selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W} and R_{Y}, each as delineated in Table I.A, (b) a side chain moiety selected from and having a structure of a non-natural amino acid side chain as delineated in Table I.B.1 or in Table I.C.1 and (c) a protected derivative of the foregoing side chain moieties [excludes PEG/PPG side-chains].

Additionally, the following descriptors are generally applicable in this first general embodiment of the first aspect, and in preferred embodiments thereof (in each case unless otherwise noted). R¹′, each R²′, R³′ and R⁴′ are each independently selected from hydrocarbyl or substituted hydrocarbyl, and are preferably each independently selected from the group consisting of H, C₁-C₃ alkyl and substituted C₁-C₃ alkyl. Each R¹⁰ is generally being independently selected from hydrogen, hydrocarbyl or substituted hydrocarbyl; more preferably each R¹⁰ is independently selected from the group consisting of H, C₁-C₈ alkyl and substituted C₁-C₈ alkyl, even more preferably each R¹⁰ being independently selected from the group consisting of H, C₁-C₃ alkyl and substituted C₁-C₃ alkyl. In each case, R¹³ can optionally form one or more ring structures with adjacent side chain moieties or with an atom on the backbone of the polyethioid moiety. The polyethoid of this first embodiment of this aspect of the invention can optionally include one or more amide moieties and additionally or alternatively or one or more other isosteres in addition to ethoid isosteres. Hence, each V is independently selected from the group consisting of —C(O)NH— and -ψ[ ]-. Y and Z are each generally independently selected from the group consisting of H, hydrocarbyl and substituted hydrocarbyl. Y and Z can be, independently selected, linking moieties (e.g., connecting the depicted compound to another compound or to another moiety of the same compound) or terminal groups (e.g., a moiety representing the end terminals of the depicted compound, either as a final compound or as an intermediate compound (e.g., as a functional group or a protected functional group). In preferred embodiments, Y and Z can each be independently selected from the group consisting —V—, -functional group, -protected functional group, -linking moiety, -conjugate and -terminal group. Examples of Y and Z as linking moieties include linking moieties which connect the depicted polyethoid moiety to another polyethoid moiety, to a polypeptide moiety, to a polyethoidpeptide moiety, to a support (e.g., a solid support). In some embodiments, one or both of Y and Z can be terminal groups. For example, Y can be a terminal group selected from the group consisting of H—, H₂N—, AcNH—, R²⁰C(O)NH—, R²²OC(O)NH—, HO—, R²⁰O—, and protected derivatives thereof, R²⁰ and R²² each being independently selected from the group consisting of H, hydrocarbyl and substituted hydrocarbyl. For example, Z can be a terminal group selected from the group consisting of —H, —R²⁰OH, —C(O)O R²⁰, —C(O)H, —C(O)R²⁰, —R²⁰OR²², —C(O)NHR²⁰ and protected derivatives thereof, R²⁰ and R²² each being independently selected from the group consisting of H, hydrocarbyl and substituted hydrocarbyl.

In a first preferred embodiment of the first general embodiment of the first aspect, the invention is directed to a compound comprising a polyethoid moiety having a formula

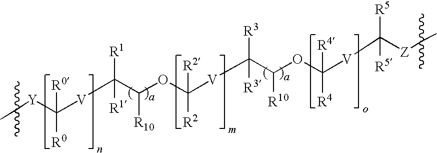

wherein the sum of n, m and o is ≧3. In this preferred embodiment, each R⁰, R¹, each R², each R⁴ and R⁵ are each an independently selected side chain moiety comprising hydrocarbyl or substituted hydrocarbyl. Generally, each R⁰, R¹, each R², each R⁴ and R⁵ can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with each R⁰′, R¹′, each R²′, each R⁴′, and R⁵′, respectively) or with adjacent side chain moieties (e.g., R¹ with a nearest R²) or with an atom on the backbone of the polyethioid moiety (e.g, R² with an adjacent N atom in an embodiment where a V is an N-substituted methyleneamine isostere. Preferably, each R⁰, R¹, each R², each R⁴ and R⁵ can each be an independently selected side chain moiety having a structure of an amino acid side chain; and each R⁰′, R¹′, each R²′, R³′, each R⁴′, and R⁵′ are each independently selected from the group consisting of H, C₁-C₃ alkyl and substituted C₁-C₃ alkyl. More preferably, in this preferred embodiment: each a=1; each R⁰′, R¹′, each R²′, R³′, each R⁴′, and R⁵′ are each independently selected from the group consisting of H and methyl; each R¹⁰ is independently selected from the group consisting of H, methyl and substituted methyl; each V is independently selected from the group consisting of —C(O)NH— and -ψ[ ]-; and Y and Z are each independently selected from the group consisting of —V—, -functional group, -protected functional group, -linking moiety, -conjugate and -terminal group.

In another, second preferred embodiment of the first general embodiment of the first aspect, the invention is directed to a fully-ethoid-substituted polyethoid moiety, where for example, with reference to the formula of the polyethoid moiety as depicted in connection with the first preferred embodiment of the first general embodiment of the first aspect: each of n, m and o is an integer ranging from 1 to 5, and each —V— is an ethoid moiety, preferably an ethoid moiety having a formula

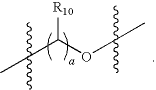

In this second preferred embodiment of the first general embodiment of the first aspect, it is preferable that when any of R⁰, R¹, each R² (other than R² nearest R¹), R³, each R⁴ (other than R⁴ nearest R³) or R⁵ are proline, R_{P} as delineated in Table I.A, or are a proline analog (e.g., as selected from and having a structure of a side chain moiety delineated in Table I.C.1), then —V— is a methyleneamine moiety, preferably a methyleneamine moiety having a formula

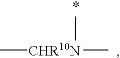

each * representing a bond linking the nitrogen atom to an adjacent side chain moiety. Alternatively, —V— in such instances can be a proline analog generally being a C₃ to C₁₂ hydrocarbyl or substituted hydrocarbyl comprising a ring structure such as a five-member ring.

In this second preferred embodiment of the first general embodiment of the first aspect, it is also preferable that Y and Z are each an independently selected terminal group.

In another, third preferred embodiment of the first general embodiment of the first aspect, the invention is directed to a polyethoidpeptide moiety, where for example, with reference to the formula of the polyethoid moiety as depicted in connection with the first preferred embodiment of the first general embodiment of the first aspect: each of n, m and o is an integer ranging from 1 to 5; and each —V— is an ethoid moiety having a formula

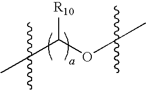

or an amide moiety having a formula

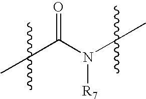

where at least one —V— is the amide moiety, and each R⁷ is independently selected from the group consisting of —H and a side chain moiety having a structure of an amino acid side chain. In this third preferred embodiment of the first general embodiment of the first aspect, it is preferable that when any of each R⁰, R¹, each R² (other than R² nearest R¹), R³, each R⁴ (other than R⁴ nearest R³) or R⁵ are proline, R_{P} as delineated in Table I.A, or are a proline analog (e.g., such as selected from and having a structure of a side chain moiety delineated in Table I.C.1), then —V— is the amide moiety or a methyleneamine moiety having a formula

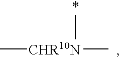

each * representing a bond linking the nitrogen atom to an adjacent side chain moiety. Alternatively, —V— in such instances can be a proline analog generally being a C₃ to C₁₂ hydrocarbyl or substituted hydrocarbyl comprising a ring structure such as a five-member ring. In this third preferred embodiment of the first general embodiment of the first aspect, it is also preferable that Y and Z are each an independently selected terminal group.

In a second general embodiment of the first aspect, the invention is directed to a compound comprising an ethoid moiety having a formula

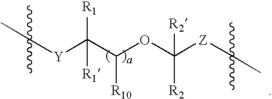

In this embodiment, the integer a, each R¹⁰, R^{1′}, R^{2′}, Y and Z are each as described above in connection with the first general embodiment of the first aspect of the invention (and are to be considered the same as if such were expressly reproduced in this paragraph); moreover, these are generally applicable in this second general embodiment of the first aspect, and in preferred embodiments thereof (in each case unless otherwise noted).

In this second general embodiment of the first aspect, R¹ and R² are generally each an independently selected side chain moiety having a structure of an amino acid side chain (including natural amino acid side chains, and non-natural amino acid side chains), with the proviso that specific known combinations of R¹ and R² are excluded therefrom in specific combination.

In some preferred embodiments of this second general embodiment of the first aspect, each of R¹ and R² are selected in various specific combinations. Generally, R¹ is selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W}, R_{Y}, and protected derivatives thereof. (Each of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y} are delineated in Table I.A).

In a first such preferred embodiment, when R¹ is R_{A}, then R² is selected from the group consisting of R_{C}, R_{D}, R_{E}, R_{F}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof.

In a second such preferred embodiment, when R¹ is R_{C}, R_{E}, R_{H}, R_{K}, R_{M}, R_{N}, R_{Q}, R_{T}, R_{U} or R_{W}, then R² is selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof.

In a third such preferred embodiment, when R¹ is R_{D} or R_{S}, then R² is selected from the group consisting of R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof.

In a fourth such preferred embodiment, when R¹ is R_{F}, then R² is selected from the group consisting of R_{C}, R_{D}, R_{E}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof.

In a fifth such preferred embodiment, when R¹ is R_{G}, then R² is selected from the group consisting of R_{C}, R_{E}, R_{I}, R_{K}, R_{L}, R_{M}, R_{N}, R_{Q}, R_{R}, R_{T}, R_{U}, R_{W}, R_{Y}, and protected derivatives thereof.

In a sixth such preferred embodiment, when R¹ is R_{I}, then R² is selected from the group consisting of R_{C}, R_{D}, R_{E}, R_{H}, R_{I}, R_{K}, R_{L}, R_{M}, R_{N}, R_{Q}, R_{R}, R_{S}, R_{T}, R_{U}, R_{W}, R_{Y}, and protected derivatives thereof.

In a seventh such preferred embodiment, when R¹ is R_{L}, then R² is selected from the group consisting of R_{C}, R_{D}, R_{E}, R_{H}, R_{I}, R_{K}, R_{M}, R_{N}, R_{Q}, R_{R}, R_{S}, R_{T}, R_{U}, R_{W}, R_{Y}, and protected derivatives thereof.

In a eighth such preferred embodiment, when R¹ is R_{P}, then R² is selected from the group consisting of R_{C}, R_{D}, R_{E}, R_{H}, R_{I}, R_{K}, R_{M}, R_{N}, R_{Q}, R_{R}, R_{S}, R_{U}, R_{V}, R_{W}, R_{Y}, and protected derivatives thereof.

In a ninth such preferred embodiment, when R¹ is R_{R}, then R² is selected from the group consisting of R_{A}, R_{C}, R_{D}, R_{E}, R_{F}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof.

In a tenth such preferred embodiment, when R₁ is R_{V}, then R₂ is selected from the group consisting of R_{C}, R_{D}, R_{E}, R_{F}, R_{H}, R_{I}, R_{K}, R_{M}, R_{N}, R_{Q}, R_{R}, R_{S}, R_{T}, R_{U}, R_{V}, R_{W}, R_{Y}, and protected derivatives thereof.

In a eleventh such preferred embodiment, when R¹ is R_{Y}, then R² is selected from the group consisting of R_{A}, R_{C}, R_{E}, R_{H}, R_{K}, R_{L}, R_{M}, R_{N}, R_{Q}, R_{R}, R_{S}, R_{T}, R_{U}, R_{V}, R_{W}, R_{Y} and protected derivatives thereof.

In another, independent, twelfth preferred embodiment of this second general embodiment of the first aspect, the invention is directed to an ethoid moiety having a formula

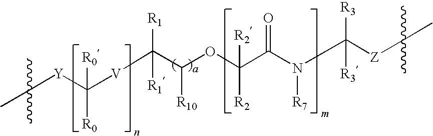

In this twelfth preferred embodiment, m and n are each an independently selected integer ≧0, and the sum of m and n (i.e., m+n) is ≧1. In each case, R₁ is independently selected, as described above in connection with the second general embodiment of this first aspect. When m≧1, then: the R₂ nearest R₁ (i.e., the R₂ adjacent the ethoid moiety opposite R₁) is independently selected in combination with R₁ as described above in connection with the second general embodiment of this first aspect; and each R₀, each R₂ other than the R₂ nearest R₁, and R₃ are each an independently selected side chain moiety having a structure of an amino acid side chain. When m=0: then each R₀ is an independently selected side chain moiety having a structure of an amino acid side chain; and R³=R² (i.e., R³ is the same as R² as described above in connection with the second general embodiment of this first aspect) and is independently selected in combination with R₁ as described above in connection with the second general embodiment of this first aspect. In this twelfth preferred embodiment, each R₀′, and R₃′ are each the same as R¹′ and R²′ as described above in connection with this second general embodiment of the first aspect of the invention. Each R⁷ is independently selected from the group consisting of —H and a side chain moiety having a structure of an amino acid side chain from the group consisting of H. Each V is independently selected from the group consisting of —C(O)NH— and -ψ[ ]-.

In a further, thirteenth preferred embodiment of this second general embodiment of the first aspect, the invention is directed to an ethoid moiety having a formula

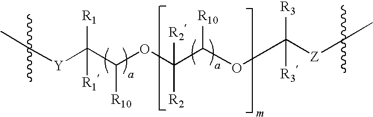

In this thirteenth preferred embodiment: m is an integer ≧1; the R² nearest R₁ (i.e., the R² adjacent the ethoid moiety opposite R₁) is independently selected in combination with R₁ as described above in connection with the second general embodiment of this first aspect; and each R² other than the R² nearest R₁ is an independently selected side chain moiety having a structure of an amino acid side chain. R₃ is an independently selected side chain moiety having a structure of an amino acid side chain. R³′ is the same as R¹′ and R²′ as described above in connection with this second general embodiment of the first aspect of the invention.

In another fourteenth preferred embodiment of this second general embodiment of the first aspect, the invention is directed to a ethoid moiety having a formula

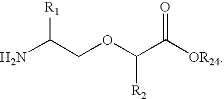

In this preferred embodiment, R¹ is independently selected, and R² is independently selected in combination with R₁, in each case as described above in connection with the second general embodiment of this first aspect. R²⁴ is selected from the group consisting of H, alkyl and substituted alkyl.

In a third general embodiment of the first aspect, the invention is directed to a compound comprising a polyethoid moiety having a formula

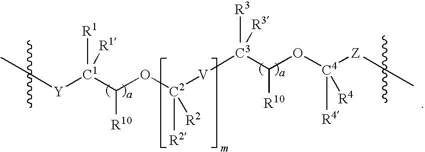

In this third general embodiment, the integer a, each R¹⁰, R^{1′}, each R^{2′}, R^{3′} and R^{4′}, each V, Y and Z are each as described above in connection with the first general embodiment of the first aspect of the invention (and are to be considered the same as if such were expressly reproduced in this paragraph); moreover, these are generally applicable in this third general embodiment of the first aspect, and in preferred embodiments thereof (in each case unless otherwise noted). In this general embodiment, m is an integer ≧0. The pendant side chains, R¹, each R², R³ and R⁴, are each independently selected from the group consisting of hydrocarbyl and substituted hydrocarbyl; preferably each are independently selected from the group consisting of alkyl, substituted alkyl, heteroalkyl, substituted heteroalkyl, aryl, substituted aryl, heteroaryl, substituted heteroaryl, alicyclic, substituted alicyclic, heterocyclic, substituted heterocyclic, including in each case one or more ring structures formed between adjacent pendant moieties selected from R¹, each R², R³ and R⁴, or one or more ring structures formed with a respective opposing pendant moiety, R^{1′}, each R^{2′}, R^{3′} and R^{4′}, or with an atom on the backbone of the polyethioid moiety (e.g, R² with an adjacent N atom in an embodiment where a V is an N-substituted methyleneamine isostere. The pendant side chain moieties R¹, each R², R³ and R⁴ can in some preferred embodiments be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, each R²′, R³′, and R⁴′, respectively) or with adjacent side chain moieties (e.g., R¹ with a nearest R²) or with an atom on the backbone of the polyethioid moiety (e.g, R² with an adjacent N atom in an embodiment where a V is an N-substituted methyleneamine isostere). Preferably, R¹, each R², R³ and R⁴ can each be an independently selected most preferably each are an independently selected side chain moiety having a structure of an amino acid side chain.

In this third general embodiment, the ethoid moiety of the invention comprises two or more chiral carbon centers. More specifically, when m=0, then at least two carbons selected from C¹, C³ and C⁴ are chiral and having an enantiomeric excess of at least (about) 20%. When m≧1, at least three carbons selected from C¹, each C², C³ and C⁴ are chiral and having an enantiomeric excess of at least (about) 20%. In a first preferred embodiment of this third general embodiment, a higher number of chiral carbons are included within the polyethoid moiety. In this regard, therefore, preferably at least 50% of the carbons selected from C¹, each C², C³ and C⁴ are chiral and have an enantiomeric excess of at least (about) 20%; more preferably, at least 70%, at least 90%, of the carbons selected from C¹, each C², C³ and C⁴ are chiral and have an enantiomeric excess of at least (about) 20%; in some embodiments, each of the carbons selected from C¹, each C², C³ and C⁴ are chiral and have an enantiomeric excess of at least (about) 20%. In a second preferred embodiment of this third general embodiment, chiral carbons within the polyethoid moiety can have a higher degree of chirality: preferably the chiral carbons have an enantiomeric excess of at least (about) 50%, and more preferably, the chiral carbons have an enantiomeric excess of at least (about) 80%. In some embodiments the chiral carbons can have an enantiomeric excess of at least (about) 90% or at least about 95% or at least (about) 98%.

A fourth general embodiment of the first aspect, the invention is directed to a compound comprising a polyethoid moiety having a formula

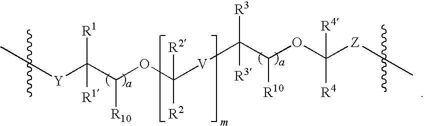

In this fourth general embodiment, the integer m, the integer a, each R¹⁰, R^{1′}, each R^{2′}, R^{3′} and R^{4′}, each V, Y and Z are each as described above in connection with the first general embodiment of the first aspect of the invention (and are to be considered the same as if such were expressly reproduced in this paragraph); moreover, these are generally applicable in this fourth general embodiment of the first aspect, and in preferred embodiments thereof (in each case unless otherwise noted). In this general embodiment, the pendant side chain moieties, R¹, each R², R³ and R⁴, are each independently selected side chain moiety comprising hydrocarbyl or substituted hydrocarbyl, with the proviso, however, that such side chain moieties include structural diversity (as compared with each other). Generally, R¹, each R², R³ and R⁴ can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, each R²′, R³′ and R⁴′, respectively) or with adjacent side chain moieties (e.g., R¹ with a nearest R²) or with an atom on the backbone of the polyethioid moiety (e.g, R² with an adjacent N atom in an embodiment where a V is an N-substituted methyleneamine isostere; in each case with the proviso, however, that such side chain moieties include structural diversity (as compared with each other). Preferably, R¹, each R², R³ and R⁴ can each be an independently selected side chain moiety having a structure of an amino acid side chain, with the proviso, however, that such side chain moieties include structural diversity (as compared with each other). Generally, at least two side chain moieties selected from R¹, each R², and R³ are structurally distinct from each other. In a first preferred embodiment of this fourth general embodiment of the first aspect of the invention, m is an integer ≧2, and at least three side chain moieties selected from R¹, each R², and R³ are structurally distinct from each other. In a second preferred embodiment of this fourth general embodiment, m is an integer ≧3, and at least four side chain moieties selected from R¹, each R², and R³ are structurally distinct from each other. In a third preferred embodiment of this fourth general embodiment, m is an integer ≧7, and at least five side chain moieties selected from R¹, each R², and R³ are structurally distinct from each other.

In a fifth general embodiment of the first aspect, the invention is directed to a polyaminoacid analog. The polyaminoacid analog can be a polyaminoacid compound comprising a structural moiety which includes one or more ethoid isosteres (e.g., one or more ethoid moieties as (substitutive) isosteric replacements for a corresponding one or more amide moieties of the structural moiety of the polyaminoacid). Compounds of the fifth general embodiment can be a structural analog of the polyaminoacid, and can comprise a ethoid moiety or a polyethoid moiety—including for example a polyethoidpeptide or a fully-ethoid-substituted polyethoid). The polyaminoacid analog compounds of the invention of the invention can further comprise one or more isosteres other than ethoids.

Generally, in this fifth general embodiment, the polyaminoacid can be a polypeptide or a protein (e.g., comprising α-amino acid residues (e.g., L-α-amino acid residues, D-α-amino acid residues) linked by amide moieties, such as are derived from coupling of α-amino acids). Generally, the structural moiety of the polyaminoacid comprises three or more amino acid residues linked by amide moieties. The polyaminoacid can be a polyaminoacid having the formula

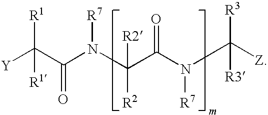

In this fifth general embodiment, m is an integer ≧1, preferably ≧3, and in some embodiments ≧5. The integer m can range from 1 to 1000, and more preferably from 1 to 500. In some embodiments, the integer m can range from 1 to 250, from 1 to 150, from 1 to 100, from 1 to 75, from 1 to 50, from 1 to 30, from 1 to 15, from 1 to 10 or from 1 to 5. In preferred embodiments, m can range from 3 to 250, from 3 to 150, from 3 to 100, from 3 to 75, from 3 to 50, from 3 to 30, from 3 to 15, from 3 to 10 or from 3 to 5. Also in this fifth general embodiment: R¹, each R², and R³ are each an independently selected side chain moiety comprising hydrocarbyl or substituted hydrocarbyl. Generally, R¹, each R², and R³ can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, each R²′ and R³′, respectively) or with adjacent side chain moieties (e.g., R¹ with a nearest R²) or with an atom on the backbone of the polyethioid moiety (e.g, R² with an adjacent N atom in an amide moiety). Preferably, R¹, each R², and R³ can each be an independently selected side chain moiety having a structure of an amino acid side chain; R^{1′}, each R^{2′}, and R^{3′} are each independently selected from the group consisting of H, C₁-C₈ alkyl and substituted C₁-C₈ alkyl, and preferably are selected from the group consisting of H, C₁-C₃ alkyl and substituted C₁-C₃ alkyl. Each R⁷ can be independently selected from the group consisting of —H and a side chain moiety having a structure of an amino acid side chain.

Each of Y and Z can be as described above in connection with the first general embodiment of the first aspect of the invention (and are to be considered the same as if such were expressly reproduced in this paragraph); moreover, these are generally applicable in this fifth general embodiment of the first aspect, and in preferred embodiments thereof (in each case unless otherwise noted).

In a first preferred embodiment of this fifth general embodiment of the first aspect, the invention is directed to an improvement in a polyaminoacid compound where the polyaminoacid comprises a structural moiety including three or more amino acid residues linked by amide moieties. The improvement generally comprises at least two ethoid isosteres, each having a formula

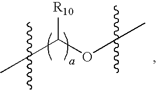

And being an isosteric, substitutive replacement for an amide moiety of the polyaminoacid. In some preferred subembodiments hereof, the improvement can comprise at least three ethoid isosteres, each being a substitutive replacement for an amide moiety of the polyaminoacid. In this first preferred embodiment (and in other described preferred embodiments or subembodiments) of this fifth general embodiment, the integer a, and each R¹⁰ are each as described above in connection with the first general embodiment of the first aspect of the invention (and are to be considered the same as if such were expressly reproduced in this paragraph); moreover, these are generally applicable in this first preferred embodiment (and in other preferred embodiments) of this fifth general embodiment of the first aspect, and in preferred embodiments thereof (in each case unless otherwise noted).

In another second preferred embodiment of this fifth general embodiment of the first aspect, the invention is directed to a polyaminoacid analog or to an improvement in a polyaminoacid, in each case, where one or more of the ethoid isosteres is a substitutive replacement for a proteolytic-susceptible amide moiety of the polyaminoacid. Preferably, least two ethoid isosteres, more preferably at least three ethoid isosteres, are a substitutive replacement for a corresponding at least two proteolytic-susceptible amide moieties of the polyaminoacid.

In another third preferred embodiment of this fifth general embodiment of the first aspect, the invention is directed to a polyaminoacid analog or to an improvement in a polyaminoacid, in each case, where a the number of ethoid isosteres, N_{ETHOID}, considered as a ratio relative to the number of amide moieties, N_{AMIDE}, is at least about 1:99. In some preferred subembodiments, the ratio of N_{ETHOID} to N_{AMIDE} can be at least about 1:49, at least about 1:39, at least about 1:29, at least about 1:19, at least about 1:9, at least about 1:4, at least about 1:3 or at least about 2:3. In some embodiments, the ratio of N_{ETHOID} to N_{AMIDE} can be at least about 1:1, at least about 3:2, at least about 3:1, at least about 4:1, at least about 9:1, or at least about 19:1.

In a fourth preferred embodiment of this fifth general embodiment of the first aspect, the invention is directed to a polyaminoacid analog or to an improvement in a polyaminoacid, in each case, where the analog of a polyaminoacid or the improved polyaminoacid has a property of interest, and preferably has at least one property of interest in common with the polyaminoacid. A polyaminoacid analog or an improved polyaminoacid compound, having commonality of a property of interest with the polyaminoacid can be considered a functional analog of the polyaminoacid. The property of interest can be a biological property, an organoleptic property, a chemical property, or a physical property.

In a sixth general embodiment of the first aspect, the invention is directed to polyaminoacid analogs and to improvements in polyaminoacids, where the (unimproved) polyaminoacid compound (or an analog thereof) is known and has a known biological activity as a pharmaceutical (referred to herein as a polyaminoacid pharmaceutical). Generally, an invention of the sixth general embodiment is directed to an analog of a polyaminoacid pharmaceutical or to an improvement in a polyaminoacid pharmaceutical, in each case, where one or more of the ethoid isosteres is an isosteric, substitutive replacement for a corresponding one or more amide moieties of the polyaminoacid pharmaceutical.

In preferred embodiments of the sixth general embodiment of the first aspect, the invention is directed to specific polyaminoacid analogs and to improvements in specific polyaminoacids. Among the polyaminoacid pharmaceuticals for which the generally described invention of the sixth embodiment is directed are include: GHRH; PR1 (T-cell epitope); Protease-3 peptide (1); Protease-3 peptide (2); Protease-3 peptide (3); Protease-3 peptide (4); Protease-3 peptide (5); Protease-3 peptide (6); Protease-3 peptide (7); Protease-3 peptide (8); Protease-3 peptide (9); Protease-3 peptide (10); Protease-3 peptide (11); P3, B-cell epitope; P3, B-cell epitope: (with spacer); GLP1; LHRH; PTH; Substance P; Neurokinin A; Neurokinin B; Bombesin; CCK-8; Leucine Enkephalin; Methionine Enkephalin; [Des Ala20, Gln34] Dermaseptin; Antimicrobial Peptide (Surfactant); Antimicrobial Anionic Peptide (Surfactant-associated AP); Apidaecin IA; Apidaecin IB; OV-2; 1025, Acetyl-Adhesin Peptide (1025-1044) amide; Theromacin (49-63); Pexiganan (MSI-78); Indolicidin; Apelin-15 (63-77); CFP10 (71-85); Lethal Factor (LF) Inhibitor Anthrax related; Bactenecin; Hepatitis Virus C NS3 Protease Inhibitor 2; Hepatitis Virus C NS3 Protease Inhibitor 3; Hepatitis Virus NS3 Protease Inhibitor 4; NS4A-NS4B Hepatitis Virus C(NS3 Protease Inhibitor 1); HIV-1, HIV-2 Protease Substrate; Anti-Flt1 Peptide; Bak-BH3; Bax BH3 peptide (55-74) (wild type); Bid BH3-r8; CTT (Gelatinase Inhibitor); E75 (Her-2/neu) (369-377); GRP78 Binding Chimeric Peptide Motif; p53(17-26); EGFR2/KDR Antagonist; Colivelin AGA-(C8R) HNG17 (Humanin derivative); Activity-Dependent Neurotrophic Factor (ADNF); Beta-Secretase Inhibitor 1; Beta-Secretase Inhibitor 2; chβ-Amyloid (30-16); Humanun (HN); sHNG, [Gly14]-HN, [Gly14]-Humanin; Angiotensin Converting Enzyme Inhibitor (BPP); Renin Inhibitor III; Annexin 1 (ANXA-1; Ac2-12); Anti-Inflammatory Peptide 1; Anti-Inflammatory Peptide 2; Anti-Inflammatory Apelin 12; [D-Phe12, Leu14]-Bombesin; Antennapedia Peptide (acid) (penetratin); Antennepedia Leader Peptide (CT); Mastoparan; [Thr28, Nle31]-Cholecystokinin (25-33) sulfated; Nociceptin (1-13) (amide); Fibrinolysis Inhibiting Factor; Gamma-Fibrinogen (377-395); Xenin; Obestatin (human); [His1, Lys6]-GHRP (GHRP-6); [Ala5, β-Ala8]-Neurokinin A (4-10); Neuromedin B; Neuromedin C; Neuromedin N; Activity-Dependent Neurotrophic Factor (ADNF-14); Acetalin 1 (Opioid Receptor Antagonist 1); Acetalin 2 (Opioid Receptor Antagonist 2); Acetalin 3 (Opioid Receptor Antagonist 3); ACTH (1-39) (human); ACTH (7-38) (human); Sauvagine; Adipokinetic Hormone (Locusta Migratoria); Myristoylated ADP-Ribosylation Factor 6, myr-ARF6 (2-13); PAMP (1-20) (Proadrenomedullin (1-20) human); AGRP (25-51); Amylin (8-37) (human); Angiotensin I (human); Angiotensin II (human); Apstatin (Aminopeptidase P Inhibitor); Brevinin-1; Magainin 1; RL-37; LL-37 (Antimicrobial Peptide) (human); Cecropin A; Antioxidant peptide A; Antioxidant peptide B; L-Carnosine; Bcl 9-2; NPVF; Neuropeptide AF (hNPAF) (Human); Bax BH3 peptide (55-74); bFGF Inhibitory Peptide; bFGF inhibitory Peptide II; Bradykinin; [Des-Arg10]-HOE I40; Caspase 1 Inhibitor II; Caspase 1 Inhibitor VIII; Smac N7 Protein; MEK1 Derived Peptide Inhibitor 1; hBD-1 (β-Defensin-1) (human); hBD-3 (β-Defensin-3) (human); hBD-4 (β-Defensin-4) (human); HNP-1 (Defensin Human Neutrophil Peptide 1); HNP-2 (Defensin Human neutrophil Peptide-2 Dynorphin A (1-17)); Endomorphin-1; β-Endorphin (human porcine); Endothelin 2 (human); Fibrinogen Binding Inhibitor Peptide; Cyclo(-GRGDSP); TP508 (Thrombin-derived Peptide); Galanin (human); GIP (human); Gastrin Releasing Peptide (human); Gastrin-1 (human); Ghrelin (human); PDGF-BB peptide; [D-Lys3]-GHRP-6; HCV Core Protein (1-20); a3β1 Integrin Peptide Fragment (325) (amide); Laminin Pentapeptide (amide); Melanotropin Potentiating Factor (MPF); VA-β-MSH, Lipotropin-Y (Proopiomelanocortin-derived); Atrial Natriuretic Peptide (1-28) (human); Vasonatrin Peptide (1-27); [Ala5, β-Ala8]-Neurokinin A (4-10); Neuromedin L (NKA); Ac-(Leu28, 31)-Neuropeptide Y (24-26); Alytesin; Brain Neuropeptide II; [D-tyr11]-Neurotensin; IKKy NEMO Binding Domain (NBD) Inhibitory Peptide; PTD-p50 (NLS) Inhibitory Peptide; Orexin A (bovine, human, mouse, rat); Orexin B (human); Aquaporin-2(254-267) (human Pancreastatin) (37-52); Pancreatic Polypeptide (human); Neuropeptide; Peptide YY (3-36) (human); Hydroxymethyl-Phytochelatin 2; PACAP (1-27) (amide, human, bovine, rat); Prolactin Releasing Peptide (1-31) (human); Salusin-alpha; Salusin-beta; Saposin C22; Secretin (human); L-Selectin; Endokinin A/B; Endokinin C (Human); Endokinin D (Human); Thrombin Receptor (42-48) Agonist (human); LSKL (Inhibitor of Thrombospondin); Thyrotropin Releasing Hormone (TRH); P55-TNFR Fragment; Urotensin II (human); VIP (human, porcine, rat); VIP Antagonist; Helodermin; Exenatide; ZP10 (AVE00100); Pramlinitide; AC162352 (PYY) (3-36); PYY; Obinepitide; Glucagon; GRP; Ghrelin (GHRP6); Leuprolide; Histrelin; Oxytocin; Atosiban (RWJ22164); Sermorelin; Nesiritide; bivalirudin (Hirulog); Icatibant; Aviptadil; Rotigaptide (ZP123, GAP486); Cilengitide (EMD-121924, RGD Peptides); AlbuBNP; BN-054; Angiotensin II; MBP-8298; Peptide Leucine Arginine; Ziconotide; AL-208; AL-108; Carbeticon; Tripeptide; SAL; Coliven; Humanin; ADNF-14; VIP (Vasoactive Intestinal Peptide); Thymalfasin; Bacitracin (USP); Gramidicin (USP); Pexiganan (MSI-78); P113; PAC-113; SCV-07; HLF1-11 (Lactoferrin); DAPTA; TRI-1144; Tritrpticin; Antiflammin 2; Gattex (Teduglutide, ALX-0600); Stimuvax (L-BLP25); Chrysalin (TP508); Melanonan II; Spantide II; Ceruletide; Sincalide; Pentagastin; Secretin; Endostatin peptide; E-selectin; HER2; IL-6; IL-8; IL-10; PDGF; Thrombospondin; uPA (1); uPA (2); VEGF; VEGF (2); Pentapeptide-3; Glutathione; XXLRR; Beta-Amyloid Fibrillogenesis; Endomorphin-2; TIP 39 (Tuberoinfundibular Neuropeptide); PACAP (1-38) (amide, human, bovine, rat); TGFβ activating peptide; Insulin sensitizing factor (ISF402); Transforming Growth Factor β1 Peptide (TGF-β1); Caerulein Releasing Factor; IELLQAR (8-branch MAPS); Tigapotide PK3145; Goserelin; Abarelix; Cetrorelix; Ganirelix; Degarelix (Triptorelin); Barusiban (FE 200440); Pralmorelin; Octreotide; Eptifibatide; Netamiftide (INN-00835); Daptamycin; Spantide II (1); Delmitide (RDP-58); AL-209; Enfuvirtide; IDR-1; Hexapeptide-6; Insulin-A chain; Lanreotide; Hexapeptide-3; Insulin B-chain; Glargine-A chain; Glargine-B chain; Insulin-L isPro B-chain analog; Insulin-Aspart B-chain analog; Insulin-Glulisine B chain analog; Insulin-Determir B chain analog; Somatostatin; Somatostatin Tumor Inhibiting Analog; Pancreastatin (37-52); Vasoactive Intestinal Peptide fragment (KKYL-NH2); and Dynorphin A; or analogs thereof of any of the foregoing. An exemplary GHRH is represented by SEQ ID NO: 1; an exemplary PR1 T-cell epitope is represented by SEQ ID NO: 2; an exemplary Protease-3 peptide 1 is represented by SEQ ID NO: 3; an exemplary Protease-3 peptide 2 is represented by SEQ ID NO: 4; an exemplary Protease-3 peptide 3 is represented by SEQ ID NO: 5; an exemplary Protease-3 peptide 4 is represented by SEQ ID NO: 6; an exemplary Protease-3 peptide 5 is represented by SEQ ID NO: 7; an exemplary Protease-3 peptide 6 is represented by SEQ ID NO: 8; an exemplary an exemplary Protease-3 peptide 7 is represented by SEQ ID NO: 9; an exemplary Protease-3 peptide 8 is represented by SEQ ID NO: 10; an exemplary Protease-3 peptide 9 is represented by SEQ ID NO: 11; an exemplary Protease-3 peptide 10 is represented by SEQ ID NO: 12; an exemplary Protease-3 peptide 11 is represented by SEQ ID NO: 13; an exemplary an exemplary P3, B-cell epitope is represented by SEQ ID NO: 14; an exemplary P3, B-cell epitope: with spacer: is represented by SEQ ID NO: 15; an exemplary GLP1 is represented by SEQ ID NO: 16; an exemplary LHRH is represented by SEQ ID NO: 17; an exemplary PTH is represented by SEQ ID NO: 18; an exemplary Substance P is represented by SEQ ID NO: 19; an exemplary Neurokinin A is represented by SEQ ID NO: 20; an exemplary Neurokinin B is represented by SEQ ID NO: 21; an exemplary Bombesin is represented by SEQ ID NO: 22; an exemplary CCK-8 is represented by SEQ ID NO: 23; an exemplary Leucine Enkephalin is represented by SEQ ID NO: 24; an exemplary Methionine Enkephalin is represented by SEQ ID NO: 25; an exemplary [Des Ala20, Gln34] Dermaseptin is represented by SEQ ID NO: 26; an exemplary antimicrobial Peptide Surfactant is represented by SEQ ID NO: 27; an exemplary antimicrobial anionic Peptide Surfactant-associated AP is represented by SEQ ID NO: 28; an exemplary Apidaecin IA is represented by SEQ ID NO: 29; an exemplary Apidaecin IB is represented by SEQ ID NO: 30; an exemplary OV-2 is represented by SEQ ID NO: 31; an exemplary 1025, Acetyl-Adhesin Peptide 1025-1044 amide is represented by SEQ ID NO: 32; an exemplary Theromacin 49-63 is represented by SEQ ID NO: 33; an exemplary Pexiganan MSI-78 is represented by SEQ ID NO: 34; an exemplary Indolicidin is represented by SEQ ID NO: 35; an exemplary Apelin-15 63-77 is represented by SEQ ID NO: 36; an exemplary CFP10 71-85 is represented by SEQ ID NO: 37; an exemplary Lethal Factor LF Inhibitor anthrax related is represented by SEQ ID NO: 38; an exemplary Bactenecin is represented by SEQ ID NO: 39; an exemplary Hepatitis Virus C NS3 Protease Inhibitor 2 is represented by SEQ ID NO: 40; an exemplary Hepatitis Virus C NS3 Protease Inhibitor 3 is represented by SEQ ID NO: 41; an exemplary an exemplary Hepatitis Virus NS3 Protease Inhibitor 4 is represented by SEQ ID NO: 42; an exemplary NS4A-NS4B Hepatitis Virus C NS3 Protease Inhibitor 1 is represented by SEQ ID NO: 43; an exemplary HIV-1, HIV-2 Protease Substrate is represented by SEQ ID NO: 44; an exemplary anti-Flt1 Peptide is represented by SEQ ID NO: 45; an exemplary Bak-BH3 is represented by SEQ ID NO: 46; an exemplary Bax BH3 peptide 55-74 wild type is represented by SEQ ID NO: 47; an exemplary Bid BH3-r8 is represented by SEQ ID NO: 48; an exemplary CTT Gelatinase Inhibitor is represented by SEQ ID NO: 49; an exemplary E75 Her-2/neu 369-377 is represented by SEQ ID NO: 50; an exemplary GRP78 Binding Chimeric Peptide Motif is represented by SEQ ID NO: 51; an exemplary p5317-26 is represented by SEQ ID NO: 52; an exemplary EGFR2/KDR antagonist is represented by SEQ ID NO: 53; an exemplary Colivelin is represented by SEQ ID 54; an exemplary AGA-C8R HNG17 Humanin derivative is represented by SEQ ID NO: 55; an exemplary Activity-Dependent Neurotrophic Factor ADNF is represented by SEQ ID NO: 56; an exemplary Beta-Secretase Inhibitor 1 is represented by SEQ ID NO: 57; an exemplary Beta-Secretase Inhibitor 2 is represented by SEQ ID NO: 58; an exemplary chβ-Amyloid 30-16 is represented by SEQ ID NO: 59; an exemplary Humanun HN is represented by SEQ ID NO: 60; an exemplary sHNG, [Gly14]-HN, [Gly14]-Humanin is represented by SEQ ID NO: 61; an exemplary angiotensin Converting Enzyme Inhibitor BPP is represented by SEQ ID NO: 62; an exemplary Renin Inhibitor III is represented by SEQ ID NO: 63; an exemplary annexin 1 ANXA-1; an exemplary Ac2-12 is represented by SEQ ID NO: 64; an exemplary anti-Inflammatory Peptide 1 is represented by SEQ ID NO: 65; an exemplary anti-Inflammatory Peptide 2 is represented by SEQ ID NO: 66; an exemplary anti-Inflammatory Apelin 12 is represented by SEQ ID NO: 67; an exemplary [D-Phe12, Leu14]-Bombesin is represented by SEQ ID NO: 68; an exemplary antennapedia Peptide acid penetratin is represented by SEQ ID NO: 69; an exemplary antennepedia Leader Peptide CT is represented by SEQ ID NO: 70; an exemplary Mastoparan is represented by SEQ ID NO: 71; an exemplary [Thr28, Nle31]-Cholecystokinin 25-33 sulfated is represented by SEQ ID NO: 72; an exemplary Nociceptin 1-13 amide is represented by SEQ ID NO: 73; an exemplary an exemplary Fibrinolysis Inhibiting Factor is represented by SEQ ID NO: 74; an exemplary Gamma-Fibrinogen 377-395 is represented by SEQ ID NO: 75; an exemplary Xenin is represented by SEQ ID NO: 76; an exemplary Obestatin human is represented by SEQ ID NO: 77; an exemplary [His1, Lys6]-GHRP GHRP-6 is represented by SEQ ID NO: 78; an exemplary [Ala5, β-Ala8]-Neurokinin A 4-10 is represented by SEQ ID NO: 79; an exemplary an exemplary Neuromedin B is represented by SEQ ID NO: 80; an exemplary Neuromedin C is represented by SEQ ID NO: 81; an exemplary Neuromedin N is represented by SEQ ID NO: 82; an exemplary Activity-Dependent Neurotrophic Factor ADNF-14 is represented by SEQ ID NO: 83; an exemplary Acetalin 1 Opioid Receptor antagonist 1 is represented by SEQ ID NO: 84; an exemplary Acetalin 2 Opioid Receptor antagonist 2 is represented by SEQ ID NO: 85; an exemplary Acetalin 3 Opioid Receptor antagonist 3 is represented by SEQ ID NO: 86; an exemplary ACTH 1-39 human is represented by SEQ ID NO: 87; an exemplary ACTH 7-38 human is represented by SEQ ID NO: 88; an exemplary an exemplary Sauvagine is represented by SEQ ID NO: 89; an exemplary Adipokinetic Hormone Locusta Migratoria is represented by SEQ ID NO: 90; an exemplary Myristoylated ADP-Ribosylation Factor 6, myr-ARF6 2-13 is represented by SEQ ID NO: 91; an exemplary PAMP 1-20 Proadrenomedullin 1-20 human is represented by SEQ ID NO: 92; an exemplary AGRP 25-51 is represented by SEQ ID NO: 93; an exemplary Amylin 8-37 human is represented by SEQ ID NO: 94; an exemplary angiotensin I human is represented by SEQ ID NO: 95; an exemplary angiotensin II human is represented by SEQ ID NO: 96; an exemplary Apstatin Aminopeptidase P Inhibitor is represented by SEQ ID NO: 97; an exemplary Brevinin-1 is represented by SEQ ID NO: 98; an exemplary Magainin 1 is represented by SEQ ID NO: 99; an exemplary RL-37 is represented by SEQ ID NO: 100; an exemplary LL-37 antimicrobial Peptide human is represented by SEQ ID NO: 101; an exemplary Cecropin A is represented by SEQ ID NO: 102; an exemplary antioxidant peptide A is represented by SEQ ID NO: 103; an exemplary antioxidant peptide B is represented by SEQ ID NO: 104; an exemplary L-Carnosine is represented by SEQ ID NO: 105; an exemplary Bcl 9-2 is represented by SEQ ID NO: 106; an exemplary NPVF is represented by SEQ ID NO: 107; an exemplary Neuropeptide AF hNPAF Human is represented by SEQ ID NO: 108; an exemplary Bax BH3 peptide 55-74 is represented by SEQ ID NO: 109; an exemplary bFGF Inhibitory Peptide is represented by SEQ ID NO: 110; an exemplary bFGF inhibitory Peptide II is represented by SEQ ID NO: 111; an exemplary Bradykinin is represented by SEQ ID NO: 112; an exemplary [Des-Arg10]-HOE I40 is represented by SEQ ID NO: 113; an exemplary Caspase 1 Inhibitor II is represented by SEQ ID NO: 114; an exemplary Caspase 1 Inhibitor VIII is represented by SEQ ID NO: 115; an exemplary Smac N7 Protein is represented by SEQ ID NO: 116; an exemplary MEK1 Derived Peptide Inhibitor 1 is represented by SEQ ID NO: 117; an exemplary hBD-1 β-Defensin-1 human is represented by SEQ ID NO: 118; an exemplary hBD-3 β-Defensin-3 human is represented by SEQ ID NO: 119; an exemplary hBD-4 β-Defensin-4 human is represented by SEQ ID NO: 120; an exemplary HNP-1 Defensin Human Neutrophil Peptide 1 is represented by SEQ ID NO: 121; an exemplary HNP-2 Defensin Human neutrophil Peptide-2 Dynorphin A 1-17 is represented by SEQ ID NO: 122; an exemplary Endomorphin-1 is represented by SEQ ID NO: 123; an exemplary β-Endorphin human porcine is represented by SEQ ID NO: 124; an exemplary Endothelin 2 human is represented by SEQ ID NO: 125; an exemplary Fibrinogen Binding Inhibitor Peptide is represented by SEQ ID NO: 126; an exemplary Cyclo-GRGDSP is represented by SEQ ID NO: 127; an exemplary TP508 Thrombin-derived Peptide is represented by SEQ ID NO: 128; an exemplary Galanin human is represented by SEQ ID NO: 129; an exemplary GIP human is represented by SEQ ID NO: 130; an exemplary Gastrin Releasing Peptide human is represented by SEQ ID NO: 131; an exemplary Gastrin-1 human is represented by SEQ ID NO: 132; an exemplary Ghrelin human is represented by SEQ ID NO: 133; an exemplary PDGF-BB peptide is represented by SEQ ID NO: 134; an exemplary [D-Lys3] GHRP-6 is represented by SEQ ID NO: 135; an exemplary HCV Core Protein 1-20 is represented by SEQ ID NO: 136; an exemplary a3β1 Integrin Peptide Fragment 325 amide is represented by SEQ ID NO: 137; an exemplary Laminin Pentapeptide amide is represented by SEQ ID NO: 138; an exemplary Melanotropin-Potentiating Factor MPF is represented by SEQ ID NO: 139; an exemplary VA-β-MSH, Lipotropin-Y Proopiomelanocortin-derived is represented by SEQ ID NO:140; an exemplary Atrial Natriuretic Peptide 1-28 human is represented by SEQ ID NO: 141; an exemplary Vasonatrin Peptide 1-27 is represented by SEQ ID NO: 142; an exemplary [Ala5, β-Ala8]-Neurokinin A 4-10 is represented by SEQ ID NO: 143; an exemplary Neuromedin L NKA is represented by SEQ ID NO: 144; an exemplary Ac-Leu28, 31-Neuropeptide Y 24-26 is represented by SEQ ID NO: 145; an exemplary Alytesin is represented by SEQ ID NO: 146; an exemplary Brain Neuropeptide II is represented by SEQ ID NO: 147; an exemplary [D-tyr11]-Neurotensin is represented by SEQ ID NO: 148; an exemplary IKKy NEMO Binding Domain NBD Inhibitory Peptide is represented by SEQ ID NO: 149; an exemplary PTD-p50 NLS Inhibitory Peptide is represented by SEQ ID NO: 150; an exemplary Orexin A bovine, human, mouse, rat is represented by SEQ ID NO: 151; an exemplary Orexin B human is represented by SEQ ID NO: 152; an exemplary Aquaporin-2254-267 human Pancreastatin 37-52 is represented by SEQ ID NO: 153; an exemplary Pancreatic Polypeptide human is represented by SEQ ID NO: 154; an exemplary Neuropeptide is represented by SEQ ID NO: 155; an exemplary Peptide YY 3-36 human is represented by SEQ ID NO: 156; an exemplary Hydroxymethyl-Phytochelatin 2 is represented by SEQ ID NO: 157; an exemplary PACAP 1-27 amide, human, bovine, rat is represented by SEQ ID NO: 158; an exemplary Prolactin Releasing Peptide 1-31 human is represented by SEQ ID NO: 159; an exemplary Salusin-alpha is represented by SEQ ID NO: 160; an exemplary Salusin-beta is represented by SEQ ID NO: 161; an exemplary Saposin C22 is represented by SEQ ID NO: 162; an exemplary Secretin human is represented by SEQ ID NO: 163; an exemplary L-Selectin is represented by SEQ ID NO: 164; an exemplary Endokinin A/B is represented by SEQ ID NO: 165; an exemplary Endokinin C Human is represented by SEQ ID NO: 166; an exemplary Endokinin D Human is represented by SEQ ID NO: 167; an exemplary Thrombin Receptor 42-48 Agonist human is represented by SEQ ID NO: 168; an exemplary LSKL Inhibitor of Thrombospondin is represented by SEQ ID NO: 169; an exemplary Thyrotropin Releasing Hormone TRH is represented by SEQ ID NO: 170; an exemplary P55-TNFR Fragment is represented by SEQ ID NO: 171; an exemplary Urotensin II human is represented by SEQ ID NO: 172; an exemplary VIP human, porcine, rat is represented by SEQ ID NO: 173; an exemplary VIP antagonist is represented by SEQ ID NO: 174; an exemplary Helodermin is represented by SEQ ID NO: 175; an exemplary Exenatide is represented by SEQ ID NO: 176; an exemplary ZP10 AVE00100 is represented by SEQ ID NO: 177; an exemplary Pramlinitide is represented by SEQ ID NO: 178; an exemplary AC162352 PYY3-36 is represented by SEQ ID NO: 179; an exemplary PYY is represented by SEQ ID NO: 180; an exemplary Obinepitide is represented by SEQ ID NO: 181; an exemplary Glucagon is represented by SEQ ID NO: 182; an exemplary GRP is represented by SEQ ID NO: 183; an exemplary Ghrelin GHRP6 is represented by SEQ ID NO: 184; an exemplary Leuprolide is represented by SEQ ID NO: 185; an exemplary Histrelin is represented by SEQ ID NO: 186; an exemplary Oxytocin is represented by SEQ ID NO: 187; an exemplary Atosiban RWJ22164 is represented by SEQ ID NO: 188; an exemplary Sermorelin is represented by SEQ ID NO: 189; an exemplary Nesiritide is represented by SEQ ID NO: 190; an exemplary bivalirudin Hirulog is represented by SEQ ID NO: 191; an exemplary Icatibant is represented by SEQ ID NO: 192; an exemplary Aviptadil is represented by SEQ ID NO: 193; an exemplary Rotigaptide ZP123, GAP486 is represented by SEQ ID NO: 194; an exemplary Cilengitide EMD-121924, RGD Peptides is represented by SEQ ID NO: 195; an exemplary AlbuBNP is represented by SEQ ID NO: 196; an exemplary BN-054 is represented by SEQ ID NO: 197; an exemplary angiotensin II is represented by SEQ ID NO: 198; an exemplary MBP-8298 is represented by SEQ ID NO: 199; an exemplary Peptide Leucine Arginine is represented by SEQ ID NO: 200; an exemplary Ziconotide is represented by SEQ ID NO: 201; an exemplary AL-208 is represented by SEQ ID NO: 202; an exemplary AL-108 is represented by SEQ ID NO: 203; an exemplary Carbeticon is represented by SEQ ID NO: 204; an exemplary Tripeptide is represented by SEQ ID NO: 205; an exemplary SAL is represented by SEQ ID NO: 206; an exemplary Coliven is represented by SEQ ID NO: 207; an exemplary Humanin is represented by SEQ ID NO: 208; an exemplary ADNF-14 is represented by SEQ ID NO: 209; an exemplary VIP Vasoactive Intestinal Peptide is represented by SEQ ID NO: 210; an exemplary Thymalfasin is represented by SEQ ID NO: 211; an exemplary Bacitracin USP is represented by SEQ ID NO: 212; an exemplary Gramidicin USP is represented by SEQ ID NO: 213; an exemplary Pexiganan MSI-78 is represented by SEQ ID NO: 214; an exemplary P113 is represented by SEQ ID NO: 215; an exemplary PAC-113 is represented by SEQ ID NO: 216; an exemplary SCV-07 is represented by SEQ ID NO: 217; an exemplary HLF1-11 Lactoferrin is represented by SEQ ID NO: 218; an exemplary DAPTA is represented by SEQ ID NO: 219; an exemplary TRI-1144 is represented by SEQ ID NO: 220; an exemplary Tritrpticin is represented by SEQ ID NO: 221; an exemplary antiflammin 2 is represented by SEQ ID NO: 222; an exemplary Gattex Teduglutide, ALX-0600 is represented by SEQ ID NO: 223; an exemplary Stimuvax L-BLP25 is represented by SEQ ID NO: 224; an exemplary Chrysalin TP508 is represented by SEQ ID NO: 225; an exemplary Melanonan II is represented by SEQ ID NO: 226; an exemplary Spantide II is represented by SEQ ID NO: 227; an exemplary Ceruletide is represented by SEQ ID NO: 228; an exemplary Sincalide is represented by SEQ ID NO: 229; an exemplary Pentagastin is represented by SEQ ID NO: 230; an exemplary Secretin is represented by SEQ ID NO: 231; an exemplary Endostatin peptide is represented by SEQ ID NO: 232; an exemplary E-selectin is represented by SEQ ID NO: 233; an exemplary HER2 is represented by SEQ ID NO: 234; an exemplary IL-6 is represented by SEQ ID NO: 235; an exemplary IL-8 is represented by SEQ ID NO: 236; an exemplary IL-10 is represented by SEQ ID NO: 237; an exemplary PDGF is represented by SEQ ID NO: 238; an exemplary Thrombospondin is represented by SEQ ID NO: 239; an exemplary uPA 1 is represented by SEQ ID NO: 240; an exemplary uPA 2 is represented by SEQ ID NO: 241; an exemplary VEGF is represented by SEQ ID NO: 242; an exemplary VEGF 2 is represented by SEQ ID NO: 243; an exemplary Pentapeptide-3 is represented by SEQ ID NO: 244; an exemplary Glutathione is represented by SEQ ID NO: 245; an exemplary XXLRR is represented by SEQ ID NO. 246; an exemplary Beta-Amyloid Fibrillogenesis is represented by SEQ ID NO: 247; an exemplary Endomorphin-2 is represented by SEQ ID NO: 248; an exemplary TIP 39 Tuberoinfundibular Neuropeptide is represented by SEQ ID NO: 249; an exemplary PACAP 1-38 amide, human, bovine, rat is represented by SEQ ID NO: 250; an exemplary TGFβ activating peptide is represented by SEQ ID NO: 251; an exemplary Insulin sensitizing factor ISF402 is represented by SEQ ID NO: 252; an exemplary Transforming Growth Factor 131 Peptide TGF-β1 is represented by SEQ ID NO: 253; an exemplary Caerulein Releasing Factor is represented by SEQ ID NO: 254; an exemplary IELLQAR 8-branch MAPS is represented by SEQ ID NO: 255; an exemplary Tigapotide PK3145 is represented by SEQ ID NO: 256; an exemplary Goserelin is represented by SEQ ID NO: 257; an exemplary Abarelix is represented by SEQ ID NO: 258; an exemplary Cetrorelix is represented by SEQ ID NO: 259; an exemplary Ganirelix is represented by SEQ ID NO: 260; an exemplary Degarelix Triptorelin is represented by SEQ ID NO: 261; an exemplary Barusiban FE 200440 is represented by SEQ ID NO: 262; an exemplary Pralmorelin is represented by SEQ ID NO: 263; an exemplary Octreotide is represented by SEQ ID NO: 264; an exemplary Eptifibatide is represented by SEQ ID NO: 265; an exemplary Netamiftide INN-00835 is represented by SEQ ID NO: 266; an exemplary Daptamycin is represented by SEQ ID NO: 267; an exemplary Spantide II 1 is represented by SEQ ID NO: 268; an exemplary Delmitide RDP-58 is represented by SEQ ID NO: 269; an exemplary AL-209 is represented by SEQ ID NO: 270; an exemplary Enfuvirtide is represented by SEQ ID NO: 271; an exemplary IDR-1 is represented by SEQ ID NO: 272; an exemplary Hexapeptide-6 is represented by SEQ ID NO: 272; an exemplary Insulin-A chain is represented by SEQ ID NO: 274; an exemplary Lanreotide is represented by SEQ ID NO: 275; an exemplary Hexapeptide-3 is represented by SEQ ID NO: 276; an exemplary Insulin B-chain is represented by SEQ ID NO: 277; an exemplary Glargine-A chain is represented by SEQ ID NO: 278; an exemplary Glargine-B chain is represented by SEQ ID NO: 279; an exemplary Insulin-LisPro B-chain analog is represented by SEQ ID NO: 280; an exemplary Insulin-Aspart B-chain analog is represented by SEQ ID NO: 281; an exemplary Insulin-Glulisine B chain analog is represented by SEQ ID NO: 282; an exemplary Insulin-Determir B chain analog is represented by SEQ ID NO: 283; an exemplary Somatostatin is represented by SEQ ID NO: 284; an exemplary Somatostatin Tumor Inhibiting analog is represented by SEQ ID NO: 285; an exemplary Pancreastatin 37-52 is represented by SEQ ID NO: 286; an exemplary Vasoactive Intestinal Peptide fragment KKYL-NH2 is represented by SEQ ID NO: 287; an exemplary Dynorphin A is represented by SEQ ID NO: 288; or analogs thereof of any of the foregoing.

In some embodiments, the polyaminoacid pharmaceuticals include PYY; Obinepitide; PTH; Leuprolide; Atosiban; Sermorelin; Pralmorelin; Nesiritide; Rotigaptide; Cilengitide; MBP-8298; AL-108; Enfuvirtide; Thymalfasin; Daptamycin; HLF1-11; Lactoferrin; Gattex; Teduglutide; ALX-0600; Delmitide; RDP-58; pentapeptide-3; hexapeptide-6; L-carnosine; and glutathione; or analogs thereof of any of the foregoing. An exemplary PYY is represented by SEQ ID NO: 181; an exemplary Obinepitide is represented by SEQ ID NO: 183; an exemplary PTH is represented by SEQ ID NO:18; an exemplary Leuprolide is represented by SEQ ID NO: 187; an exemplary Atosiban is represented by SEQ ID NO: 190; an exemplary Sermorelin is represented by SEQ ID NO:191; an exemplary Pralmorelin is represented by SEQ ID NO:268; an exemplary Nesiritide is represented by SEQ ID NO: 192; an exemplary Rotigaptide is represented by SEQ ID NO:196; an exemplary Cilengitide is represented by SEQ ID NO: 197; an exemplary MBP-8298 is represented by SEQ ID NO:202; an exemplary AL-108 is represented by SEQ ID NO:206; an exemplary Enfuvirtide is represented by SEQ ID NO: 278; an exemplary Thymalfasin is represented by SEQ ID NO: 214; an exemplary Daptamycin is represented by SEQ ID NO: 272; an exemplary HLF1-11 is represented by SEQ ID NO: 222; an exemplary Lactoferrin is represented by SEQ ID NO:222; an exemplary Gattex is represented by SEQ ID NO: 227; an exemplary Teduglutide is represented by SEQ ID NO: 227; an exemplary ALX-0600 is represented by SEQ ID NO:227; an exemplary Delmitide is represented by SEQ ID NO: 274; an exemplary RDP-58 is represented by SEQ ID NO: 274; an exemplary pentapeptide-3 is represented by SEQ ID NO:248; an exemplary hexapeptide-6 is represented by SEQ ID NO: 107; an exemplary L-carnosine is represented by SEQ ID NO: 107; an exemplary glutathione is represented by SEQ ID NO:249; or analogs thereof of any of the foregoing. In some embodiments the polyaminoacid pharmaceuticals include GLP-1; LHRH; PTH; Substance P; Neurokinin A; Neurokinin B; Bombesin; CCK-8; Leucine Enkephalin ENKEPHALIN; Methionine Enkephalin; GHRH; PR1 (T-cell epitope); P3 (B-cell epitope) and Somatostatin; or analogs thereof. An exemplary GLP-1 is represented by SEQ ID NO: 16; an exemplary LHRH is represented by SEQ ID NO: 17; an exemplary PTH is represented by SEQ ID NO: 18; an exemplary Substance P is represented by SEQ ID NO: 19; an exemplary Neurokinin A is represented by SEQ ID NO: 20; an exemplary Neurokinin B is represented by SEQ ID NO: 21; an exemplary Bombesin is represented by SEQ ID NO: 22; an exemplary CCK-8 is represented by SEQ ID NO: 23; an exemplary Leucine Enkephalin is represented by SEQ ID NO: 24; an exemplary Methionine Enkephalin is represented by SEQ ID NO: 25; an exemplary GHRH is represented by SEQ ID NO: 1; an exemplary PR1 (T-cell epitope) is represented by SEQ ID NO: 2; an exemplary P3 (B-cell epitope) is represented by SEQ ID NO: 14; and an exemplary Somatostatin is represented by SEQ ID NO: 284; or analogs thereof of any of the foregoing.

In a seventh general embodiment of the first aspect, the invention is directed to a compound comprising an ethoid moiety or a polyethoid moiety prepared by a process which includes a method of any of the inventions within the second aspect of the invention, as summarized below and described in further detail hereinafter.

A number of general features, summarized below and described in further detail hereinafter, are specifically contemplated in combination with the first aspect of the invention, including in combination with each of the general embodiments of the first aspect, as well as each preferred embodiment of each of the general embodiments. Moreover, a number of subembodiments are contemplated as summarized above and further described hereinafter, in which various features as generally described are more specifically contemplated, independently and in various combinations.

### Methods for Preparing Ethoid-Containing Compounds

The methods of the invention are varied, and include general approaches, more particularly directed reaction schema, and specific reaction chemistries.

Among the general approaches, the methods of the invention include modular, universal, reproducible, flexible approaches and schema (e.g., stepwise chain extension reactions) for preparing compounds comprising polyethoid moieties. Significantly, the structural diversity of such polyethoid moieties can be controllable and reproducibly varied (e.g., for preparing macromolecules comprising ethoid isosteres and having different side chain moieties corresponding to different amino-acid side chain moieties) by the approaches and schema of the invention. Further, such modular approaches and reaction schema can be readily integrated with known chain extension approaches and reaction schema for preparing polypeptides and proteins, thereby providing a modular system for which can be used to flexibly prepare diverse macromolecules comprising polyethoidpeptides.

The general approaches and more particularly directed schema of the invention are preferably and advantageously implemented with the specific reaction chemistries of the invention.

Although various concepts and features of the invention have been introduced and described in the preceding paragraphs in the context of methods for preparing ethoid-containing compounds (e.g., compounds comprising one or more ethoid moieties, such as polyethoids or polyethoidpeptides), such concepts and features are equally applicable to other aspects and embodiments of the invention, and are expressly contemplated in connection therewith.

In a second aspect, therefore, the invention is directed to methods for preparing compounds comprising an ethoid moiety or a polyethoid moiety.

In a first general embodiment of the second aspect, the invention is directed to a method for preparing a compound comprising a polyethoid, which method comprises synthesizing a polyethoid moiety through a series of controlled stepwise reactions. The series of controlled stepwise reactions (also referred to collectively as chain extension reactions) can, in general, comprise (i) a first addition reaction(s), in which a first side chain moiety (e.g., R¹) (provided to a reaction mixture as a first reagent) is added to a functional group covalently linked, directly or indirectly, to a solid support (for example, a functional group of a starting moiety (e.g., a solid support) or of an intermediate moiety (e.g., a polyethoid intermediate, such as a polyethoidpeptide intermediate)) with the formation of an ethoid moiety, (ii) a first transformation reaction(s), in which a moiety of the reaction product of (i) is functionalized for subsequent second addition reaction(s), and (iii) a second addition reaction(s), in which a second side chain moiety (e.g., R²) (provided to a reaction mixture as a second reagent) is added to the functional group of (ii) with the formation of a second ethoid moiety. Each of such side chain moieties (e.g., R¹, R²) are independently selected side chain moiety having a structure of an amino acid side chain (including natural amino acid side chains, and non-natural amino acid side chains). In generally preferred embodiments of this first general embodiment of the second aspect, the two or more addition reactions (and independently, multiple transformation reactions for three or more addition reactions) can each be effected using substantially the same reaction schema. Also generally preferably, a set of reagents comprising various structurally distinct side chain moieties can be provided with common reactive functional groups (protected or unprotected) as compared between reagents, that help enable such common reaction schema. Such approach thereby provides a modular system for a series of chain extension reactions to flexibly create diverse macromolecules comprising polyethoids. Further, such approach can be readily integrated with known chain extension reactions for preparing polypeptides and proteins, thereby providing a modular system for a series of chain extension reactions which can be used to flexibly prepare diverse macromolecules comprising polyethoidpeptides.

In a first preferred embodiment of the first general embodiment of the second aspect, the invention is directed to a method for preparing a compound comprising a polyethoid moiety. This method comprises synthesizing a polyethoid moiety having a formula II.B.0 or a polyethoid moiety having a formula II.B.6

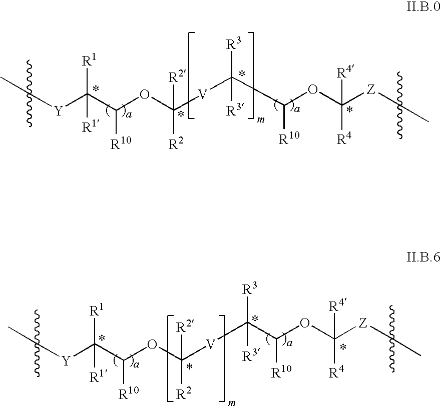

through a series of controlled stepwise reactions. The series of controlled reactions can comprise (i) a first addition reaction, (ii) a first transformation reaction, and (iii) a second addition reaction, in each case as described above in connection with the first general embodiment of the second aspect of the invention.

The following descriptors are generally applicable in this first general embodiment of the second aspect, and in preferred embodiments thereof (in each case unless otherwise noted). Each m is an integer ≧0, and each a is an independently selected integer=1 or =2. The symbol “*” denotes an optionally chiral carbon. R¹, each R², each R³, and R⁴ are each an independently selected side chain moiety comprising hydrocarbyl or substituted hydrocarbyl. Generally, R¹, each R², each R³, and R⁴ can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, each R²′, each R³′, and R⁴′, respectively) or with adjacent side chain moieties (e.g., R¹ with a nearest R²) or with an atom on the backbone of the polyethioid moiety (e.g, R² with an adjacent N atom in an embodiment where a V is an N-substituted methyleneamine isostere). Preferably, R¹, each R², each R³ and R⁴ can each be an independently selected side chain moiety having a structure of an amino acid side chain. R₁′, each R²′, R₃′ and R⁴′ are each independently selected from the group consisting of H, C₁-C₈ alkyl and substituted C₁-C₈ alkyl, and are preferably selected from H, C₁-C₃ alkyl and substituted C₁-C₃ alkyl. Each R¹⁰ is generally independently selected from hydrogen, hydrocarbyl or substituted hydrocarbyl; more preferably each R¹⁰ is independently selected from the group consisting of H, C₁-C₈ alkyl and substituted C₁-C₈ alkyl, even more preferably each R¹⁰ being independently selected from the group consisting of H, C₁-C₃ alkyl and substituted C₁-C₃ alkyl. In each case, R¹⁰ can optionally form one or more ring structures with adjacent side chain moieties or with an atom on the backbone of the polyethioid moiety. The polyethoid synthesized in this first embodiment of this aspect of the invention can optionally include one or more amide moieties and additionally or alternatively or one or more other isosteres in addition to ethoid isosteres. Hence, each V is independently selected from the group consisting of —C(O)NH— and -ψ[ ]-. Y and Z are each generally independently selected from the group consisting of H, hydrocarbyl and substituted hydrocarbyl. Y and Z can be, independently selected, linking moieties (e.g., connecting the depicted compound to another compound or to another moiety of the same compound) or terminal groups (e.g., a moiety representing the end terminals of the depicted compound, either as a final compound or as an intermediate compound (e.g., as a functional group or a protected functional group). In preferred embodiments, Y and Z can each be independently selected from the group consisting —V—, -functional group, -protected functional group, -linking moiety, -conjugate and -terminal group. Examples of Y and Z as linking moieties include linking moieties which connect the depicted polyethoid moiety to another polyethoid moiety, to a polypeptide moiety, to a polyethoidpeptide moiety, to a support (e.g., a solid support). In some embodiments, one or both of Y and Z can be terminal groups. For example, Y can be a terminal group selected from the group consisting of H—, H₂N—, AcNH—, R²⁰C(O)NH—, R²²OC(O)NH—, HO—, R²⁰O—, and protected derivatives thereof, R²⁰ and R²² each being independently selected from the group consisting of H, hydrocarbyl and substituted hydrocarbyl. For example, Z can be a terminal group selected from the group consisting of —H, —R²⁰OH, —C(O)OR²⁰, —C(O)H, —C(O)R²⁰, —R²⁰OR²², —C(O)NHR²⁰ and protected derivatives thereof, R²⁰ and R²² each being independently selected from the group consisting of H, hydrocarbyl and substituted hydrocarbyl.

In a second preferred embodiment of the first general embodiment of the second aspect, the invention is directed to a synthesis scheme involving a series of controlled stepwise reactions in which the compound of formula II.B.0 (above) is prepared. For context and reference purpose only, and without limitation, this scheme generally involves a Y→Z (e.g., left-to-right as depicted in formula II.B.O) synthesis approach, analogous to and integratable with known N→C synthesis approaches for polypeptide and protein synthesis. In this method, the polyethoid moiety having a formula II.B.0 is synthesized by a process comprising: (i) forming a compound II.B.3 comprising an ethoid and having a formula

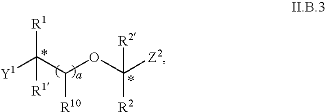

through one or more reactions including reacting a first chiral compound having a formula II.B.1 with a second chiral compound having a formula II.B.2,

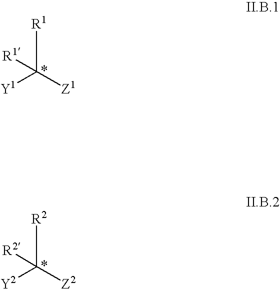

wherein: (a) Y₁ is selected from the group consisting of —V—, -functional group, -protected functional group, -linking moiety-, -conjugate, and -terminal group; (b) Z₁ is a functional group selected from —CHR¹⁰OH, —CH₂CHR¹⁰OH, —C(O)H, —C(O)R¹⁰, —CH₂C(O)H, —CH₂C(O)R¹⁰, —C(O)OH, —CH₂C(O)OH, (c) Y² is a functional group reactive with Z¹ and is selected from —X, —OH, —CH₂OH, —O-silyl, —CH₂O-silyl, —O⁻M⁺, and —CH₂O⁻M⁺, X is halogen, M is an alkali or alkaline earth and (d) Z² is a functional group or protected functional group. The method further comprises (ii) optionally forming a compound II.B.4 comprising an ethoid and one or more one or more —V— and having a formula

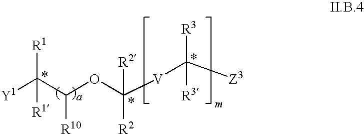

through one or more reactions, wherein Z³ is a functional group or protected functional group. The method further comprises (iii) converting (A) when m=0, the —Z² functional group or protected functional group of compound II.B.3 or (B) when m≧1, the —Z³ functional group or protected functional group of compound II.B.4, in each case to a —Z¹ functional group through one or more reactions, thereby forming a compound of formula II.B.5 having a formula

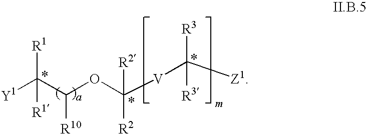

The method further comprises (iv) forming the compound comprising the polyethoid of formula II.B.0 through one or more reactions including reacting the compound of formula II.B.5 with the compound of formula II.B.8

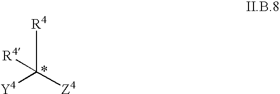

wherein (a) Y⁴ is a functional group reactive with Z¹ and is selected from —X, —OH, —CH₂OH, —O-silyl, —CH₂O-silyl, —O⁻M⁺, and —CH₂O⁻M⁺, X is halogen, M is a metal cation, and (b) Z⁴ is selected from the group consisting of —V—, -functional group, -protected functional group, -linking moiety-, -conjugate, and -terminal group.

In a third preferred embodiment of the first general embodiment of the second aspect, the invention is directed to a synthesis scheme involving a series of controlled stepwise reactions in which the compound of formula II.B.6 (see above, first general embodiment of second aspect) is prepared. For context and reference purpose only, and without limitation, this scheme generally involves a Z→Y (e.g., right-to-left as depicted in formula II.B.6) synthesis approach, analogous to and integratable with known C→N synthesis approaches for polypeptide and protein synthesis. In this method, the polyethoid moiety having a formula II.B.6 is synthesized by a process comprising: (i) forming a compound II.B.9 comprising an ethoid and having a formula

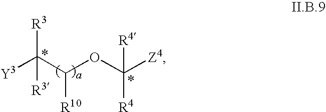

through one or more reactions including reacting a first chiral compound having a formula II.B.7 with a second chiral compound having a formula II.B.8

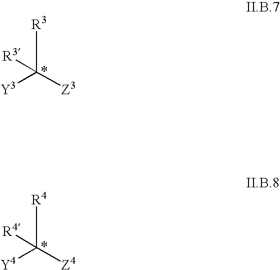

wherein (a) Y₃ is a functional group or protected functional group, (b) Z₃ is a functional group reactive with Y⁴ and is selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH, (c) Y⁴ is a functional group selected from —X, —OH, —CH₂OH, —O-silyl, —CH₂O-silyl, —O⁻M⁺, and —CH₂O⁻M⁺, X is halogen, M is an alkali or alkaline earth cation, and (c) Z⁴ is selected from the group consisting of —V—, -functional group, -protected functional group, -linking moiety-, -conjugate, and -terminal group. The method further comprises (ii) optionally forming a compound II.B.10 comprising an ethoid and one or more one or more —V— and having a formula

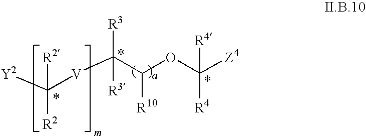

through one or more reactions, wherein Y² is a functional group or protected functional group. The method further comprises (iii) converting (A) when m=0, the —Y³ functional group or protected functional group of compound II.B.9 or (B) when m≧1, the —Y² functional group or protected functional group of compound II.B.10, in each case to a —Y⁴ functional group through one or more reactions, thereby forming a compound of formula II.B.11 having a formula

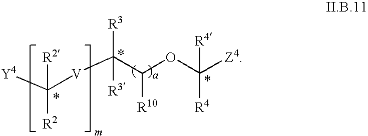

The method further comprises (iv) forming the compound comprising the polyethoid of formula II.B.6 through one or more reactions including reacting the compound of formula II.B.11 with the compound of formula II.B.1

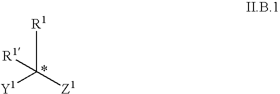

wherein (a) Y¹ is selected from the group consisting of —V—, -functional group, -protected functional group, -linking moiety-, -conjugate, and -terminal group, and (b) Z¹ is a functional group reactive with Y⁴ and is selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH.

In a second general embodiment of the second aspect, the invention is directed to a method for preparing a compound comprising a polyethoid, which method comprises synthesizing a polyethoid moiety on a support (e.g., a solid support) through a series of controlled stepwise reactions, and optionally cleaving the polyethoid moiety from the support. The series of controlled reactions can generally comprise (i) a first addition reaction, (ii) a first transformation reaction, and (iii) a second addition reaction, in each case as described above in connection with the first general embodiment of the second aspect of the invention.

In a first preferred embodiment of the second general embodiment of the second aspect, the invention is directed to a method for preparing a compound comprising a polyethoid, the method comprising synthesizing a polyethoid moiety having a formula

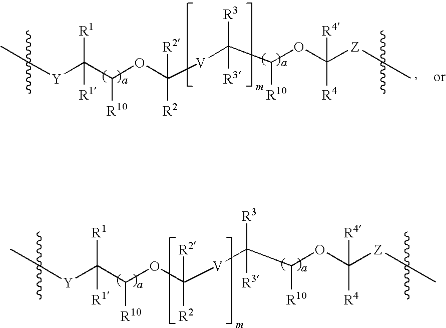

on a support through a series of controlled stepwise reactions, and optionally cleaving the polyethoid moiety from the support. In this embodiment, the integer m, the integer a, each R¹⁰, R¹, each R², each R³, and R⁴, R^{1′}, each R^{2′}, each R^{3′} and R^{4′}, each V, Y and Z are each as described above in connection with the first preferred embodiment of the first general embodiment of the second aspect of the invention (and are to be considered the same as if such were expressly reproduced in this paragraph); moreover, these are generally applicable in preferred embodiments of this second general embodiment of the second aspect (in each case unless otherwise noted).

In a second preferred embodiment of the second general embodiment of the second aspect, the invention is directed to a solid-phase synthesis scheme involving a series of controlled stepwise reactions in which, for context and reference purpose only, and without limitation, involving a Y→Z synthesis approach (e.g., left-to-right as depicted in the formula shown in the first preferred embodiment of second general embodiment of second aspect), analogous to and integratable with known N→C synthesis approaches for polypeptide and protein synthesis. In this method, a polyethoid moiety having a formula

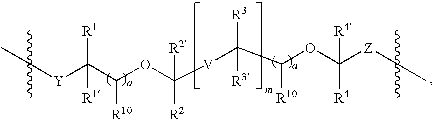

where Y is a linking moiety covalently bonded (directly or indirectly) to the support, with the linking moiety optionally comprising —V—, is synthesized by a process comprising (i) forming a first moiety comprising an ethoid and having a formula

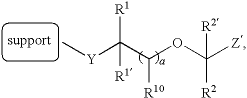

through one or more reactions, wherein Z′ is a functional group or a protected functional group. The method further comprises (ii) optionally forming a second moiety having a formula

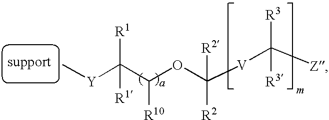

through one or more further reactions with the first moiety, wherein Z″ is a functional group or a protected functional group. The method further comprises (iii) forming a third moiety comprising at least two ethoids and having a formula

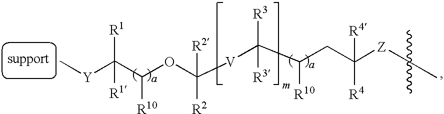

Through one or more further reactions with the second moiety when m≧1, or with the first moiety when m=0.

In a third preferred embodiment of the second general embodiment of the second aspect, the invention is directed to a solid-phase synthesis scheme involving a series of controlled stepwise reactions in which, for context and reference purpose only, and without limitation, involving a Z→Y synthesis approach (e.g., right to left as depicted in the formula shown in the first preferred embodiment of second general embodiment of second aspect), analogous to and integratable with known C→N synthesis approaches for polypeptide and protein synthesis. In this method, a polyethoid moiety having a formula

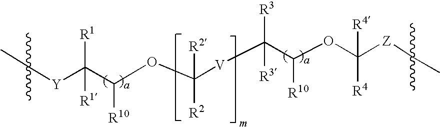

where Z is a linking group covalently bonded (directly or indirectly) to the support, the linking moiety optionally comprising —V—, is synthesized by a process comprising (i) forming a first moiety comprising an ethoid and having a formula

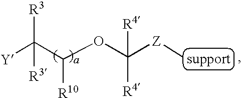

through one or more reactions, wherein Y′ is a functional group or a protected functional group. The process further comprises (ii) optionally forming a second moiety having a formula

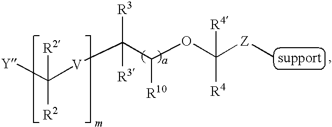

through one or more further reactions with the first moiety, wherein Y″ is a functional group or a protected functional group. The process further comprises (iii) forming a third moiety comprising at least two ethoids and having a formula

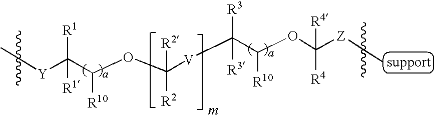

Through one or more further reactions with the second moiety when m≧1, or with the first moiety when m=0.

In a third general embodiment of the second aspect, the invention is directed to a method for preparing a compound, and preferably to a method for preparing a compound comprising an ethoid or a polyethoid. This method comprises reacting compounds of formulas III.B.2 and III.C.2

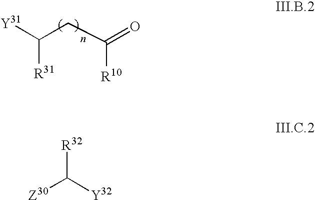

to prepare a compound of formula III.A

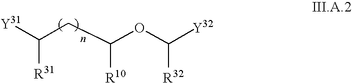

wherein R¹⁰ is a hydrogen, C₁-C₃ alkyl, or substituted alkyl, n is zero or one and R³¹ and R³² are ea wherein R¹⁰ is a hydrogen, C₁-C₃ alkyl, or substituted alkyl, n is an integer=zero or an integer=one, and R³¹ and R³² are each independently selected side chain moiety comprising hydrocarbyl or substituted hydrocarbyl. Generally, each R³¹ and R³² can be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with each R^{31′} and R^{32′}—each of which is not shown above but is optionally included in place of the opposing —H moiety pendant from the backbone carbone as depicted in III.A.2, III.B.2, and III.C.2. See formula III.A.1 by way of example.) or with adjacent side chain moieties (e.g., R³¹ and R³²) or with an atom on the backbone of the ethoid moiety. Preferably, each R³¹ and R³² can each be an independently selected side chain moieties have a structure of an amino acid side chain, and Y³¹ is a substituted nitrogen or oxygen, preferably as a hydroxyl, amine, amide, ether or ester, Y³² is a substituted or unsubstituted carbon, preferably as the carbonyl carbon of an amide or a methyleneoxy linkage. Y³¹ and Y³² can each optionally carry a polyethoid or polyaminoacid chain. Z³⁰ is typically a hydroxyl group, optionally as the alcohol, the alkoxide, or the silyl ether. The reaction of III.B.2 with III.C.2 to give III.A can occur under any conditions known to form an ether bond. When the reaction occurs in the present of a catalyst, provided to the reaction mixture as a compound, and a reducing agent, provided to the reaction mixture as the same or a different compound, the reaction of called a reductive etherification. In one embodiment, the catalyst that is provided to the reaction mixture can be a Lewis acid or a bronsted acid, preferably selected from a group consisting of BiX₃, FeX₃, CuX₂, TMSX, B(C₆F₅)₃, HX, XBi═O, Et₃SiX, or trityl perchlorate, preferably, BiBr₃, BiCl₃, FeCl₃, Cu(OS(O)CF₃)₂, Me₃SiO₃SCF₃, B(C₆F₅)₃, HBr, BrBi═O, Et₃SiI or Et₃SiBr. In another embodiment, the reducing agent provided to the reaction mixture is a silane, siloxane or silicon hydride source. This reductive etherification reaction is suitable for use under a variety of conditions, temperatures, and solvents, as a solution phase reaction and also in combination with chemistries conducted on a solid support.ch independently selected side chain moieties have a structure of an amino acid side chain, and Y³¹ is a substituted nitrogen or oxygen, preferably as a hydroxyl, amine, amide, ether or ester, Y³² is a substituted or unsubstituted carbon, preferably as the carbonyl carbon of an amide or a methyleneoxy linkage. Y³¹ and Y³² can each optionally carry a polyethoid or polyaminoacid chain. Z³⁰ is typically a hydroxyl group, optionally as the alcohol, the alkoxide, or the silyl ether. The reaction of III.B.2 with III.C.2 to give III.A can occur under any conditions known to form an ether bond. When the reaction occurs in the present of a catalyst, provided to the reaction mixture as a compound, and a reducing agent, provided to the reaction mixture as the same or a different compound, the reaction of called a reductive etherification. In one embodiment, the catalyst that is provided to the reaction mixture can be a Lewis acid or a bronsted acid, preferably selected from a group consisting of BiX₃, FeX₃, CuX₂, TMSX, B(C₆F₅)₃, HX, XBi═O, Et₃SiX, or trityl perchlorate, preferably, BiBr₃, BiCl₃, FeCl₃, Cu(OS(O)CF₃)₂, Me₃SiO₃SCF₃, B(C₆F₅)₃, HBr, BrBi═O, Et₃SiI or Et₃SiBr. In another embodiment, the reducing agent provided to the reaction mixture is a silane, siloxane or silicon hydride source. This reductive etherification reaction is suitable for use under a variety of conditions, temperatures, and solvents, as a solution phase reaction and also in combination with chemistries conducted on a solid support.

A number of general features, summarized below and described in further detail hereinafter, are specifically contemplated in combination with the second aspect of the invention, including in combination with each of the general embodiments of the second aspect, as well as each preferred embodiment of each of the general embodiments. Moreover, a number of sub-embodiments are contemplated as summarized above and further described hereinafter, in which various features as generally described are more specifically contemplated, independently and in various combinations.

### Methods for Using Ethoid-Containing Compounds

The ethoid-containing compounds of the invention can be used in various applications and in multiple industries.

In a third aspect, therefore, the invention is directed to methods for using compounds comprising an ethoid moiety or a polyethoid moiety. Generally, in all general and preferred embodiments of the third aspect of the invention (including all sub-embodiments thereof), the compound comprises an ethoid moiety or a polyethoid moiety as described in connection with the first aspect of the invention, including any general or preferred embodiments, as well as all sub-embodiments, thereof.

In a first general embodiment of the third aspect, the invention is directed to use of a compound comprising an ethoid or a polyethoid as a diagnostic agent. The diagnostic agent can be used in an assay such as an epitope in an assay comprising a monoclonal antibody.

In a second general embodiment of the third aspect, the invention is directed to use of a compound comprising an ethoid or a polyethoid as an imaging agent.

In a third general embodiment of the third aspect, the invention is directed to use of a compound comprising an ethoid or a polyethoid as an affinity reagent in affinity chromatography.

In a fourth general embodiment of the third aspect, the invention is directed to use of a compound comprising an ethoid or a polyethoid as a pharmaceutical.

In a fifth general embodiment of the third aspect, the invention is direct to use of a compound comprising an ethoid or a polyethoid as a food additive.

In a sixth general embodiment of the third aspect, the invention is directed to use of a compound comprising an ethoid or a polyethoid as a cosmetic ingredient.

In a seventh general embodiment of the third aspect, the invention is directed to use of a compound comprising an ethoid or a polyethoid as a research reagent.

### Ethoid Scanning

Ethoid-containing compounds, and especially ethoid-containing compounds of the first aspect of the invention can be advantageously used in methods to identify ethoid-containing polyaminoacid analogs, especially functional analogs such as analogs of polyaminoacid pharmaceuticals. Sets of ethoid-containing compounds, preferably including particular patterns of ethoid-isostere substitutions, can be especially advantageous to identify such ethoid-containing polyaminoacid analogs. Such sets of ethoid-containing compound can be advantageously prepared according to the methods of the second aspect of the invention.

In a fourth aspect, therefore, the invention is directed to methods for identifying ethoid-containing polyaminoacid analogs having a property of interest. In one example, the method is directed to identifying analogs of polyaminoacid pharmaceuticals. In this method the polyaminoacid comprises a structural moiety having three or more amino acid residues linked by amide moieties, and in some embodiments generally preferably five or more amino acid residues linked by amide moieties. This method comprises (i) providing a set of ethoid-containing compounds, the set comprising (a) a first compound comprising the structural moiety of the polyaminoacid with an ethoid isostere at a first sequence position, and (b) a second compound comprising the structural moiety of the polyaminoacid with an ethoid isostere at a second sequence position, the second sequence position being different from the first sequence position, each of the ethoid isosteres having a formula

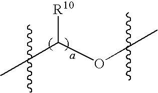

and being a substitutive replacement for an amide moiety within the structural moiety of the polyaminoacid. The method further comprises (ii) evaluating the first ethoid-containing compound and the second ethoid-containing compound for the property of interest.

In this fourth aspect, and in the below-described fifth, sixth and seventh aspects of the invention, generally, unless otherwise noted: a is an integer=1 or =2. Moreover, generally therein, each R¹⁰ is generally being independently selected from hydrogen, hydrocarbyl or substituted hydrocarbyl; more preferably each R¹⁰ is independently selected from the group consisting of H, C₁-C₈ alkyl and substituted C₁-C₈ alkyl, even more preferably each R¹⁰ being independently selected from the group consisting of H, C₁-C₃ alkyl and substituted C₁-C₃ alkyl. In each case, R¹⁰ can optionally form one or more ring structures with adjacent side chain moieties or with an atom on the backbone of the polyethioid moiety.

In a fifth aspect, the invention is directed to a data set derived from evaluating ethoid-containing compounds. The data set is stored on a tangible medium, and comprises data derived from evaluating a set of ethoid-containing analogs of a polyaminoacid for a property of interest, such as a polyaminoacid pharmaceutical. In this fifth aspect, the polyaminoacid comprises a structural moiety having three or more amino acid residues linked by amide moieties, and preferably in some embodiments five or more amino acid residues linked by amide moieties. The set of ethoid-containing analogs comprises (a) a first compound comprising the structural moiety of the polyaminoacid with an ethoid isostere at a first sequence position, and (b) a second compound comprising the structural moiety of the polyaminoacid with an ethoid isostere at a second sequence position, the second sequence position being different from the first sequence position, each of the ethoid isosteres having a formula

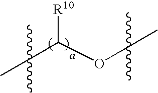

and being a substitutive replacement for an amide moiety within the structural moiety of the polyaminoacid. Each integer a and each R¹⁰ are as described above in connection with the fourth aspect of the invention.

In a sixth aspect, the invention is directed to methods for preparing a set of ethoid-containing compounds which are analogs of a polyaminoacid, such a polyaminoacid pharmaceutical of interest. In this method, the polyaminoacid comprises a structural moiety having three or more amino acid residues linked by amide moieties, and in some preferred embodiments, comprises five of more amino acids linked by amide moieties. This method comprises (i) obtaining an amino acid sequence identity for the structural moiety of the polyaminoacid, (ii) identifying a first amide moiety for isosteric replacement at a first sequence position within the structural moiety of the polyaminoacid, (iii) identifying a second amide moiety for isosteric replacement at a second sequence position within the structural moiety of the polyaminoacid, the second sequence position being different from the first sequence position, (iv) forming a first compound comprising the structural moiety of the polyaminoacid with an ethoid isostere at the first sequence position, and (v) forming a second compound comprising the structural moiety of the polyaminoacid with an ethoid isostere at a second sequence position. In this method, each of the ethoid isosteres has a formula

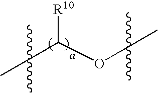

and is a substitutive replacement for the identified amide moiety of the polyaminoacid. Each integer a and each R¹⁰ are as described above in connection with the fourth aspect of the invention.

In a seventh aspect, the invention is directed to a set of ethoid-containing compounds which are ethoid-containing polyaminoacid analogs, preferably analogs of polyaminoacid pharmaceuticals. In this method, the polyaminoacid comprises a structural moiety having three or more amino acid residues linked by amide moieties, and in some embodiments, preferably five or more amino acids linked by amide moieties. The set comprises (a) a first compound comprising the structural moiety of the polyaminoacid with at least one ethoid isostere at a first sequence position, and (b) a second compound comprising the structural moiety of the polyaminoacid with at least one ethoid isostere at a second sequence position, the second sequence position being different from the first sequence position. Each of the ethoid isosteres has a formula

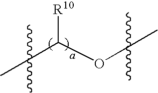

and is a substitutive replacement for an amide moiety of the polyaminoacid. Each integer a and each R¹⁰ are as described above in connection with the fourth aspect of the invention.

### General Features

Various features of the inventions, including features defining each of the various aspects of the invention, including general and preferred embodiments thereof, can be used in various combinations and permutations with other features of the invention. In particular, the following features are considered general features of the invention, and are expressly contemplated to be used in each possible combination and permutation with each of the aspects of the invention and with each general embodiment and preferred embodiments thereof: side chain moieties (scope and structure of various side chain moieties, generally referred to as one or more of R⁰, R¹, R², R³, R⁴, R⁵, in the various formulas, as applicable); further isosteres ψ[ ] (scope and structure of various isosteres which can be effected in combination with one or more ethoid isosteres, or in some embodiments two or more ethoid isosteres, etc.); Y group substituents, Z group substituents; chirality (number of chiral centers in an ethoid-containing moiety); chirality (extent of chirality for each chiral center, typically expressed as extent of enantiomeric excess); extent of ethoid isosteric substitution (expressed typically as a ratio of the number of ethoid moieties to the number of amide moieties, for example, in a partially-ethoid-substituted ethoid-peptide compound); size (or length) of macromolecule (e.g., number of side-chain-containing (amino-acid) residues linked by ethoid or amide or other isostere); property of interest, especially biological properties such as improved resistance to enzymatic digestion; structural diversity (e.g., of side chain moieties) of the macromolecule; chiral modular synthesis methods; supported synthesis methods; reductive etherification synthesis methods; uses of any of the compounds or methods described herein; and data sets derived from compounds or methods or any uses thereof. Each of such features are described in greater detail herein.

Additional features and advantages are described herein, and will be apparent from, the following Detailed Description.

### DETAILED DESCRIPTION

Novel compounds are disclosed that comprise one or more ethoid moieties. In generally preferred embodiments, ethoid-containing compounds of the invention comprise two or more ethoid moieties, or three or more ethoid moieties. Such ethoid-containing compounds are preferably structural analogs of polyaminoacids, such as proteins or polypeptides, in which the one or more ethoid moieties are isosteres for a corresponding one or more amide moieties of the polyaminoacid. Advantageously, as described more fully below, ethoid-containing compounds can have the same chemical structure as polyaminoacids with respect to pendant side chain groups (the amino-acid residues), thereby allowing for conservation of side-chain functionality. Significantly, however, the ethoid-containing compounds have a different chemical structure than polyaminoacids with respect to the portion of the backbone chain connecting adjacent amino-acid residues—namely where amide moieties of the polyaminoacids have been all, or partially replaced with ethoid moieties such as methyleneoxy moieties. This structural difference in the linkages between successive amino acid residues provides for significant biological and chemical advantages, including enhanced protease resistance and improved chemical stability. For the purposes of this disclosure, an ethoid moiety refers to a moiety that comprises an ether bond—the carbon-oxygen bond defined by a substituted or unsubstituted methyleneoxy linkage. Preferred ethoid moieties are described more fully herein. The ethoid moiety (alternatively optionally referred to in this application and in the priority application as an ethoid bond) can link two monomers to form an analog of a polyaminoacid polymer in which the ethoid moiety (for example, Ψ[CH₂O]) is an isosteric replacement of an amide moiety. Ethoid moieties are also contemplated in other contexts, for example in intramolecular bridges or to link other moieties to polyaminoacids or other analogs of polyaminoacid polymers.

As a conceptual example, and without limitation as to scope of the invention, a representative structure of some compounds of this disclosure can be represented schematically by Scheme 1A. With reference to Scheme 1A, one or more isosteres can be a substitutive replacement for an amide moiety of a polyaminoacid (labeled as a “peptide” in Scheme 1A). The ethoid-containing compounds (labeled as “non-peptide structures” in Scheme 1A) can generally comprise a structural moiety of a polyaminoacid having one or more ethoid isosteres (e.g, Ψ[CH₂O] as shown in Scheme 1A) at a corresponding one or more sequence positions, each as a substitutive replacement for an amide moiety. The ethoid isosteres link amino acid residues (depicted as rectangles between amide moieties or between isosteres, each rectangle having a pendant side chain labeled as “—R” in Scheme 1A). For the purposes of this disclosure, an ethoid is an ethoid-containing compound comprising one or more ethoid moieties, preferably as isosteres. Ethoids which are polyaminoacid analogs can comprise an amino acid residue (derived from monomer units such as alpha-amino acids or derivatives thereof) linked by an ethoid moiety such as ψ[CH₂O] (e.g. methyleneoxy). Ethoids can include an ethoidpeptide (e.g., a partially-ethoid-substituted polyaminoacid moiety including one or more ethoid moieties as isosteres, and additionally comprising one or more amide moieties). (In this application and in the priority application, ethoidpeptide compounds are alternatively referred to as mixed ethoid-peptides).

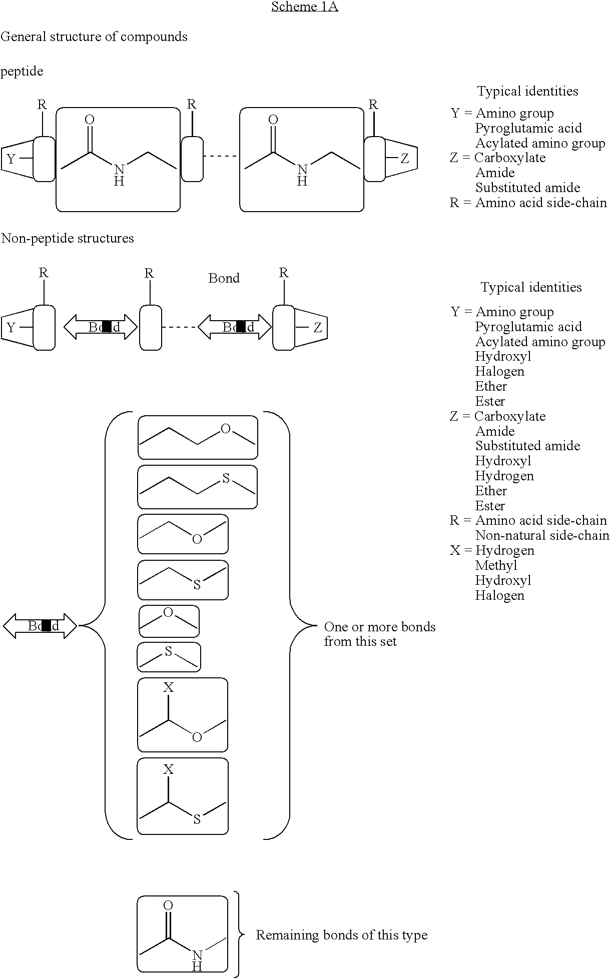

Similarly, ethoids can include a polyethoid. A polyethoid is a compound which includes two or more ethoid moieties, or in some embodiments three or more ethoid moieties, preferably as isosteres. A polyethoid can be a polyethoidpeptide. A polyethoidpeptide compound (or a polyethiodpeptide moiety) comprises a moiety which includes two or more ethoid moieties, or in some embodiments three or more ethoid moieties, and in each case additionally comprising one or more amide moieties). An ethoidpeptide can also be a polyethoidpeptide. A polyethoid compound can also include a fully-ethoid-substituted moiety (e.g., a moiety including two or more ethoid moieties, or in some embodiments three or more ethoid moieties, and in each case to the exclusion of amide moieties. In a fully-ethoid-substituted moiety, the amide moieties have been substitutively replaced by ethoid isosteres, alone or in combination with other isosteres, ψ[ ], including for example other isosteres depicted in Scheme 1A.

Significantly, ethoid-containing compounds demonstrate biological activity—as partially-ethoid-substituted compounds as well as fully-ethoid-substituted compounds. For example, and without limitation, Example 20, demonstrates that various ethoid analogs of the polypeptide LHRH agonists are active, including ethoid analogs having a single ethoid isostere and an ethoid analog in which ethoid isosteres substitutively replace each amide moiety of the LHRH agonists. Other ethoid analogs which are fully-ethoid-substituted also have biological activity, including for example, fully-ethoid-substituted analogs of Bombesin (see Example 27), CCK-8 (see Example 28), Substance P (see Example 24), Neurokinin B (see Example 26), Neurokinin A (see Example 25), [Leu]enkephalin (see Example 29) and [Met]enkephalin (see Example 29). Other ethoid analogs which are partially-ethoid-substituted likewise demonstrate biological activity, including for example, partially-ethoid-substituted analogs of GLP-1 (see Example 21), GHRH (see Example 18) and PTH (see Example 22). Moreover, ethoid-containing compounds comprising non-natural amino acid residues linked by ethoid moieties are shown to have biological activity. (See, for example, Example 16 involving LHRH agonists for which fully-ethoid-substituted analogs were evaluated with D2Nal as a non-natural amino acid analog for glycine).

It is likewise significant that various ethoid compounds have improved biological stability, including stability to protease activity such as DPP-IV protease. For example and without limitation, ethoid-containing GHRH analogs have demonstrated protease stability against DPP-IV. (See Example 9). The ethoid-containing analog for LHRH has demonstrated stability to multiple proteases (Example 10).

### Ethoid Isosteres

As noted above, an ethoid moiety is generally a moiety that comprises an ether bond—the carbon-oxygen bond defined by a substituted or unsubstituted methyleneoxy linkage. In generally preferred aspects and embodiments of the invention, an ethoid moiety can be a substituted or unsubstituted methyleneoxy. In some aspects and embodiments, an ethoid moiety can be a substituted or unsubstituted ethyleneoxy moiety (also referred in this application and in the priority application as a homoethoid). Hence, in generally preferred aspects and embodiments, an ethoid moiety can have a formula

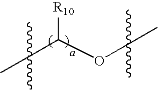

In this formula, a is an integer=1 or an integer=2. R¹⁰ is generally selected from hydrogen, hydrocarbyl or substituted hydrocarbyl. More preferably R¹⁰ can be selected from the group consisting of H, C₁-C₈ alkyl and substituted C₁-C₈ alkyl. In even more preferred embodiments of the various general embodiments and aspects of the invention, R¹⁰ can be selected from the group consisting of H, C₁-C₃ alkyl and substituted C₁-C₃ alkyl. In each case, R¹⁰ can optionally form one or more ring structures with adjacent side chain moieties or with an atom on the backbone of the polyethioid moiety.

As described above, any of such ethoid moieties can be isosteres in an analog of a polyaminoacid. For discussion (and as a non-limiting example), a compound comprising an ethoid moiety can have a formula

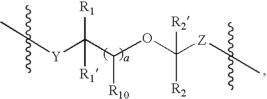

in which the integer a, and R¹⁰ are as described above in the immediately preceding paragraph in connection with the ethiod moiety. R¹ and R² each represent independently selected side chain moiety comprising hydrocarbyl or substituted hydrocarbyl. Generally, R¹, and R² can each be side chain moieties which are each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, R²′, respectively) or with adjacent side chain moieties (e.g., R¹ with R²) or with an atom on the backbone of the polyethioid moiety (e.g, R¹ with an adjacent N included within Y). Preferably, R¹ and R² can each be an independently selected side chain groups (or moieties) pendant from carbon atoms which are part of a predominantly carbon backbone chain. The carbon atoms from which such side chain moieties are pendant can be, and often are, chiral carbons. These side chain moieties are each independently selected side chain moiety and generally can have a structure which is the same structure as an amino acid side chain (including natural amino acid side chains, and non-natural amino acid side chains), including in protected or unprotected forms; preferred side chain moieties are more fully described herein. The respective opposing side chain moieties, R^{1′} and R^{2′}, are not narrowly critical, and can be independently selected from hydrocarbyl or substituted hydrocarbyl. In generally preferred embodiments, these moieties are each independently selected from the group consisting of H, C₁-C₃ alkyl and substituted C₁-C₃ alkyl. Y and Z are likewise not narrowly critical, and can generally be hydrocarbyl or substituted hydrocarbyl, and are more fully described herein. In the immediately-precending formula, the portion of the ethoid-containing compound which includes the pendant side chain group R¹ (or R²) and the respective opposing side chain groups R^{1′} (or R^{2′}) together with the backbone carbon atom from which they are pendant, is generally referred to herein as an amino acid residue. Such reference to amino acid residue derives from the synthesis protocol for polyaminoacids involving sequential condensation of alpha amino acids (or derivatives thereof), typically provided as alpha amino acid monomers. Such nomenclature is adopted herein in connection with ethoid-containing compounds. Without limitation, such nomenclature is apt in view of preferred synthesis schemes (discussed herein) for preparing ethoid-containing compounds that likewise involve amino acids, preferably alpha amino acids or derivatives thereof. Hence, as used in this application (and in the priority application), the term “monomer” in the context of synthesis of ethoid-containing compounds refers to a building block, unit or moiety that can be linked into a linear sequence, through one or more reactions, with formation of an ethoid moiety. Monomers can include, for example and without limitation, amino acids, amino aldehydes, amino ketones, α-hydroxy acids, α-hydroxy aldehydes, α-hydroxy ketones, α-halo (e.g., bromo) acids, α-halo (e.g., bromo) aldehydes, α-halo (e.g., bromo) ketones and protected derivatives thereof. Monomers can include side chain groups, and in some embodiments can include a single sidechain R group (e.g, R¹ or R² as depicted in the immediately-preceding formula) that can be alkyl or aryl, branched, linear or cyclical, and contain zero or more functional groups, including alcohol, ether, carboxylic acid, thiol, thioether, amide, phenol, heterocycle, aryl or alkyl carbocycle, and can further include protecting groups thereof, with the other side chain group being hydrogen. In cases where the monomer sidechains correspond to amino acid side chains or protected versions thereof, they can be described by Rₗₑₜₜₑᵣ, where the subscript letter corresponds to the amino acid with analogous sidechain component, or by three letter codes commonly assigned to the amino acid, or by description as corresponding in structure to that of an amino acid side chain moiety, or an amino acid residue.

### Side Chain Moieties

Side chain moieties (e.g., R¹ and R² as shown in the immediately-preceding formula) are generally independently selected side chain moieties and generally can be an independently selected side chain moiety comprising hydrocarbyl or substituted hydrocarbyl. Generally, such side chain moieties can be each independently selected from the group consisting of H, C₁-C₁₀ alkyl and substituted C₁-C₁₀ alkyl, and which in each case can optionally form one or more ring structures, for example with respective opposing side chain moieties (e.g., with R¹′, and R²′) or with adjacent side chain moieties (e.g., R¹ with R²) or with an atom on the backbone of the polyethioid moiety. Preferably, each R¹ and R² can each have an independently selected structure which is the same structure as an amino acid side chain (including natural amino acid side chains, and non-natural amino acid side chains), including in protected or unprotected forms. A natural alpha amino acid (or side chain thereof) (or natural polyaminoacid comprising the same) refers to an alpha amino acid (or polyaminoacid) which occurs in nature; notably, however, physical quantities of the natural amino acid (or side chain) (or polyaminoacid) can be from a natural source or a synthetic (man made) source. Conversely, a non-natural alpha amino acid (or side chain thereof) (or non-natural polyaminoacid) refers to an alpha amino acid (or polyaminoacid) which does not occur in nature; physical quantities of such amino acid (or polyaminoacid or side chain) are synthetic (man made). A list of abbreviations for natural amino acid side chains as used in this application, as well as the corresponding natural amino acid from which they are known, are set forth in Table I.A.

A list of abbreviations for natural amino acid side chains as used in this application are set forth in Table I.A.

TABLE I.A

| R | Side chain | Parent amino acid |
|---|---|---|
| R^{A} | —CH₃ | Alanine |
| R^{C} | —CH₂SH | Cysteine |
| R^{D} | —CH₂COOH | Aspartate |
| R^{E} | —CH₂CH₂COOH | Glutamate |
| R^{F} | —CH₂C₆H₅ | Phenylalanine |
| R^{G} | —H | Glycine |
| R^{H} | —CH₂—C₃H₃N₂ | Histidine |
| R^{I} | —CH(CH₃)CH₂CH₃ | Isoleucine |
| R^{K} | —(CH₂)₄NH₂ | Lysine |
| R^{L} | —CH₂CH(CH₃)₂ | Leucine |
| R^{M} | —CH₂CH₂SCH₃ | Methionine |
| R^{N} | —CH₂CONH₂ | Asparagine |
| R^{P} | —CH₂CH₂CH₂— | Proline |
| R^{Q} | —CH₂CH₂CONH₂ | Glutamine |
| R^{R} | CH₂)₃NH—C(NH)NH₂ | Arginine |
| R^{S} | —CH₂OH | Serine |
| R^{T} | —CH(OH)CH₃ | Threonine |
| R^{U} | —CH₂SeH | Selenocysteine |
| R^{V} | —CH(CH₃)₂ | Valine |
| R^{W} | —CH₂C₈H₆N | Tryptophan |
| R^{Y} | —CH₂—C₆H₄OH | Tyrosine |

Side chains from nonnatural amino acids can also be used. Non-natural amino acids are non-participants in genetically encoded protein synthesis; however, such amino acid side chains/residues are commonly used in the industry of protein synthesis and protein analogs to prepare peptides that contain replacements in a natural amino acid sequence. The side chains of these non-natural amino acid can be any chemical structure known to be used in synthetic peptides including, but is not limited to, the structures listed in Table I.B.1 and Table I.B.2. Non-natural amino acid residues may also be used as an isosteric substitute for a proline residue. Generally, for example, a ψ[ ] for use as a proline analog can generally be a C₃ to C₁₂ hydrocarbyl or substituted hydrocarbyl comprising a ring structure such as a five-member ring. Examples of such proline analogs can include those set forth in Table I.C.1 (where as shown in the table, the side chain moiety is understood to be derived from the monomer as listed, or from a derivative thereof), and Table I.C.2 wherein the group acting as a substitute would correspond to one or two residues, can also be used in the disclosure. Furthermore, other non-natural amino acids in which the C_{α} is di-substitute can be used in the disclosure, an exemplary set of which is set forth in Table I.D. In Tables I.B.1 and I.C.1, the three letter abbreviation for an amino acid side chain is included if one is commonly known.

TABLE I.B.1

| | |
|---|---|
| —CH₂CH═CH₂, | —(CH₂)₄NMe₂, |
| -(2-indanyl), | —(CH₂)₄NMe₃⁺Cl⁻, |
| -(2-thiazoyl), | —(CH₂)₅CH₃, |
| -(2-thienyl), | —(CH₂)₅COOH, |
| -(3-thienyl), | -4-(1-benzyl)imidazolyl, |
| —(CH₂)₂CONHCH₂CH₃, | —C(CH₃)₂S(CH₂)₂-(₄-pyridyl), |
| —(CH₂)₂NH₂, | —C(CH₃)₂SCH₂-(4-methoxyphenyl), |
| —(CH₂)₂OBzl, | —C(CH₃)₂SCH₂-(4-methylphenyl), |
| —(CH₂)₂Ph, | —C(CH₃)₂SCH₂NHCOCH₃, |
| —(CH₂)₂S(═O)(═NH)((CH₂)₃CH₃), | —C(CH₃)₂SH, |
| —(CH₂)₂S(CH₂)₃CH₃, | —C(CH₃)₃, |
| —(CH₂)₂S(CH₃)₂⁺Cl⁻, | —C(OCH₃)CH₃, |
| —(CH₂)₂SCH₂CH₃, | —C(OH)(CH₃)₂, |
| —(CH₂)₂SCH₂Ph, | —C(OH)CH₂CH₃, |
| —(CH₂)₂SeCH₃, | —CFMe₂, |
| —(CH₂)₃CH₃ (Nle) | —CH(CH₃)(COOH), |
| —(CH₂)₃COOH, | —CH(CH₃)CH₂COOH, |
| —(CH₂)₃NH₂, (Orn) | —CH(CH₃)OCH₂CH₃, |
| —(CH₂)₃NHC(═NH)NHNO₂, | —CH(CH₃)OCH₃, |
| —(CH₂)₃NHCONH₂, (Cit) | —CH(CH₃)Ph, |
| —(CH₂)4CH₃, | —CH(Me)(CF₃), |
| —(CH₂)₄COOH, | —CH(OH)CH(CH₃)₂, |
| —(CH₂)₄N₃, | —CH(OH)CH(CH₃)CH₂CH═CHCH₃, |
| —(CH₂)₄NHC(═NEt)NHEt, | —CH(OH)CH(OH)Ph, |
| —CH(OH)CH₂CH₂NH₂, | —CH₂-(3-(6-chloroindolyl)), |
| —CH(OH)Ph, | —CH₂-(3-(6-fluoroindolyl)), |
| —CH(Ph)₂, (Dif) | —CH₂-(3-(6-methylindolyl)), |
| —CH═CCl₂, | —CH₂-(3-(7-benzyloxyindolyl)), |
| —CH₂-(1-naphthyl), | —CH₂-(3-(7-bromoindolyl)), |
| —CH₂-(1-pyrenyl), | —CH₂-(3-(7-methylindolyl)), |
| —CH₂-(2,4-dichlorophenyl), | —CH₂-(3,4,5-trifluorophenyl), |
| —CH₂-(2,4-dihydroxyphenyl), | —CH₂-(3,4-dichlorophenyl), |
| —CH₂-(2-bromophenyl), | —CH₂-(3,4-difluorophenyl), |
| —CH₂-(2-chlorophenyl), | —CH₂-(3,4-dimethoxyphenyl), |
| —CH₂-(2-cyanophenyl), | —CH₂-(3,5-diiodo-4-hydroxy-phenyl), |
| —CH₂-(2-fluorophenyl), | —CH₂-(3-amino-4-hydroxy-phenyl), |
| —CH₂-(2-guanidinophenyl), | —CH₂-(3-benzothienyl), |
| —CH₂-(2-hydroxyphenyl), | —CH₂-(3-bromophenyl), |
| —CH₂-(2-methylphenyl), | —CH₂-(3-chlorophenyl), |
| —CH₂-(2-naphthyl), | —CH₂-(3-cyanophenyl), |
| —CH₂-(2-nitrophenyl), | —CH₂-(3-fluorophenyl), |
| —CH₂-(2-pyridyl), | —CH₂-(3-guanidinophenyl), |
| —CH₂-(2-pyridyl), | —CH₂-(3-hydroxyphenyl), |
| —CH₂-(2-thienyl), (Thi) | —CH₂-(3-iodophenyl), |
| —CH₂-(2-trifluoromethyl-phenyl), | —CH₂-(3-methoxy-4-hydroxy-phenyl), |
| —CH₂-(3-(1-methylindolyl)), | —CH₂-(3-methylphenyl), |
| —CH₂-(3-(1-phenylthiocarbamate- | —CH₂-(3-nitro-4-hydroxy-phenyl), |
| indolyl)), | —CH₂-(3-nitrophenyl), |
| —CH₂-(3-(4-methylindolyl)), | —CH₂-(3-pyridyl), |
| —CH₂-(₃-(₅-benzyloxyindolyl)), | —CH₂-(3-styryl), |
| —CH₂-(₃-(₅-bromoindolyl)), | —CH₂-(3-trifluoromethyl-phenyl), |
| —CH₂-(3-(5-chloroindolyl)), | —CH2-(4-(4-hydroxyphenoxy)-phenyl), |
| —CH₂-(3-(5-fluoroindolyl)), | —CH₂-(4-(bis-2- |
| —CH₂-(3-(5-hydroxyindolyl)), | chloroethyl)aminophenyl), |
| —CH₂-(3-(5-methoxy-2-methyl- | —CH₂-(4-aminomethyl-phenyl), |
| indolyl)), | —CH₂-(4-aminophenyl), |
| —CH₂-(3-(6-bromoindolyl)), | —CH₂-(4-benzoylphenyl) (Bpa, |
| —CH₂-(4-bromophenyl), | —CH₂CN, |
| —CH₂-(4-chlorophenyl), | —CH₂CO-(2-amino-phenyl), |
| —CH₂-(4-cyanophenyl), | —CH₂-cyclohexyl (Cha) |
| —CH₂-(4-ethoxyphenyl), | —CH₂-cyclopentyl, |
| —CH₂-(4-fluorophenyl), | —CH₂-cyclopropyl, |
| —CH₂-(4-guanidinophenyl), | —CH₂NH₂, |
| —CH₂-(4-iodophenyl), | —CH₂NHC(═NH)NH₂, |
| —CH₂-(4-methoxyphenyl), | —CH₂NHCONH₂, |
| —CH₂-(4-methylphenyl), | —CH₂O-octanoyl, |
| —CH₂-(4-nitrophenyl), | —CH₂S(CH₂)₂-(4-pyridyl), |
| —CH₂-(4-pyridyl), | —CH₂S(CH₂)₂COOH, |
| —CH₂-(4-sulfoxy-phenyl), | —CH₂S(CH₂)₂OH, |
| —CH₂-(4-trifluoromethyl-phenyl), | —CH₂S(CH₂)₃NH₂, |
| —CH₂-(5-bromo-2-methoxyphenyl), | —CH₂SC(CH₃)₃, |
| —CH₂-(cyclopenten-1-yl), | —CH₂SCH(Ph)₂, |
| —CH₂-(pentafluorophenyl), | —CH₂SCH₂CH₃, |
| —CH₂C(═CH₂)(COOH), | —CH₂SCH₂COOH, |
| —CH₂CCH, | —CH₂SCH₂Ph, |
| —CH₂CF₃, | —CH₂SCH₃, |
| —CH₂CH(CH₃)(CF₃), | —CH₂SCONH₂, |
| —CH₂CH(COOH)₂, | —CH₂S-farnesyl, |
| —CH₂CH₂CH₃, | —CH₂^{t}Bu. |
| —CH₂CH₂-cyclohexyl, | |

TABLE I.B.2

| | |
|---|---|
| -Ph | —CH₂OBzl |
| -cyclohexyl | —CH₂O^{t}Bu |
| —CH₂SO₃H | —CH₂OCH₂CH₃ |
| —CH₂CH₃ | —CH₂N(CH₃)₂ |
| —CH₂Cl | -(2,4-dinitro)phenyl |
| —CH₂OCH₃ | -(2,5-dihydro)phenyl |
| -(2-bromo)phenyl | |
| -(2-fluoro)phenyl | |
| -(2-methoxy)phenyl | |
| -(2-methyl)phenyl | |
| -(4-fluoro)phenyl | |
| -(4-hydroxy)phenyl | |
| -cyclopentyl | |

TABLE I.C.1*

1,2,3,4-tetrahydroisoquino-line-3-carboxylic acid (Tic)
7-hydroxy-1,2,3,4-tetrahydroisoquinoline-3-carboxylic acid
1,2,3,4-tetrahydro-norharman-3-carboxylic acid
6-methoxy-1,2,3,4-tetrahydronorharman-1-carboxylic acid
4-hydroxypyrrolidine-2-carboxylic acid
Octahydro-1H-indole-2-carboxylic acid (Oic)
5-phenyl-pyrrolidine-2-carboxylic acid
4-benzyl-pyrrolidine-2-carboxylic acid
3-phenylpyrrolidine-2-carboxylic acid
3-hydroxypyrrolidine-2-carboxylic acid
4-phenyl-pyrrolidine-2-carboxylic acid
2-ethylthiazolidine-4-carboxylic acid
2-methylthiazolidine-4-carboxylic acid
2-phenylthiazolidine-4-carboxylic acid
5,5-dimethylthiazolidine-4-carboxylic acid
thiazolidine-2-carboxylic acid
azetidine-2-carboxylic acid
thiazolidine-4-carboxylic acid
3,4-dehydro-proline
4-Fluoro-proline
*Side chains are derived from the monomers listed above (see the claim)
Remove fmoc

TABLE I.C.2

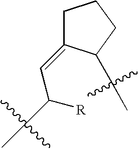

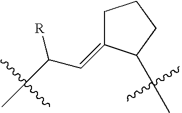

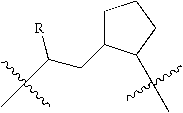

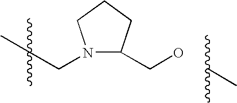

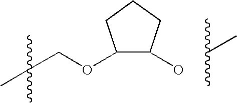

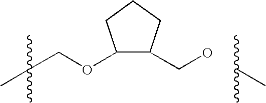

TABLE I.D

| Rⁿ | R^{n,} |
|---|---|
| —CH₂CH═CH₂ | Me |
| —CH₃ | Me |
| —CH₂CH(CH₃)₂ | Me |
| —(CH2)₃NH₂ | Me |
| —CH₂COOH | Me |
| —CH₂CH₂COOH | Me |
| —CH₂CH₂SCH₃ | Me |
| —CH₂-(3-methoxyphenyl) | Me |
| —CH₂Ph | Me |
| —CH₂OH | Me |
| —CH₂C₈H₆N | Me |

As specific examples not inconsistent with the general description above, the terms “conventional” and “naturally occurring” as applied to polyaminoacids such as polypeptides refer to polypeptides (also referred to as proteins) constructed from naturally-occurring amino acids: Ala, Cys, Asp, Glu, Phe, Gly, His, Ile, Lys, Leu, Met, Asn, Pro, Gln, Arg, Ser, Thr, Val, Trp, and Tyr and other less common but still naturally occurring amino acids. A compound of the disclosure “corresponds” to a natural peptide if it has a biological activity characteristic of or associated with the biological activity of the natural protein. The biological activity can be the same as, greater than or less than that of the natural protein and can provide an agonistic or antagonistic effect. Such a compound can have an essentially corresponding monomer sequence, where a natural amino acid is replaced by a monomer that resembles the original amino acid in hydrophilicity, hydrophobicity, polarity, etc. The correspondence need not be exact. Thus, for the following set of theoretical peptide sequences (Ia, IIa, Ma), the associated polyethoid (Ib, IIb, Mb) would be considered “corresponding”:

Ia: ABCDEFGH

Ib: (ABΨ[CH₂O]CDΨ[CH₂O]EFGH)

IIa: ABCDEFGHI

IIb: (ABCDΨ[CH₂O]EΨ[CH₂O]EGΨ[CH₂O]HI)

IIIa: ABCDEFGHIJKLM

IIIb: (ABΨ[CH₂O]CDΨ[CH₂O]EFGΨ[CH₂O]HIΨ[CH₂O]JKΨ[CH₂O] LM)

Furthermore, the moieties or units making up the backbone need not be exact, but could include a homo-peptide structure with an additional CH₂ in the backbone, a des peptide wherein a CH₂ is removed from the backbone, or a substituted peptide, wherein a CH₂ or CHR has been replaced with a CHR′ or CRR′. For example, a homo-ethoid bond, ψ[CH₂CH₂O] could be in a backbone.

Stability to Enzymatic Digestion

As noted above, the ethoid-containing compounds preferably can be biological stability in their environment characteristic of intended use, including being stabile to enzymatic digestion. Moreover, such compounds can have improved biological stability, such as to enzymatic digestion, relative to a corresponding polyaminoacid without the ethoid isosteres, in an environment characteristic of intended use For example, the compound can have improved resistance to protease enzymes or peptidases. Such biological stability can be more fully characterized with respect to particular classes of enzymes or particular classes enzyme-containing biological fluids, or correspondingly, with respect to particular enzymes or enzyme-containing biological fluids.

Generally, two peptidases are classified in one of two sets of sub-subclasses of peptidases, those of the exopeptidases (EC 3.4.11-19) and those of the endopeptidases (EC 3.4.21-24 and EC 3.4.99). (See Nomenclature Committee of the International Union of Biochemistry and Molecular Biology, http://www.chem.qmul.ac.uk/iubmb/enzyme/EC34/index.html.) The exopeptidases act only near the ends of polypeptide chains, and those acting at a free N-terminus liberate a single amino-acid residue (aminopeptidases, EC 3.4.11), or a dipeptide or a tripeptide (dipeptidyl-peptidases and tripeptidyl-peptidases, EC 3.4.14). The exopeptidases acting at a free C-terminus liberate a single residue (carboxypeptidases, EC 3.4.16-18) or a dipeptide (peptidyl-dipeptidases, EC 3.4.15). The carboxypeptidases are allocated to four groups on the basis of catalytic mechanism: the serine-type carboxypeptidases (EC 3.4.16), the metallocarboxypeptidases (EC 3.4.17) and the cysteine-type carboxypeptidases (EC 3.4.18). Other exopeptidases are specific for dipeptides (dipeptidases, EC 3.4.13), or remove terminal residues that are substituted, cyclized or linked by isopeptide bonds (peptide linkages other than those of α-carboxyl to α-amino groups) (omega peptidases, EC 3.4.19). The endopeptidases are divided into sub-subclasses on the basis of catalytic mechanism, and specificity is used only to identify individual enzymes within the groups. These are the sub-subclasses of serine endopeptidases (EC 3.4.21), cysteine endopeptidases (EC 3.4.22), aspartic endopeptidases (EC 3.4.23), metalloendopeptidases (EC 3.4.24) and threonine endopeptidases (EC 3.4.25). Endopeptidases that could not be assigned to any of the sub-subclasses EC 3.4.21-25 were listed in sub-subclass EC 3.4.99.

In preferred embodiments, an ethoid-containing compound can be resistant to, or show improved resistance (as compared to corresponding polyaminoacid) to one or more of enzyme selected from Table II.

TABLE II
Protease or Peptidase Enzymes

EC 3.4.11.1 leucyl aminopeptidase
EC 3.4.11.2 membrane alanyl aminopeptidase
EC 3.4.11.3 cystinyl aminopeptidase
EC 3.4.11.4 tripeptide aminopeptidase
EC 3.4.11.5 prolyl aminopeptidase
EC 3.4.11.6 aminopeptidase B
EC 3.4.11.7 glutamyl aminopeptidase
EC 3.4.11.9 Xaa-Pro aminopeptidase
EC 3.4.11.10 bacterial leucyl aminopeptidase
EC 3.4.11.13 clostridial aminopeptidase
EC 3.4.11.14 cytosol alanyl aminopeptidase
EC 3.4.11.15 aminopeptidase Y
EC 3.4.11.16 Xaa-Trp aminopeptidase
EC 3.4.11.17 tryptophanyl aminopeptidase
EC 3.4.11.18 methionyl aminopeptidase
EC 3.4.11.19 D-stereospecific aminopeptidase
EC 3.4.11.20 aminopeptidase Ey
EC 3.4.11.21 aspartyl aminopeptidase
EC 3.4.11.22 aminopeptidase I
EC 3.4.11.23 pepB aminopeptidase
EC 3.4.14.1 dipeptidyl-peptidase I
EC 3.4.14.2 dipeptidyl-peptidase II
EC 3.4.14.4 dipeptidyl-peptidase III
EC 3.4.14.5 dipeptidyl-peptidase IV
EC 3.4.14.6 dipeptidyl-dipeptidase
EC 3.4.14.9 tripeptidyl-peptidase I
EC 3.4.14.10 tripeptidyl-peptidase II
EC 3.4.14.11 Xaa-Pro dipeptidyl-peptidase
EC 3.4.14.12 prolyltripeptidyl aminopeptidase
EC 3.4.15.1 peptidyl-dipeptidase A
EC 3.4.15.4 peptidyl-dipeptidase B
EC 3.4.15.5 peptidyl-dipeptidase Dcp
EC 3.4.16.2 lysosomal Pro-Xaa carboxypeptidase
EC 3.4.16.4 serine-type D-Ala-D-Ala carboxypeptidase
EC 3.4.16.5 carboxypeptidase C
EC 3.4.16.6 carboxypeptidase D
EC 3.4.17.1 carboxypeptidase A
EC 3.4.17.2 carboxypeptidase B
EC 3.4.17.3 lysine carboxypeptidase
EC 3.4.17.4 Gly-Xaa carboxypeptidase
EC 3.4.17.6 alanine carboxypeptidase
EC 3.4.17.8 muramoylpentapeptide carboxypeptidase
EC 3.4.17.10 carboxypeptidase E
EC 3.4.17.11 glutamate carboxypeptidase
EC 3.4.17.12 carboxypeptidase M
EC 3.4.17.13 muramoyltetrapeptide carboxypeptidase
EC 3.4.17.14 zinc D-Ala-D-Ala carboxypeptidase
EC 3.4.17.15 carboxypeptidase A2
EC 3.4.17.16 membrane Pro-Xaa carboxypeptidase
EC 3.4.17.17 tubulinyl-Tyr carboxypeptidase
EC 3.4.17.18 carboxypeptidase T
EC 3.4.17.19 carboxypeptidase Taq
EC 3.4.17.20 carboxypeptidase U
EC 3.4.17.21 glutamate carboxypeptidase II
EC 3.4.17.22 metallocarboxypeptidase D
EC 3.4.18.1 Cathepsin X
EC 3.4.13.3 Xaa-His dipeptidase
EC 3.4.13.4 Xaa-Arg dipeptidase
EC 3.4.13.5 Xaa-Methyl-His dipeptidase
EC 3.4.13.7 Glu-Glu dipeptidase
EC 3.4.13.9 Xaa-Pro dipeptidase
EC 3.4.13.12 Met-Xaa dipeptidase
EC 3.4.13.17 non-stereospecific dipeptidase
EC 3.4.13.18 cytosol nonspecific dipeptidase
EC 3.4.13.19 membrane dipeptidase
EC 3.4.13.20 β-Ala-His dipeptidase
EC 3.4.13.21 dipeptidase E
EC 3.4.13.22 D-Ala-D-Ala dipeptidase
EC 3.4.19.1 acylaminoacyl-peptidase
EC 3.4.19.2 peptidyl-glycinamidase
EC 3.4.19.3 pyroglutamyl-peptidase I
EC 3.4.19.5 β-aspartyl-peptidase
EC 3.4.19.6 pyroglutamyl-peptidase II
EC 3.4.19.7 N-formylmethionyl-peptidase
EC 3.4.19.9 γ-glutamyl hydrolase
EC 3.4.19.11 γ-D-glutamyl-meso-diaminopimelate peptidase
EC 3.4.19.12 ubiquitinyl hydrolase 1
EC 3.4.21.1 chymotrypsin
EC 3.4.21.2 chymotrypsin C
EC 3.4.21.3 metridin
EC 3.4.21.4 trypsin
EC 3.4.21.5 thrombin
EC 3.4.21.6 coagulation factor Xa
EC 3.4.21.7 plasmin
EC 3.4.21.9 enteropeptidase
EC 3.4.21.10 acrosin
EC 3.4.21.12 α-lytic endopeptidase
EC 3.4.21.19 glutamyl endopeptidase
EC 3.4.21.20 cathepsin G
EC 3.4.21.21 coagulation factor VIIa
EC 3.4.21.22 coagulation factor IXa
EC 3.4.21.25 cucumisin
EC 3.4.21.26 prolyl oligopeptidase
EC 3.4.21.27 coagulation factor XIa
EC 3.4.21.32 brachyurin
EC 3.4.21.34 plasma kallikrein
EC 3.4.21.35 tissue kallikrein
EC 3.4.21.36 pancreatic elastase
EC 3.4.21.37 leukocyte elastase
EC 3.4.21.38 coagulation factor XIIa
EC 3.4.21.39 chymase
EC 3.4.21.41 complement subcomponent C ^̅{̅l̅r̅}̅
EC 3.4.21.42 complement subcomponent C ^̅{̅l̅s̅}̅
EC 3.4.21.43 classical-complement-pathway C3/C5 convertase
EC 3.4.21.45 complement factor I
EC 3.4.21.46 complement factor D
EC 3.4.21.47 alternative-complement-pathway C3/C5 convertase
EC 3.4.21.48 cerevisin
EC 3.4.21.49 hypodermin C
EC 3.4.21.50 lysyl endopeptidase
EC 3.4.21.53 endopeptidase La
EC 3.4.21.54 γ-renin
EC 3.4.21.55 venombin AB
EC 3.4.21.57 leucyl endopeptidase
EC 3.4.21.59 tryptase
EC 3.4.21.60 scutelarin
EC 3.4.21.61 kexin
EC 3.4.21.62 subtilisin
EC 3.4.21.63 oryzin
EC 3.4.21.64 peptidase K
EC 3.4.21.65 thermomycolin
EC 3.4.21.66 thermitase
EC 3.4.21.67 endopeptidase So
EC 3.4.21.68 t-plasminogen activator
EC 3.4.21.69 protein C (activated)
EC 3.4.21.70 pancreatic endopeptidase E
EC 3.4.21.71 pancreatic elastase II
EC 3.4.21.72 IgA-specific serine endopeptidase
EC 3.4.21.73 u-plasminogen activator
EC 3.4.21.74 venombin A
EC 3.4.21.75 furin
EC 3.4.21.76 myeloblastin
EC 3.4.21.77 semenogelase
EC 3.4.21.78 granzyme A
EC 3.4.21.79 granzyme B
EC 3.4.21.80 streptogrisin A
EC 3.4.21.81 streptogrisin B
EC 3.4.21.82 glutamyl endopeptidase II
EC 3.4.21.83 oligopeptidase B
EC 3.4.21.84 limulus clotting factor C̅
EC 3.4.21.85 limulus clotting factor B̅
EC 3.4.21.86 limulus clotting enzyme
EC 3.4.21.87 omptin
EC 3.4.21.88 repressor LexA
EC 3.4.21.89 signal peptidase I
EC 3.4.21.90 togavirin
EC 3.4.21.91 flavivirin
EC 3.4.21.92 endopeptidase Clp
EC 3.4.21.93 proprotein convertase 1
EC 3.4.21.94 proprotein convertase 2
EC 3.4.21.95 snake venom factor V activator
EC 3.4.21.96 lactocepin
EC 3.4.21.97 assemblin
EC 3.4.21.98 hepacivirin
EC 3.4.21.99 spermosin
EC 3.4.21.100 pseudomonalisin
EC 3.4.21.101 xanthomonalisin
EC 3.4.21.102 C-terminal processing peptidase
EC 3.4.21.103 physarolisin
EC 3.4.21.104 mannan-binding lectin-associated serine protease-2
EC 3.4.21.105 rhomboid protease
EC 3.4.21.106 hepsin
EC 3.4.21.107 peptidase Do
EC 3.4.21.108 HtrA2 peptidase
EC 3.4.21.109 matriptase
EC 3.4.21.110 C5a peptidase
EC 3.4.21.111 aqualysin 1
EC 3.4.21.112 site-1 protease
EC 3.4.21.113 pestivirus NS3 polyprotein peptidase
EC 3.4.21.114 equine arterivirus serine peptidase
EC 3.4.21.115 infectious pancreatic necrosis birnavirus Vp4 peptidase
EC 3.4.21.116 SpoIVB peptidase
EC 3.4.21.117 stratum corneum chymotryptic enzyme
EC 3.4.21.118 kallikrein 8
EC 3.4.21.119 kallikrein 13
EC 3.4.22.1 cathepsin B
EC 3.4.22.2 papain
EC 3.4.22.3 ficain
EC 3.4.22.6 chymopapain
EC 3.4.22.7 asclepain
EC 3.4.22.8 clostripain
EC 3.4.22.10 streptopain
EC 3.4.22.14 actinidain
EC 3.4.22.15 cathepsin L
EC 3.4.22.16 cathepsin H
EC 3.4.22.24 cathepsin T
EC 3.4.22.25 glycyl endopeptidase
EC 3.4.22.26 cancer procoagulant
EC 3.4.22.27 cathepsin S
EC 3.4.22.28 picornain 3C
EC 3.4.22.29 picornain 2A
EC 3.4.22.30 caricain
EC 3.4.22.31 ananain
EC 3.4.22.32 stem bromelain
EC 3.4.22.33 fruit bromelain
EC 3.4.22.34 legumain
EC 3.4.22.35 histolysain
EC 3.4.22.36 caspase-1
EC 3.4.22.37 gingipain R
EC 3.4.22.38 cathepsin K
EC 3.4.22.39 adenain
EC 3.4.22.40 bleomycin hydrolase
EC 3.4.22.41 cathepsin F
EC 3.4.22.42 cathepsin O
EC 3.4.22.43 cathepsin V
EC 3.4.22.44 nuclear-inclusion-a endopeptidase
EC 3.4.22.45 helper-component proteinase
EC 3.4.22.46 L-peptidase
EC 3.4.22.47 gingipain K
EC 3.4.22.48 staphopain
EC 3.4.22.49 separase
EC 3.4.22.50 V-cath endopeptidase
EC 3.4.22.51 cruzipain
EC 3.4.22.52 calpain-1
EC 3.4.22.53 calpain-2
EC 3.4.23.1 pepsin A
EC 3.4.23.2 pepsin B
EC 3.4.23.3 gastricsin
EC 3.4.23.4 chymosin
EC 3.4.23.5 cathepsin D
EC 3.4.23.12 nepenthesin
EC 3.4.23.15 renin
EC 3.4.23.16 HIV-1 retropepsin
EC 3.4.23.17 Pro-opiomelanocortin converting enzyme
EC 3.4.23.18 aspergillopepsin I
EC 3.4.23.19 aspergillopepsin II
EC 3.4.23.20 penicillopepsin
EC 3.4.23.21 rhizopuspepsin
EC 3.4.23.22 endothiapepsin
EC 3.4.23.23 mucorpepsin
EC 3.4.23.24 candidapepsin
EC 3.4.23.25 saccharopepsin
EC 3.4.23.26 rhodotorulapepsin
EC 3.4.23.28 acrocylindropepsin
EC 3.4.23.29 polyporopepsin
EC 3.4.23.30 pycnoporopepsin
EC 3.4.23.31 scytalidopepsin A
EC 3.4.23.32 scytalidopepsin B
EC 3.4.23.34 cathepsin E
EC 3.4.23.35 barrierpepsin
EC 3.4.23.36 signal peptidase II
EC 3.4.23.38 plasmepsin I
EC 3.4.23.39 plasmepsin II
EC 3.4.23.40 phytepsin
EC 3.4.23.41 yapsin 1
EC 3.4.23.42 thermopsin
EC 3.4.23.43 prepilin peptidase
EC 3.4.23.44 nodavirus endopeptidase
EC 3.4.23.45 memapsin 1
EC 3.4.23.46 memapsin 2
EC 3.4.23.47 HIV-2 retropepsin
EC 3.4.23.48 plasminogen activator Pla
EC 3.4.23.49 omptin
EC 3.4.24.1 atrolysin A
EC 3.4.24.3 microbial collagenase
EC 3.4.24.6 leucolysin
EC 3.4.24.7 interstitial collagenase
EC 3.4.24.11 neprilysin
EC 3.4.24.12 envelysin
EC 3.4.24.13 IgA-specific metalloendopeptidase
EC 3.4.24.14 procollagen N-endopeptidase
EC 3.4.24.15 thimet oligopeptidase
EC 3.4.24.16 neurolysin
EC 3.4.24.17 stromelysin 1
EC 3.4.24.18 meprin A
EC 3.4.24.19 procollagen C-endopeptidase
EC 3.4.24.20 peptidyl-Lys metalloendopeptidase
EC 3.4.24.21 astacin
EC 3.4.24.22 stromelysin 2
EC 3.4.24.23 matrilysin
EC 3.4.24.24 gelatinase A
EC 3.4.24.25 vibriolysin
EC 3.4.24.26 pseudolysin
EC 3.4.24.27 thermolysin
EC 3.4.24.28 bacillolysin
EC 3.4.24.29 aureolysin
EC 3.4.24.30 coccolysin
EC 3.4.24.31 mycolysin
EC 3.4.24.32 β-lytic metalloendopeptidase
EC 3.4.24.33 peptidyl-Asp metalloendopeptidase
EC 3.4.24.34 neutrophil collagenase
EC 3.4.24.35 gelatinase B
EC 3.4.24.36 leishmanolysin
EC 3.4.24.37 saccharolysin
EC 3.4.24.38 gametolysin
EC 3.4.24.39 deuterolysin
EC 3.4.24.40 serralysin
EC 3.4.24.41 atrolysin B
EC 3.4.24.42 atrolysin C
EC 3.4.24.43 atroxase
EC 3.4.24.44 atrolysin E
EC 3.4.24.45 atrolysin F
EC 3.4.24.46 adamalysin
EC 3.4.24.47 horrilysin
EC 3.4.24.48 ruberlysin
EC 3.4.24.49 bothropasin
EC 3.4.24.50 bothrolysin
EC 3.4.24.51 ophiolysin
EC 3.4.24.52 trimerelysin I
EC 3.4.24.53 trimerelysin II
EC 3.4.24.54 mucrolysin
EC 3.4.24.55 pitrilysin
EC 3.4.24.56 insulysin
EC 3.4.24.57 O-sialoglycoprotein endopeptidase
EC 3.4.24.58 russellysin
EC 3.4.24.59 mitochondrial intermediate peptidase
EC 3.4.24.60 dactylysin
EC 3.4.24.61 nardilysin
EC 3.4.24.62 magnolysin
EC 3.4.24.63 meprin B
EC 3.4.24.64 mitochondrial processing peptidase
EC 3.4.24.65 macrophage elastase
EC 3.4.24.66 choriolysin L
EC 3.4.24.67 choriolysin H
EC 3.4.24.68 tentoxilysin
EC 3.4.24.69 bontoxilysin
EC 3.4.24.70 oligopeptidase A
EC 3.4.24.71 endothelin-converting enzyme
EC 3.4.24.72 fibrolase
EC 3.4.24.73 jararhagin
EC 3.4.24.74 fragilysin
EC 3.4.24.75 lysostaphin
EC 3.4.24.76 flavastacin
EC 3.4.24.77 snapalysin
EC 3.4.24.78 gpr endopeptidase
EC 3.4.24.79 pappalysin-1
EC 3.4.24.80 membrane-type matrix metalloproteinase-1
EC 3.4.24.81 ADAM10 endopeptidase
EC 3.4.24.82 ADAMTS-4 endopeptidase
EC 3.4.24.83 anthrax lethal factor endopeptidase
EC 3.4.24.84 Ste24 endopeptidase
EC 3.4.24.85 S2P endopeptidase
EC 3.4.24.86 ADAM 17 endopeptidase
EC 3.4.25.1 proteasome endopeptidase complex

In an embodiment the polyethoid comprising the improvement is biologically active. It can be have an increased resistance to a peptidase or protease

### Synthesis Approaches—General Schema

In alternative preferred embodiments, an ethoid-containing compound can be resistant to, or show improved resistance (as compared to corresponding polyaminoacid) to one or more biological fluids, preferably selected from the group consisting of: gastric milieu; plasma; serum; human liver microsomes; human hepatocytes; intestinal microsomes; intestinal homogenates; S9 fractions; cell culture extracts; cell culture homogenates; artificial immobilized membranes; lipid preparations including monolayers, bilayers, and vesicles; expressed enzymes; purified enzymes; cell fractions including microsomal fractions; cultured cells; transwell cell culture preparations for permeability; hepatocytes; liver slices; intestinal slices; tissue preparations; and perfused organs.

Construction of the ethoid bonds in the linkage can be achieved via several routes. In one embodiment, the ethoid bonds can be created in a stepwise fashion, thereby allowing a modular chemical approach to selection and incorporation of monomers, sidechains, and the amide bond or ethoid bond. Furthermore, because each residue added to the growing chain maintains a chirality, i.e., an enantiomeric excess, at its chiral center in each step, sequences of more than 3 residues containing ethoid bonds can be constructed in a stereocontrolled manner. The maintenance of chirality is a key advantage for the polyethoids prepared in this disclosure.

In one embodiment, the stepwise use of a α-halo acid, e.g. α-bromo acid, by addition of a hydroxyl, creates an ethoid bond. The acid-terminal group can be reduced to a terminal alcohol, and another α-halo acid can be added. Repetition of this addition-reduction sequence provides for modular synthesis of a full ethoid. Scheme 1 provides a demonstration of this stepwise process to give a polyethoid with six residues R₁ to R₆.

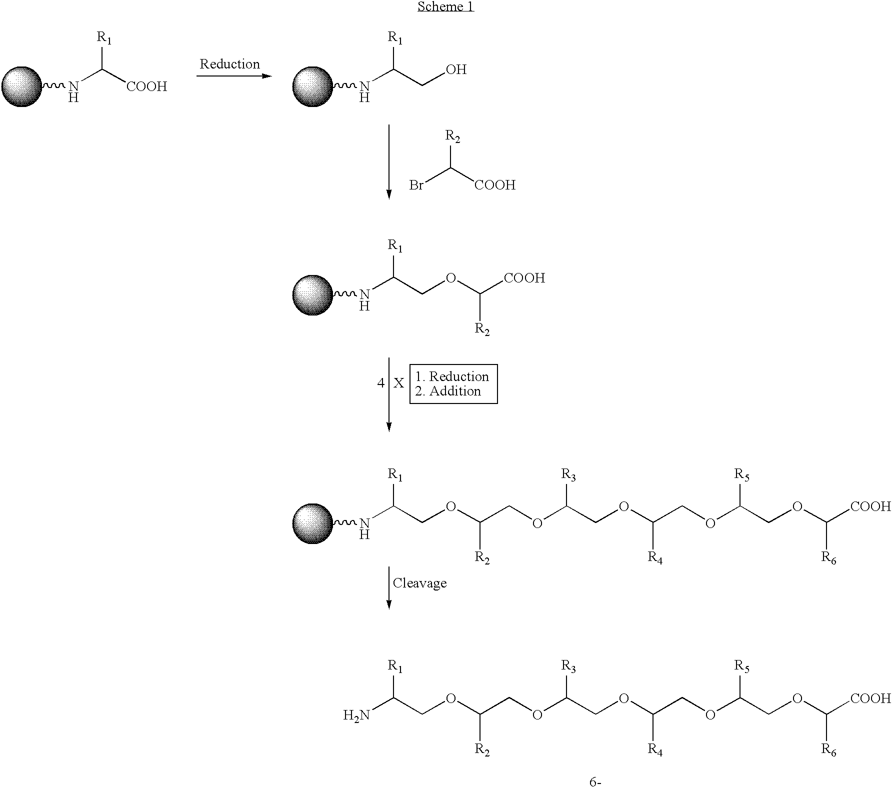

In another embodiment, control of the end groups and head groups of the polyethoid can be achieved. In Scheme 2, the end group can be a hydroxyl rather than an acid. Alternatively, in Scheme 3, the head group could be a hydroxyl rather than an amine. One of skill in the art would appreciate that both groups can by hydroxyl as well.

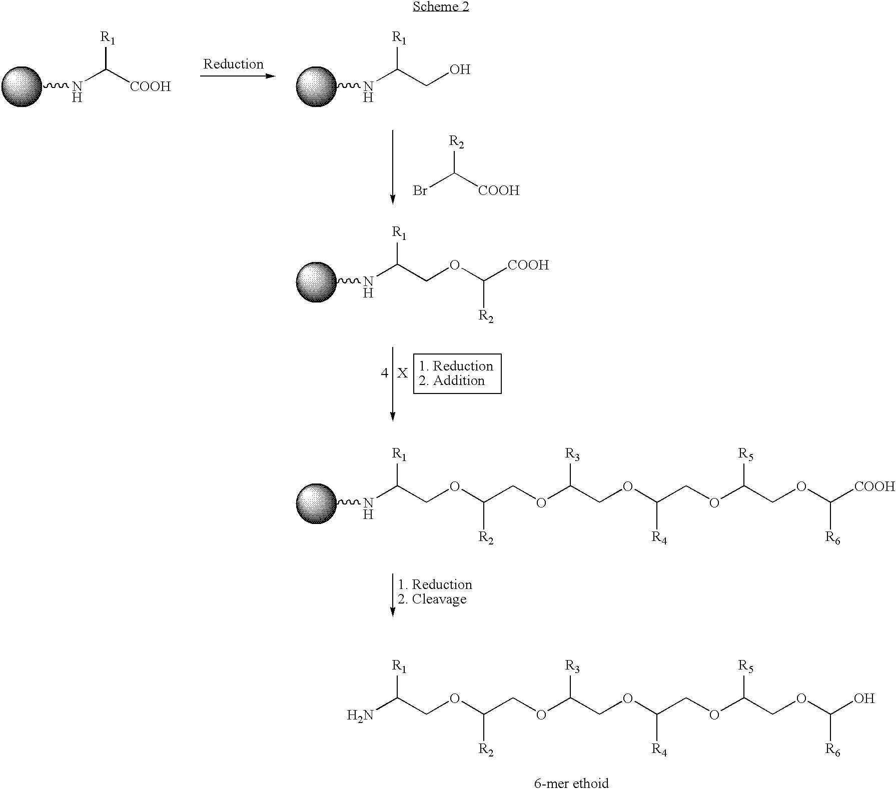

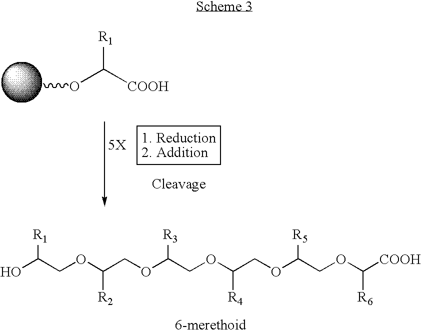

In one embodiment, the growing polyethoid compound can be prepared modularly from left to right, as shown in the schemes above. In an alternative embodiment, the polyethoid compound can be prepared from right to left, as shown for example in Scheme 4. The terminal amine of a growing polymer chain can be converted to, for example a halide, and treated with an alcohol-amine to synthesize an ethoid bond. Repeated deprotection, bromination and addition produces the polyethoid. Alternatively, the amine group can be converted to a hydroxyl and reacted with an incoming haloamine to synthesize the ethoid bond. Repeated deprotection, hydroxylation, and addition produces the polyethoid. Conversion of the amine to a bromide, i.e. bromination, or to a hydroxyl, i.e. hydroxylation, can be achieved using the chemistry described in PCT/US2007/008221.

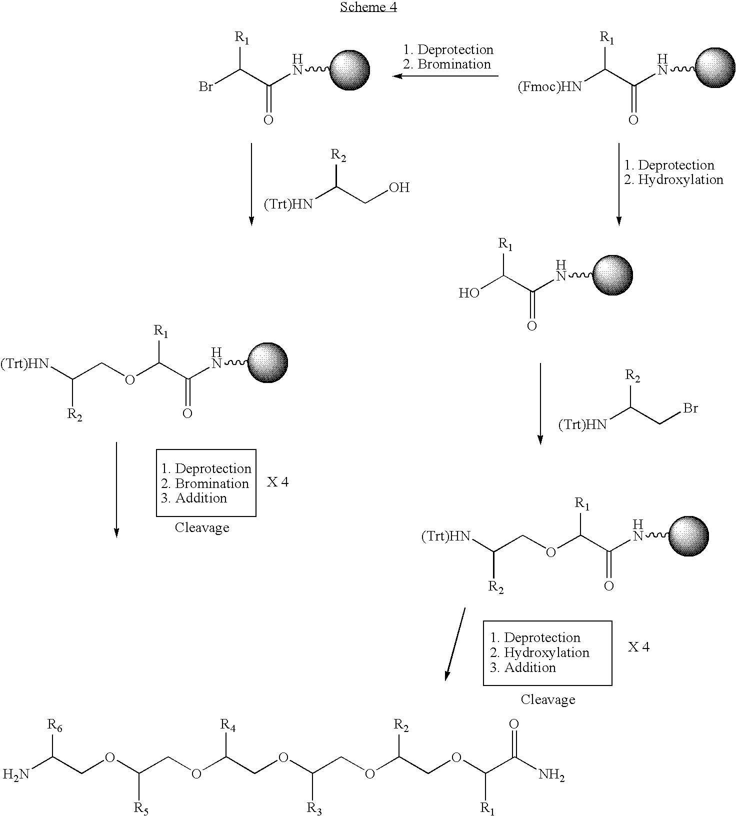

Alternatively, preparation of the ethoid bond can be achieved by preparing the ester first, then reducing the ester to an ether, as shown in Scheme 5

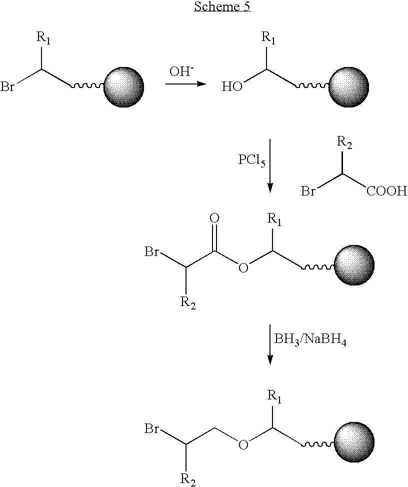

In an alternate embodiment, synthesis of the ethoid bond can be achieved using thio ethers as a monomer that is added in the modular synthesis, as shown in scheme 6. Addition to the thio ether, reaction with an acid anhydride and hydrolysis gives an aldehyde which can be reduced to a terminal alcohol, which is available for the next stepwise coupling.

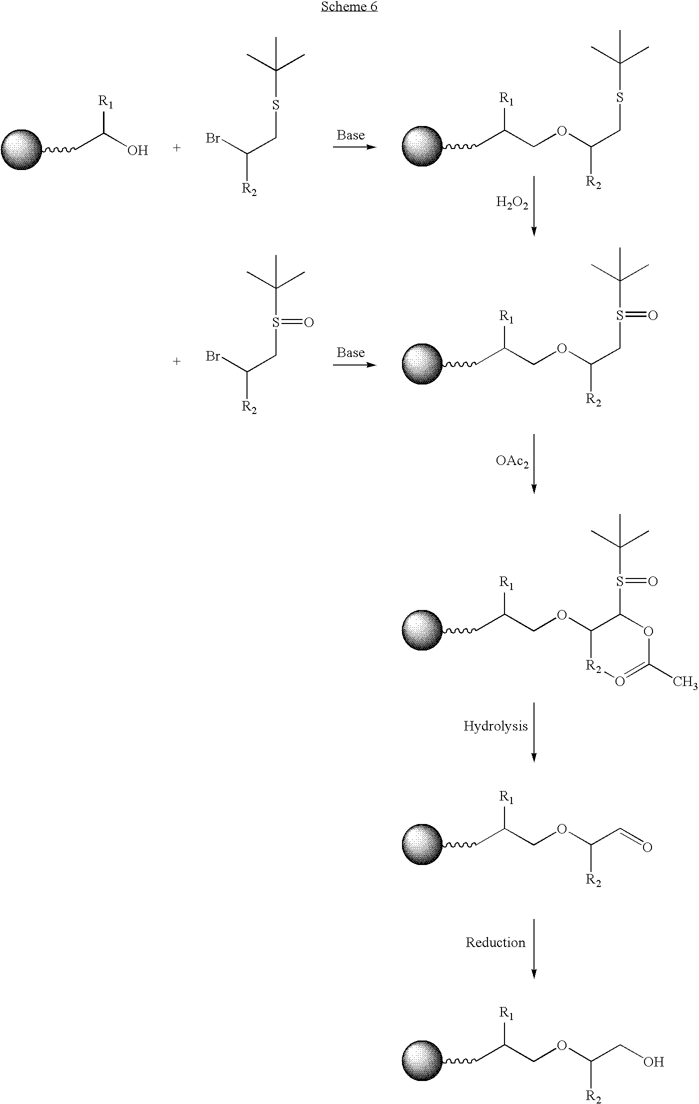

Using the chemistries set forth above, a polyethoidpeptide can be constructed in a modular fashion, alternating traditional amide synthesis for the ethoid bond syntheses. Scheme 7 is presented as an example of a six residue polyethoidpeptide, e.g. a mixed ethoid-peptide.

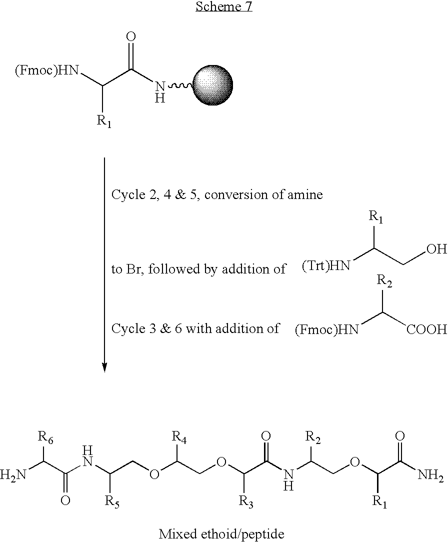

Des-ethoid and homo-ethoid compound are also accessible using the chemistries described above, as shown in Scheme 8 and Scheme 9

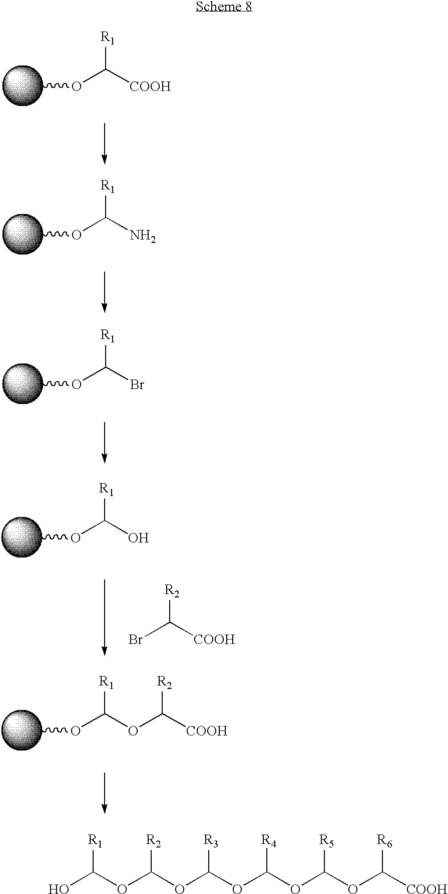

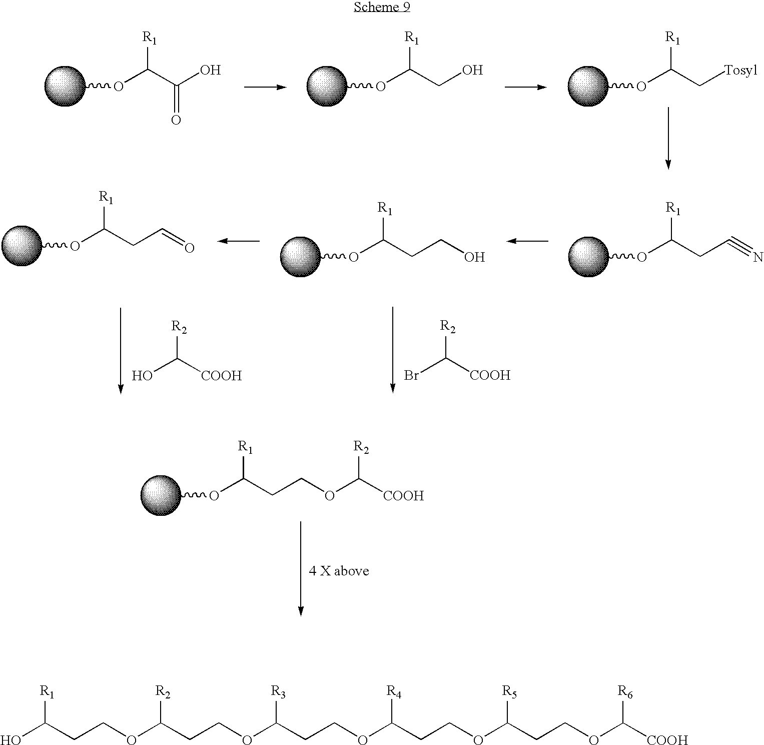

### Polyethoid Compounds

A number of the general features are further described herein in the context of Formula I.A, or in the context of other formula, in the following paragraphs. Although described in the specific context of such Formula I.A or other formula, such features are expressly contemplated as being generally applicable to any compound of the first aspect of the invention (including any general embodiment or preferred embodiment thereof, or any subembodiment of the foregoing), as being generally applicable to any method of preparing ethoid-containing compounds of the second aspect of the invention (including any general embodiment or preferred embodiment thereof, or any subembodiment of the foregoing), as being generally applicable to any methods for use of the ethoid-containing compounds (including any general embodiment or preferred embodiment thereof, or any subembodiment of the foregoing), and as being generally applicable to each of the fourth through seventh aspects of the invention (including any general embodiment or preferred embodiment thereof, or any subembodiment of the foregoing).

Novel compounds are provided according to the general formula I.A

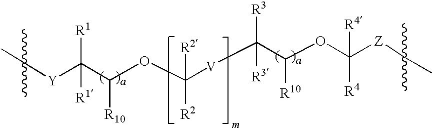

wherein:

m is an integer ≧0;

each a is an independently selected integer=1 or =2;

R¹, each R² and R⁴ are each (i) an independently selected side chain moiety selected from the group consisting of H, C₁-C₁₀ alkyl and substituted C₁-C₁₀ alkyl, which in each case can optionally form one or more ring structures, or (ii) an independently selected side chain moiety having a structure of an amino acid side chain;

R³ is (a) a side chain moiety selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W} and R_{Y}, each as delineated in Table I.A, (b) a side chain moiety selected from and having a structure of a non-natural amino acid side chain as delineated in Table I.B.1 or in Table I.C.1 or (c) a protected derivative of the foregoing side chain moieties;

R¹′, each R²′, R³′ and R⁴′ are each independently selected from the group consisting of H, C₁-C₃ alkyl and substituted C₁-C₃ alkyl;

each R¹⁰ is independently selected from the group consisting of H, C₁-C₃ alkyl and substituted C₁-C₃ alkyl;

each V is independently selected from the group consisting of —C(O)NH— and -ψ[ ]-; and

Y and Z are each independently selected from the group consisting of H, hydrocarbyl and substituted hydrocarbyl.

As formula I.A demonstrates, compounds of the disclosure can contain at least two ethoid bonds and be described as a polyethoid. In an embodiment, an ethoid is present when a=1, and a homoethoid is present when a=2. Compounds of formula I.A can have at least one a=1, each a=1, at least one a=2 or each a=2. In a embodiment, each a=1, R¹′, each R²′, R³′ and R⁴′ are each independently selected from the group consisting of H and methyl, each R¹⁰ is independently selected from the group consisting of H, methyl and substituted methyl, each V is independently selected from the group consisting of —C(O)NH— and -ψ[ ]-, and Y and Z are each independently selected from the group consisting of —V—, -functional group, -protected functional group, -linking moiety, -conjugate and -terminal group.

In an embodiment, R¹⁰ can be H, C₁-C₃ alkyl or substituted C₁-C₃ alkyl, preferably H, methyl and substituted methyl. More preferably, R¹⁰ can be H, C₁-C₃ alkyl and C₁-C₃ alkyl substituted with a group selected from -halogen, -hydroxy or —C₁-C₃ alkoxy. Most preferably R¹⁰ can be hydrogen.

R¹, R², R³, R⁴ can each be (i) an independently selected side chain moiety selected from the group consisting of H, C₁-C₁₀ alkyl and substituted C₁-C₁₀ alkyl, which in each case can optionally form one or more ring structures, or (ii) side chain moiety having the structure of an amino acid side chain, including natural amino acid side chains and non-natural amino acid side chains. In an embodiment, R³ can be (a) a side chain moiety selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W} and R_{Y}, (b) a side chain moiety selected from and having a structure of a non-natural amino acid side chain as delineated in Table I.B.1 or in Table I.C.1 or (c) a protected derivative of the foregoing side chain moieties.

In an embodiment, R¹, each R² and R⁴ can be each an independently selected side chain moiety having a structure of a natural amino acid side chain or a protected derivative thereof, preferably R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W}, R_{Y}, and protected derivative of the foregoing side chain moieties. In an alternate embodiment, R¹, each R² and R⁴ can be each an independently selected side chain moiety having a structure of a non-natural amino acid side chain. Preferably, R¹, each R² and R⁴ can be each independently selected and having a structure of a non-natural amino acid side chain as delineated in Table I.B.1, in Table I.B.2, or in Table I.C.1, or a protected derivative thereof.

In a preferred embodiment, R¹, and each R² other than R² nearest R¹, are each (a) independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1, in Table I.B.2, or in Table I.C.1, or (c) a protected derivative of the foregoing;

R⁴, and R² nearest R¹, are each (a) independently selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1 or in Table I.B.2, or (c) a protected derivative of the foregoing,

R³ when m≧1 is selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W} and R_{Y} each as delineated in Table I.A, (b) a side chain moiety having a structure of a non-natural amino acid side chain as delineated in Table I.B.1, in Table I.B.2, or in Table I.C.1, or (c) a protected derivative of the foregoing,

R³ when m=0 is selected from the group consisting of R_{C}, R_{D}, R_{E}, R_{F}, R_{H}, R_{I}, R_{K}, R_{L}, R_{M}, R_{N}, R_{Q}, R_{R}, R_{T}, R_{U}, R_{V}, R_{W} and R_{Y}, each as delineated in Table I.A, (b) a side chain moiety having a structure of a non-natural amino acid side chain as delineated in Table I.B.1, or (c) a protected derivative of the foregoing

More preferably, R¹, and each R² other than R² nearest R¹, are each independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W}, R_{Y} each as delineated in Table I.A, and a protected derivative thereof,

R⁴, and R² nearest R¹, are each independently selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, each as delineated in Table I.A, and a protected derivative thereof,

R³ when m≧1 is selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W}, R_{Y}, each as delineated in Table I.A, and a protected derivative thereof,

R³ when m=0 is selected from the group consisting of R_{C}, R_{D}, R_{E}, R_{F}, R_{H}, R_{I}, R_{K}, R_{L}, R_{M}, R_{N}, R_{Q}, R_{R}, R_{T}, R_{U}, R_{V}, R_{W}, R_{Y}, each as delineated in Table I.A, and a protected derivative thereof.

In an embodiment, R¹′, each R²′, R³′ and R⁴′ can each be independently selected from the group consisting of H, C₁-C₃ alkyl and substituted C₁-C₃ alkyl. Preferably, R¹′, each R²′, R³′ and R⁴′ can each be independently H, methyl or substituted methyl. More preferably, R¹′, each R²′, R³′ and R⁴′ can each be H. An exemplary set of amino acid residues is set forth in Table I.A.1, where Rⁿ is an typically an amino acid sidechain, and Rⁿ′ is methyl.

Compounds of formula I.A that have an m≧1 have at least four Rⁿ groups. In one embodiment, at least two of R¹, R², R³, or R⁴ are structurally different from each other, preferably at least three of R¹, R², R³, or R⁴ are structurally different from each other, more preferably at least four of R¹, R², R³, or R⁴ are structurally different from each other.

In an alternate embodiment, when m≧2, at least one V and its adjacent R² can have a structure selected from a moiety delineated in Table I.C.2.

Compounds of formula I.A can have m=0, in which case at least two ethoid moieties are on consecutive residues, or in which case at least one intervening residue is present between the at least two ethoid residues. That intervening residue can contain another ethoid bond, an amide bond, or another isostere -ψ[ ].

Other Isosteres

Compounds of the disclosure are not limited to ethoid bond linkages (or to ethoid bond linkages in combination with amide bond linkages), but when m≧1 can further include other isosteres -ψ[ ]. Compounds can further include one or more amide bond replacements to incorporate a -ψ[ ] that is independently selected from the group consisting of —CHR¹⁰O—, —CH₂CHR¹⁰O—, —C(O)NR⁷—, —CH₂C(O)NR⁷—, —CHR¹⁰NH—,

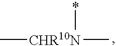

—CHR¹⁰OCHR¹⁰—, —CH₂CH₂—, —CH═CH—, —O—, —C(O)CH₂—, —C(O)O—, —CH(OH)CH₂—, —CH(OH)CH₂NH—, —CHR¹⁰S—, —CHR¹⁰S(O)—, —CHR¹⁰S(O₂)—, —CH₂CHR¹⁰S—, —CH₂CHR¹⁰S(O)—, —CH₂CHR¹⁰S(O₂)—, —CH(CH₃)S—, —C(O)S—, —C(S)NH—, —NHC(O)NH—, —OC(O)NH—, and retroinverso analogs thereof, each R⁷ being independently selected from the group consisting of —H and a side chain moiety having a structure of an amino acid side chain, each * representing a bond linking the nitrogen atom to R¹, an adjacent R², or R³. In an alternate embodiment, when each m≧1, each ψ[ ]- is independently selected from the group consisting of —CHR¹⁰O—, —CH₂CHR¹⁰O—, —C(O)NR⁷—, —CHR¹⁰NH—,

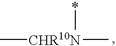

—CH₂CH₂—, —CH═CH—, —O—, —C(O)CH₂—, —C(O)O—, —CH(OH)CH₂—, —CHR¹⁰S—, —CHR¹⁰S(O)—, —CHR¹⁰S(O₂)—, —C(O)S—, —C(S)NH—, and retroinverso analogs thereof, each R⁷ being independently selected from the group consisting of —H and a side chain moiety having a structure of an amino acid side chain, each * representing a bond linking the nitrogen atom to R¹, an adjacent R², or R³. Preferably, each -ψ[ ]- is independently selected from the group consisting of —CHR¹⁰O—, —CH₂CHR¹⁰O—, —C(O)NR⁷—, —CHR¹⁰NH—,

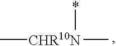

—CH₂CH₂—, —CH═CH—, —O— —C(O)CH₂—, —C(O)O—, —CH(OH)CH₂—, and retroinverso analogs thereof. More preferably, wherein each a=1, m is an integer ≧1, and each -ψ[ ]- is independently selected from the group consisting of —CHR¹⁰O—, —C(O)N C(O)NR⁷—, —CHR¹⁰NH—,

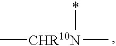

—CH₂CH₂—, —CH═CH—, —O—, —C(O)CH₂—, —C(O)O—, —CH(OH)CH₂—, and retroinverso analogs thereof. Most preferably, wherein each a=1, m is an integer ≧1, and each -ψ[ ]- is independently selected from the group consisting of —CHR¹⁰O—, —C(O)NR⁷—, —CHR¹⁰NH—,

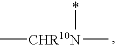

and retroinverso analogs thereof.

In one embodiment, when m≧1 and R¹⁰=H, -ψ[ ] can be —CH₂O—, —CH₂CH₂O—, —CH₂C(O)NH—, —CH₂NH—,

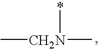

—CH₂OCH₂—, —CH₂CH₂—, —CH═CH—, —O—, —C(O)CH₂—, —C(O)O—, —CH(OH)CH₂—, —CH(OH)CH₂NH—, —CH₂S—, —CH₂S(O)—, —CH₂S(O₂)—, —CH₂CH₂S—, —CH₂CH₂S(O)—, —CH₂CH₂S(O₂)—, —CH(CH₃)S—, —C(O)S—, —C(S)NH—, —NHC(O)NH—, —OC(O)NH—, and retroinverso analogs thereof; each * representing a bond linking the nitrogen atom to R¹, an adjacent R², or R³. Alternatively, each -ψ[ ]- can be independently selected from the group consisting of —CH₂O—, —CH₂CH₂O—, —CH₂NH—,

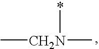

—CH₂CH₂—, —CH═CH—, —O—, —C(O)CH₂—, —C(O)O—, —CH(OH)CH₂—, —CH₂S—, —CH₂S(O)—, —CH₂S(O₂)—, —C(O)S—, —C(S)NH—, and retroinverso analogs thereof. Preferably, each -ψ[ ]- is independently selected from the group consisting of —CH₂O—, —CH₂CH₂O—, —CH₂NH—,

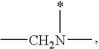

—CH₂CH₂—, —CH═CH—, —O—, —C(O)CH₂—, —C(O)O—, —CH(OH)CH₂—, and retroinverso analogs thereof. More preferably, each a=1 and each -ψ[ ]- is independently selected from the group consisting of —CH₂O—, —CH₂NH—,

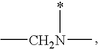

—CH₂CH₂—, —CH═CH—, —O—, —C(O)CH₂—, —C(O)O—, —CH(OH)CH₂—, and retroinverso analogs thereof. Most preferably, each a=1, and each -ψ[ ]- is independently selected from the group consisting of —CH₂O—, —CH₂NH—,

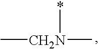

and retroinverso analogs.

The isosteres can be incorporated by any known method. For example, when the isostere is —CH₂NH—, the linkage can be synthesized by reductive amination of an aldehyde and an amine in NaCNBH₃. The isosteres can be added in combination with an individual monomer, or can be incorporated as a dimer, e.g. addition of NH₂CHR—CH₂CH₂—CHR′—COOH in an standard amide bond formation reaction.

In an aspect, R⁷ can be any group that can be attached to a nitrogen. In one embodiment, R⁷ can be hydrogen, alkyl, acyl, or a sidechain moiety having a structure of an amino acid side chain. Preferably, R⁷ is hydrogen, a side chain moiety independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y}, each as delineated in Table I.A; a side chain moiety having a structure of a non-natural amino acid side chain as delineated in Table I.B.1, in Table I.B.2, or in Table I.C.1; or a protected derivative of the foregoing. More preferably, R⁷ is hydrogen.

In an embodiment, when m is greater than or equal to 1, at least two of R¹, R², R³, and R⁴ in formula I.A are structurally distinct from each, preferably at least three of R¹, R², R³, and R⁴, more preferably at four of R¹, R³, R², and R⁴.

The polyethoids of the disclosure can have at least 1 ethoid bond. In an aspect, for the compound of formula I, where m is an integer greater than or equal to one, each —V— can be an ethoid moiety having the formula I.C

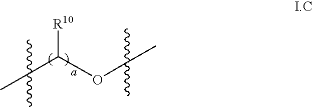

with any nature or non-natural amino acid side chain as set forth in Table I.A or Table I.B.1, with the proviso that if R¹, each R² (other than the R² nearest R¹) or R³ are R_{P} as delineated in Table I.A or are selected from and have a structure of a side chain moiety delineated in Table I.C.1, then —V— is a methyleneamine moiety having a formula

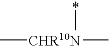

each * representing a bond linking the nitrogen atom to an adjacent side chain moiety. Preferably, each * represents a bond linking the nitrogen atom to an adjacent R_{P}, or to an adjacent side chain having the structure of proline, as set forth in Tables I.C.1 and I.C.2.

In an aspect, for the compound of formula I, where m is an integer greater than or equal to one, each —V— can be an ethoid moiety having the formula I.C

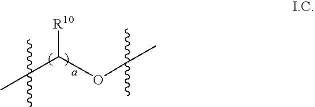

or an amide moiety having a formula I.D

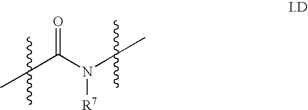

each R⁷ being independently selected from the group consisting of —H and a side chain moiety having a structure of an amino acid side chain, provided that at least one —V— is the amide moiety; and provided further that when R¹, each R² (other than the R² nearest R¹) or R³ are R_{P} as delineated in Table I.A or are selected from and have a structure of a side chain moiety delineated in Table I.C.1, then —V— is the amide moiety or a methyleneamine moiety having a formula

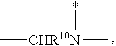

each * representing a bond linking the nitrogen atom to an adjacent side chain moiety. Preferably, when R¹=R_{P}, each R² (other than the R² nearest R¹)=R_{P}, or R³=R_{P}, then —V— can be the amide moiety or a methyleneamine moiety having a formula

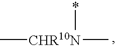

each * representing a bond linking the nitrogen atom to an adjacent R_{P}.

In an aspect, a compound of formula I can be represented by the polyethoid of formula I.B

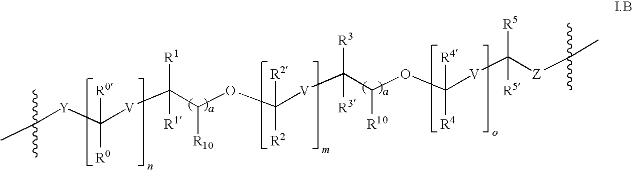

wherein:

the sum of n, m and o is ≧3;

each R⁰, R¹, each R², each R⁴ and R⁵ are each (i) an independently selected side chain moiety selected from the group consisting of H, C₁-C₁₀ alkyl and substituted C₁-C₁₀ alkyl, which in each case can optionally form one or more ring structures, or (ii) an independently selected side chain moiety having a structure of an amino acid side chain; and

each R⁰′, R¹′, each R²′, R³′, each R⁴′, and R⁵′ are each independently selected from the group consisting of H, C₁-C₃ alkyl and substituted C₁-C₃ alkyl.

Preferably

each a=1;

each R⁰′, R¹′, each R²′, R³′, each R⁴′, and R⁵′ are each independently selected from the group consisting of H and methyl;

each R¹⁰ is independently selected from the group consisting of H, methyl and substituted methyl;

each V is independently selected from the group consisting of —C(O)NH— and -ψ[ ]-; and

Y and Z are each independently selected from the group consisting of —V—, -functional group, -protected functional group, -linking moiety, -conjugate and -terminal group.

More preferably, each R⁰′, R¹′, each R²′, R³′, each R⁴′, and R⁵′ are each —H.

In a preferred embodiment, R¹⁰ is H.

Each R⁰, R¹, each R², each R⁴ and R⁵ can each be an independently selected side chain moiety having a structure of a natural or non-natural amino acid side chain or a protected derivative thereof, preferably a natural amino acid side chain or protected version thereof, preferably, R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W}, R_{Y}, each as delineated in Table I.A, and protected derivative of the foregoing side chain moieties. Alternatively each R⁰, R¹, each R², each R⁴ and R⁵ can each be independently selected side chain moiety having a structure of a non-natural amino acid side chain, preferably, having a structure of a non-natural amino acid side chain as delineated in Table I.B.1, in Table I.B.2, or in Table I.C.1, or a protected derivative thereof.

Preferably, in the polyethoid of formula I.B,
each R⁰, R¹, each R² other than R² nearest R¹, each R⁴ other than R⁴ nearest R³, and R⁵, are each (a) independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1, in Table I.B.2, or in Table I.C.1, or (c) a protected derivative of the foregoing,
R² nearest R¹, and R⁴ nearest R³ are each (a) independently selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1 or in Table I.B.2, or (c) a protected derivative of the foregoing,
R³ when m≧1 is selected from the group consisting of R_{C}, R_{D}, R_{E}, R_{F}, R_{H}, R_{I}, R_{K}, R_{L}, R_{M}, R_{N}, R_{Q}, R_{R}, R_{T}, R_{U}, R_{V}, R_{W} and R_{Y}, each as delineated in Table I.A, (b) a side chain moiety having a structure of a non-natural amino acid side chain as delineated in Table I.B.1, in Table I.B.2, or in Table I.C.1, or (c) a protected derivative of the foregoing, and
R³ when m=0 is selected from the group consisting of R_{C}, R_{D}, R_{E}, R_{F}, R_{H}, R_{I}, R_{K}, R_{L}, R_{M}, R_{N}, R_{Q}, R_{R}, R_{T}, R_{U}, R_{V}, R_{W} and R_{Y}, each as delineated in Table I.A, (b) a side chain moiety having a structure of a non-natural amino acid side chain as delineated in Table I.B.1, or (c) a protected derivative of the foregoing

More preferably, in the polyethoid of formula I,
each R⁰, R¹, each R² other than R² nearest R¹, each R⁴ other than R⁴ nearest R³, and R⁵, are each independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W}, R_{Y}, each as delineated in Table I.A, and a protected derivative thereof,
R² nearest R¹, and R⁴ nearest R³ are each independently selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, each as delineated in Table I.A, and a protected derivative thereof,
R³ when m≧1 is selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W}, R_{Y}, each as delineated in Table I.A, and a protected derivative thereof, and
R³ when m=0 is selected from the group consisting of R_{C}, R_{D}, R_{E}, R_{F}, R_{H}, R_{I}, R_{K}, R_{L}, R_{M}, R_{N}, R_{Q}, R_{R}, R_{T}, R_{U}, R_{V}, R_{W}, R_{Y}, each as delineated in Table I.A, and a protected derivative thereof.

In an embodiment, when n+m+o is greater than or equal to 3, at least three of R⁰, R¹, R², R⁴ or R⁵ in formula I.B are structurally distinct from each, preferably at least four of R⁰, R¹, R², R⁴ or R⁵, more preferably at least 5 of R⁰, R¹, R², R⁴ or R⁵.

In an embodiment, when each of n, m and o is an integer ranging from 1 to 5, each —V— in formula I.B can be an ethoid moiety having a formula

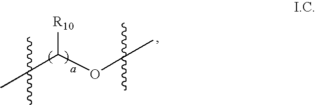

provided that when each R⁰, R¹, each R² (other than R² nearest R¹), R³; each R⁴ (other than R⁴ nearest R³) or R⁵ are R_{P} as delineated in Table I.A or are selected from and have a structure of a side chain moiety delineated in Table I.C.1, then —V— is a methyleneamine moiety having a formula I.E

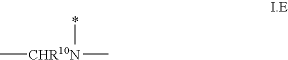

each * representing a bond linking the nitrogen atom to an adjacent side chain moiety, and Y and Z are each an independently selected terminal group. More preferably, when each R⁰=R_{P}, R¹=R_{P}, each R² (other than R² nearest R¹)=R_{P}, R³=R_{P}, each R⁴ (other than R⁴ nearest R³)=R_{P}, or R⁵=R_{P}, then —V— is a methyleneamine moiety having a formula

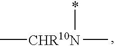

each * representing a bond linking the nitrogen atom to an adjacent side chain moiety.

In an embodiment, in formula X, when each of n, m and o is an integer ranging from 1 to 5, each —V— is an ethoid moiety having a formula I.C

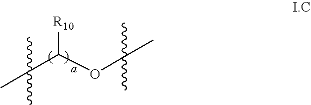

or an amide moiety having a formula I.D

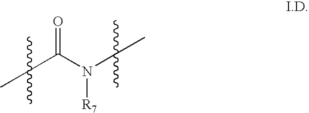

each R⁷ being independently selected from the group consisting of —H and a side chain moiety having a structure of an amino acid side chain; provided that at least one —V— is the amide moiety; and provided further that when each R⁰, R¹, each R² (other than R² nearest R¹), R³, each R⁴ (other than R⁴ nearest R³) or R⁵ are R_{P} as delineated in Table I.A or are selected from and have a structure of a side chain moiety delineated in Table I.C.1, then —V— is the amide moiety or a methyleneamine moiety having a formula I.E

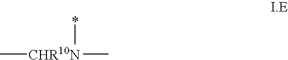

each * representing a bond linking the nitrogen atom to an adjacent side chain moiety, and Y and Z are each an independently selected terminal group. More preferably, when each R⁰=R_{P}, R¹=R_{P}, each R² (other than R² nearest R¹)=R_{P}, R³=R_{P}, each R⁴ (other than R⁴ nearest R³)=R_{P}, or R⁵=R_{P}, then —V— is a methyleneamine moiety having a formula

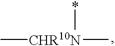

each * representing a bond linking the nitrogen atom to an adjacent side chain moiety.

### Extent of Ethoid Isosteric Substitutions

Compounds of the disclosure can be described by a ratio of the number of ethoid bonds, N_{ETHOID}, having the formula I.C

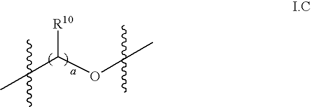

to the number of amide bonds, N_{AMIDE} having the formula I.D

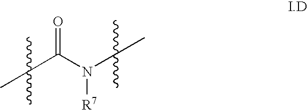

Due to the modular synthesis and flexibility present in this disclosure, any number of amide bonds can be replaced with any number of ethoid bonds. Consequently, the ratio of N_{ETHOID}:N_{AMIDE} can be the ratio of any integer or fractional portion thereof. In an embodiment, the ratio of N_{ETHOID}:N_{AMIDE} is at least about 1:99. Preferably, the ratio can be any number greater than at least about 1:99, including at least about 1:49, at least about 1:39, at least about 1:29, at least about 1:19, at least about 1:9, at least about 1:4, at least about 1:3, at least about 2:3, at least about 1:1, at least about 3:2, at least about 3:1, at least about 4:1, at least about 9:1, and any whole number or fractional number in between. Furthermore, the ratio can be higher than 9:1, up to a full ethoid where N_{AMIDE} is zero.

### Size/Length of Ethoid-Containing Compounds

The disclosure also provides for a polyethoid, for example the polyethoid section shown by Formula I.A and formula I.B above, having any length. The polyethoid can but need not represent the entire sequence of the backbone chain, and can be located anywhere along the chain, including near the C-terminus, near the N-terminus or between the two. The length of the backbone depends in part on the number of m, and optionally n and o, units present in the formula. In an embodiment, m can be any integer between 0 and 1000. Preferably, m can be any integer between 0 and 500, or 0 and 250. In a preferred embodiment, m can be greater than or equal to 1, including 1 to 250, 1 to 200, 1 to 150, 1 to 100, 1 to 75, 1 to 50, 1 to 30, and 1 to 15. Alternatively m can be greater than or equal to 3, including 3 to 250, 3 to 200, 3 to 150, 3 to 100, 3 to 75, 3 to 50, 3 to 40, 3 to 30, 3 to 20, 3 to 15 and 3 to 10. Alternatively, m can be less than or equal to 50, including 1 to 50, 2 to 50, 3 to 50, 4 to 50, 5 to 50, and 6 to 50. Furthermore, m can be 6 to 30. In an embodiment, m+n+o can be greater than or equal to 3 including 3 to 1000, 3 to 500, 3 to 250, 3 to 200, 3 to 150, 3 to 100, 3 to 75, 3 to 50, 3 to 40, 3 to 30, 3 to 20, 3 to 15 and 3 to 10. Alternatively, m+n+o can be less than or equal to 50, including 3 to 50, 4 to 50, 5 to 50, and 6 to 50. Furthermore, m+n+o can be 6 to 30.

### Y and Z Groups

Compounds of the disclosure can contain groups Y and Z. The groups Y and Z can be any group that attaches to a carbon atom. In one aspect, Y and Z can be each independently selected from hydrogen, hydrocarbyl and substituted hydrocarbyl. Y and Z can be each independently O-hydrocarbyl, —N-hydrocarbyl, —C(O)-hydrocarbyl, —O-(substituted hydrocarbyl), —N-(substituted hydrocarbyl), or —C(O)-(substituted hydrocarbyl). Y and Z can each independently be —V—. Y and Z can each independently be —V—, -functional group, -protected functional group, -linking moiety, -conjugate and -terminal group, or Y and Z can be each independently be -functional group, -protected functional group, -linking moiety, -conjugate and -terminal group, each optionally comprising —V—. Y and Z can each independently be a linking moiety covalently bonded to a support, the linking moiety optionally comprising —V. Alternatively, one or both of Y and Z can be —V—, each such —V— being covalently bonded to a moiety independently selected from a polyaminoacid and a polyethoid moiety, or alternatively a polypeptide or protein, or alternatively a polyethoidpeptide.

Compounds of the disclosure can also contain additional isosteric replacements for amide bond. These isosteric replacements, denoted -ψ[ ]- can include —CHR¹⁰O—, —CH₂CHR¹⁰O—, —C(O)NR⁷—, —CH₂C(O)NR⁷—, —CHR¹⁰NH—,

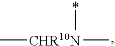

—CHR¹⁰OCHR¹⁰—, —CH₂CH₂—, —CH═CH—, —O—, —C(O)CH₂—, —C(O)O—, —CH(OH)CH₂—, —CH(OH)CH₂NH—, —CHR¹⁰S—, —CHR¹⁰S(O)—, —CHR¹⁰S(O₂)—, —CH₂CHR¹⁰S—, —CH₂CHR¹⁰S(O)—, —CH₂CHR¹⁰S(O₂)—, —CH(CH₃)S—, —C(O)S—, —C(S)NH—, —NHC(O)NH—, and —OC(O)NH—, and retroinverso analogs thereof. As previously noted, R⁷ can be selected from the group consisting of —H and a side chain moiety having a structure of an amino acid side chain. Each * represents a bond linking the nitrogen atom to an adjacent amino acid side chain moiety, thereby forming a ring structure. This type of structure, while not strictly limited to such, can be exemplied by a proline type ring structure.

Functional groups can include any functional group known in the art, including but not limited to hydroxyl, amines, acids, aldehydes, ethers, ketones, amides, esters, thiols, thioethers, and disulfides. Preferably, the functional groups can be an amine, amide, ester, acid, ether, aldehyde, ketone, thiol, or hydroxyl.

Protected functional groups can include any functional group that has been protected with a typical protecting group in order to prevent or limit its reactivity. A variety of methods for protecting groups for the most functional groups, including amines, aldehyes, ketone, and hydroxyls, including adding and removing the protecting groups, and the synthesis thereof can be found in “Protective Groups in Organic Synthesis” by T. W. Greene and P. G. M. Wuts, John Wiley & Sons, 1999.

600. Linking moieties can include any typical moiety, e.g. hydrocarbyl, heteroalkyl, that would connect or join, either directly or indirectly, an ethoid-containing compound to another group through one or more covalent bonds. A linking moiety could be a substituted or unsubstituted hydrocarbyl of C₁ to C₂₀ in size, and could optionally include —V—. A linking moiety could be an O-hydrocarbyl, —N-hydrocarbyl, —C(O)-hydrocarbyl, —O-(substituted hydrocarbyl), —N-(substituted hydrocarbyl), or —C(O)-(substituted hydrocarbyl). The other group that the polyethoid is linked to via the linking group can include a solid support, a terminal group, a conjugate, or any other compound that would provide a benefit to, or derive a benefit from a linked polyethoid, or provide for the preparation of the polyethoid.

Conjugates can include a moiety, group, compound or other molecule that can be attached to a polyethoid compound via a suitable linking group, such that the polyethoid-conjugate compound now has one or more different properties compared to the polyethoid or conjugate alone. Such characteristics could include but are not limited to biological transport, biological half-life, physical or chemical characteristics, cell or organelle specific delivery, or stabilization. By way of example but without limitation, the conjugate could provide a specific benefit to the polyethoid such as for example, modified binding to serum albumin by addition of a fatty acid. Alternative, the polyethoid could provide a benefit to the conjugate, for example by conjugation to a drug molecule. The benefit could also be synergistic. Several non-limiting examples of conjugates would include cholic acid, cholic acid analogs, glycocholate, taurocholate, polyethylene glycols, fatty acids, fatty alcohols, polyglycols, sugar molecules, proteins in the MPG family, Pep-1, Tat sequences from HIV-1, antibodies including humanized monoclonal antibodies, DNA, RNA, aptamers, pharmaceutical or drug compounds, metal complexes, nanoparticles, and quantum dots. Other examples can be identified from the scientific literature, including for example, Bioconjugates, published by the American Chemical Society.

Terminal groups can include any atom or group that would covalently bind to the polyethoid chain, including but not limited to carbon, oxygen, hydrogen, nitrogen, sulfur, phosphorous, a halide, an alkyl, hydrocarbyl, hydroxyl, amine, thiol, amide, ethers and the like.

Preferably when Y is a terminal group, Y is H—, H₂N—, AcNH—, R²⁰C(O)NH—, R²²OC(O)NH—, HO—, R²⁰O—, and protected derivatives thereof, R²⁰ and R²² each being independently selected from the group consisting of H, hydrocarbyl and substituted hydrocarbyl. In an embodiment, R²⁰ and R²² can be each independently selected from the group consisting of H, alkyl, substituted alkyl, heterocycle, and substituted heterocycle, the group consisting of H, C₁-C₈ alkyl and substituted C₁-C₈ alkyl, the group consisting of H —CH₃, —CH₂CH₃, —CH₂CH₂CH₃ and —CH₂CH(CH₃)₂, the group consisting of H, HC(O)NH—, and CH₂C(O)NH—, the group consisting of H, C₁-C₁₂ heterocycle and substituted C₁-C₁₂ heterocycle. Or the group consisting of H and pyridyl.

Y can also be selected from the group consisting of pyroglutamate, trans-cinnamoyl-NH—, cinnamoyl-NH—, palmitoyl-NH—, and 4-(2-pyrrolidinonyl)CH₂O—, and protected derivatives thereof.

Preferably, when Z is a terminal group, Z can be selected from the group consisting of —H, —R²⁰OH, —C(O)OR²⁰, —C(O)H, —C(O)R²⁰, —R²⁰OR²², —C(O)NHR²⁰ and protected derivatives thereof, where R²⁰ and R²² can be each being independently selected from the group consisting of H, hydrocarbyl and substituted hydrocarbyl. In an embodiment, R²⁰ and R²² are each independently selected from the group consisting of H, alkyl, substituted alkyl, heterocycle, and substituted heterocycle, the group consisting of H, C₁-C₈ alkyl and substituted C₁-C₈ alkyl, the group consisting of H, C₁-C₁₂ heterocycle and substituted C₁-C₁₂ heterocycle, the group consisting of —H, —CH₂OH, —C(O)OH, —C(O)H, —C(O)NH₂, —C(O)CH₃, —C(O)NHNHC(O)NH, —C(O)NHCH₂CH₂OH, —C(O)NHCH₂CH₃, and protected derivatives thereof.

Compounds in this disclosure can further include one or more amide bond replacements to incorporate another isostere into the a -ψ[ ] that is independently selected from the group consisting of —CHR¹⁰O—, —CH₂CHR¹⁰O—, —C(O)NR⁷—, —CH₂C(O)NR⁷—, —CHR¹⁰NH—,

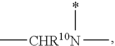

—CHR¹⁰OCHR¹⁰—, —CH₂CH₂—, —CH═CH—, —O—, —C(O)CH₂—, —C(O)O—, —CH(OH)CH₂—, —CH(OH)CH₂NH—, —CHR¹⁰S—, —CHR¹⁰S(O)—, —CHR¹⁰S(O₂)—, —CH₂CHR¹⁰S—, —CH₂CHR¹⁰S(O)—, —CH₂CHR¹⁰S(O₂)—, —CH(CH₃)S—, —C(O)S—, —C(S)NH—, —NHC(O)NH—, —OC(O)NH—, and retroinverso analogs thereof, each R⁷ being independently selected from the group consisting of —H and a side chain moiety having a structure of an amino acid side chain, each * representing a bond linking the nitrogen atom to R¹, an adjacent R², or R³.

Ethoid Compounds

The synthetic schemes disclosed above and further set forth below are generally applicable to the preparation of ethoid bonds between all possible monomers as described above. Therefore, for the first time a method has been disclosed that can incorporate substituted or unsubstituted methyleneoxy isosteric replacements in any combination at any sequence position in a polyaminoacid polymer. Significantly, the modular chemistry and specific synthetic routes disclosed herein allow for, for example and without limitation, two or more XaaΨ[CH₂O]Yaa methyleneoxy for amide bond replacements to be included in a single peptide. The present synthetic strategy facilitates the preparation of polypeptides having two consecutive or non-consecutive ethoid bonds. Further, at least half of the possible dipeptide-like combinations of amino acids containing one ethoid bond between the residues were not previously synthesized. Table II lists some of the novel ethoid compounds having one or more ethoid moieties between adjacent, preferably chiral, carbon centers, each with a pendant side chain group. For convenience of nomenclature, and without limitation, the various side chain R groups in this Table I are disclosed with reference to the R side chain moiety of the natural amino acids.

TABLE II

| 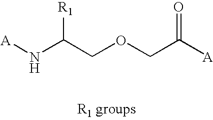 | 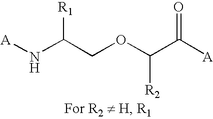 | 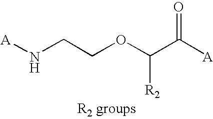 | 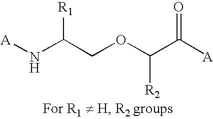 |
|---|---|---|---|
| E | E | C | C |
| H | H | E | E |
| K | K | K | K |
| M | M | M | M |
| N | N | Q | N |
| Q | Q | R | Q |
| W | R | Y | R |
| | W | W | S |
| | | | T |
| | | | Y |
| | | | W |

Novel dipeptide isosteres that can be created by the present methods include those with single ethoid bond replacements with either C, E, H, K, M, N, Q, or W monomer at the N-terminal position; with single ethoid bond replacements with an R monomer at the N-terminal position and non-G monomer at the C-terminal position; those having single ethoid bond replacements with either C, E, K, M, Q, R, or W monomer at the C-terminal position; those with single ethoid bond replacements with an N or S monomer at the C-terminal position and a non-G monomer at the N-terminal position; those with single ethoid bond replacements with either C, E, H, K, M, N, Q, R or W monomer at the N-terminal position and either C, E, K, M, N, Q, R, S or W monomer at the C-terminal position.

In an embodiment, a compound comprising an ethoid moiety having a formula

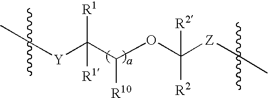

wherein

a is an independently selected integer=1 or =2,

R¹ selected from the group consisting of R_{A}, R_{C}, R_{D}, R_{E}, R_{F}, R_{G}, R_{H}, R_{I}, R_{K}, R_{L}, R_{N}, R_{P}, R_{Q}, R_{R}, R_{S}, R_{T}, R_{U}, R_{V}, R_{W}, R_{Y} and protected derivatives thereof,

when R¹ is R_{A}, then R² is selected from the group consisting of R_{C}, R_{D}, R_{E}, R_{F}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof,

when R¹ is R_{C}, R_{E}, R_{H}, R_{K}, R_{M}, R_{N}, R_{Q}, R_{T}, R_{U} or R_{W}, then R₂ is selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof,

when R¹ is R_{D} or R_{S}, then R² is selected from the group consisting of R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y}, and protected derivatives thereof,

when R¹ is R_{F}, then R² is selected from the group consisting of R_{C}, R_{D}, R_{E}, R_{G}, R_{H}, R_{I}, R_{K}, R_{L}, R_{M}, R_{N}, R_{Q}, R_{R}, R_{S}, R_{T}, R_{U}, R_{V}, R_{W}, R_{Y} and protected derivatives thereof, when R₁ is R_{G}, then R₂ is selected from the group consisting of R_{C}, R_{E}, R_{I}, R_{K}, R_{L}, R_{M}, R_{N}, R_{Q}, R_{R}, R_{T}, R_{U}, R_{W}, R_{Y}, and protected derivatives thereof,

when R¹ is R_{I}, then R² is selected from the group consisting of R_{C}, R_{D}, R_{E}, R_{H}, R_{I}, R_{K}, R_{L}, R_{M}, R_{N}, R_{Q}, R_{R}, R_{S}, R_{T}, R_{U}, R_{W}, R_{Y}, and protected derivatives thereof,

when R¹ is R_{L}, then R² is selected from the group consisting of R_{C}, R_{D}, R_{E}, R_{H}, R_{I}, R_{K}, R_{M}, R_{N}, R_{Q}, R_{R}, R_{S}, R_{T}, R_{U}, R_{W}, R_{Y} and protected derivatives thereof,

when R¹ is R_{P}, then R² is selected from the group consisting of R_{C}, R_{D}, R_{E}, R_{H}, R_{I}, R_{K}, R_{M}, R_{N}, R_{Q}, R_{R}, R_{S}, R_{U}, R_{V}, R_{W}, R_{Y}, and protected derivatives thereof,

when R¹ is R_{R}, then R² is selected from the group consisting of R_{A}, R_{C}, R_{D}, R_{E}, R_{F}, R_{H}, R_{K}, R_{L}, R_{M}, R_{N}, R_{Q}, R_{R}, R_{S}, R_{T}, R_{U}, R_{V}, R_{W}, R_{Y}, and protected derivatives thereof,

when R¹ is R_{V}, then R² is selected from the group consisting of R_{C}, R_{D}, R_{E}, R_{F}, R_{H}, R_{I}, R_{K}, R_{M}, R_{N}, R_{Q}, R_{R}, R_{S}, R_{T}, R_{U}, R_{V}, R_{W}, R_{Y}, and protected derivatives thereof,

when R¹ is R_{Y}, then R² is selected from the group consisting of R_{A}, R_{C}, R_{E}, R_{H}, R_{K}, R_{L}, R_{M}, R_{N}, R_{Q}, R_{R}, R_{S}, R_{T}, R_{U}, R_{V}, R_{W}, R_{Y}, and protected derivatives thereof,

each of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y} being delineated in Table I.A,

R¹′ and R²′ are each independently selected from the group consisting of H, C₁-C₃ alkyl and substituted C₁-C₃ alkyl,

R¹⁰ is selected from the group consisting of H, C₁-C₃ alkyl and substituted C₁-C₃ alkyl, and

Y and Z are each independently selected from the group consisting of H, hydrocarbyl and substituted hydrocarbyl.

In an embodiment, a=1. Alternatively, a=2. Preferably, a=1, R₁′ and R₂′ are each independently selected from the group consisting of H and methyl; each R¹⁰ is independently selected from the group consisting of H, methyl and substituted methyl; and Y and Z are each independently selected from the group consisting of —V—, -functional group, -protected functional group, -linking moiety, -conjugate and -terminal group, each V being independently selected from the group consisting of —C(O)NH— and -ψ[ ]-. More preferably R₁′ and R₂′ are each H. Alternatively, R₁′ and R₂′ are each H.

The groups Y and Z can be any group that attaches to a carbon atom. In one aspect, Y and Z can be each independently selected from hydrogen, hydrocarbyl and substituted hydrocarbyl. Y and Z can be each independently O-hydrocarbyl, —N-hydrocarbyl, —C(O)-hydrocarbyl, —O-(substituted hydrocarbyl), —N-(substituted hydrocarbyl), or —C(O)-(substituted hydrocarbyl). Y and Z can each independently be —V—. Y and Z can each independently be —V—, -functional group, -protected functional group, -linking moiety, -conjugate and -terminal group, or Y and Z can be each independently be -functional group, -protected functional group, -linking moiety, -conjugate and -terminal group, each optionally comprising —V—. Y and Z can each independently be a linking moiety covalently bonded to a support, the linking moiety optionally comprising —V. Alternatively, one or both of Y and Z can be —V—, each such —V— being covalently bonded to a moiety independently selected from a polyaminoacid and a polyethoid moiety, or alternatively a polypeptide or protein, or alternatively a polyethoidpeptide.

Alternatively, Y can be a functional group selected from the group consisting of —R⁷N—, —C(O)R⁷N—, and —R²⁰O—, and Z can be a functional group selected from the group consisting of —C(O)—, —C(O)O—, —C(O)NR⁷—, and CH₂OR²⁰—, each R⁷ being independently selected from the group consisting of —H and a side chain moiety having a structure of an amino acid side chain, each R²⁰ being independently selected from the group consisting of H, hydrocarbyl and substituted hydrocarbyl. Preferably, Y can be a functional group selected from the group consisting of —HN—, —C(O)HN—, and —CH₂O—, and Z can be a functional group selected from the group consisting of —C(O)—, —C(O)O—, —C(O)NH—, —CH₂O—.

In the alternative, Y and Z can each be an independently selected protected functional group; can each be an independently selected linking moiety; or can each be an independently selected conjugate.

In an embodiment, when one or both of Y and Z are —V—, each -ψ[ ]- can be independently selected from the group consisting of —CHR¹⁰O—, —CH₂CHR¹⁰O—, —C(O)NR⁷—, —CH₂C(O)NR⁷—, —CHR¹⁰NH—,

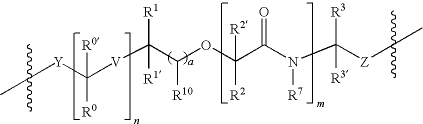

—CHR¹⁰OCHR¹⁰—, —CH₂CH₂—, —CH═CH—, —O—, —C(O)CH₂—, —C(O)O—, —CH(OH)CH₂—, —CH(OH)CH₂NH—, —CHR¹⁰S—, —CHR¹⁰S(O)—, —CHR¹⁰S(O₂)—, —CH₂CHR¹⁰S—, —CH₂CHR¹⁰S(O)—, —CH₂CHR¹⁰S(O₂)—, —CH(CH₃)S—, —C(O)S—, —C(S)NH—, —NHC(O)NH—, —OC(O)NH—, and retroinverso analogs thereof, each R⁷ being independently selected from the group consisting of —H and a side chain moiety having a structure of an amino acid side chain, each * representing a bond linking the nitrogen atom to R¹, an adjacent R², or R³.

In an aspect, the compound can have the formula

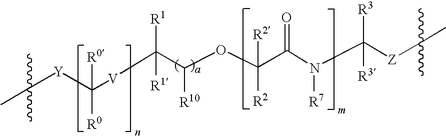

wherein

m and n are each an independently selected integer ≧0, and the sum of m and n is ≧1,

when m≧1, then: the R² nearest R¹ (i.e., the R² adjacent the ethoid moiety opposite R¹) is independently selected in combination with R¹ as described below in connection with the second general embodiment of this first aspect; and each R⁰, each R² other than the R² nearest R¹, and R³ are each an independently selected side chain moiety having a structure of an amino acid side chain.

when m=0: then each R⁰ is an independently selected side chain moiety having a structure of an amino acid side chain; and R³=R² (i.e., R³ is the same as R² as described above in connection with the second general embodiment of this first aspect) and is independently selected in combination with R¹ as described above in connection with the second general embodiment of this first aspect, each R² is independently selected,

each R₀ and R₃ is an independently selected side chain moiety having a structure of an amino acid side chain,

each R⁰′, and R³′ is selected from the group consisting of H, C₁-C₃ alkyl and substituted C₁-C₃ alkyl,

each R⁷ being independently selected from the group consisting of —H and a side chain moiety having a structure of an amino acid side chain from the group consisting of H, and

each V is independently selected from the group consisting of —C(O)NH— and -ψ[ ]-.

Preferably, each R⁰ and R³ can be each (a) independently selected from the group consisting of R_{A}, R_{C}, R_{D}, R_{E}, R_{F}, R_{G}, R_{H}, R_{I}, R_{K}, R_{L}, R_{M}, R_{N}, R_{P}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1, in Table I.B.2, or in Table I.C.1, or (c) a protected derivative of the foregoing.

Preferably, m+n can be an integer from about 1 to about 15, about 1, to about 10, or about 1 to about 5, and an integer or fraction of an integer in between.

In an aspect, the compound can have the formula,

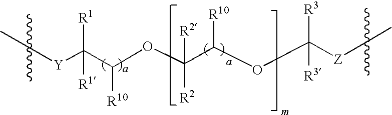

wherein

m is an integer ≧1,

each R₂ is independently selected,

R₃ is a side chain moiety having a structure of an amino acid side chain,

R₃′ is selected from the group consisting of H, C₁-C₃ alkyl and substituted C₁-C₃ alkyl, and

R⁷ being independently selected from the group consisting of —H and a side chain moiety having a structure of an amino acid side chain.

Preferably, R₃ can be (a) independently selected from the group consisting of R_{A}, R_{C}, R_{D}, R_{E}, R_{F}, R_{G}, R_{H}, R_{I}, R_{K}, R_{L}, R_{M}, R_{N}, R_{Q}, R_{R}, R_{S}, R_{T}, R_{U}, R_{V}, R_{W} and R_{Y}, each as delineated in Table I.A, (b) a side chain moiety having a structure of a non-natural amino acid side chain as delineated in Table I.B.1 or in Table I.B.2, or (c) a protected derivative of the foregoing.

Preferably, m can be an integer from about 1 to about 15, about 1, to about 10, or about 1 to about 5, and an integer or fraction of an integer in between.

In a preferred embodiment, the compound can be described as

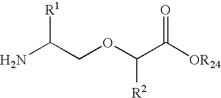

with R²⁴ selected from the group consisting of H, alkyl and substituted alkyl.

Polyaminoacid Analogs

With the ability to create ethoid bonds between any two residues, it now becomes possible to create a polyethoid version of any polypeptide or polyaminoacid. The polyethoid could contain one ethoid bond or could be a full replacement of every amide bond in the polyaminoacid. In a compound comprising a polyaminoacid, wherein the polyaminoacid comprises three or more amino acid residues linked by amide moieties, the improvement comprising at least two ethoid isosteres, each having a formula

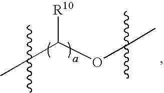

and being a substitutive replacement for an amide moiety of the polyaminoacid, wherein

each a is an independently selected integer=1 or =2, each R¹⁰ is independently selected from the group consisting of H, C₁-C₃ alkyl and substituted C₁-C₃ alkyl. The compound comprising the polyaminoacid to which the improvement can be made can have a formula I-D-I

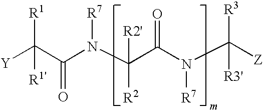

wherein m can be an integer ranging from 1 to 500, R¹, each R², and R³ can be each an independently selected side chain moiety having a structure of an amino acid side chain; R^{1′}, each R^{2′}, and R^{3′} can be each independently selected from the group consisting of H, C₁-C₃ alkyl and substituted C₁-C₃ alkyl, each R⁷ can be independently selected from the group consisting of —H and a side chain moiety having a structure of an amino acid side chain, Y can be a terminal group selected from the group consisting of H—, H₂N—, AcNH—, R²⁰C(O)NH—, R²²OC(O)NH—, HO—, —R²⁰O—, and protected derivatives thereof, R²⁰ and R²² each being independently selected from the group consisting of H, hydrocarbyl and substituted hydrocarbyl, and Z can be selected from the group consisting of —H, —R²⁰OH, —C(O)OR²⁰, —C(O)H, —C(O)R²⁰, —R²⁰OR²², —C(O)NHR²⁰ and protected derivatives thereof, R²⁰ and R²² each being independently selected from the group consisting of H, hydrocarbyl and substituted hydrocarbyl.

In an embodiment, the ethoid isosteres in the improvement can have at least one a=1, each a=1, at least one a=2 or each a=2. Preferably, each a=1 and each R¹⁰ is independently selected from the group consisting of H, methyl and substituted methyl.

In an embodiment, R^{1′}, each R^{2′}, and R^{3′} can be each independently selected from the group consisting of H and methyl, and R²⁰ and R²² can be each independently selected from the group consisting of alkyl, substituted alkyl, heteroalkyl, substituted heteroalkyl, aryl, substituted aryl, heteroaryl, substituted heteroaryl, alicyclic, substituted alicyclic, heterocyclic, and substituted heterocyclic. Preferably, R^{1′}, each R^{2′}, and R^{3′} are each H, and R²⁰ and R²² are each independently selected from the group consisting of H, C₁-C₈ alkyl and substituted C₁-C₈ alkyl. More preferably, R¹⁰ can be H.

In an embodiment, the improvement can comprise at least 3 ethoid replacements, or at least 4 ethoid replacements.

In an embodiment of the improvement, a first ethoid isostere can be at a first sequence position of the polyaminoacid, and a second ethoid isostere can be at a second sequence position of the polyaminoacid, the second sequence position being different from the first sequence position.

In an embodiment, the ethoid isostere subtitutively replaces a proteolytic-susceptible amide moiety of the polyaminoacid. Preferably, at least two ethoid isosteres subtitutively replace at least two proteolytic-susceptible amide moieties.

In an embodiment of the improvement of a polyaminoacid with at least four ethoid isosteres as a substitutive replacement for at least four amide moieties of the polyaminoacid, the at least four ethoid isosteres can include (a) a first ethoid isostere as a substitutive replacement for a proteolytic-susceptible first amide moiety at a first sequence position of the polyaminoacid, (b) a second ethoid isostere as a substitutive replacement for a second amide moiety at a second sequence position of the polyaminoacid, the second sequence position being adjacent the first sequence position, (c) a third ethoid isostere as a substitutive replacement for a proteolytic-susceptible third amide moiety at a third sequence position of the polyaminoacid, and (d) a fourth ethoid isostere as a substitutive replacement for a fourth amide moiety at a fourth sequence position of the polyaminoacid, the fourth sequence position being adjacent the third sequence.

In an alternate embodiment of the improvement of a polyaminoacid with at least six ethoid isosteres as a substitutive replacement for at least six amide moieties of the polyaminoacid, the at least six ethoid isosteres can include (a) a first ethoid isostere as a substitutive replacement for a proteolytic-susceptible first amide moiety at a first sequence position of the polyaminoacid, (b) second and third ethoid isosteres as a substitutive replacement for second and third amide moieties at a second and third sequence position of the polyaminoacid, respectively, each of the second and third sequence positions being adjacent the first sequence position, (c) a fourth ethoid isostere as a substitutive replacement for a proteolytic-susceptible fourth amide moiety at a forth sequence position of the polyaminoacid, and (d) fifth and sixth ethoid isosteres as a substitutive replacement for fifth and sixth amide moieties at a fifth and sixth sequence position of the polyaminoacid, respectively, each of the fifth and sixth sequence positions being adjacent the second sequence position

The improvement of a polyaminoacid can further include at least one methyleneamine isostere having a formula selected from the group consisting of —CHR¹⁰NH— and

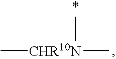

that replaces at least one amide moiety of the polyaminoacid, * representing a bond linking the nitrogen atom to an adjacent side chain moiety. In a preferred embodiment, R¹⁰ can be H. In one embodiment, * represents a bond linking the nitrogen atom to an adjacent R_{P} as delineated in Table I.A or to an adjacent side chain moiety selected from and having a structure as delineated in Table I.C.1.

In one embodiment, the polyaminoacid can be biologically active. It can be a substrate for a peptidase or protease. It can be an imaging or diagnostic agent, a receptor agonist or receptor antagonist, a therapeutic agent, derived from an α-amino acid, a protein, or a polypeptide. The polyaminoacid can further be a multiple combination of each of these.

In a preferred embodiment, the polyaminoacid is a protein or polypeptide analogs selected from the group consisting of: GHRH; PR1 (T-cell epitope); Protease-3 peptide (1); Protease-3 peptide (2); Protease-3 peptide (3); Protease-3 peptide (4); Protease-3 peptide (5); Protease-3 peptide (6); Protease-3 peptide (7); Protease-3 peptide (8); Protease-3 peptide (9); Protease-3 peptide (10); Protease-3 peptide (11); P3, B-cell epitope; P3, B-cell epitope: (with spacer); GLP1; LHRH; PTH; Substance P; Neurokinin A; Neurokinin B; Bombesin; CCK-8; Leucine Enkephalin; Methionine Enkephalin; [Des Ala20, Gln34] Dermaseptin; Antimicrobial Peptide (Surfactant); Antimicrobial Anionic Peptide (Surfactant-associated AP); Apidaecin IA; Apidaecin IB; OV-2; 1025, Acetyl-Adhesin Peptide (1025-1044) amide; Theromacin (49-63); Pexiganan (MSI-78); Indolicidin; Apelin-15 (63-77); CFP10 (71-85); Lethal Factor (LF) Inhibitor Anthrax related; Bactenecin; Hepatitis Virus C NS3 Protease Inhibitor 2; Hepatitis Virus C NS3 Protease Inhibitor 3; Hepatitis Virus NS3 Protease Inhibitor 4; NS4A-NS4B Hepatitis Virus C(NS3 Protease Inhibitor 1); HIV-1, HIV-2 Protease Substrate; Anti-Flt1 Peptide; Bak-BH3; Bax BH3 peptide (55-74) (wild type); Bid BH3-r8; CTT (Gelatinase Inhibitor); E75 (Her-2/neu) (369-377); GRP78 Binding Chimeric Peptide Motif; p53(17-26); EGFR2/KDR Antagonist; Colivelin AGA-(C8R) HNG17 (Humanin derivative); Activity-Dependent Neurotrophic Factor (ADNF); Beta-Secretase Inhibitor 1; Beta-Secretase Inhibitor 2; chβ-Amyloid (30-16); Humanun (HN); sHNG, [Gly14]-HN, [Gly14]-Humanin; Angiotensin Converting Enzyme Inhibitor (BPP); Renin Inhibitor III; Annexin 1 (ANXA-1; Ac2-12); Anti-Inflammatory Peptide 1; Anti-Inflammatory Peptide 2; Anti-Inflammatory Apelin 12; [D-Phe12, Leu14]-Bombesin; Antennapedia Peptide (acid) (penetratin); Antennepedia Leader Peptide (CT); Mastoparan; [Thr28, Nle31]-Cholecystokinin (25-33) sulfated; Nociceptin (1-13) (amide); Fibrinolysis Inhibiting Factor; Gamma-Fibrinogen (377-395); Xenin; Obestatin (human); [His1, Lys6]-GHRP (GHRP-6); [Ala5, β-Ala8]-Neurokinin A (4-10); Neuromedin B; Neuromedin C; Neuromedin N; Activity-Dependent Neurotrophic Factor (ADNF-14); Acetalin 1 (Opioid Receptor Antagonist 1); Acetalin 2 (Opioid Receptor Antagonist 2); Acetalin 3 (Opioid Receptor Antagonist 3); ACTH (1-39) (human); ACTH (7-38) (human); Sauvagine; Adipokinetic Hormone (Locusta Migratoria); Myristoylated ADP-Ribosylation Factor 6, myr-ARF6 (2-13); PAMP (1-20) (Proadrenomedullin (1-20) human); AGRP (25-51); Amylin (8-37) (human); Angiotensin I (human); Angiotensin II (human); Apstatin (Aminopeptidase P Inhibitor); Brevinin-1; Magainin 1; RL-37; LL-37 (Antimicrobial Peptide) (human); Cecropin A; Antioxidant peptide A; Antioxidant peptide B; L-Carnosine; Bcl 9-2; NPVF; Neuropeptide AF (hNPAF) (Human); Bax BH3 peptide (55-74); bFGF-Inhibitory Peptide; bFGF inhibitory Peptide II; Bradykinin; [Des-Arg10]-HOE I40; Caspase 1 Inhibitor II; Caspase 1 Inhibitor VIII; Smac N7 Protein; MEK1 Derived Peptide Inhibitor 1; hBD-1 (β-Defensin-1) (human); hBD-3 (β-Defensin-3) (human); hBD-4 (β-Defensin-4) (human); HNP-1 (Defensin Human Neutrophil Peptide 1); HNP-2 (Defensin Human neutrophil Peptide-2 Dynorphin A (1-17)); Endomorphin-1; β-Endorphin (human porcine); Endothelin 2 (human); Fibrinogen Binding Inhibitor Peptide; Cyclo(-GRGDSP); TP508 (Thrombin-derived Peptide); Galanin (human); GIP (human); Gastrin Releasing Peptide (human); Gastrin-1 (human); Ghrelin (human); PDGF-BB peptide; [D-Lys3]-GHRP-6; HCV Core Protein (1-20); a3β1 Integrin Peptide Fragment (325) (amide); Laminin Pentapeptide (amide); Melanotropin-Potentiating Factor (MPF); VA-β-MSH, Lipotropin-Y (Proopiomelanocortin-derived); Atrial Natriuretic Peptide (1-28) (human); Vasonatrin Peptide (1-27); [Ala5, β-Ala8]-Neurokinin A (4-10); Neuromedin L (NKA); Ac-(Leu28, 31)-Neuropeptide Y (24-26); Alytesin; Brain Neuropeptide II; [D-tyr11]-Neurotensin; IKKy NEMO Binding Domain (NBD) Inhibitory Peptide; PTD-p50 (NLS) Inhibitory Peptide; Orexin A (bovine, human, mouse, rat); Orexin B (human); Aquaporin-2(254-267) (human Pancreastatin) (37-52); Pancreatic Polypeptide (human); Neuropeptide; Peptide YY (3-36) (human); Hydroxymethyl-Phytochelatin 2; PACAP (1-27) (amide, human, bovine, rat); Prolactin Releasing Peptide (1-31) (human); Salusin-alpha; Salusin-beta; Saposin C22; Secretin (human); L-Selectin; Endokinin A/B; Endokinin C (Human); Endokinin D (Human); Thrombin Receptor (42-48) Agonist (human); LSKL (Inhibitor of Thrombospondin); Thyrotropin Releasing Hormone (TRH); P55-TNFR Fragment; Urotensin II (human); VIP (human, porcine, rat); VIP Antagonist; Helodermin; Exenatide; ZP10 (AVE00100); Pramlinitide; AC162352 (PYY) (3-36); PYY; Obinepitide; Glucagon; GRP; Ghrelin (GHRP6); Leuprolide; Histrelin; Oxytocin; Atosiban (RWJ22164); Sermorelin; Nesiritide; bivalirudin (Hirulog); Icatibant; Aviptadil; Rotigaptide (ZP123, GAP486); Cilengitide (EMD-121924, RGD Peptides); AlbuBNP; BN-054; Angiotensin II; MBP-8298; Peptide Leucine Arginine; Ziconotide; AL-208; AL-108; Carbeticon; Tripeptide; SAL; Coliven; Humanin; ADNF-14; VIP (Vasoactive Intestinal Peptide); Thymalfasin; Bacitracin (USP); Gramidicin (USP); Pexiganan (MSI-78); P113; PAC-113; SCV-07; HLF1-11 (Lactoferrin); DAPTA; TRI-1144; Tritrpticin; Antiflammin 2; Gattex (Teduglutide, ALX-0600); Stimuvax (L-BLP25); Chrysalin (TP508); Melanonan II; Spantide II; Ceruletide; Sincalide; Pentagastin; Secretin; Endostatin peptide; E-selectin; HER2; IL-6; IL-8; IL-10; PDGF; Thrombospondin; uPA (1); uPA (2); VEGF; VEGF (2); Pentapeptide-3; Glutathione; XXLRR; Beta-Amyloid Fibrillogenesis; Endomorphin-2; TIP 39 (Tuberoinfundibular Neuropeptide); PACAP (1-38) (amide, human, bovine, rat); TGFβ activating peptide; Insulin sensitizing factor (ISF402); Transforming Growth Factor β1 Peptide (TGF-β1); Caerulein Releasing Factor; IELLQAR (8-branch MAPS); Tigapotide PK3145; Goserelin; Abarelix; Cetrorelix; Ganirelix; Degarelix (Triptorelin); Barusiban (FE 200440); Pralmorelin; Octreotide; Eptifibatide; Netamiftide (INN-00835); Daptamycin; Spantide II (1); Delmitide (RDP-58); AL-209; Enfuvirtide; IDR-1; Hexapeptide-6; Insulin-A chain; Lanreotide; Hexapeptide-3; Insulin B-chain; Glargine-A chain; Glargine-B chain; Insulin-LisPro B-chain analog; Insulin-Aspart B-chain analog; Insulin-Glulisine B chain analog; Insulin-Determir B chain analog; Somatostatin; Somatostatin Tumor Inhibiting Analog; Pancreastatin (37-52); Vasoactive Intestinal Peptide fragment (KKYL-NH2); and Dynorphin A; or analogs thereof of any of the foregoing. An exemplary GHRH is represented by SEQ ID NO: 1; an exemplary PR1 T-cell epitope is represented by SEQ ID NO: 2; an exemplary Protease-3 peptide 1 is represented by SEQ ID NO: 3; an exemplary Protease-3 peptide 2 is represented by SEQ ID NO: 4; an exemplary Protease-3 peptide 3 is represented by SEQ ID NO: 5; an exemplary Protease-3 peptide 4 is represented by SEQ ID NO: 6; an exemplary Protease-3 peptide 5 is represented by SEQ ID NO: 7; an exemplary Protease-3 peptide 6 is represented by SEQ ID NO: 8; an exemplary an exemplary Protease-3 peptide 7 is represented by SEQ ID NO: 9; an exemplary Protease-3 peptide 8 is represented by SEQ ID NO: 10; an exemplary Protease-3 peptide 9 is represented by SEQ ID NO: 11; an exemplary Protease-3 peptide 10 is represented by SEQ ID NO: 12; an exemplary Protease-3 peptide 11 is represented by SEQ ID NO: 13; an exemplary an exemplary P3, B-cell epitope is represented by SEQ ID NO: 14; an exemplary P3, B-cell epitope: with spacer: is represented by SEQ ID NO: 15; an exemplary GLP1 is represented by SEQ ID NO: 16; an exemplary LHRH is represented by SEQ ID NO: 17; an exemplary PTH is represented by SEQ ID NO: 18; an exemplary Substance P is represented by SEQ ID NO: 19; an exemplary Neurokinin A is represented by SEQ ID NO: 20; an exemplary Neurokinin B is represented by SEQ ID NO: 21; an exemplary Bombesin is represented by SEQ ID NO: 22; an exemplary CCK-8 is represented by SEQ ID NO: 23; an exemplary Leucine Enkephalin is represented by SEQ ID NO: 24; an exemplary Methionine Enkephalin is represented by SEQ ID NO: 25; an exemplary [Des Ala20, Gln34] Dermaseptin is represented by SEQ ID NO: 26; an exemplary antimicrobial Peptide Surfactant is represented by SEQ ID NO: 27; an exemplary antimicrobial anionic Peptide Surfactant-associated AP is represented by SEQ ID NO: 28; an exemplary Apidaecin IA is represented by SEQ ID NO: 29; an exemplary Apidaecin IB is represented by SEQ ID NO: 30; an exemplary OV-2 is represented by SEQ ID NO: 31; an exemplary 1025, Acetyl-Adhesin Peptide 1025-1044 amide is represented by SEQ ID NO: 32; an exemplary Theromacin 49-63 is represented by SEQ ID NO: 33; an exemplary Pexiganan MSI-78 is represented by SEQ ID NO: 34; an exemplary Indolicidin is represented by SEQ ID NO: 35; an exemplary Apelin-15 63-77 is represented by SEQ ID NO: 36; an exemplary CFP10 71-85 is represented by SEQ ID NO: 37; an exemplary Lethal Factor LF Inhibitor anthrax related is represented by SEQ ID NO: 38; an exemplary Bactenecin is represented by SEQ ID NO: 39; an exemplary Hepatitis Virus C NS3 Protease Inhibitor 2 is represented by SEQ ID NO: 40; an exemplary Hepatitis Virus C NS3 Protease Inhibitor 3 is represented by SEQ ID NO: 41; an exemplary an exemplary Hepatitis Virus NS3 Protease Inhibitor 4 is represented by SEQ ID NO: 42; an exemplary NS4A-NS4B Hepatitis Virus C NS3 Protease Inhibitor 1 is represented by SEQ ID NO: 43; an exemplary HIV-1, HIV-2 Protease Substrate is represented by SEQ ID NO: 44; an exemplary anti-Flt1 Peptide is represented by SEQ ID NO: 45; an exemplary Bak-BH3 is represented by SEQ ID NO: 46; an exemplary Bax BH3 peptide 55-74 wild type is represented by SEQ ID NO: 47; an exemplary Bid BH3-r8 is represented by SEQ ID NO: 48; an exemplary CTT Gelatinase Inhibitor is represented by SEQ ID NO: 49; an exemplary E75 Her-2/neu 369-377 is represented by SEQ ID NO: 50; an exemplary GRP78 Binding Chimeric Peptide Motif is represented by SEQ ID NO: 51; an exemplary p5317-26 is represented by SEQ ID NO: 52; an exemplary EGFR2/KDR antagonist is represented by SEQ ID NO: 53; an exemplary Colivelin is represented by SEQ ID 54; an exemplary AGA-C8R HNG17 Humanin derivative is represented by SEQ ID NO: 55; an exemplary Activity-Dependent Neurotrophic Factor ADNF is represented by SEQ ID NO: 56; an exemplary Beta-Secretase Inhibitor 1 is represented by SEQ ID NO: 57; an exemplary Beta-Secretase Inhibitor 2 is represented by SEQ ID NO: 58; an exemplary chβ-Amyloid 30-16 is represented by SEQ ID NO: 59; an exemplary Humanun HN is represented by SEQ ID NO: 60; an exemplary sHNG, [Gly14]-HN, [Gly14]-Humanin is represented by SEQ ID NO: 61; an exemplary angiotensin Converting Enzyme Inhibitor BPP is represented by SEQ ID NO: 62; an exemplary Renin Inhibitor III is represented by SEQ ID NO: 63; an exemplary annexin 1 ANXA-1; an exemplary Ac2-12 is represented by SEQ ID NO: 64; an exemplary anti-Inflammatory Peptide 1 is represented by SEQ ID NO: 65; an exemplary anti-Inflammatory Peptide 2 is represented by SEQ ID NO: 66; an exemplary anti-Inflammatory Apelin 12 is represented by SEQ ID NO: 67; an exemplary [D-Phe12, Leu14]-Bombesin is represented by SEQ ID NO: 68; an exemplary antennapedia Peptide acid penetratin is represented by SEQ ID NO: 69; an exemplary antennepedia Leader Peptide CT is represented by SEQ ID NO: 70; an exemplary Mastoparan is represented by SEQ ID NO: 71; an exemplary [Thr28, Nle31]-Cholecystokinin 25-33 sulfated is represented by SEQ ID NO: 72; an exemplary Nociceptin 1-13 amide is represented by SEQ ID NO: 73; an exemplary an exemplary Fibrinolysis Inhibiting Factor is represented by SEQ ID NO: 74; an exemplary Gamma-Fibrinogen 377-395 is represented by SEQ ID NO: 75; an exemplary Xenin is represented by SEQ ID NO: 76; an exemplary Obestatin human is represented by SEQ ID NO: 77; an exemplary [His1, Lys6]-GHRP GHRP-6 is represented by SEQ ID NO: 78; an exemplary [Ala-5, β-Ala8]-Neurokinin A 4-10 is represented by SEQ ID NO: 79; an exemplary an exemplary Neuromedin B is represented by SEQ ID NO: 80; an exemplary Neuromedin C is represented by SEQ ID NO: 81; an exemplary Neuromedin N is represented by SEQ ID NO: 82; an exemplary Activity-Dependent Neurotrophic Factor ADNF-14 is represented by SEQ ID NO: 83; an exemplary Acetalin 1 Opioid Receptor antagonist 1 is represented by SEQ ID NO: 84; an exemplary Acetalin 2 Opioid Receptor antagonist 2 is represented by SEQ ID NO: 85; an exemplary Acetalin 3 Opioid Receptor antagonist 3 is represented by SEQ ID NO: 86; an exemplary ACTH 1-39 human is represented by SEQ ID NO: 87; an exemplary ACTH 7-38 human is represented by SEQ ID NO: 88; an exemplary an exemplary Sauvagine is represented by SEQ ID NO: 89; an exemplary Adipokinetic Hormone Locusta Migratoria is represented by SEQ ID NO: 90; an exemplary Myristoylated ADP-Ribosylation Factor 6, myr-ARF6 2-13 is represented by SEQ ID NO: 91; an exemplary PAMP 1-20 Proadrenomedullin 1-20 human is represented by SEQ ID NO: 92; an exemplary AGRP 25-51 is represented by SEQ ID NO: 93; an exemplary Amylin 8-37 human is represented by SEQ ID NO: 94; an exemplary angiotensin I human is represented by SEQ ID NO: 95; an exemplary angiotensin II human is represented by SEQ ID NO: 96; an exemplary Apstatin Aminopeptidase P Inhibitor is represented by SEQ ID NO: 97; an exemplary Brevinin-1 is represented by SEQ ID NO: 98; an exemplary Magainin 1 is represented by SEQ ID NO: 99; an exemplary RL-37 is represented by SEQ ID NO: 100; an exemplary LL-37 antimicrobial Peptide human is represented by SEQ ID NO: 101; an exemplary Cecropin A is represented by SEQ ID NO: 102; an exemplary antioxidant peptide A is represented by SEQ ID NO: 103; an exemplary antioxidant peptide B is represented by SEQ ID NO: 104; an exemplary L-Carnosine is represented by SEQ ID NO: 105; an exemplary Bcl 9-2 is represented by SEQ ID NO: 106; an exemplary NPVF is represented by SEQ ID NO: 107; an exemplary Neuropeptide AF hNPAF Human is represented by SEQ ID NO: 108; an exemplary Bax BH3 peptide 55-74 is represented by SEQ ID NO: 109; an exemplary bFGF Inhibitory Peptide is represented by SEQ ID NO: 110; an exemplary bFGF inhibitory Peptide II is represented by SEQ ID NO: 111; an exemplary Bradykinin is represented by SEQ ID NO: 112; an exemplary [Des-Arg10]-HOE I40 is represented by SEQ ID NO: 113; an exemplary Caspase 1 Inhibitor II is represented by SEQ ID NO: 114; an exemplary Caspase 1 Inhibitor VIII is represented by SEQ ID NO: 115; an exemplary Smac N7 Protein is represented by SEQ ID NO: 116; an exemplary MEK1 Derived Peptide Inhibitor 1 is represented by SEQ ID NO: 117; an exemplary hBD-1 β-Defensin-1 human is represented by SEQ ID NO: 118; an exemplary hBD-3 β-Defensin-3 human is represented by SEQ ID NO: 119; an exemplary hBD-4 β-Defensin-4 human is represented by SEQ ID NO: 120; an exemplary HNP-1 Defensin Human Neutrophil Peptide 1 is represented by SEQ ID NO: 121; an exemplary HNP-2 Defensin Human neutrophil Peptide-2 Dynorphin A 1-17 is represented by SEQ ID NO: 122; an exemplary Endomorphin-1 is represented by SEQ ID NO: 123; an exemplary β-Endorphin human porcine is represented by SEQ ID NO: 124; an exemplary Endothelin 2 human is represented by SEQ ID NO: 125; an exemplary Fibrinogen Binding Inhibitor Peptide is represented by SEQ ID NO: 126; an exemplary Cyclo-GRGDSP is represented by SEQ ID NO: 127; an exemplary TP508 Thrombin-derived Peptide is represented by SEQ ID NO: 128; an exemplary Galanin human is represented by SEQ ID NO: 129; an exemplary GIP human is represented by SEQ ID NO: 130; an exemplary Gastrin Releasing Peptide human is represented by SEQ ID NO: 131; an exemplary Gastrin-1 human is represented by SEQ ID NO: 132; an exemplary Ghrelin human is represented by SEQ ID NO: 133; an exemplary PDGF-BB peptide is represented by SEQ ID NO: 134; an exemplary [D-Lys3]-GHRP-6 is represented by SEQ ID NO: 135; an exemplary HCV Core Protein 1-20 is represented by SEQ ID NO: 136; an exemplary a3β1 Integrin Peptide Fragment 325 amide is represented by SEQ ID NO: 137; an exemplary Laminin Pentapeptide amide is represented by SEQ ID NO: 138; an exemplary Melanotropin-Potentiating Factor MPF is represented by SEQ ID NO: 139; an exemplary VA-β-MSH, Lipotropin-Y Proopiomelanocortin-derived is represented by SEQ ID NO:140; an exemplary Atrial Natriuretic Peptide 1-28 human is represented by SEQ ID NO: 141; an exemplary Vasonatrin Peptide 1-27 is represented by SEQ ID NO: 142; an exemplary [Ala5, β-Ala8]-Neurokinin A 4-10 is represented by SEQ ID NO: 143; an exemplary Neuromedin L NKA is represented by SEQ ID NO: 144; an exemplary Ac-Leu28, 31-Neuropeptide Y 24-26 is represented by SEQ ID NO: 145; an exemplary Alytesin is represented by SEQ ID NO: 146; an exemplary Brain Neuropeptide II is represented by SEQ ID NO: 147; an exemplary [D-tyr11]-Neurotensin is represented by SEQ ID NO: 148; an exemplary IKKy NEMO Binding Domain NBD Inhibitory Peptide is represented by SEQ ID NO: 149; an exemplary PTD-p50 NLS Inhibitory Peptide is represented by SEQ ID NO: 150; an exemplary Orexin A bovine, human, mouse, rat is represented by SEQ ID NO: 151; an exemplary Orexin B human is represented by SEQ ID NO: 152; an exemplary Aquaporin-2254-267 human Pancreastatin 37-52 is represented by SEQ ID NO: 153; an exemplary Pancreatic Polypeptide human is represented by SEQ ID NO: 154; an exemplary Neuropeptide is represented by SEQ ID NO: 155; an exemplary Peptide YY 3-36 human is represented by SEQ ID NO: 156; an exemplary Hydroxymethyl-Phytochelatin 2 is represented by SEQ ID NO: 157; an exemplary PACAP 1-27 amide, human, bovine, rat is represented by SEQ ID NO: 158; an exemplary Prolactin Releasing Peptide 1-31 human is represented by SEQ ID NO: 159; an exemplary Salusin-alpha is represented by SEQ ID NO: 160; an exemplary Salusin-beta is represented by SEQ ID NO: 161; an exemplary Saposin C22 is represented by SEQ ID NO: 162; an exemplary Secretin human is represented by SEQ ID NO: 163; an exemplary L-Selectin is represented by SEQ ID NO: 164; an exemplary Endokinin A/B is represented by SEQ ID NO: 165; an exemplary Endokinin C Human is represented by SEQ ID NO: 166; an exemplary Endokinin D Human is represented by SEQ ID NO: 167; an exemplary Thrombin Receptor 42-48 Agonist human is represented by SEQ ID NO: 168; an exemplary LSKL Inhibitor of Thrombospondin is represented by SEQ ID NO: 169; an exemplary Thyrotropin Releasing Hormone TRH is represented by SEQ ID NO: 170; an exemplary P55-TNFR Fragment is represented by SEQ ID NO: 171; an exemplary Urotensin II human is represented by SEQ ID NO: 172; an exemplary VIP human, porcine, rat is represented by SEQ ID NO: 173; an exemplary VIP antagonist is represented by SEQ ID NO: 174; an exemplary Helodermin is represented by SEQ ID NO: 175; an exemplary Exenatide is represented by SEQ ID NO: 176; an exemplary ZP10 AVE00100 is represented by SEQ ID NO: 177; an exemplary Pramlinitide is represented by SEQ ID NO: 178; an exemplary AC162352 PYY3-36 is represented by SEQ ID NO: 179; an exemplary PYY is represented by SEQ ID NO: 180; an exemplary Obinepitide is represented by SEQ ID NO: 181; an exemplary Glucagon is represented by SEQ ID NO: 182; an exemplary GRP is represented by SEQ ID NO: 183; an exemplary Ghrelin GHRP6 is represented by SEQ ID NO: 184; an exemplary Leuprolide is represented by SEQ ID NO: 185; an exemplary Histrelin is represented by SEQ ID NO: 186; an exemplary Oxytocin is represented by SEQ ID NO: 187; an exemplary Atosiban RWJ22164 is represented by SEQ ID NO: 188; an exemplary Sermorelin is represented by SEQ ID NO: 189; an exemplary Nesiritide is represented by SEQ ID NO: 190; an exemplary bivalirudin Hirulog is represented by SEQ ID NO: 191; an exemplary Icatibant is represented by SEQ ID NO: 192; an exemplary Aviptadil is represented by SEQ ID NO: 193; an exemplary Rotigaptide ZP123, GAP486 is represented by SEQ ID NO: 194; an exemplary Cilengitide EMD-121924, RGD Peptides is represented by SEQ ID NO: 195; an exemplary AlbuBNP is represented by SEQ ID NO: 196; an exemplary BN-054 is represented by SEQ ID NO: 197; an exemplary angiotensin II is represented by SEQ ID NO: 198; an exemplary MBP-8298 is represented by SEQ ID NO: 199; an exemplary Peptide Leucine Arginine is represented by SEQ ID NO: 200; an exemplary Ziconotide is represented by SEQ ID NO: 201; an exemplary AL-208 is represented by SEQ ID NO: 202; an exemplary AL-108 is represented by SEQ ID NO: 203; an exemplary Carbeticon is represented by SEQ ID NO: 204; an exemplary Tripeptide is represented by SEQ ID NO: 205; an exemplary SAL is represented by SEQ ID NO: 206; an exemplary Coliven is represented by SEQ ID NO: 207; an exemplary Humanin is represented by SEQ ID NO: 208; an exemplary ADNF-14 is represented by SEQ ID NO: 209; an exemplary VIP Vasoactive Intestinal Peptide is represented by SEQ ID NO: 210; an exemplary Thymalfasin is represented by SEQ ID NO: 211; an exemplary Bacitracin USP is represented by SEQ ID NO: 212; an exemplary Gramidicin USP is represented by SEQ ID NO: 213; an exemplary Pexiganan MSI-78 is represented by SEQ ID NO: 214; an exemplary P113 is represented by SEQ ID NO: 215; an exemplary PAC-113 is represented by SEQ ID NO: 216; an exemplary SCV-07 is represented by SEQ ID NO: 217; an exemplary HLF1-11 Lactoferrin is represented by SEQ ID NO: 218; an exemplary DAPTA is represented by SEQ ID NO: 219; an exemplary TRI-1144 is represented by SEQ ID NO: 220; an exemplary Tritrpticin is represented by SEQ ID NO: 221; an exemplary antiflammin 2 is represented by SEQ ID NO: 222; an exemplary Gattex Teduglutide, ALX-0600 is represented by SEQ ID NO: 223; an exemplary Stimuvax L-BLP25 is represented by SEQ ID NO: 224; an exemplary Chrysalin TP508 is represented by SEQ ID NO: 225; an exemplary Melanonan II is represented by SEQ ID NO: 226; an exemplary Spantide II is represented by SEQ ID NO: 227; an exemplary Ceruletide is represented by SEQ ID NO: 228; an exemplary Sincalide is represented by SEQ ID NO: 229; an exemplary Pentagastin is represented by SEQ ID NO: 230; an exemplary Secretin is represented by SEQ ID NO: 231; an exemplary Endostatin peptide is represented by SEQ ID NO: 232; an exemplary E-selectin is represented by SEQ ID NO: 233; an exemplary HER2 is represented by SEQ ID NO: 234; an exemplary IL-6 is represented by SEQ ID NO: 235; an exemplary IL-8 is represented by SEQ ID NO: 236; an exemplary IL-10 is represented by SEQ ID NO: 237; an exemplary PDGF is represented by SEQ ID NO: 238; an exemplary Thrombospondin is represented by SEQ ID NO: 239; an exemplary uPA 1 is represented by SEQ ID NO: 240; an exemplary uPA 2 is represented by SEQ ID NO: 241; an exemplary VEGF is represented by SEQ ID NO: 242; an exemplary VEGF 2 is represented by SEQ ID NO: 243; an exemplary Pentapeptide-3 is represented by SEQ ID NO: 244; an exemplary Glutathione is represented by SEQ ID NO: 245; an exemplary XXLRR is represented by SEQ ID NO. 246; an exemplary Beta-Amyloid Fibrillogenesis is represented by SEQ ID NO: 247; an exemplary Endomorphin-2 is represented by SEQ ID NO: 248; an exemplary TIP 39 Tuberoinfundibular Neuropeptide is represented by SEQ ID NO: 249; an exemplary PACAP 1-38 amide, human, bovine, rat is represented by SEQ ID NO: 250; an exemplary TGFβ activating peptide is represented by SEQ ID NO: 251; an exemplary Insulin sensitizing factor ISF402 is represented by SEQ ID NO: 252; an exemplary Transforming Growth Factor β1 Peptide TGF-β1 is represented by SEQ ID NO: 253; an exemplary Caerulein Releasing Factor is represented by SEQ ID NO: 254; an exemplary IELLQAR 8-branch MAPS is represented by SEQ ID NO: 255; an exemplary Tigapotide PK3145 is represented by SEQ ID NO: 256; an exemplary Goserelin is represented by SEQ ID NO: 257; an exemplary Abarelix is represented by SEQ ID NO: 258; an exemplary Cetrorelix is represented by SEQ ID NO: 259; an exemplary Ganirelix is represented by SEQ ID NO: 260; an exemplary Degarelix Triptorelin is represented by SEQ ID NO: 261; an exemplary Barusiban FE 200440 is represented by SEQ ID NO: 262; an exemplary Pralmorelin is represented by SEQ ID NO: 263; an exemplary Octreotide is represented by SEQ ID NO: 264; an exemplary Eptifibatide is represented by SEQ ID NO: 265; an exemplary Netamiftide INN-00835 is represented by SEQ ID NO: 266; an exemplary Daptamycin is represented by SEQ ID NO: 267; an exemplary Spantide II 1 is represented by SEQ ID NO: 268; an exemplary Delmitide RDP-58 is represented by SEQ ID NO: 269; an exemplary AL-209 is represented by SEQ ID NO: 270; an exemplary Enfuvirtide is represented by SEQ ID NO: 271; an exemplary IDR-1 is represented by SEQ ID NO: 272; an exemplary Hexapeptide-6 is represented by SEQ ID NO: 272; an exemplary Insulin-A chain is represented by SEQ ID NO: 274; an exemplary Lanreotide is represented by SEQ ID NO: 275; an exemplary Hexapeptide-3 is represented by SEQ ID NO: 276; an exemplary Insulin B-chain is represented by SEQ ID NO: 277; an exemplary Glargine-A chain is represented by SEQ ID NO: 278; an exemplary Glargine-B chain is represented by SEQ ID NO: 279; an exemplary Insulin-LisPro B-chain analog is represented by SEQ ID NO: 280; an exemplary Insulin-Aspart B-chain analog is represented by SEQ ID NO: 281; an exemplary Insulin-Glulisine B chain analog is represented by SEQ ID NO: 282; an exemplary Insulin-Determir B chain analog is represented by SEQ ID NO: 283; an exemplary Somatostatin is represented by SEQ ID NO: 284; an exemplary Somatostatin Tumor Inhibiting analog is represented by SEQ ID NO: 285; an exemplary Pancreastatin 37-52 is represented by SEQ ID NO: 286; an exemplary Vasoactive Intestinal Peptide fragment KKYL-NH2 is represented by SEQ ID NO: 287; an exemplary Dynorphin A is represented by SEQ ID NO: 288; or analogs thereof of any of the foregoing.

More preferably, the polyaminoacid is a protein or polypeptide selected from the group comprising: PYY; Obinepitide; PTH; Leuprolide; Atosiban; Sermorelin; Pralmorelin; Nesiritide; Rotigaptide; Cilengitide; MBP-8298; AL-108; Enfuvirtide; Thymalfasin; Daptamycin; HLF1-11; Lactoferrin; Gattex; Teduglutide; ALX-0600; Delmitide; RDP-58; pentapeptide-3; hexapeptide-6; L-carnosine; and glutathione or analogs thereof. An exemplary PYY is represented by SEQ ID NO: 181; an exemplary Obinepitide is represented by SEQ ID NO: 183; an exemplary PTH is represented by SEQ ID NO:18; an exemplary Leuprolide is represented by SEQ ID NO: 187; an exemplary Atosiban is represented by SEQ ID NO: 190; an exemplary Sermorelin is represented by SEQ ID NO:191; an exemplary Pralmorelin is represented by SEQ ID NO:268; an exemplary Nesiritide is represented by SEQ ID NO: 192; an exemplary Rotigaptide is represented by SEQ ID NO:196; an exemplary Cilengitide is represented by SEQ ID NO: 197; an exemplary MBP-8298 is represented by SEQ ID NO:202; an exemplary AL-108 is represented by SEQ ID NO:206; an exemplary Enfuvirtide is represented by SEQ ID NO: 278; an exemplary Thymalfasin is represented by SEQ ID NO: 214; an exemplary Daptamycin is represented by SEQ ID NO: 272; an exemplary HLF1-11 is represented by SEQ ID NO: 222; an exemplary Lactoferrin is represented by SEQ ID NO: 222; an exemplary Gattex is represented by SEQ ID NO: 227; an exemplary Teduglutide is represented by SEQ ID NO: 227; an exemplary ALX-0600 is represented by SEQ ID NO: 227; an exemplary Delmitide is represented by SEQ ID NO: 274; an exemplary RDP-58 is represented by SEQ ID NO: 274; an exemplary pentapeptide-3 is represented by SEQ ID NO: 248; an exemplary hexapeptide-6 is represented by SEQ ID NO: 107; an exemplary L-carnosine is represented by SEQ ID NO: 107; an exemplary and glutathione is represented by SEQ ID NO:249; or analogs thereof of any of the foregoing. In some embodiments the polyaminoacid pharmaceuticals include GLP-1, LHRH, PTH, Substance P, Neurokinin A, Neurokinin B, Bombesin, CCK-8, Leucine Enkephalin ENKEPHALIN, Methionine Enkephalin, GHRH, PR1 (T-cell epitope), P3 (B-cell epitope) and Somatostatin; or analogs thereof of any of the foregoing. An exemplary GLP-1 is represented by SEQ ID NO: 16; an exemplary LHRH is represented by SEQ ID NO: 17; an exemplary PTH is represented by SEQ ID NO: 18; an exemplary Substance P is represented by SEQ ID NO: 19; an exemplary Neurokinin A is represented by SEQ ID NO: 20; an exemplary Neurokinin B is represented by SEQ ID NO: 21; an exemplary Bombesin is represented by SEQ ID NO: 22; an exemplary CCK-8 is represented by SEQ ID NO: 23; an exemplary Leucine Enkephalin is represented by SEQ ID NO: 24; an exemplary Methionine Enkephalin is represented by SEQ ID NO: 25; an exemplary GHRH is represented by SEQ ID NO: 1; an exemplary PR1 (T-cell epitope) is represented by SEQ ID NO: 2; an exemplary P3 (B-cell epitope) is represented by SEQ ID NO: 14; and an exemplary Somatostatin is represented by SEQ ID NO: 284; or analogs thereof of any of the foregoing.

Even more preferably, the polyaminoacid is a protein or polypeptide selected from the group comprising: GLP-1, LHRH, PTH, Substance P, Neurokinin A, Neurokinin B, Bombesin, CCK-8, Leucine Enkephalin ENKEPHALIN, Methionine Enkephalin, GHRH, PR1 (T-cell epitope), P3 (B-cell epitope) and Somatostatin; or analogs thereof of any of the foregoing. An exemplary GLP-1 is represented by SEQ ID NO: 16; an exemplary LHRH is represented by SEQ ID NO: 17; an exemplary PTH is represented by SEQ ID NO: 18; an exemplary Substance P is represented by SEQ ID NO: 19; an exemplary Neurokinin A is represented by SEQ ID NO: 20; an exemplary Neurokinin B is represented by SEQ ID NO: 21; an exemplary Bombesin is represented by SEQ ID NO: 22; an exemplary CCK-8 is represented by SEQ ID NO: 23; an exemplary Leucine Enkephalin is represented by SEQ ID NO: 24; an exemplary Methionine Enkephalin is represented by SEQ ID NO: 25; an exemplary GHRH is represented by SEQ ID NO: 1; an exemplary PR1 (T-cell epitope) is represented by SEQ ID NO: 2; an exemplary P3 (B-cell epitope) is represented by SEQ ID NO: 14; and an exemplary Somatostatin is represented by SEQ ID NO: 284; or analogs thereof of any of the foregoing.

Analogs of the polyaminoacids described herein may comprise one or more amino acid substitutions, deletions, inversions, or additions when compared with the polyaminoacid. Analogs may include molecules with one or more conservative amino acid substitutions. Conservative amino acid substitutions, including preferred amino acid substitutions of interest are shown below.

| Original | Conservative | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine, ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

If such substitutions result in a change in biological activity, then additional changes, including those in reference to amino acid classes, may be introduced. Naturally occurring residues are divided into amino acid classes or groups based on common side-chain properties: (1) hydrophobic: norleucine, met, ala, val, leu, ile; (2) neutral hydrophilic cys, ser, thr; (3) acidic: asp, glu; (4) basic: asn, gin, his, lys, arg; (5) residues that influence chain orientation: gly, pro; and (6) aromatic: trp, tyr, phe. Non-conservative substitutions may entail exchanging a member of one of these classes for another class. Such substituted residues also may be introduced into the conservative substitution sites or, more preferably, into the remaining (non-conserved) sites.

In an embodiment the polyethoid comprising the improvement is biologically active. It can be have an increased resistance to a peptidase or protease, It can be an imaging or diagnostic agent, a receptor agonist or receptor antagonist, a therapeutic agent, derived from an α-amino acid, a protein, or a polypeptide. The polyaminoacid can further be a multiple combination of each of these.

Chirality is often important to the specificity of bioactive peptides and previously known ethoid synthetic methodologies based upon reactions at the chiral center were characterized by poor chiral control and degradation of chiral integrity. The present methods provide polyethoids and polyethoidpeptides that retain the chirality at the chiral centers. Notably, the disclosed reductive etherification reaction occurs at a non chiral center and can make use of chiral α-hydroxy acid building blocks so that chirality is not affected. In the disclosed reductive etherification reactions the integrity of any neighboring chiral centers is retained, preferably absolutely retained, and the reaction is tolerant of neighboring substituents.

Polyethoids and polyamides in this disclosure can contain one or more chiral centers. Each chiral center can be racemic, enantiomerically enriched, or enantiomerically pure. Preferably each chiral center can be enantiomerically enriched. More preferably, the chiral center can be substantially enantiomerically pure. Enantiomerically pure, as understood in the art is nearly 100% of one stereocenter, but because measuring absolute pure stereochemistry is not analytically possible, it is appreciated that enantiomerically pure means nearly 100% of a single stereocenter.

One way to describe the amount of enantiomerically enriched compound is by enantiomeric excess. The phrase “enantiomeric excess” or “ee” is a measure, for a given sample, of the excess of one enantiomer over a racemic sample of a chiral compound and is expressed as a percentage. Enantiomeric excess is defined as 100×(er−1)/(er+1), where “er” is the ratio of the more abundant enantiomer to the less abundant enantiomer. the ratio of the more abundant enantiomer to the less abundant enantiomer. Enantiomeric excess can also be defined as (R−S)/(R+S) where R represents the amount of one enantiomer and S represents the amount of another enantiomer.

The phrases “enantiomerically pure” or “enantiopure” refer to a sample of an enantiomer having an ee of about 99% or greater.

Therefore an aspect of this disclosure is a compound described by formula XI

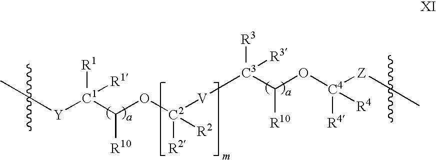

Where each m can be an integer ≧0 and each a can be an independently selected integer=1 or =2, such that when m=0, at least two carbons selected from C¹, C³ and C⁴ are chiral and have an enantiomeric excess of at least about 20%, or when m≧1, at least three carbons selected from C¹, each C², C³ and C⁴ are chiral and have an enantiomeric excess of at least about 20%, R¹, each R², R³ and R⁴ are each independently selected from the group consisting of hydrocarbyl and substituted hydrocarbyl, R^{1′}, each R^{2′}, R^{3′} and R^{4′} are each independently selected from the group consisting of hydrocarbyl and substituted hydrocarbyl, each R¹⁰ is independently selected from the group consisting of H, C₁-C₃ alkyl and substituted C₁-C₃ alkyl, each V is independently selected from the group consisting of —C(O)NH— and -ψ[ ]-, and Y and Z are each independently selected from the group consisting hydrocarbyl and substituted hydrocarbyl. In a preferred embodiment, at least one a=1, more preferably each a=1. Alternatively, at least one a=2, or each a=2.

In an embodiment, R¹, each R², R³ and R⁴ are each independently selected from the group consisting of alkyl, substituted alkyl, heteroalkyl, substituted heteroalkyl, aryl, substituted aryl, heteroaryl, substituted heteroaryl, alicyclic, substituted alicyclic, heterocyclic, substituted heterocyclic, including in each case one or more ring structures formed between adjacent pendant moieties selected from R¹, each R², R³ and R⁴, and R^{1′}, each R^{2′}, R^{3′} and R^{4′} are each independently selected from the group consisting of alkyl, substituted alkyl, heteroalkyl, substituted heteroalkyl, aryl, substituted aryl, heteroaryl, substituted heteroaryl, alicyclic, substituted alicyclic, heterocyclic, substituted heterocyclic, including in each case one or more ring structures formed between adjacent pendant moieties selected from R¹, each R², R³ and R⁴. Y and Z can be each independently selected from the group consisting of —V—, -functional group, -protected functional group, -linking moiety, -conjugate and -terminal group. Preferably, R¹, each R², R³ and R⁴ in Formula XI can be each an independently selected side chain moiety having a structure of an amino acid side chain, and R^{1′}, each R^{2′}, R^{3′} and R^{4′} can be each independently selected from the group consisting of H, C₁-C₃ alkyl and substituted C₁-C₃ alkyl. More preferably, a=1, R¹′, each R²′, R³′ and R⁴′ can be each independently selected from the group consisting of H and methyl, each R¹⁰ can be independently selected from the group consisting of H, methyl and substituted methyl, each V can be independently selected from the group consisting of —C(O)NH— and -ψ[ ]-, and Y and Z can be each independently selected from the group consisting of —V—, functional group, -protected functional group, -linking moiety, -conjugate and -terminal group.

In an embodiment, the enantiomeric excess of any chiral backbone carbon attached to an side chain moiety is at least about 20%, preferably at least about 50%, more preferably at least about 80%, even more preferably at least about 90% and most preferably at least about 98%. In an embodiment of formula xx, R¹, each R², R³ and R⁴ are each pendant to a backbone carbon which is chiral and has an enantiomeric excess of at least about 20%, preferably at least about 50%, more preferably at least about 80%, even more preferably at least about 90% and most preferably at least about 98%. In an embodiment of Formula X, each R⁰, R¹, each R², R³, each R⁴ and R⁵ are each pendant to a backbone carbon which is chiral and has an enantiomeric excess of at least about 20%, preferably at least about 50%, more preferably at least about 80%, even more preferably at least about 90% and most preferably at least about 98%.

In an embodiment, a backbone carbon, generally the carbon bonded to a aminoacid like side chain, more specially denoted C¹, C², C³ or C⁴, can be chiral and have an enantiomeric excess of at least about 20%, preferably at least about 50%, more preferably at least about 80%, even more preferably at least about 90% and most preferably at least about 98%. Alternatively, in a compound containing backbone carbons, at least 50% of the backbone carbons that are chiral have an enantiomeric excess of at least about 20%, preferably, at least 70% of the backbone carbons that are chiral have an enantiomeric excess of at least about 20%, more preferably about 90% of the backbone carbons that are chiral have an enantiomeric excess of at least about 20%, most preferably, each of the backbone carbons that are chiral have an enantiomeric excess of at least about 20%.

More particularly in a compound containing C¹, C², C³ or C⁴ at least 50% of the carbons selected from C¹, C², C³ or C⁴ can be chiral and have an enantiomeric excess of at least about 20%, preferably, at least 70% of the carbons selected from C¹, C², C³ or C⁴ can be chiral and have an enantiomeric excess of at least about 20%, more preferably about 90% of the carbons selected from C¹, C², C³ or C⁴ can be chiral and have an enantiomeric excess of at least about 20%, most preferably, each of the carbons selected from C¹, C², C³ or C⁴ can be chiral and have an enantiomeric excess of at least about 20%.

### Synthesis Aproaches—Modular Chiral Synthesis; Solid Phase Synthesis

The compounds of the disclosure can be prepared using any method for preparing amide and ethoid compounds, including but not limited to the methods set forth in the previous Schemes. The methods can be conducted on a solid support using solid phase chemical techniques, or can be conducted in solution phase using solution phase techniques. The compounds of the disclosure can be prepared using both solid and solution phase techniques. The synthesis can be stepwise using individual monomer units, but dimeric or trimeric units or even higher units can be used as needed.

In an embodiment, a compound comprising a polyethoid of formula

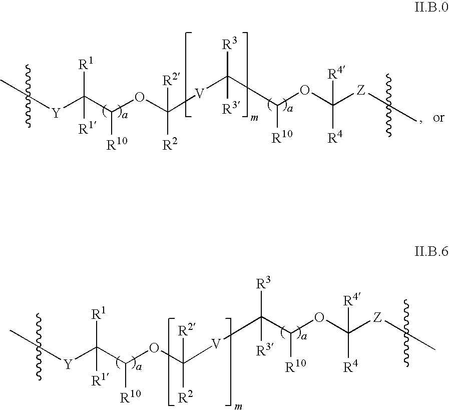

can be synthesized on a support through a series of controlled stepwise reactions, and optionally cleaving the polyethoid moiety from the support,
wherein

m is an integer ≧0,

each a is an independently selected integer=1 or =2,

R¹, each R², each R³, and R⁴ are each an independently selected side chain moiety having a structure of an amino acid side chain,

R^{1′}, each R^{2′}, each R^{3′} and R^{4′} are each independently selected from the group consisting of H, C₁-C₃ alkyl and substituted C₁-C₃ alkyl,

each R¹⁰ is independently selected from the group consisting of H, C₁-C₃ alkyl and substituted C₁-C₃ alkyl,

V is selected from the group consisting of —C(O)NH— and -ψ[ ]-, and

Y and Z are each independently selected from the group consisting hydrocarbyl and substituted hydrocarbyl.

The support can be any support known in the art for conducting solid phase synthesis. In an embodiment, the support can be a polymeric support covalently linked to at least one of Y or Z through a linking moiety. The polymer support can be any solid support used in solid phase synthesis, including but not limited to Rink resin, Wang resin, trityl chloride resin, HMBA AM resin, Merrifield resin, Oxime resin, BAL resin, Any derivatized chloromethylpolystyrene resin, any derivatized aminomethylpolystyrene resin, any derivatized NovaSyn TG resin, any derivatized PEGA resin, any derivatized Novagel resin, any molded grafted polyethylene support, any molded grafted polypropylene support, Rink SynPhase PS lanterns, Rink SynPhase PA lanterns, hydroxymethyl SynPhase PS lanterns, hydroxymethyl SynPhase PA lanterns, or any derivatized SynPhase lanterns.

In the polyethoid, Y can be a linking moiety covalently bonded to the support, the linking moiety optionally comprising —V—, and the polyethoid moiety is synthesized by a process comprising forming a first moiety comprising an ethoid and having a formula

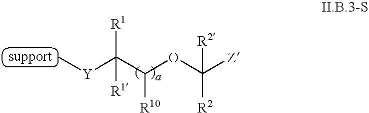

through one or more reactions, wherein Z′ is a functional group or a protected functional group; optionally forming a second moiety having a formula

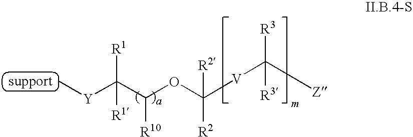

through one or more further reactions with the first moiety, wherein Z″ is a functional group or a protected functional group; and forming a third moiety comprising at least two ethoids and having a formula

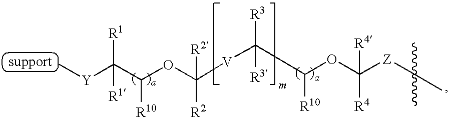

through one or more further reactions with the second moiety when m≧1, or with the first moiety when m=0.

In the polyethoid, Z can be a linking group covalently bonded to the support, the linking moiety optionally comprising —V—, and the polyethoid moiety is synthesized by a process comprising forming a first moiety comprising an ethoid and having a formula

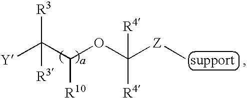

through one or more reactions, wherein Y′ is a functional group or a protected functional group; optionally forming a second moiety having a formula

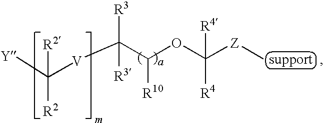

through one or more further reactions with the first moiety, wherein Y″ is a functional group or a protected functional group; and forming a third moiety comprising at least two ethoids and having a formula

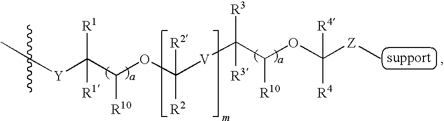

As before, in an embodiment, at least one a=1, or each a=1, or at least one a=2 or each a=2. In an alternate embodiment, at least one of Y or Z can comprise a —V—; and each Y and Z are each independently selected from the group consisting of —V—, -functional group, -protected functional group, -linking moiety, -conjugate and -terminal group. In an embodiment, R¹, each R², each R³ and R⁴ are each an independently selected side chain moiety having a structure of an amino acid side chain; R^{1′}, each R^{2′}, each R^{3′} and R^{4′} are each independently selected from the group consisting of H, C₁-C₃ alkyl and substituted C₁-C₃ alkyl, preferably H or methyl, and more preferably H; Y and Z are each independently selected from the group consisting of —V—, -functional group, -protected functional group, -linking moiety, -conjugate and -terminal group. Each —V_ can independently be selected —C(O)NH— and -ψ[ ]-. R¹⁰ can be as defined before and is preferably H.

In an embodiment, the first moiety having a formula II.B.3-S

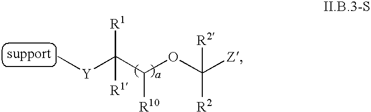

can be formed through one or more reactions including reacting a first chiral compound having a formula II.B.1 with a second chiral compound having a formula II.B.2

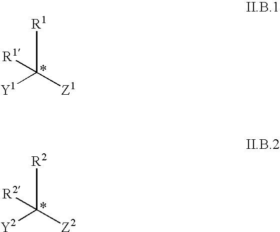

wherein Y¹ is the linking moiety, Y, covalently bonded to the support, Z¹ is a functional group selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH, Y² is a functional group reactive with Z¹ and is selected from —X, —OH, —CH₂OH, —O-silyl, —CH₂O-silyl, —O⁻M⁺, and —CH₂O⁻M⁺, X is halogen, M is an alkali or alkaline earth, and Z² is the functional group or protected functional group, Z′.

In an embodiment, a third moiety having the formula II.B.0-S

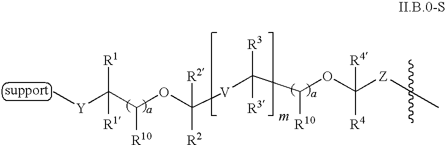

can be formed through one or more reactions including

(i) optionally forming the second moiety having a formula II.B.4-S

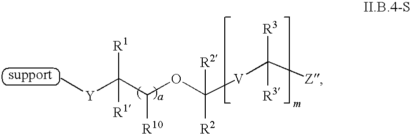

(ii) converting (A) when m=0, the —Z′ functional group or protected functional group of compound II.B.3-S or (B) when m≧1, the —Z″ functional group or protected functional group of compound II.B.4-S, in each case to a —Z¹ functional group through one or more reactions, thereby forming a compound of formula II.B.5-S having a formula

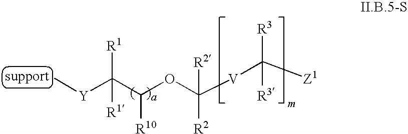

and

(iv) forming the third moiety II.B.0-S through one or more reactions including reacting the compound of formula II.B.5-S with the compound of formula II.B.8

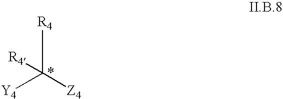

wherein Y₄ is a functional group reactive with Z₁ and is selected from —X, —OH, —CH₂OH, —O-silyl, —CH₂O-silyl, —O⁻M⁺, and —CH₂O⁻M⁺, X is halogen, M is a metal cation, and Z⁴ is the Z group, and is selected from the group consisting of —V—, -functional group, -protected functional group, -linking moiety-, -conjugate, and -terminal group.

In an embodiment, a first moiety having a formula II.B.9-S

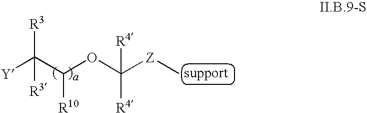

can be formed through one or more reactions including reacting a first chiral compound having a formula II.B.7 with a second chiral compound having a formula II.B.8

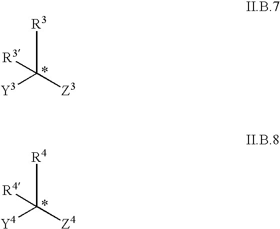

Wherein Y³ is the functional group or protected functional group, Y′; Z³ is a functional group reactive with Y⁴ and is selected from —CHR¹⁰OH, —CH₂CHR¹₀OH, —C(O)H, —C(O)R¹⁰, —CH₂C(O)H, —CH₂C(O)R¹⁰, —C(O)OH, and —CH₂C(O)OH; Y⁴ is a functional group selected from —X, —OH, —CH₂OH, —O-silyl, —CH₂O-silyl, —O⁻M⁺, and —CH₂O⁻M⁺, X is halogen, M is a an alkali or alkaline earth; and Z⁴ is the linking moiety, Z, covalently bonded to the support.

In an embodiment, the third moiety having the formula II.B.6-S

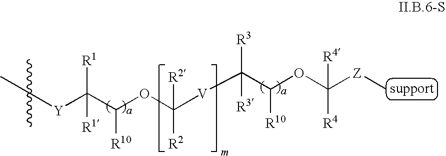

is formed through one or more reactions including:

(i) optionally forming the second moiety having a formula II.B.10-S

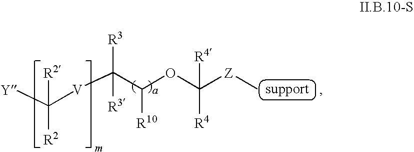

(ii) converting (A) when m=0, the —Y′ functional group or protected functional group of compound II.B.9-S or (B) when m≧1, the —Y″ functional group or protected functional group of compound II.B.10-S, in each case to a —Y⁴ functional group through one or more reactions, thereby forming a compound of formula II.B.11-S having a formula

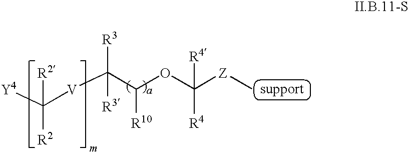

and

(iv) forming the third moiety II.B.6-S through one or more reactions including reacting the compound of formula II.B.11 with the compound of formula II.B.1-S

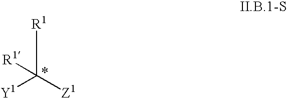

wherein Y¹ is the Y group, and is selected from the group consisting of —V—, -functional group, -protected functional group, -linking moiety-, -conjugate, and -terminal group, and Z¹ is a functional group reactive with Y⁴ and is selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH; Y⁴ is a functional group selected from —X, —OH, —CH₂OH, —O-silyl, —CH₂O-silyl, —O⁻M⁺, and —CH₂O⁻.

Compounds in the disclosure can be prepared with chirality retained at the backbone carbon positions using modular stepwise coupling. In an embodiment, a polyethoid having a formula II.B.0 or a polyethoid moiety having a formula II.B.6

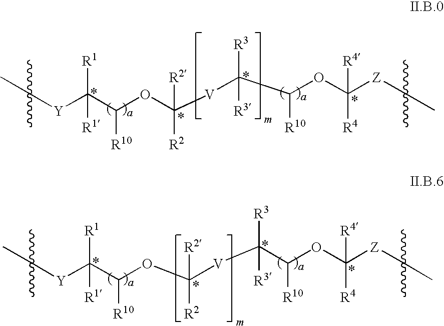

can be prepared with a method using a series of controlled stepwise reactions,

wherein each m is an integer ≧0, each a is an independently selected integer=1 or =2, the symbol “*” denotes an optionally chiral carbon, R¹, each R², each R³, and R⁴ are each an independently selected side chain moiety having a structure of an amino acid side chain, R¹′, each R²′, R³′ and R⁴′ are each independently selected from the group consisting of H, C₁-C₃ alkyl and substituted C₁-C₃ alkyl, each R¹⁰ is independently selected from the group consisting of H, C₁-C₃ alkyl and substituted C₁-C₃ alkyl, V is selected from the group consisting of —C(O)NH— and -ψ[ ]-, and Y and Z are each independently selected from the group consisting hydrocarbyl and substituted hydrocarbyl.

In an aspect the polyethoid moiety having a formula II.B.0 can be synthesized by a process comprising:

(i) forming a compound II.B.3 comprising an ethoid and having a formula

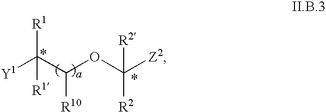

through one or more reactions including reacting a first chiral compound having a formula II.B.1 with a second chiral compound having a formula II.B.2

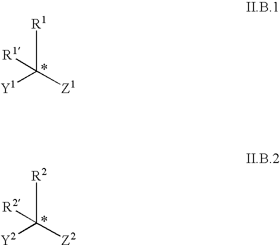

Wherein Y₁ is selected from the group consisting of —V—, -functional group, -protected functional group, -linking moiety-, -conjugate, and -terminal group, Z¹ is a functional group selected from —CHR¹⁰OH, —CH₂CHR¹⁰OH, —C(O)H, —C(O)R¹⁰, —CH₂C(O)H, —CH₂C(O)R¹⁰, —C(O)OH, —CH₂C(O)OH, Y² is a functional group reactive with Z¹ and is selected from —X, —OH, —CH₂OH, —O-silyl, —CH₂O-silyl, —O⁻M⁺, and —CH₂O⁻M⁺, X is halogen, M is an alkali or alkaline earth, and Z₂ is a functional group or protected functional group,

(ii) optionally forming a compound II.B.4 comprising an ethoid and one or more one or more —V— and having a formula

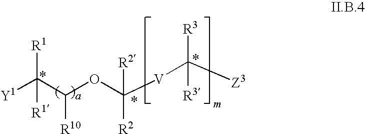

through one or more reactions, wherein Z³ is a functional group or protected functional group,

(iii) converting (A) when m=0, the —Z² functional group or protected functional group of compound II.B.3 or (B) when m≧1, the —Z³ functional group or protected functional group of compound II.B.4, in each case to a —Z¹ functional group through one or more reactions, thereby forming a compound of formula II.B.5 having a formula

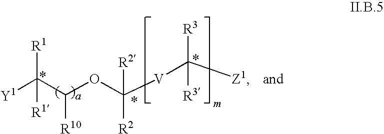

(iv) forming the compound comprising the polyethoid of formula II.B.0 through one or more reactions including reacting the compound of formula II.B.5 with the compound of formula II.B.8

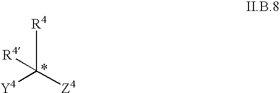

wherein Y₄ is a functional group reactive with Z¹ and is selected from —X, —OH, —CH₂OH, —O-silyl, —CH₂O-silyl, —O⁻M⁺, and —CH₂O⁻M⁺, X is halogen, M is an alkali or alkaline earth, and Z⁴ is selected from the group consisting of —V—, -functional group, -protected functional group, -linking moiety-, -conjugate, and -terminal group.

In an aspect, the polyethoid having a formula II.B.6 can be synthesized by a process comprising:

(i) forming a compound II.B.9 comprising an ethoid and having a formula

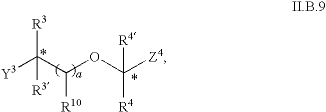

through one or more reactions including reacting a first chiral compound having a formula II.B.7 with a second chiral compound having a formula II.B.8

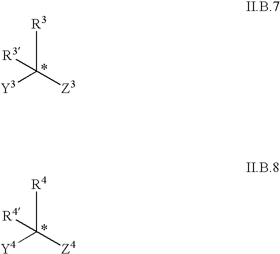

wherein Y³ is a functional group or protected functional group, Z³ is a functional group reactive with Y⁴ and is selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH, Y⁴ is a functional group selected from —X, —OH, —CH₂OH, —O-silyl, —O⁻M⁺, and —CH₂O⁻M⁺, X is halogen, M is an alkali or alkaline earth, and Z⁴ is selected from the group consisting of —V—, -functional group, -protected functional group, -linking moiety-, -conjugate, and -terminal group,

(ii) optionally forming a compound II.B.10 comprising an ethoid and one or more one or more —V— and having a formula

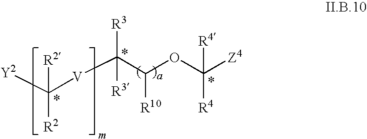

through one or more reactions, wherein Y² is a functional group or protected functional group,

(iii) converting (A) when m=0, the —Y³ functional group or protected functional group of compound II.B.9 or (B) when m≧1, the —Y² functional group or protected functional group of compound II.B.10, in each case to a —Y⁴ functional group through one or more reactions, thereby forming a compound of formula II.B.11 having a formula

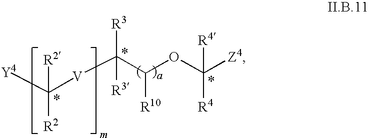

and (iv) forming the compound comprising the polyethoid of formula II.B.6 through one or more reactions including reacting the compound of formula II.B.11 with the compound of formula II.B.1

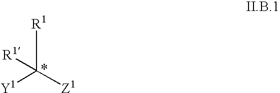

Wherein Y¹ is selected from the group consisting of —V—, -functional group, -protected functional group, -linking moiety-, -conjugate, and -terminal group, and Z₁ is a functional group reactive with Y⁴ and is selected from —CHR¹⁰OH, —CH₂CHR¹⁰OH, —C(O)H, —C(O)R¹⁰, —CH₂C(O)H, —CH₂C(O)R¹⁰, —C(O)OH, and —CH₂C(O)OH.

As before, in an embodiment, at least one a=1, or each a=1, or at least one a=2 or each a=2. In an alternate embodiment, at least one of Y or Z can comprise a —V—; and each Y and Z are each independently selected from the group consisting of —V—, -functional group, -protected functional group, -linking moiety, -conjugate and -terminal group. In an embodiment, R¹, each R², each R³ and R⁴ are each an independently selected side chain moiety having a structure of an amino acid side chain; R^{1′}, each R^{2′}, each R^{3′} and R^{4′} are each independently selected from the group consisting of H, C₁-C₃ alkyl and substituted C₁-C₃ alkyl, preferably H or methyl, and more preferably H; Y and Z are each independently selected from the group consisting of —V—, -functional group, -protected functional group, -linking moiety, -conjugate and -terminal group. Each —V— can independently be selected —C(O)NH— and -ψ[ ]-. R¹⁰ can be as defined before and is preferably H.

In an embodiment, the method provides for chiral carbons in the backbone of the polyethoid. In an embodiment, at least 50% of the backbone carbons having pendant R¹, R², R³ or R⁴ (designated with *), preferably at least about 70% of the backbone carbons, and more preferably at least about 90% of the backbone carbons, can be chiral and have an enantiomeric excess of at least about 20%, preferably at least about 50%, more preferably at least about 80%, even more preferably at least about 90% and most preferably at least about 98%.

In aspects of the methods, when Z¹ is —CHR¹⁰OH, or —CH₂CHR¹⁰OH, Y² can be —X. When Z¹ is —CHR¹⁰OH, or —CH₂CHR¹⁰OH, Y⁴ can be —X. When Z³ is selected from —CHR¹⁰OH, and —CH₂CHR¹⁰OH, Y⁴ can be —X. When Z¹ is selected from —CHR¹⁰OH, and —CH₂CHR¹⁰OH, Y⁴ can be —X. When Z¹ is selected from —C(O)H, —C(O)R¹⁰, —CH₂C(O)H, and —CH₂C(O)R¹⁰, Y² can be selected from —OH, —CH₂OH, —O-silyl, —CH₂O-silyl, —O⁻M⁺, and —CH₂O⁻M⁺. When Z¹ is selected from —C(O)H, —C(O)R¹⁰, —CH₂C(O)H, and —CH₂C(O)R¹⁰, Y⁴ can be selected from —OH, —CH₂OH, —O-silyl, —CH₂O-silyl, —O⁻M⁺, and —CH₂O⁻M⁺. When Z³ is selected from —C(O)H, —C(O)R¹⁰, —CH₂C(O)H, and —CH₂C(O)R¹⁰, Y⁴ can be selected from —OH, —CH₂OH, —O-silyl, —CH₂O-silyl, —O⁻M⁺, and —CH₂O⁻M⁺. When Z¹ is selected from —C(O)H, —C(O)R¹⁰, —CH₂C(O)H, and —CH₂C(O)R¹⁰, Y⁴ can be selected from —OH, —CH₂OH, —O-silyl, —CH₂O-silyl, —O⁻M⁺, and —CH₂O⁻M⁺. When Z¹ is selected from —CH₂C(O)R¹⁰, —C(O)OH, and —CH₂C(O)OH Y² can be selected from —OH, and —CH₂OH. When Z¹ is selected from —CH₂C(O)R¹⁰, —C(O)OH, and —CH₂C(O)OHY₄ can be selected from —OH, and —CH₂OH When Z³ is selected from —CH₂C(O)R¹⁰, —C(O)OH, and —CH₂C(O)OHY⁴ can be selected from —OH, and —CH₂OH. When Z¹ is selected from —CH₂C(O)R¹⁰, —C(O)OH, and —CH₂C(O)OHY⁴ can be selected from —OH, and —CH₂OH

### Synthesis Approch—Reductive Etherificaton

In an aspect of the disclosure, a process for preparing a compound of Formula III.A.1

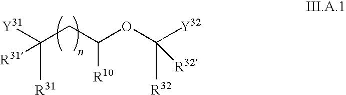

can comprise reacting a compound of Formula III.B.1

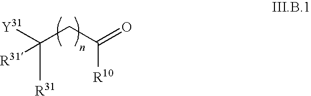

with a compound of Formula III.C.1

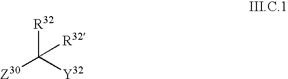

wherein R³¹ and R³² are each independently a) R_{A}, R_{C}, R_{D}, R_{E}, R_{F}, R_{G}, R_{H}, R_{E}, R_{K}, R_{L}, R_{M}, R_{N}, R_{Q}, R_{R}, R_{S}, R_{T}, R_{U}, R_{V}, R_{W}, R_{Y}, or a protected derivative thereof, b) a side chain moiety selected from and having a structure of a non-natural amino acid side chain as delineated in Table I.B.1 or in Table I.C.1 or (c) a protected derivative of the foregoing side chain moieties;

n=0 or 1

Y³¹ is —NHR³⁴, NR³⁴R³⁷, —NHC(O)R³⁵, —NR³⁷C(O)R³⁵, —OR³⁴, —OR³⁵, —OCH₂R³⁵, or ψ[ ]R³⁵

Y³² is —CH₂OR³⁶, —C(O)NHR³⁶—C(O)OR³⁶, or ψ[ ]R³⁶

And R^{31′} and R^{32′} can be each independently selected from the group consisting of H, C₁-C₃ alkyl and substituted C₁-C₃ alkyl.

Preferably, a process for preparing a compound of Formula III.A.2,

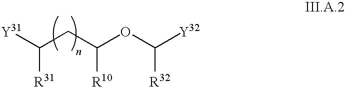

can comprise reacting a compound of Formula III.B.2

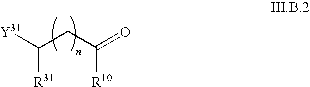

with a compound of Formula III.C.2

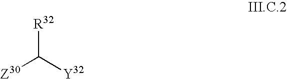

wherein R³¹ and R³² are each independently a) R_{A}, R_{C}, R_{D}, R_{E}, R_{F}, R_{G}, R_{H}, R_{E}, R_{K}, R_{L}, R_{M}, R_{N}, R_{Q}, R_{R}, R_{S}, R_{T}, R_{U}, R_{V}, R_{W}, R_{Y}, or a protected derivative thereof, b) a side chain moiety selected from and having a structure of a non-natural amino acid side chain as delineated in Table I.B.1 or in Table I.C.1 or (c) a protected derivative of the foregoing side chain moieties;

n=0 or 1

Y³¹ is —NHR³⁴, NR³⁴R³⁷, —NHC(O)R³⁵, —NR³⁷C(O)R³⁵, —OR³⁴, —OR³⁵, —OCH₂R³⁵, or ψ[ ]R³⁵

Y³² is —CH₂OR³⁶, —C(O)NHR³⁶—C(O)OR³⁶, or ψ[ ]R³⁶
each -ψ[ ]- being independently selected from the group consisting of —CHR¹⁰O—, —CH₂CHR¹⁰O—, —C(O)NR⁷—, —CH₂C(O)NR⁷—, —CHR¹⁰NH—,

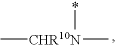

—CHR¹⁰OCHR¹⁰—, —CH₂CH₂—, —CH═CH—, —O—, —C(O)CH₂—, —C(O)O—, —CH(OH)CH₂—, —CH(OH)CH₂NH—, —CHR¹⁰S—, —CHR¹⁰S(O)—, —CHR¹⁰S(O₂)—, —CH₂CHR¹⁰S—, —CH₂CHR¹⁰S(O)—, —CH₂CHR¹⁰S(O₂)—, —CH(CH₃)S—, —C(O)S—, —C(S)NH—, —NHC(O)NH—, —OC(O)NH—, and retroinverso analogs thereof, * representing a bond linking the nitrogen atom to R³¹ along with the other bonds connecting them to form the 5-member heterocyclic ring

R³⁴ is a hydrogen or a protecting group,

R³⁵ and R³⁶ are each independently H, alkyl, substituted alkyl, aryl, substituted aryl, heteroaryl, substituted heteroaryl, heteroalkyl, substituted heteroalkyl, a polyethoid, or a polyaminoacid

each R³⁷ being independently selected from the group consisting of —H, a side chain moiety having a structure of an amino acid side chain, or part of the heterocyclic ring structure with R³¹ above

each R¹⁰ is independently H, C₁-C₃ alkyl, or substituted alkyl,

and Z is a —OH, —O—SiR₃, or —O⁻M⁺, where M⁺ is a metal salt

The compound of formula III.B.2 can be reacted with a compound of formula III.C.2 in a reaction mixture, wherein the reaction mixture includes a catalyst. The catalyst can be provided to the reaction mixture as a metal salt, a lewis acid or a bronsted acid. The catalyst can be provided to the reaction mixture as BiX₃, FeX₃, CuX₂, TMSX, B(C₆F₅)₃, HX, XBi═O, Et₃SiX, or trityl perchlorate, wherein X=I, Br, Cl, F, or O₃SCF₃, preferably as BiBr₃, BiCl₃, FeCl₃, Cu(OS(O)CF₃)₂, Me₃SiO₃SCF₃, B(C₆F₅)₃, HBr, BrBi═O, Et₃SiI or Et₃SiBr. Alternatively, the catalyst can be provided to the reaction mixture as a metal triflate, preferably Cu(OTf)₂, Sm(OTf)₃, Yb(OTf)₃, Sc(OTf)₃, VO(OTf)₂, In(OTf)₃, or Zn(OTf)₂. More preferably the catalyst can be provided to the reaction mixture as BiBr₃ or FeCl₃.

The compound of formula III.B.2 can be reacted with a compound of formula III.C.2 in a reaction mixture, wherein the reaction mixture includes a reducing agent. Tithe reducing agent can be provided to the reaction mixture as a silane, siloxane, or silicon hydride source. In a preferred embodiment, the reducing agent can be provided to the reaction mixture as a trialkylsilane or chlorodialkylsilane, more preferably, triethylsilane. In an alternate embodiment, the reducing agent can be provided to the reaction mixture as any silicon hydride source, preferably polymethylhydrosiloxane.

In a preferred embodiment, the compound of formula III.B can be reacted with a compound of Formula III.C.2 in a reaction mixture, wherein the reaction mixture includes a reducing agent and a catalyst. Preferably, reducing agent can be provided to the reaction mixture as triethylsilane and the catalyst can be provided to the reaction mixture as BiBr₃.

In an embodiment of the process, the compound of formula III.B.2 can be reacted with a compound of formula III.C.2 in any solvent. Preferably, a solvent selected from the group that is tetrahydrofuran, diethyl ether, acetonitrile, propionitrile, methylene chloride, nitromethane, or toluene, more preferably acetonitrile. Preferably the solvent is anhydrous.

M⁺ can be any metal cation commonly found on hydroxide anions. Preferably, the metal cation can be selected from alkali and alkaline earth metals.

The process can be used to create polyethoids and polyethoidpeptides in a modular stepwise process. If can also be used to link two compounds together. In an embodiment, at least one R³⁵ and R³⁶ is polyethoid or a polyaminoacid. Preferably, at least one R³⁵ and R³⁶ is a polyethoid or a polyaminoacid of at least three residues in length. Alternatively, at least one R³⁵ and R³⁶ is a polyethoid, preferably at least two residues in length. The polyethoid can be a polyethoidpeptide. Alternatively, at least one R³⁵ and R³⁶ is a polyaminoacid, preferably at least three residues in length.

The process can be used in solid phase reactions. In an embodiment, either R³⁵ and R³⁶ can attached via covalent bonds to a solid support.

In a embodiment of the process at least one of R³¹ and R³² can be R_{A}, R_{C}, R_{D}, R_{E}, R_{F}, R_{G}, R_{H}, R_{E}, R_{K}, R_{L}, R_{M}, R_{N}, R_{Q}, R_{R}, R_{S}, R_{T}, R_{U}, R_{V}, R_{W}, R_{Y}, or a protected derivative thereof. Alternatively, R³¹ and R³² can be each independently R_{A}, R_{C}, R_{D}, R_{E}, R_{F}, R_{G}, R_{H}, R_{E}, R_{K}, R_{L}, R_{M}, R_{N}, R_{Q}, R_{R}, R_{S}, R_{T}, R_{U}, R_{V}, R_{W}, R_{Y}, or a protected derivative thereof.

In an alternate embodiment, at least one of R³¹ and R³² can be a side chain moiety selected from and having a structure of a non-natural amino acid side chain as delineated in Table I.B.1 or in Table I.C.1, or a protected derivative thereof.

In an embodiment of the process, n can be 0 and R¹⁰ can be H. Preferably, R¹⁰=H; n=0; Y³¹ can be —NHR³⁴, —NHC(O)R³⁵, —OR³⁴, —OR³⁵, —OCH₂R³⁵, or ψ[ ]R³⁵; Y² is —CH₂OR³⁶, —C(O)NHR³⁶ or —C(O)OR²⁶; R³⁴ can be a hydrogen or a protecting group; R³⁵ and R³⁶ can be each independently H, alkyl, substituted alkyl, aryl, substituted aryl, heteroaryl, substituted heteroaryl, heteroalkyl, substituted heteroalkyl, a polyethoid, or a polyaminoacid; and Z can be —OH or —OSiMe₂^{t}Bu. More preferably, Y³¹ can be —NHR³⁴, —NHC(O)R³⁵, —OR³⁴, —OR³⁵, or —OCH₂R³⁵; Y³² can be —CH₂OR³⁶, —C(O)NHR³⁶ or —C(O)OR³⁶; R³⁴ can be a hydrogen or a protecting group; R³⁵ and R³⁶ can be each independently H, alkyl, substituted alkyl, aryl, substituted aryl, heteroaryl, substituted heteroaryl, heteroalkyl, substituted heteroalkyl, a polyethoid, or a polyaminoacid; and Z can be —OH or —OSiMe₂^{t}Bu.

In an embodiment of the process, R³¹ can further include R_{P} when Y³¹ is NR³⁴R³⁷ or —NR³⁷C(O)R³⁵ and R³⁷ together with R³¹ and the atoms connecting them form the 5-member heterocyclic ring.

In an embodiment of the process, the compound of Formula III.C.2 can be α-hydroxy acid, α-hydroxy ester, α-hydroxy Weinreb amide, α-hydroxy aldehyde, α-hydroxy ketone or a protected derivative thereof. Alternatively, the compound of Formula III.C.2 can be α-trialkylsilyloxy acid, α-trialkylsilyloxy ester, α-trialkylsilyloxy Weinreb amide, α-trialkylsilyloxy aldehyde, α-trialkylsilyloxy ketone or a protected derivative thereof.

In an embodiment of the process, the compound of Formula III.B.2 can be an α-amino-aldehyde, α-amino-ketone or amine-protected derivatives thereof, or an α-hydroxy-aldehyde, α-hydroxy-ketone or hydroxy-protected derivatives thereof.

In an embodiment of the process R³⁵ can be a polyethoid or a polyaminoacid, Y³² can be —CH₂OR³⁶, —C(O)NHR³⁶, or —C(O)OR³⁶, and R³⁶ can be a H, alkyl, or substituted alkyl. Preferably, when R³⁵ is polyethoid or a polyaminoacid, the compound of Formula III.C.2 can be an α-hydroxy acid, α-hydroxy ester, α-hydroxy Weinreb amide, α-hydroxy aldehyde, α-hydroxy ketone or a protected derivative thereof.

In an embodiment of the process, R³⁶ can be polyethoid or a polyaminoacid, Y³¹ can be NHR³⁴, NHC(O)R³⁵, —OR³⁴, —OR³⁵, or OCH₂R³⁵, and R³⁵ can be a H, alkyl, or substituted alkyl, aryl, substituted aryl, heteroaryl, substituted heteroaryl, heteroalkyl, substituted heteroalkyl. Preferably, when R³⁶ is polyethoid or a polyaminoacid, the compound of Formula III.B.2 can be an α-amino-aldehyde, α-amino-ketone or amine-protected derivatives thereof, or an α-hydroxy-aldehyde, α-hydroxy-ketone or hydroxy-protected derivatives thereof.

The process can further comprise preparing the compound of Formulat III.C.1 from a compound of Formula III.D.1

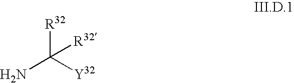

or, preparing a compound Formula III.C.2 from the compound of Formula III.D.2:

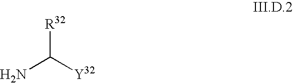

wherein R³², R^{32′} and Y³² are as defined above. In an embodiment, the compound of Formula III.C.2 can be prepared by reacting the compound of Formula III.D.2 in a reaction mixture, wherein the reaction mixture comprises an activating agent and a nitrosyl agent, with a) the activating agent is provided to the reaction mixture as an alkyl nitrite and Br₂; BrNO₂; HOBr; an alkyl hypohalite; cyanogen bromide; NO₂—BF₄ and Br₂; or and N-halosuccinimide; and b) the nitrosyl agent is provided to the reaction mixture as NO₂—BBr₄ and trifluoroacetic acid; HONO; NaNO₂ and trifluoroacetic acid; or NaNO₂ and acetic acid. Preferably, the activating agent can be provided to the reaction mixture as N-bromosuccinimide and the nitrosyl agent can be provided to the reaction mixture as HNO₂. In an alternate embodiment, the compound of Formula III.C.2 can prepared by reacting the compound of Formula III.D.2 in a reaction mixture comprising a diazotization reagent. Preferably, the diazotization reagent can be provided to the reaction as HNO₂, NaNO₂ and sulfuric acid, NaNO₂ and HCl, or NaNO₂ and acetic acid, more preferably as NaNO₂ and acetic acid.

### Ethoid Scanning

A method is also disclosed for optimizing a biological property of a peptide that is to contain ethoid isosteric replacements. Ethoid scanning is a process of synthesizing analogs of a peptide derived by stepping through a peptide and replacing each amide bond in turn by a ψ[CH₂O] bond. This ethoid scan can be conducted by preparing a series of peptide-like polymers with at least one amide bond replaced by an ethoid bond at different positions along the polymer chain. A biological property can then be measured for each compound or groups of compounds in the series in the relevant biological assay. In this way a determination can be made as to which amide bonds to replace. This process can be repeated by replacements with two, three and incrementally higher amide bond replacements until either the amide bonds are completely replaced by ethoid bonds or the biological activity has been optimized. Of course the same method can be used in reverse, that is to make peptide-like polymers containing all ethoid bonds and substitute back in a single amide bond and increasing numbers of amide bonds at each ethoid bond position. Scheme 10 demonstrates an ethoid scan of a theoretical six amino acid peptide.

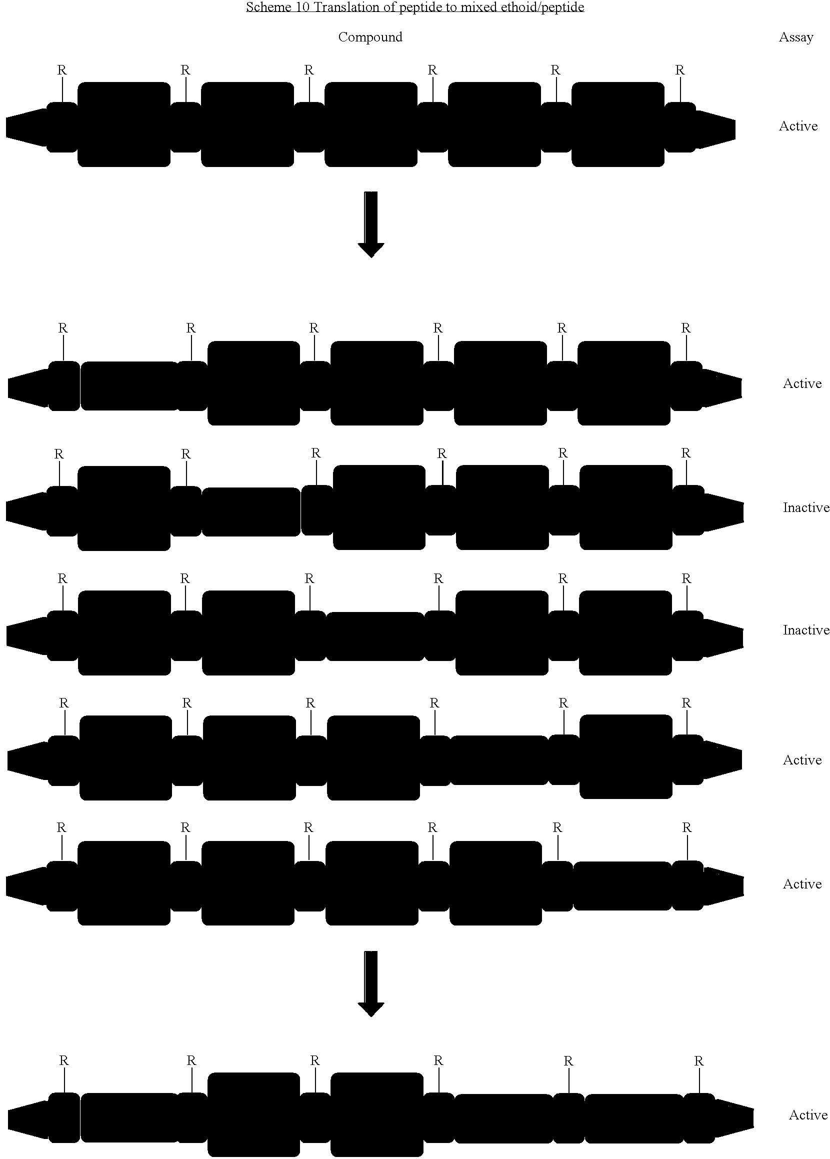

A method is disclosed for identifying an analog of a polyaminoacid having property of interest, the polyaminoacid comprising a structural moiety having three or more amino acid residues linked by amide moieties, the method comprising providing a set of ethoid-containing compounds, the set comprising (a) a first compound comprising the structural moiety of the polyaminoacid with an ethoid isostere at a first sequence position, and (b) a second compound comprising the structural moiety of the polyaminoacid with an ethoid isostere at a second sequence position, the second sequence position being different from the first sequence position, each of the ethoid isosteres having a formula

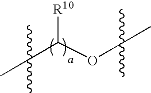

and being a substitutive replacement for an amide moiety within the structural moiety of the polyaminoacid, wherein each a is an integer=1 or =2, and each R¹⁰ is independently selected from the group consisting of H, C₁-C₃ alkyl and substituted C₁-C₃ alkyl, and evaluating the first ethoid-containing compound and the second ethoid-containing compound for the property of interest.

In the method the structural moiety of the polyaminoacid can include four or more amino acid residues linked by amide moieties, and the set of ethoid-containing compounds further comprises (c) a third compound comprising the structural moiety of the polyaminoacid with an ethoid isostere at a third sequence position, the third sequence position being different from each of the first sequence position and the second sequence position, the method further comprising evaluating the third ethoid-containing compound for the property of interest.

In the method the structural moiety of the structural moiety of the polyaminoacid can include five or more amino acid residues linked by amide moieties, and the set of ethoid-containing compounds further comprises (d) a fourth compound comprising the structural moiety of the polyaminoacid with an ethoid isostere at a fourth sequence position, the fourth sequence position being different from each of the first sequence position, the second sequence position, and the third sequence position, the method further comprising evaluating the fourth ethoid-containing compound for the property of interest.

In the method the structural moiety of the structural moiety of the polyaminoacid can include six or more amino acid residues linked by amide moieties, and the set of ethoid-containing compounds further comprises (e) a fifth compound comprising the structural moiety of the polyaminoacid with an ethoid isostere at a fifth sequence position, the fifth sequence position being different from each of the first sequence position, the second sequence position, the third sequence position and the fourth sequence position, the method further comprising evaluating the fifth ethoid-containing compound for the property of interest.

In the method the structural moiety of the structural moiety of the polyaminoacid can include ten or more amino acid residues linked by amide moieties, and the set of ethoid-containing compounds further comprises (f) a sixth compound comprising the structural moiety of the polyaminoacid with an ethoid isostere at a sixth sequence position, (g) a seventh compound comprising the structural moiety of the polyaminoacid with an ethoid isostere at a seventh sequence position, (h) an eighth compound comprising the structural moiety of the polyaminoacid with an ethoid isostere at an eighth sequence position, and (i) a ninth compound comprising the structural moiety of the polyaminoacid with an ethoid isostere at a ninth sequence position, each of the sixth, seventh, eighth and ninth sequence positions being different from each other and different from each of the first, second, third, fourth and fifth sequence positions, the method further comprising evaluating each of the sixth, seventh, eighth and ninth ethoid-containing compounds for the property of interest.

In an embodiment, each of the ethoid-containing compounds can include only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere. When the structural moiety of the polyaminoacid includes a number of amino acid residues, N_{AA}, linked by amide moieties, and the set of ethoid-containing compounds comprises (N_{AA}−1) ethoid-containing compounds, each of the ethoid-containing compounds including only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere, the method further comprising evaluating each of the (N_{AA}−1) ethoid-containing compounds for the property of interest.

In an embodiment, at least one of the ethoid-containing compounds comprises two or more ethoid isosteres as substitutive replacements for two or more amide moieties within the structural moiety of the polyaminoacid, respectively. Preferably, the set of ethoid-containing compounds further comprises at least one fully-ethoid-substituted compound comprising the structural moiety of the polyaminoacid with ethoid isosteres as substitutive replacements for each of the amide moieties within the structural moiety of the polyaminoacid.

The ethoid-containing compounds can be evaluated by any technique known in the art. The ethoid-containing compounds can be evaluated by a method which includes analyzing the ethoid-containing compounds for a detectable analytical property. The ethoid-containing compounds can be evaluated by a method which includes allowing the ethoid-containing compounds to interact with one or more components of a test environment, and analyzing the ethoid-containing compounds, the test environment, or one or more components of the test environment for a detectable analytical property. The ethoid-containing compounds can be evaluated by a method which includes allowing the ethoid-containing compounds to interact with one or more components of a test environment, and analyzing the ethoid-containing compounds, the test environment, or one or more components of the test environment for a detectable analytical property.

In an embodiment, the method then further comprises correlating a detectable analytical property to the property of interest, and then further comprising determining a relative rank of the ethoid-containing compounds based on the evaluation. In one embodiment, the method also further comprises evaluating the polyaminoacid for the property of interest, and comparing the ethoid-containing compounds to the polyaminoacid with respect to the property of interest, and furthermore selecting an ethoid-containing compound from among the set of ethoid-containing compounds based on maintenance of or improvement of the property of interest relative to the polyaminoacid.

After a first property of interest has been evaluated, the method can further comprise evaluating the ethoid-containing compounds for a second property of interest. An ethoid-containing compounds can then be selected based upon an improvement of at least one of the first property of interest or the second property of interest, relative to the polyaminoacid, or can be based upon maintenance of the first property of interest relative to the polyaminoacid, and improvement of the second property of interest, relative to the polyaminoacid. The ethoid-containing compounds can be evaluated by a method which include analyzing each of the ethoid-containing compounds in series, or in parallel. The ethoid the ethoid-containing compounds can be provided with encoded identifiers, and can be evaluated by a method that includes deconvoluting the encoded identifiers. The encoded identifiers can be deconvoluated to determine a correspondence between a particular compound being evaluated and a particular ethoid-containing compound.

Any property of interest can be evaluated. The property of interest can be a biological property, biological activity, receptor agonism, receptor antagonism, selectivity for a target receptor, enzyme inhibition, receptor binding affinity, antibody binding affinity, binding affinity to an epitope, binding affinity to a toxin, stability to an enzyme, stability to a peptidase or protease, stability to an exopeptidase, stability to an endopeptidase, a pharmokinetic property, bioavailability, cell permeability, transportation across a cell membrane, transport across a cell membrane, extent of systemic absorption from the gastrointestinal tract, extent of excretion, metabolism, pharmaceutical or biological half-life, distribution, efficacy, tolerability, an organoleptic property, or taste. The property of interest can be any chemical property, including chemical stability under various conditions, including but not limited to stability at temperatures greater than 30° C., environments having greater than 50% relative humidity, having pH lower than 6, having pH higher than 8, oxidation, reduction, photostability, photoreactivity, crystallinity, and polymorphism.

By creating and evaluating a series of ethoid-containing compounds, a data set can be created. In an embodiment, a data set can be stored on a tangible medium, the data set comprising data derived from evaluating a set of ethoid-containing analogs of a polyaminoacid for a property of interest, the polyaminoacid comprising a structural moiety having three or more amino acid residues linked by amide moieties, the set of ethoid-containing analogs comprising (a) a first compound comprising the structural moiety of the polyaminoacid with an ethoid isostere at a first sequence position, and (b) a second compound comprising the structural moiety of the polyaminoacid with an ethoid isostere at a second sequence position, the second sequence position being different from the first sequence position, each of the ethoid isosteres having a formula

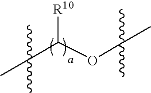

and being a substitutive replacement for an amide moiety within the structural moiety of the polyaminoacid, wherein each a is an integer=1 or =2, and each R¹⁰ is independently selected from the group consisting of H, C₁-C₃ alkyl and substituted C₁-C₃ alkyl.

The data set can be derived from the structural moiety of a polyaminoacid with four or more amino acid residues linked by amino acid bonds, and the set of ethoid-containing analogs further comprising a third compound comprising a structural moiety of the polyaminoacid with an ethoid isostere at a third sequence position, the third sequence position being different from each of the first sequence position and the second sequence position. Similarly, for a polyaminoacid with 5 or more amino acid residues, 6 or more amino acid residues, ten or more amino acid residues, and so forth as described above.

In an embodiment, a method is disclosed for preparing a set of ethoid-containing analogs of a polyaminoacid, the polyaminoacid comprising a structural moiety having three or more amino acid residues linked by amide moieties, the method comprising

obtaining an amino acid sequence identity for the structural moiety of the polyaminoacid,

identifying a first amide moiety for isosteric replacement at a first sequence position within the structural moiety of the polyaminoacid,

identifying a second amide moiety for isosteric replacement at a second sequence position within the structural moiety of the polyaminoacid, the second sequence position being different from the first sequence position,

forming a first compound comprising the structural moiety of the polyaminoacid with an ethoid isostere at the first sequence position, and

forming a second compound comprising the structural moiety of the polyaminoacid with an ethoid isostere at a second sequence position,

each of the ethoid isosteres having a formula

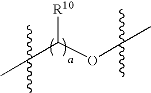

and being a substitutive replacement for the identified amide moiety of the polyaminoacid, wherein each a is an integer=1 or =2, and each R¹⁰ is independently selected from the group consisting of H, C₁-C₃ alkyl and substituted C₁-C₃ alkyl.

In an embodiment, at least one of the first amide moiety and the second amide moiety are identified for isosteric replacement at their respective sequence positions based on proteolytic susceptibility at the sequence position. The amino acid sequence identity for the structural moiety of the polyaminoacid can be obtained by sequence analysis. The amide bonds that might be susceptible to proteolysis can then be predicted by any method in the art. Replacement of these amide bond that are susceptible to proteolysis by, for example, a specific enzyme, can produce a polyethoid that has increased resistance to a protease. Cleavage sites are known for a multiplicity of proteases or peptidase enzymes, including those described in Table II. For example, trypsin (EC 3.4.21.4) cleaves amide bonds on the C-terminal side of lysine (K) and arginine (R) residues which suggests that these amide bonds may be suitable for isosteric replacement. In an alternate embodiment, the least one of the first amide moiety and the second amide moiety can be identified for isosteric replacement at their respective sequence positions based on random selection or based on patterned selection.

In an embodiment, each of the first compound and the second compound are formed by a method that includes a series of reaction cycles, each reaction cycle including sequential addition of an amino acid residue linked by an ethoid isostere, an amide moiety, or -ψ[ ]-. This method can include a series of reaction cycles, at least one reaction cycle of the series including sequential addition of an amino acid residue linked by an ethoid isostere, and at least one reaction cycle of the series including sequential addition of an amino acid residue linked by an amide moiety. One or more of the ethoid bonds can be formed by the synthetic methods disclosed herein. In a preferred embodiment, one or more of the ethoid bonds can be formed by a method that includes reductive etherification, or from a method that includes a Williamson ether reaction.

In the method disclosed for preparing a set of ethoid-containing analogs of a polyaminoacid, the polyaminoacid can comprise a structural moiety having more than three amino acid residues linked by amide moieties. When the structural moiety of the polyaminoacid includes four or more amino acid residues linked by amide moieties, the method further comprises identifying a third amide moiety for isosteric replacement at a third sequence position within the structural moiety of the polyaminoacid, the third sequence position being different from each of the first sequence position and the second sequence position, and forming a third compound comprising the structural moiety of the polyaminoacid with an ethoid isostere at the third sequence position.

When the structural moiety of the polyaminoacid includes five or more amino acid residues linked by amide moieties, the method further comprises identifying a fourth amide moiety for isosteric replacement at a fourth sequence position within the structural moiety of the polyaminoacid, the fourth sequence position being different from each of the first sequence position, the second sequence position, and the third sequence position, and forming a fourth compound comprising the structural moiety of the polyaminoacid with an ethoid isostere at the fourth sequence position.

When the structural moiety of the polyaminoacid includes six or more amino acid residues linked by amide moieties, the method further comprises identifying a fifth amide moiety for isosteric replacement at a fifth sequence position within the structural moiety of the polyaminoacid, the fifth sequence position being different from each of the first sequence position, the second sequence position, the third sequence position and the fourth sequence position, and forming a fifth compound comprising the structural moiety of the polyaminoacid with an ethoid isostere at the fifth sequence position.

When the structural moiety of the polyaminoacid includes ten or more amino acid residues linked by amide moieties, the method further comprising identifying a sixth, seventh, eighth and ninth amide moiety for isosteric replacement at a respective sixth, seventh, eighth and ninth sequence position within the structural moiety of the polyaminoacid, each of the sixth, seventh, eighth and ninth sequence positions being different from each other and different from each of the first, second, third, fourth, and fifth sequence positions, and forming a sixth compound comprising the structural moiety of the polyaminoacid with an ethoid isostere at the sixth sequence position, a seventh compound comprising the structural moiety of the polyaminoacid with an ethoid isostere at the seventh sequence position, an eighth compound comprising the structural moiety of the polyaminoacid with an ethoid isostere at the eighth sequence position, and a ninth compound comprising the structural moiety of the polyaminoacid with an ethoid isostere at the ninth sequence position.

In an embodiment of the method, each of the ethoid-containing compounds can include only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere. When the structural moiety of the polyaminoacid includes a number of amino acid residues, N_{AA}, linked by amide moieties, and the set of ethoid-containing compounds comprises (N_{AA}−1) ethoid-containing compounds, each of the ethoid-containing compounds including only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere, the method further comprising evaluating each of the (N_{AA}−1) ethoid-containing compounds for the property of interest.

In an embodiment of the method, at least one of the ethoid-containing compounds comprises two or more ethoid isosteres as substitutive replacements for two or more amide moieties within the structural moiety of the polyaminoacid, respectively. Preferably, the set of ethoid-containing compounds further comprises at least one fully-ethoid-substituted compound comprising the structural moiety of the polyaminoacid with ethoid isosteres as substitutive replacements for each of the amide moieties within the structural moiety of the polyaminoacid.

A set of ethoid-containing compounds can be created using any of the methods of this disclosure. A set of ethoid-contains polyaminoacid analogs, the polyaminoacid comprising a structural moiety having three or more amino acid residues linked by amide moieties, the set comprising (a) a first compound comprising the structural moiety of the polyaminoacid with at least one ethoid isostere at a first sequence position, and (b) a second compound comprising the structural moiety of the polyaminoacid with at least one ethoid isostere at a second sequence position, the second sequence position being different from the first sequence position, each of the ethoid isosteres having a formula

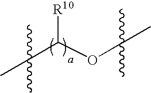

and being a substitutive replacement for an amide moiety of the polyaminoacid, wherein each a c can be an integer=1 or =2, and each R¹⁰ can be independently selected from the group consisting of H, C₁-C₃ alkyl and substituted C₁-C₃ alkyl.

When the structural moiety of the polyaminoacid includes five or more amino acid residues linked by amide moieties, the set of ethoid-containing analogs can further comprise (d) a fourth compound comprising the structural moiety of the polyaminoacid with an ethoid isostere at a fourth sequence position, the fourth sequence position being different from each of the first sequence position. When the structural moiety of the polyaminoacid includes six or more amino acid residues linked by amide moieties, the set of ethoid-containing analogs can further comprise (e) a fifth compound comprising the structural moiety of the polyaminoacid with an ethoid isostere at a fifth sequence position, the fifth sequence position being different from each of the first sequence position, the second sequence position, the third sequence position and the fourth sequence position. When the structural moiety of the polyaminoacid includes ten or more amino acid residues linked by amide moieties, the set of ethoid-containing analogs can further comprise (f) a sixth compound comprising the structural moiety of the polyaminoacid with an ethoid isostere at a sixth sequence position, (g) a seventh compound comprising the structural moiety of the polyaminoacid with an ethoid isostere at a seventh sequence position, (h) an eighth compound comprising the structural moiety of the polyaminoacid with an ethoid isostere at an eighth sequence position, and (i) a ninth compound comprising the structural moiety of the polyaminoacid with an ethoid isostere at a ninth sequence position, each of the sixth, seventh, eighth and ninth sequence positions being different from each other and different from each of the first, second, third, fourth and fifth sequence positions.

In an embodiment of the set, each of the ethoid-containing compounds can include only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere. When the structural moiety of the polyaminoacid includes a number of amino acid residues, N_{AA}, linked by amide moieties, and the set of ethoid-containing compounds comprises (N_{AA}−1) ethoid-containing compounds, each of the ethoid-containing compounds including only a single ethoid isostere as a substitutive replacement for only one of the amide moieties of the polyaminoacid, whereby each of the ethoid-containing compounds differs structurally from each of the other ethoid-containing compounds by sequence position of the single ethoid isostere, the method further comprising evaluating each of the (N_{AA}−1) ethoid-containing compounds for the property of interest.

In an embodiment of the set, at least one of the ethoid-containing compounds comprises two or more ethoid isosteres as substitutive replacements for two or more amide moieties within the structural moiety of the polyaminoacid, respectively. Preferably, the set of ethoid-containing compounds further comprises at least one fully-ethoid-substituted compound comprising the structural moiety of the polyaminoacid with ethoid isosteres as substitutive replacements for each of the amide moieties within the structural moiety of the polyaminoacid.

As is apparent, polyethoids as disclosed herein can now be prepared for any polyaminoacid. In one embodiment, one or more polyethoids can be prepared for a polyaminoacid selected from the group: GHRH (SEQ ID NO: 1); PR1 (T-cell epitope) (SEQ ID NO: 2); Protease-3 peptide (1) (SEQ ID NO: 3); Protease-3 peptide (2) (SEQ ID NO: 4); Protease-3 peptide (3) (SEQ ID NO: 5); Protease-3 peptide (4) (SEQ ID NO: 6); Protease-3 peptide (5) (SEQ ID NO: 7); Protease-3 peptide (6) (SEQ ID NO: 8); Protease-3 peptide (7) (SEQ ID NO: 9); Protease-3 peptide (8) (SEQ ID NO: 10); Protease-3 peptide (9) (SEQ ID NO: 11); Protease-3 peptide (10) (SEQ ID NO: 12); Protease-3 peptide (11) (SEQ ID NO: 13); P3, B-cell epitope (SEQ ID NO: 14); P3, B-cell epitope: (with spacer) (SEQ ID NO: 15); GLP1 (SEQ ID NO: 16); LHRH (SEQ ID NO: 17); PTH (SEQ ID NO: 18); Substance P (SEQ ID NO: 19); Neurokinin A (SEQ ID NO: 20); Neurokinin B (SEQ ID NO: 21); Bombesin (SEQ ID NO: 22); CCK-8 (SEQ ID NO: 23); Leucine Enkephalin (SEQ ID NO: 24); Methionine Enkephalin (SEQ ID NO: 25); [Des Ala20, Gln34] Dermaseptin (SEQ ID NO: 26); Antimicrobial Peptide (Surfactant) (SEQ ID NO: 27); Antimicrobial Anionic Peptide (Surfactant-associated AP) (SEQ ID NO: 28); Apidaecin IA (SEQ ID NO: 29); Apidaecin IB (SEQ ID NO: 30); OV-2 (SEQ ID NO: 31); 1025, Acetyl-Adhesin Peptide (1025-1044) amide (SEQ ID NO: 32); Theromacin (49-63) (SEQ ID NO: 33); Pexiganan (MSI-78) (SEQ ID NO: 34); Indolicidin (SEQ ID NO: 35); Apelin-15 (63-77) (SEQ ID NO: 36); CFP10 (71-85) (SEQ ID NO: 37); Lethal Factor (LF) Inhibitor Anthrax related (SEQ ID NO: 38); Bactenecin (SEQ ID NO: 39); Hepatitis Virus C NS3 Protease Inhibitor 2 (SEQ ID NO: 40); Hepatitis Virus C NS3 Protease Inhibitor 3 (SEQ ID NO: 41); Hepatitis Virus NS3 Protease Inhibitor 4 (SEQ ID NO: 42); NS4A-NS4B Hepatitis Virus C (NS3 Protease Inhibitor 1) (SEQ ID NO: 43); HIV-1, HIV-2 Protease Substrate (SEQ ID NO: 44); Anti-Flt1 Peptide (SEQ ID NO: 45); Bak-BH3 (SEQ ID NO: 46); Bax BH3 peptide (55-74) (wild type) (SEQ ID NO: 47); Bid BH3-r8 (SEQ ID NO: 48); CTT (Gelatinase Inhibitor) (SEQ ID NO: 49); E75 (Her-2/neu) (369-377) (SEQ ID NO: 50); GRP78 Binding Chimeric Peptide Motif (SEQ ID NO: 51); p53(17-26) (SEQ ID NO: 52); EGFR2/KDR Antagonist (SEQ ID NO: 53); Colivelin (SEQ ID 54) AGA-(C8R)HNG17 (Humanin derivative) (SEQ ID NO: 55); Activity-Dependent Neurotrophic Factor (ADNF) (SEQ ID NO: 56); Beta-Secretase Inhibitor 1 (SEQ ID NO: 57); Beta-Secretase Inhibitor 2 (SEQ ID NO: 58); chβ-Amyloid (30-16) (SEQ ID NO: 59); Humanun (HN) (SEQ ID NO: 60); sHNG, [Gly14]-HN, [Gly14]-Humanin (SEQ ID NO: 61); Angiotensin Converting Enzyme Inhibitor (BPP) (SEQ ID NO: 62); Renin Inhibitor 111 (SEQ ID NO: 63); Annexin 1 (ANXA-1; Ac2-12) (SEQ ID NO: 64); Anti-Inflammatory Peptide 1 (SEQ ID NO: 65); Anti-Inflammatory Peptide 2 (SEQ ID NO: 66); Anti-Inflammatory Apelin 12 (SEQ ID NO: 67); [D-Phe12, Leu14]-Bombesin (SEQ ID NO: 68); Antennapedia Peptide (acid) (penetratin) (SEQ ID NO: 69); Antennepedia Leader Peptide (CT) (SEQ ID NO: 70); Mastoparan (SEQ ID NO: 71); [Thr28, Nle31]-Cholecystokinin (25-33) sulfated (SEQ ID NO: 72); Nociceptin (1-13) (amide) (SEQ ID NO: 73); Fibrinolysis Inhibiting Factor (SEQ ID NO: 74); Gamma-Fibrinogen (377-395) (SEQ ID NO: 75); Xenin (SEQ ID NO: 76); Obestatin (human) (SEQ ID NO: 77); [His1, Lys6]-GHRP (GHRP-6) (SEQ ID NO: 78); [Ala5, β-Ala8]-Neurokinin A (4-10) (SEQ ID NO: 79); Neuromedin B (SEQ ID NO: 80); Neuromedin C (SEQ ID NO: 81); Neuromedin N (SEQ ID NO: 82); Activity-Dependent Neurotrophic Factor (ADNF-14) (SEQ ID NO: 83); Acetalin 1 (Opioid Receptor Antagonist 1) (SEQ ID NO: 84); Acetalin 2 (Opioid Receptor Antagonist 2) (SEQ ID NO: 85); Acetalin 3 (Opioid Receptor Antagonist 3) (SEQ ID NO: 86); ACTH (1-39) (human) (SEQ ID NO: 87); ACTH (7-38) (human) (SEQ ID NO: 88); Sauvagine (SEQ ID NO: 89); Adipokinetic Hormone (Locusta Migratoria) (SEQ ID NO: 90); Myristoylated ADP-Ribosylation Factor 6, myr-ARF6 (2-13) (SEQ ID NO: 91); PAMP (1-20) (Proadrenomedullin (1-20) human) (SEQ ID NO: 92); AGRP (25-51) (SEQ ID NO: 93); Amylin (8-37) (human) (SEQ ID NO: 94); Angiotensin I (human) (SEQ ID NO: 95); Angiotensin II (human) (SEQ ID NO: 96); Apstatin (Aminopeptidase P Inhibitor) (SEQ ID NO: 97); Brevinin-1 (SEQ ID NO: 98); Magainin 1 (SEQ ID NO: 99); RL-37 (SEQ ID NO: 100); LL-37 (Antimicrobial Peptide) (human) (SEQ ID NO: 101); Cecropin A (SEQ ID NO: 102); Antioxidant peptide A (SEQ ID NO: 103); Antioxidant peptide B (SEQ ID NO: 104); L-Carnosine (SEQ ID NO: 105); Bcl 9-2 (SEQ ID NO: 106); NPVF (SEQ ID NO: 107); Neuropeptide AF (hNPAF) (Human) (SEQ ID NO: 108); Bax BH3 peptide (55-74) (SEQ ID NO: 109); bFGF Inhibitory Peptide (SEQ ID NO: 110); bFGF inhibitory Peptide II (SEQ ID NO: 111); Bradykinin (SEQ ID NO: 112); [Des-Arg10]-HOE 140 (SEQ ID NO: 113); Caspase 1 Inhibitor II (SEQ ID NO: 114); Caspase 1 Inhibitor VIII (SEQ ID NO: 115); Smac N7 Protein (SEQ ID NO: 116); MEK1 Derived Peptide Inhibitor 1 (SEQ ID NO: 117); hBD-1 (β-Defensin-1) (human) (SEQ ID NO: 118); hBD-3 (β-Defensin-3) (human) (SEQ ID NO: 119); hBD-4 (β-Defensin-4) (human) (SEQ ID NO: 120); HNP-1 (Defensin Human Neutrophil Peptide 1) (SEQ ID NO: 121); HNP-2 (Defensin Human neutrophil Peptide-2 Dynorphin A (1-17)) (SEQ ID NO: 122); Endomorphin-1 (SEQ ID NO: 123); β-Endorphin (human porcine) (SEQ ID NO: 124); Endothelin 2 (human) (SEQ ID NO: 125); Fibrinogen Binding Inhibitor Peptide (SEQ ID NO: 126); Cyclo(-GRGDSP) (SEQ ID NO: 127); TP508 (Thrombin-derived Peptide) (SEQ ID NO: 128); Galanin (human) (SEQ ID NO: 129); GIP (human) (SEQ ID NO: 130); Gastrin Releasing Peptide (human) (SEQ ID NO: 131); Gastrin-1 (human) (SEQ ID NO: 132); Ghrelin (human) (SEQ ID NO: 133); PDGF-BB peptide (SEQ ID NO: 134); [D-Lys3]-GHRP-6 (SEQ ID NO: 135); HCV Core Protein (1-20) (SEQ ID NO: 136); a3β1 Integrin Peptide Fragment (325) (amide) (SEQ ID NO: 137); Laminin Pentapeptide (amide) (SEQ ID NO: 138); Melanotropin-Potentiating Factor (MPF) (SEQ ID NO: 139); VA-β-MSH, Lipotropin-Y (Proopiomelanocortin-derived) (SEQ ID NO:140); Atrial Natriuretic Peptide (1-28) (human) (SEQ ID NO: 141); Vasonatrin Peptide (1-27) (SEQ ID NO: 142); [Ala5, β-Ala8]-Neurokinin A (4-10) (SEQ ID NO: 143); Neuromedin L (NKA) (SEQ ID NO: 144); Ac-(Leu28, 31)-Neuropeptide Y (24-26) (SEQ ID NO: 145); Alytesin (SEQ ID NO: 146); Brain Neuropeptide II (SEQ ID NO: 147); [D-tyr11]-Neurotensin (SEQ ID NO: 148); IKKy NEMO Binding Domain (NBD) Inhibitory Peptide (SEQ ID NO: 149); PTD-p50 (NLS) Inhibitory Peptide (SEQ ID NO: 150); Orexin A (bovine, human, mouse, rat) (SEQ ID NO: 151); Orexin B (human) (SEQ ID NO: 152); Aquaporin-2(254-267) (human Pancreastatin) (37-52) (SEQ ID NO: 153); Pancreatic Polypeptide (human) (SEQ ID NO: 154); Neuropeptide (SEQ ID NO: 155); Peptide YY (3-36) (human) (SEQ ID NO: 156); Hydroxymethyl-Phytochelatin 2 (SEQ ID NO: 157); PACAP (1-27) (amide, human, bovine, rat) (SEQ ID NO: 158); Prolactin Releasing Peptide (1-31) (human) (SEQ ID NO: 159); Salusin-alpha (SEQ ID NO: 160); Salusin-beta (SEQ ID NO: 161); Saposin C22 (SEQ ID NO: 162); Secretin (human) (SEQ ID NO: 163); L-Selectin (SEQ ID NO: 164); Endokinin A/B SEQ ID NO: 165); Endokinin C (Human) (SEQ ID NO: 166); Endokinin D (Human) (SEQ ID NO: 167); Thrombin Receptor (42-48) Agonist (human) (SEQ ID NO: 168); LSKL (Inhibitor of Thrombospondin) (SEQ ID NO: 169); Thyrotropin Releasing Hormone (TRH) (SEQ ID NO: 170); P55-TNFR Fragment (SEQ ID NO: 171); Urotensin II (human) (SEQ ID NO: 172); VIP (human, porcine, rat) (SEQ ID NO: 173); VIP Antagonist (SEQ ID NO: 174); Helodermin (SEQ ID NO: 175); Exenatide (SEQ ID NO: 176); ZP10 (AVE00100) (SEQ ID NO: 177); Pramlinitide (SEQ ID NO: 178); AC162352 (PYY) (3-36) (SEQ ID NO: 179); PYY (SEQ ID NO: 180); Obinepitide (SEQ ID NO: 181); Glucagon (SEQ ID NO: 182); GRP (SEQ ID NO: 183); Ghrelin (GHRP6) (SEQ ID NO: 184); Leuprolide (SEQ ID NO: 185); Histrelin (SEQ ID NO: 186); Oxytocin (SEQ ID NO: 187); Atosiban (RWJ22164) (SEQ ID NO: 188); Sermorelin (SEQ ID NO: 189); Nesiritide (SEQ ID NO: 190); bivalirudin (Hirulog) (SEQ ID NO: 191); Icatibant (SEQ ID NO: 192); Aviptadil (SEQ ID NO: 193); Rotigaptide (ZP123, GAP486) (SEQ ID NO: 194); Cilengitide (EMD-121924, RGD Peptides) (SEQ ID NO: 195); AlbuBNP (SEQ ID NO: 196); BN-054 (SEQ ID NO: 197); Angiotensin II (SEQ ID NO: 198); MBP-8298 (SEQ ID NO: 199); Peptide Leucine Arginine (SEQ ID NO: 200); Ziconotide (SEQ ID NO: 201); AL-208 (SEQ ID NO: 202); AL-108 (SEQ ID NO: 203); Carbeticon (SEQ ID NO: 204); Tripeptide (SEQ ID NO: 205); SAL (SEQ ID NO: 206); Coliven (SEQ ID NO: 207); Humanin (SEQ ID NO: 208); ADNF-14 (SEQ ID NO: 209); VIP (Vasoactive Intestinal Peptide) (SEQ ID NO: 210); Thymalfasin (SEQ ID NO: 211); Bacitracin (USP) (SEQ ID NO: 212); Gramidicin (USP) (SEQ ID NO: 213); Pexiganan (MSI-78) (SEQ ID NO: 214); P113 (SEQ ID NO: 215); PAC-113 (SEQ ID NO: 216); SCV-07 (SEQ ID NO: 217); HLF1-11 (Lactoferrin) (SEQ ID NO: 218); DAPTA (SEQ ID NO: 219); TRI-1144 (SEQ ID NO: 220); Tritrpticin (SEQ ID NO: 221); Antiflammin 2 (SEQ ID NO: 222); Gattex (Teduglutide, ALX-0600) (SEQ ID NO: 223); Stimuvax (L-BLP25) (SEQ ID NO: 224); Chrysalin (TP508) (SEQ ID NO: 225); Melanonan II (SEQ ID NO: 226); Spantide II (SEQ ID NO: 227); Ceruletide (SEQ ID NO: 228); Sincalide (SEQ ID NO: 229); Pentagastin (SEQ ID NO: 230); Secretin (SEQ ID NO: 231); Endostatin peptide (SEQ ID NO: 232); E-selectin (SEQ ID NO: 233); HER2 (SEQ ID NO: 234); IL-6 (SEQ ID NO: 235); IL-8 (SEQ ID NO: 236); IL-10 (SEQ ID NO: 237); PDGF (SEQ ID NO: 238); Thrombospondin (SEQ ID NO: 239); uPA (1) (SEQ ID NO: 240); uPA (2) (SEQ ID NO: 241); VEGF (SEQ ID NO: 242); VEGF (2) (SEQ ID NO: 243); Pentapeptide-3 (SEQ ID NO: 244); Glutathione (SEQ ID NO: 245); XXLRR (SEQ ID NO. 246); Beta-Amyloid Fibrillogenesis (SEQ ID NO: 247); Endomorphin-2 (SEQ ID NO: 248); TIP 39 (Tuberoinfundibular Neuropeptide) (SEQ ID NO: 249); PACAP (1-38) (amide, human, bovine, rat) (SEQ ID NO: 250); TGFβ activating peptide (SEQ ID NO: 251); Insulin sensitizing factor (ISF402) (SEQ ID NO: 252); Transforming Growth Factor β1 Peptide (TGF-β1) (SEQ ID NO: 253); Caerulein Releasing Factor (SEQ ID NO: 254); IELLQAR (8-branch MAPS) (SEQ ID NO: 255); Tigapotide PK3145 (SEQ ID NO: 256); Goserelin (SEQ ID NO: 257); Abarelix (SEQ ID NO: 258); Cetrorelix (SEQ ID NO: 259); Ganirelix (SEQ ID NO: 260); Degarelix (Triptorelin) (SEQ ID NO: 261); Barusiban (FE 200440) (SEQ ID NO: 262); Pralmorelin (SEQ ID NO: 263); Octreotide (SEQ ID NO: 264); Eptifibatide (SEQ ID NO: 265); Netamiftide (INN-00835) (SEQ ID NO: 266); Daptamycin (SEQ ID NO: 267); Spantide II (1) (SEQ ID NO: 268); Delmitide (RDP-58) (SEQ ID NO: 269); AL-209 (SEQ ID NO: 270); Enfuvirtide (SEQ ID NO: 271); IDR-1 (SEQ ID NO: 272); Hexapeptide-6 (SEQ ID NO: 272); Insulin-A chain (SEQ ID NO: 274); Lanreotide (SEQ ID NO: 275); Hexapeptide-3 (SEQ ID NO: 276); Insulin B-chain (SEQ ID NO: 277); Glargine-A chain (SEQ ID NO: 278); Glargine-B chain (SEQ ID NO: 279); Insulin-LisPro B-chain analog (SEQ ID NO: 280); Insulin-Aspart B-chain analog (SEQ ID NO: 281); Insulin-Glulisine B chain analog (SEQ ID NO: 282); Insulin-Determir B chain analog (SEQ ID NO: 283); Somatatin (SEQ ID NO: 284); Somatostatin Tumor Inhibiting Analog (SEQ ID NO: 285); Pancreastatin (37-52) (SEQ ID NO: 286); Vasoactive Intestinal Peptide fragment (KKYL-NH2) (SEQ ID NO: 287); Dynorphin A (SEQ ID NO: 288); or analogs thereof of any of the foregoing.

In a preferred embodiment, one or more polyethoids can be prepared for a polyaminoacid selected from the group comprising: PYY (SEQ ID NO: 181); Obinepitide (SEQ ID NO: 183); PTH (SEQ ID NO:18); Leuprolide (SEQ ID NO: 187); Atosiban (SEQ ID NO: 190); Sermorelin (SEQ ID NO:191); Pralmorelin (SEQ ID NO:268); Nesiritide (SEQ ID NO: 192); Rotigaptide (SEQ ID NO:196); Cilengitide (SEQ ID NO: 197); MBP-8298 (SEQ ID NO:202); AL-108 (SEQ ID NO:206); Enfuvirtide (SEQ ID NO: 278); Thymalfasin (SEQ ID NO: 214); Daptamycin (SEQ ID NO: 272); HLF1-11 (SEQ ID NO: 222); Lactoferrin (SEQ ID NO:222); Gattex (SEQ ID NO: 227); Teduglutide (SEQ ID NO: 227); ALX-0600 (SEQ ID NO:227); Delmitide (SEQ ID NO: 274); RDP-58 (SEQ ID NO: 274); pentapeptide-3 (SEQ ID NO:248); hexapeptide-6 (SEQ ID NO: 107); L-carnosine (SEQ ID NO: 107); and glutathione (SEQ ID NO:249); or analogs thereof of any of the foregoing.

More preferably, one or more polyethoids can be prepared for a polyaminoacid selected from the group comprising: GLP-1 (SEQ ID NO: 16); LHRH (SEQ ID NO: 17); PTH (SEQ ID NO: 18); Substance P (SEQ ID NO: 19); Neurokinin A (SEQ ID NO: 20); Neurokinin B (SEQ ID NO: 21); Bombesin (SEQ ID NO: 22); CCK-8 (SEQ ID NO: 23); Leucine Enkephalin (SEQ ID NO: 24); Methionine Enkephalin (SEQ ID NO: 25); GHRH (SEQ ID NO: 1); PR1 (T-cell epitope) (SEQ ID NO: 2); P3 (B-cell epitope) (SEQ ID NO: 14); and Somatostatin (SEQ ID NO: 284); or analogs thereof of any of the foregoing.

The synthetic steps involved in building a polymer chain can include determining the end group of the growing chain and then adding the next building block in the chain. When the end group is an amine, the next building block can be a hydroxyl-carboxyl or amino-carboxyl. When the end group is a hydroxyl, the next building block can be an amino-aldehyde or hydroxyl-aldehyde. In this method when the end group is a hydroxyl, the hydroxyl can first be converted to a silyl-hydroxyl, then reacted with the amino-aldehyde or hydroxyl-aldehyde. The hydroxyl group of the hydroxyl-aldehyde building block can be protected with a non-silyl protecting group. When the end group is an aldehyde, the next building block can be a hydroxyl-carboxyl or hydroxyl-aldehyde. When the end group is an aldehyde, the hydroxyl-aldehyde building block can be protected at the aldehyde position. When the end group is an acid, the next building block can be an amino-carboxyl or amino-aldehyde. The term hydroxyl-aldehyde refers to an organic compound, which can be a monomer, having a terminal hydroxyl group at one end and an aldehyde at the opposite end, each compound optionally protected with orthogonal protecting groups. Similarly, an amino-aldehyde is an organic compound, which can be a monomer, having a terminal amine group at one end and an aldehyde at the opposite end, each compound optionally protected with orthogonal protecting groups. A hydroxyl-carboxyl is an organic compound, which can be a monomer, having a terminal hydroxyl group at one end and a carboxylic acid group at the opposite end, each compound optionally protected with orthogonal protecting groups. Lastly, an amino-carboxyl is an organic compound, which can be a monomer, having a terminal amine group at one end and a carboxylic acid group at the opposite end, each optionally protected with orthogonal protecting groups.

Retro-inverso ethoid analogs of ethoid compounds have an identical arrangement in space of sidechain moieties on a backbone compared to the parent ethoid compound. The backbones differ in the arrangement of the ethoid bond relative to the sidechains, ie. it's direction is reversed (CH2O—>OCH2). For an ethoid compound where the relative arrangement of its chiral sidechain moieties determine its function then a retroinverso ethoid analog will have the same function as the corresponding ethoid compound.

Retro-inverso polyethoids are prepared by the same general methods described for polyethoids, for example General Method 5. Typically, the order of addition of building blocks is reversed and the chirality of the building blocks are inverted relative to the corresponding polyethoid compound. The terminal groups of retroinverso polyethoids can be manipulated by any suitable method of SPPS or solid phase organic synthesis.

As described for the method of ethoid scanning, by empirical assay of libraries of compounds, it may be determined that a retroinverso ethoid bond is the most suitable replacement for an amide bond at a particular position in a parent sequence. The method described to prepare retroinverso polyethoids can be utilized to prepare compounds that contain retroinverso ethoid bonds, and amide bonds and/or other -ψ[ ]- bonds (or retroinverso versions thereof), by any suitable methods described herein.

In a method, the use of reductive etherification is disclosed for forming ether bonds in the modular synthesis of mixed ethoid-peptides. In order to be able to synthesize ethoids and mixed ethoid-peptides having anywhere from a single to all amide bonds replaced for any peptide, a modular solid phase methodology has been developed that is compatible with standard solid phase peptide synthesis (SPPS). The method can use α-hydroxy acid-derived building blocks in reductive etherification bond forming reactions between trialkylsilylethers and aldehydes, as illustrated in Synthetic Scheme 4 below. The synthetic steps described above provide a convenient method for preparing a library of ethoid-peptide analogs for virtually any bioactive peptide. The method can involve determining the amino acid sequence of the desired bioactive peptide. Then a first amino acid-like residue corresponding to the first residue of the peptide can be attached to the support or to a cleavable end group that is attached to the support. One or more coupling cycles can then be carried out by sequential addition of amino acid-like residues to the growing chain to build the library of peptide analogues. The library of analogs can then be cleaved from the solid support. The amino-acid-like residue can be amino-aldehyde, hydroxyl-aldehyde, amino-carboxyl or hydroxyl-carboxyl. The amino-acid-like residue can be an amino-aldehyde, hydroxyl-aldehyde, amino-carboxyl or hydroxyl-carboxyl derived from natural and non-natural α-amino acids and β-amino-acids. The ethoid-peptide analogs can have one, two, three, four, or any number of its amide bonds replaced by ethoid bonds, up to complete replacement of amide bonds in the peptide.

The analogs can be constructed from C-terminus to N-terminus using four vessels per coupling cycle: one vessel for coupling an α-amino acid to an immobilized amine end group, one vessel for coupling an α-amino-aldehyde to an immobilized trialkylsilylether end group, one vessel for coupling an α-hydroxy-aldehyde to an immobilized trialkylsilylether end group, and one vessel for coupling an α-trialkylsilyloxy acid to an immobilized amine end group, protected using a monomer as defined above. The end group can then be determined based upon the vessel in which the previous coupling occurred.

The analogs can be constructed from N-terminus to C-terminus using four vessels per coupling cycle: one vessel for coupling α-amino acid to immobilized carboxyl end group, one vessel for coupling α-amino-aldehyde to immobilized carboxyl end group, one vessel for coupling α-trialkylsilyloxy aldehyde to immobilized aldehyde end group, and one vessel for coupling α-trialkylsilyloxy acid to immobilized aldehyde end group. The end group can then be determined based upon the vessel in which the previous coupling occurred.

Any desired and measurable biological property can be measured in the assay or groups of activities can be measured in separate assays. For example, protease resistance, bioavailability, biological clearance time, peptide half-life in particular environments, adsorption, excretion, metabolism, binding or distribution can be measured. The biological property can be specificity, selectivity, agonism, antagonism, potency, efficacy, tolerability or it can be agonism or antagonism for the parent peptide's target. Proteases can include exoproteases, such as for example DPP4, or endoproteases such as trypsin. The measured biological activity can be a composite of two, three or more of the properties listed above, for example a composite of therapeutic bioavailability, drug half-life, and clearance time.

Ethoid bonds are stable to proteolytic cleavage and can therefore be positioned near the ends or within polymer chains to render the resultant polymers resistant to proteolytic cleavage by endo or exopeptidases. Thus, ethoid bonds can be placed at the cleavage site of protease recognition sequences to increase the stability of peptides. Ethoid bonds can be placed near the C-terminus or the N-terminus of peptides to reduce C-terminal or N-terminal exopeptidases, respectively, or they can be placed near both ends of a polymer chain. For purposes of this disclosure the phrase “near the end” includes the terminal monomer linking bond and any bond near the terminus that, when replaced by an ethoid, provides increased resistance to exopeptidase cleavage. As is now apparent other ethoid bond configurations are also possible, including for example, a configuration in which at least two ethoid bonds are adjacent to each other near the N-terminus of a polymer and at least one ethoid is near the C-terminus of the polymer.

The following definitions and methods are provided to better define the present disclosure and to guide those of ordinary skill in the art in the practice of the present disclosure. Unless otherwise noted, terms are to be understood according to conventional usage by those of ordinary skill in the relevant art.

In this specification, the terms “about” and “around” are to signify that in one embodiment, the respective exact value is designated, while in another embodiment, the approximate value is designated. Thus, for example, “at least about 1,000” shall, in one embodiment, be interpreted to mean “at least 1,000” and, in another embodiment, be interpreted to mean “at least approximately 1,000.”

The term “acyl,” as used herein alone or as part of another group, denotes the moiety formed by removal of the hydroxy group from the group —COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkyl groups described herein are preferably lower alkyl containing from one to eight carbon atoms in the principal chain and up to 20 carbon atoms. They can be straight or branched chain or cyclic and include methyl, ethyl, propyl, isopropyl, butyl, hexyl and the like.

The term “aryl” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “heteroatom” shall mean atoms other than carbon and hydrogen.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and can be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and can be bonded to the remainder of the molecule through a carbon or heteroatom. Exemplary heterocyclo groups include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

In general, hydrocarbyl and substituted hydrocarbyl can be independently selected from the group consisting of alkyl, substituted alkyl, heteroalkyl, substituted heteroalkyl, aryl, substituted aryl, heteroaryl, substituted heteroaryl, alicyclic, substituted alicyclic, heterocyclic, substituted heterocyclic, optionally including in each case one or more ring structures (e.g., formed internally, or between opposing or adjacent pendant moieties). In some embodiments, hydrocarbyl or substituted hydrocarbyl can be independently selected from the group consisting of H, C₁-C₁₀ alkyl and substituted C₁-C₁₀ alkyl, optionally including in each case one or more ring structures (e.g., formed internally or between opposing or adjacent pendant moieties).

The terms “protecting group” as used herein denote a group capable of protecting a free functional group which, subsequent to the reaction for which protection is employed, can be removed without disturbing the remainder of the molecule. A variety of protecting groups for the most functional groups, methods for adding and removing the protecting groups, and the synthesis thereof can be found in “Protective Groups in Organic Synthesis” by T. W. Greene and P. G. M. Wuts, John Wiley & Sons, 1999.

The term “polyaminoacid” as used herein means an amino acid polymer. An amino acid polymer can be derived from condensation of amino acids, such as α-amino acids (substituted and unsubstiuted) in which an amine group from one α-amino acid reacts with a carboxylic group of another α-amino acid to form an amide moiety, and thereby linking the amino acid residues. A polyaminoacid can be prepared synthetically using controlled linear stepwise coupling as known in the art or as later developed. A polyaminoacid can be prepared using cell expression systems as known in the art or later developed. The α-amino acids generally include L-α-amino acids and D-α-amino acids. A polyaminoacid comprises proteins and polypeptides. The terms protein and polypeptide are generally used interchangeable herein. In strict context, to the extent necessary in a particular context to define a distinction between a protein and a polypeptide, the term “protein” can mean an amino acid polymer having fifty or more repeat units (amino acid residues with adjacent amide), and the term “polypeptide” can mean an amino acid polymer having less than fifty repeat units (amino acid residues with adjacent amide).

The “substituted hydrocarbyl” moieties (including “substituted alkyl”, or other substituted moieties) described herein are hydrocarbyl moieties (or equivalently, alkyl moieties, as understood from context) which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, ethers, and thioethers.

### Uses

The compounds and methods disclosed herein can be broadly used across various industries. For example, polyaminoacid analogs disclosed or claimed herein are useful as food additives, as cosmetics ingredients, as research reagents, as diagnostic agents, and as therapeutic agents such as drugs, among other uses.

In particular, compounds comprising an ethoid moiety or a polyethoid moiety., generally, including as described in connection with each aspect of the invention, including all general and preferred embodiments of such aspects (and including all sub-embodiments thereof) can be used as food additives, as cosmetics ingredients, as research reagents, as diagnostic agents, of as therapeutic agents (including as prophylactic agents). For example, an ethoid or a polyethoid can be used as a diagnostic agent. The diagnostic agent can be used in an assay such as an epitope in an assay comprising a monoclonal antibody. An ethoid or a polyethoid alternatively can be used as an imaging agent (e.g., as an imaging compound comprising a radiolabled ethoid moiety or a radiolabled polyethoid moiety). A compound comprising an ethoid or a polyethoid can likewise be used as an affinity reagent in affinity chromatography. The ethoid-containing affinity reagent can be a polyaminoacid analog having a specific binding affinity for a particular epitope of interest (e.g., for a “TAG” such as “FLAGS” or other similar type of epitope). The ethoid or a polyethoid compound can be used as a pharmaceutical. Such uses as a pharmaceutical can include administration to a human subject or other mammal. Such administration can include, for example and without 1 imitation, as a topical agent, for oral administration, for nasal administration, for inhalation, for injection or other manner of administration, as part of time-release or other delivery systems, together with, including as part of, medical devices or in connection with site-specific applications during surgery, in each case as is known in the art or later developed. The ethoid or a polyethoid can be a food additive, and an ingredient in a food composition. The ethoid or a polyethoid can be an ingredient in a cosmetic composition. The ethoid or polyethoid can be used as a research reagent.

The various methods can be used, for example, to manufacture ethoids or polyethoids, including ethoidpeptides or polyethoidpeptides. The methods can also be used to identify polyaminoacid compounds having a property of interest. The methods can be used to generate a data set derived from evaluation of ethoids or polyethiods for a property of interest.

Further uses of the compounds and methods will be apparent to a person of skill in the art, in view of the substantial literature related to polyaminoacid analogs.

The following examples help demonstrate the scope and content of the disclosure herein, but are not limiting on the scope of the invention.

### EXAMPLES

### A. Synthesis

### General

### Materials and Equipment

Analytical RP-HPLC-MS was performed using a C₁₈ column (250×4.6 mm, 5 μm, 60 A), operated at 1.0 mL/min. The solvent system was: buffer A=water (0.1% TFA); buffer B=acetonitrile (0.1% TFA); typical linear gradient 10 to 100% B in 10 min. The temperature was approx. 23° C. Absorbance was monitored at 210 and 254 nm. Product percentages are given by peak areas at 210 nm. Electrospray mass spectra were collected by splitting the flow of elution solvent from the column into an Applied Biosystems API-150-EX mass spectrometer.

RP-HPLC purifications were performed on a semi-preparative C₁₈ column (250×10 mm, 5 μm, 60 A) or preparative C₁₈ column (250×21.4 mm, 60 A), operated at 5-20 mL/min with the same solvent system. Absorbance was monitored at 210 and 254 nm, and peaks were automatically collected. Collected fractions were evaporated in vacuo and on a freeze dryer.

Amino acid building blocks, resins and standard solid phase peptide synthesis reagents were purchased from Advanced Chemtech (Louisville, Ky.). For N-Fmoc protected amino acids, side chain protection can be afforded by: Trityl (Trt) for C, H, and N; tert-butyloxycarbonyl (Boc) for K and W; tert-butyl (tBu) ethers or esters for Y, T, S, D and E; 2,2,4,6,7-pentamethyldihydrobenzofuran-5-sulfonyl (Pbf) for R. Diisopropylcarbodiimide (DIC), and N-hydroxybenzotriazole (HOBT) were purchased from Advanced Chemtech, Louisville, Ky. Pyroglutamic acid, N,N-dimethylaminopyridine (DMAP), N,N-diisopropylethylamine (DIEA) and N-methylmorpholine (NMM) were purchased from Acros. Trypsin, Subtillisin and DPP4 proteases were purchased from Sigma-Aldrich. Synphase lanterns were purchased from Mimotopes (Australia). All other reagents and solvents were purchased from either Sigma-Aldrich or Fisher-Acros.

### Example 1

### General Method 1

### Williamson Ether N→C Direction

### Preparation of α-Bromo Methyl Ester Building Blocks

Sidechain protected α-amino acids were converted to the corresponding α-bromo acids by the following procedure: 5 equiv. isoamyl nitrite and 5 equiv. bromine were mixed in 1 mL/mmol CH₂Cl₂. The amino acid was then added to the mixture as a solid. The first portion was added slowly and initiates the evolution of a gas (if no gas evolution is observed a few drops of DMF are added to help initiate the reaction), after which the remaining amino acid was added more quickly. Five minutes after the addition was complete, gas evolution was finished and the solution concentrated in vacuo. The residue was dissolved in ethyl acetate (20 mL/mmol) and was washed with 0.5 M sodium thiosulfate/1% HCl three times then 2M HCl three times. The organic phase was dried over sodium sulfate and evaporated under reduced pressure.

Each α-bromo acid was converted to the corresponding methyl ester by the following procedure: Dissolve the acid in 3 mL/mmol dry methanol and cooled on ice. 1.1 equiv. of thionyl chloride was added dropwise and the mixture left to stand for 16 hours. The solution is evaporated under reduced pressure and the residue dissolved in ethyl acetate, washed three times with sat. sodium bicarbonate, dried over sodium sulfate and evaporated in vacuo to give the α-bromo methyl ester.

### Loading of the Resin

To trityl resin (1.0 mmol/g loading) was added a solution of 5 equiv. α-amino methyl ester hydrochloride (Xaa-OMe) and 10 equiv. N,N-diisopropylethylamine in DMF (15 mL/mmol. After 24 hours at room temperature the resin was washed with DMF three times and CH₂Cl₂ twice.

Repeated cycles of methyl ester reductions to alcohol and nucleophilic couplings of α-bromo methyl ester building blocks are performed to synthesize a full polyethoid from N to C terminal.

### Reduction to an Immobilized Alcohol

To the resin immobilized-methyl ester terminal was added a cooled (0° C.) solution of 3 equiv. sodium borohydride in 10 mL/mmol MeOH. The mixture was allowed to come to room temperature and shaken for 4 hours. The resin was washed with water, methanol three times, CH₂Cl₂ and hexane/THF.

### Ethoid Bond Formation

In a suitably dry environment such as a glove box, the resin immobilized-hydroxy methylene terminal was dried for 30 minutes. The resin was suspended in anhydrous THF (10 mL/mmol) and cooled to −20° C. before adding 1.5 equiv. LDA (2M solution) dropwise, and the mixture shaken for 30 minutes. The resin was filtered, washed with THF and quicky resuspended in anhydrous THF.

6 equiv. of the α-bromo methyl ester (Br—CH(R^{Xaa})—COOMe) was dissolved in anhydrous THF (2.5 mL/mmol), cooled on ice and added to the resin in a microwave tube. The mixture was allowed to warm to room temperature and then treated with microwave irradiation for 20 min at 45° C. The resin was then washed with THF, water, three times with methanol, DMF and CH₂Cl₂.

A colorimetric test for an immobilized-hydroxy group was performed by taking a few resin beads and adding a solution of 1% tosyl chloride in toluene and heating, followed by a solution of 1% pNBP and DIEA in toluene. Upon heating a red color develops in the presence of any unreacted terminal alcohol.

### Resin Cleavage and Deprotection

The resin was washed with CH₂Cl₂, and then treated with trifluoroacetic acid (TFA)/triethylsilane/CH₂Cl₂ (75:5:20) for 1.5 hours at room temperature. The resin was filtered and washed with a small portion of TFA. The volume was reduced by evaporation and the filtrates were precipitated by the addition of cold diethyl ether. The precipitate was collected by centrifugation and decanting solution from the solid.

### Example 2

### General Method 2

### Williamson Ether C→N Direction

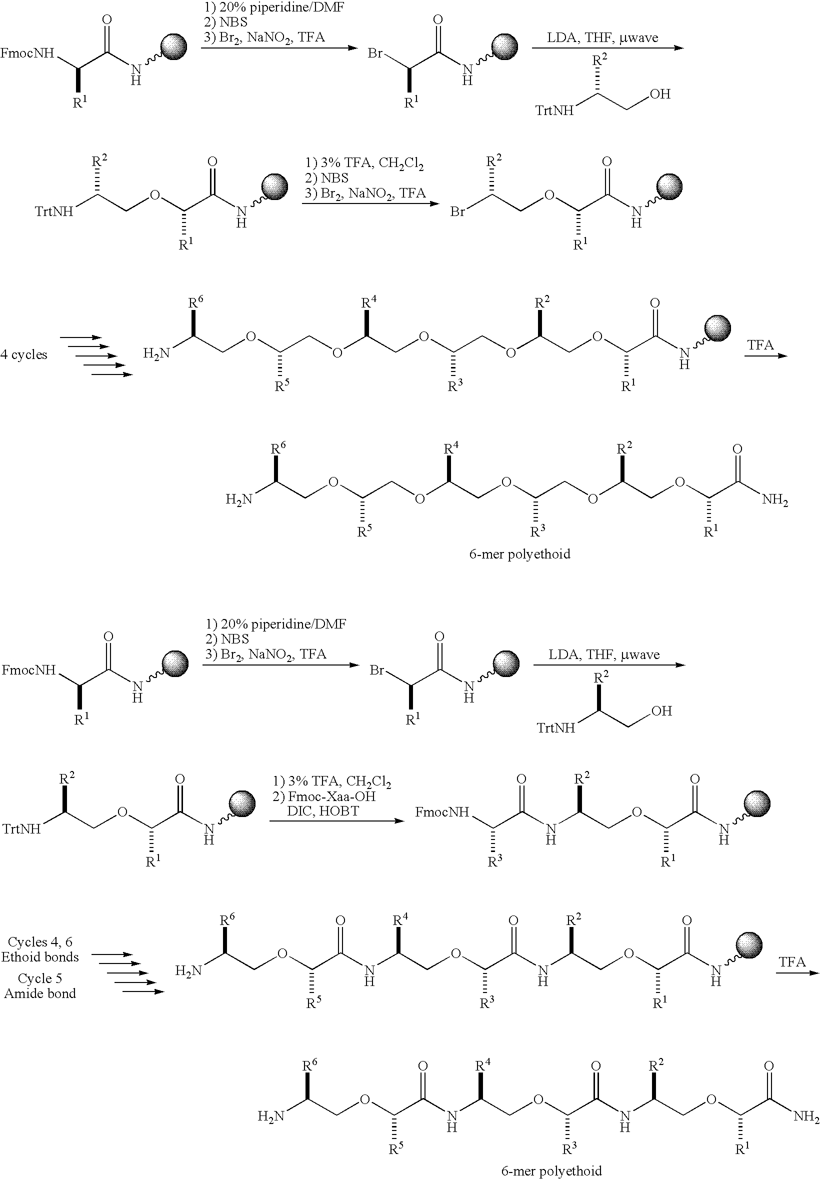

### Preparation of α-Bromo Acid Building Blocks

Sidechain protected α-amino acids of the opposite chirality to that desired in the target compound are converted to the corresponding α-bromo acids by the general method described above.

### Preparation of N-Trityl-β-Amino Alcohol Building Blocks

Method a: Sidechain protected α-amino acids were converted to the corresponding N-trityl-β-amino alcohols by the following procedure: Dissolve the α-amino acid in 3 mL/mmol dry methanol and cool on ice. 1.1 equiv. of thionyl chloride is added dropwise and the mixture left to stand for 16 hours. The solution is evaporated under reduced pressure and the residue dissolved in ethyl acetate, washed three times with sat. sodium bicarbonate, dried over sodium sulfate and evaporated in vacuo to give the α-amino methyl ester. This residue was dissolved in CH₂Cl₂ or other suitable solvent, treated with 1.1 equiv. trityl chloride and 2.2 equiv. DIEA overnight at room temperature. The reaction mixture was diluted with CH₂Cl₂, and washed three times with 0.1M HCl, sat. NaHCO₃ and brine. The organic layer was dried and evaporated in vacuo to give the N-trityl-α-amino methyl ester. The N-trityl-α-amino methyl ester was dissolved in 10 mL/mmol methanol, cooled (0° C.) and treated with 3 equiv. sodium borohydride. The mixture was allowed to come to room temperature and stirred for 4 hours. The mixture was evaporated, dissolved in ethyl acetate and washed with 5% sodium thiosulfate, three times with brine, dried (Na₂SO₄) and evaporated in vacuo to give the sidechain protected N-trityl-β-amino alcohol.

Method b: N-trityl-α-amino acid is dissolved in dry THF (1 mL/mmol), cooled (0° C.) and N-methylmorpholine (1.1 equiv) and ethyl chloroformate (1 equiv.) then added slowly. After stirring for 10 min, a white precipitate appears, and the mixture is treated with NaBH₄ (3 equiv). After a further 10 min methanol is added dropwise (1.5 mL/mmol). After complete addition of methanol the reaction allowed to come to room temperature and stirred for 30 minutes. The reaction is evaporated in vacuo, the residue dissolved in ethyl acetate, and washed with 2M hydrochloric acid (3×25 ml), and sat. NaHCO₃ solution (3×25 ml). The organic phase is dried (Na₂SO₄) and evaporated in vacuo to give the sidechain protected N-trityl-β-amino alcohol.

### Loading of the Resin

Various solid supports can be used. Procedures for coupling amino acid derivatives to suitable solid phase synthesis resins, such as a Rink resin, are found in the Novabiochem Catalog Synthesis Notes.

### Fmoc Deprotections and Amide Bond Couplings

Standard Fmoc SPPS methodologies are used such as described in the Novabiochem Catalog Synthesis Notes.

### Conversion of an Immobilized Amino Group to the Corresponding Bromide

To the resin immobilized-amino terminal is added a suspension of 3 equiv. NBS in CH₂Cl₂ with a few drops of DMF to help dissolution. After shaking for 5 min the resin is quickly rinsed with DMF and treated with 5 equiv. Br₂ in CH₂Cl₂, followed by a CH₂Cl₂ solution of 3 equiv. HNO₂ (prepared by slow addition of TFA to a stirred and cooled (0° C.) suspension of NaNO₂ in CH₂Cl₂). After gas evolution has finished, approx. 10 min, wash resin with CH₂Cl₂, DMF, MeOH, CH₂Cl₂

### Ethoid Bond Formation—Solid Phase

In a suitably dry environment such as a glove box, the resin immobilized-bromo terminal was dried for 30 minutes. To a cooled (−20° C.) solution of N-trityl-β-amino alcohol in anhydrous THF (10 mL/mmol) is added 1.1 equiv. LDA (2M solution) dropwise, and the mixture shaken for 30 minutes. The mixture was added to the resin-immobilized bromide, allowed to warm to room temperature and then treated with microwave irradiation for 20 min at 45° C. The resin was then washed with THF, water, three times with methanol, DMF and CH₂Cl₂.

### Trityl Deprotection

N-trityl-amino-terminal protecting group is removed by treatment with 3% TFA/CH₂Cl₂ solution for 3 min, draining, and repeated 3 times. The resin is washed with CH₂Cl₂ three times. The deprotection is monitored by Kaiser test on a sample of resin beads.

### Ethoid Bond Formation—Solution Phase

Sidechain protected N-trityl-β-amino alcohol is dissolved in a mixture of dry THF/dry DMF (80:20) and cooled to 0° C. Sodium hydride (2.7 equiv.) is added slowly, and when the gas evolution is finished a solution (0.5 M) of sidechain protected α-bromo acid (1.5 equiv.) in dry THF is slowly added. The mixture is treated with microwave irradiation for 30 min at 45° C. The reaction is neutralized by addition of water. The organic phase is washed with water (3×25 ml). The organic phase was dried (Na₂SO₄) and evaporated in vacuo. The residue is purified by flash chromatography.

Incorporation of Ethoid-Containing Fragments into SPPS:

Coupling: HOBT (3 equiv.) and DIC (2.1 equiv.) are added to the N-Trityl-ethoid fragment (2 equiv.) dissolved in DMF, and stirred for 15 min before adding to a resin immobilized amino group (1 equiv.). The resin mixture was shaken for 4 hours, and monitored by Kaiser test on a sample of resin beads. The resin was washed with DMF three times. If required coupling procedure was repeated.

### Example 3

### General Method 3

### Ester Reduction

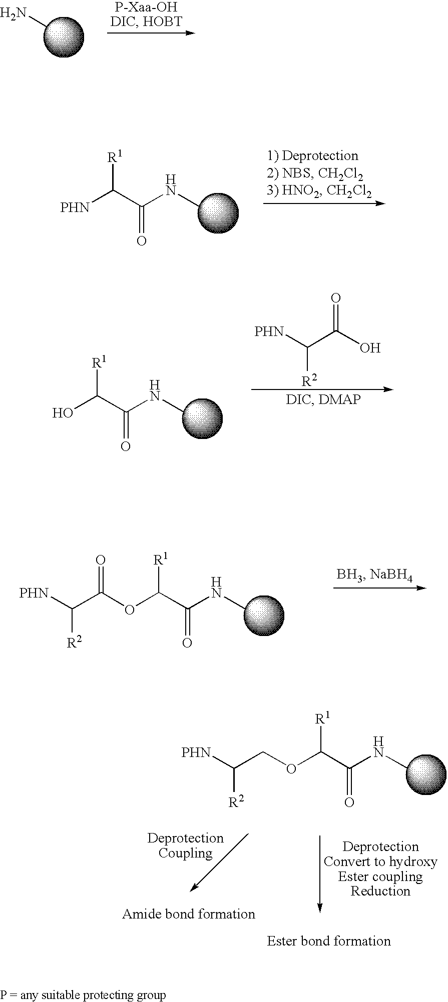

### Example 4

### Preparation of Building Blocks for General Methods 4, 5 and 6

### Reductive Etherification

### N-Fmoc-α-Amino Aldehydes Via N-Fmoc-α-Amino Weinreb Amides

Protected N-Fmoc-α-amino aldehydes can be prepared as described in review articles (Moulin, A.; Martinez, J. and Fehrentz, J.-A., Synthesis of Peptide Aldehydes. J. Peptide Science 2007, 15, 1-15. Gryko, D. et al., Synthesis and Reactivity of N-protected-α-amino aldehydes. Chirality 2003, 15, 514-541).

Fmoc-Xaa-OH was dissolved in CH₂Cl₂ (4 ml/mmol, if needed few drops of DMF were added to ensure total solubility). To the reaction vessel was added N,O-dimethylhydroxyl amine hydrochloride (1.1 eq), N-methylmorpholine (2.2 eq) and N-hydroxybenzotriazole (1.1 eq). Diisopropylcarbodiimide (DIC) was then added slowly to the reaction mixture and stirred overnight at room temperature. Completion of the reaction was confirmed by tlc. The white urea precipitate was removed by filtration and the filtrate was washed with saturated NaHCO₃ (3 times), brine and 1M HCl (3 times) and dried over Na₂SO₄. The solution was concentrated on a rotary evaporator to give the Weinreb amide.

To a cooled solution of Fmoc-Xaa-N(Me)OMe in anhydrous tetrahydrofuran (5 mL/mmol) was added 5 equiv. LiAlH₄ as a solid. The reaction mixture was stirred 30 min at −70° C. The solution was diluted with diethyl ether and quenched with 1M HCl (aq.). The aqueous layer was extracted with diethyl ether. The organic layer was then washed with saturated NaHCO₃, dried over Na₂SO₄ and then concentrated on a rotary evaporator to give the aldehyde. The aldehyde is stored dry and cold and used within a week.

### α-Hydroxy Aldehyde

N-Fmoc-α-amino Weinreb amide (Fmoc-Xaa-NMeOMe) was dissolved in 20% DMF/piperidine and stirred at room temperature for 2 hours. The solution was concentrated on a rotary evaporator. The residue was triturated using cold ether, and suspended in dichloromethane (3 mL/mmol). N-bromosuccinimide (NBS, 2.05 equiv.) was added to the suspension and stirred for five minutes until all of the α-amino Weinreb amide (H-Xaa-NMeOMe) dissolved. Meanwhile a solution of nitrous acid was prepared by adding TFA slowly to a stirred suspension of NaNO₂ in dichloromethane. The nitrous acid solution (200 mM final) was added to the reaction mixture and stirred for 10 minutes at room temperature. The reaction mixture was diluted in dichloromethane and washed with 10% HCl aqueous. The organic layer was dried over Na₂SO₄ and concentrated in vacuo to give the α-hydroxy Weinreb amide (HO—CH(R^{Xaa})CONMeOMe, approx. yield 80-90%). LiAlH₄ (1.5 eq.) was added to the α-hydroxy Weinreb amide in anhydrous THF (5 ml/mmol). The reaction was stirred 30-45 minutes at −10° C. and quenched by 5% HCl aqueous. The solution was diluted with H₂O, extracted twice with ethyl acetate and the organic layer was filtered through a plug of silica gel. The filtrate was dried and concentrated in vacuo to give the α-hydroxy aldehyde (HO—CH(R^{Xaa})CHO, approx. yield 75-90%).

### α-Silyloxy Acid

N-bromosuccinimide (NBS, 2.05 equiv.) was added to a suspension of α-amino acid (H-Xaa-OH) and stirred for five minutes until all of the α-amino acid dissolved. Meanwhile a solution of nitrous acid was prepared by adding TFA slowly to a stirred suspension of NaNO₂ in dichloromethane. The nitrous acid solution (200 mM final) was added to the reaction mixture and stirred for 10 minutes at room temperature. The reaction mixture was diluted in dichloromethane and washed with 10% HCl aqueous. The organic layer was dried over Na₂SO₄ and concentrated in vacuo to give the α-hydroxy acid (HO—CH(R^{Xaa})—COOH, approx. yield 80-95%). Alternatively, α-hydroxy acids can be obtained by methods similar to those described in the literature (Deechongkit, S.; You, S.-L.; Kelly, J., Synthesis of all nineteen appropriately protected chiral α-hydroxy acid equivalents of the α-amino acids for Boc solid-phase depsi-peptide synthesis. Organic Letters 2004, 6(4), 497-500).

To a cooled solution of α-hydroxy acid and N,N-dimethylaminopyridine (DMAP, 0.5 equiv.) in dichloromethane (or DMF) was added t-butyldimethylchlorosilane (2.4 equiv.) at 0° C. The mixture was warmed to room temperature and stirred for 12 hours. The reaction was quenched with water and the organic layer was extracted three times with ethyl acetate. The combined organic layer was washed 3 times with brine, dried over Na₂SO₄ and concentrated in vacuo. The residue was redissolved in a mixture of MeOH/THF (3/1; 10 mL/mmol) and the mixture was treated with a solution of K₂CO₃ (10 g/mL @ 2.5 mL/mmol). The reaction was stirred for 1 hour at room temperature, and then concentrated to one-quarter volume. The resulting aqueous mixture was cooled to 0° C. and the pH was adjusted to 4-5 with 1M HCl. The aqueous mixture was extracted 3 times with diethyl ether, then the organic layer was washed 3 times with brine, dried over Na₂SO₄ and concentrated in vacuo to give the α-t-butyldimethylsilyloxy acid (tBuMe₂SiO—CH(R^{Xaa})—COOH, yield 90-100%).

### α-Silyloxy Weinreb Amide

To a cooled solution of α-hydroxy Weinreb amide (HO—CH(R^{Xaa})CON(Me)OMe, as prepared above) and N,N-dimethylaminopyridine (DMAP, 0.5 equiv.) in dichloromethane (or DMF) was added t-butyldimethylchlorosilane (2.4 equiv.) at 0° C. The mixture was warmed to room temperature and stirred for 12 hours. The reaction was quenched with water and the organic layer was extracted three times with ethyl acetate. The combined organic layer was washed 3 times with brine, dried over Na₂SO₄ and concentrated in vacuo to give the α-t-butyldimethylsilyloxy Weinreb amide (tBuMe₂SiO—CH(R^{Xaa})CON(Me)OMe, yield 90-100%).

Ketone Building Blocks from Aldehyde Building Blocks

(Trimethylsilyl)—R* (1.2 equiv.) is added to a cold (0° C.) solution of ethylmagnesium bromide (2 M THF solution, 1.2 equiv.) dissolved in THF (3 mL/mmol). This solution is stirred for 1 hour at 5-15° C. and for 15 minutes at room temperature. A solution of the aldehyde (1 equiv. dissolved in THF 2 mL/mmol) is then added dropwise over a 30 min period. The reaction solution is allowed to stir for an additional 30 min before being quenched with NH₄Cl_{(satd)} and concentrated. The resulting mixture is dissolved in ether, washed NH₄Cl_{(satd)}, dried (MgSO₄), and concentrated to give a ketone (Y—CH(R^{Xaa})CO—R*, where Y is a protected amine or alcohol) that was purified as needed by flash chromatography.

### Example 5

### General Method 4

### Reductive Etherification N→C Direction

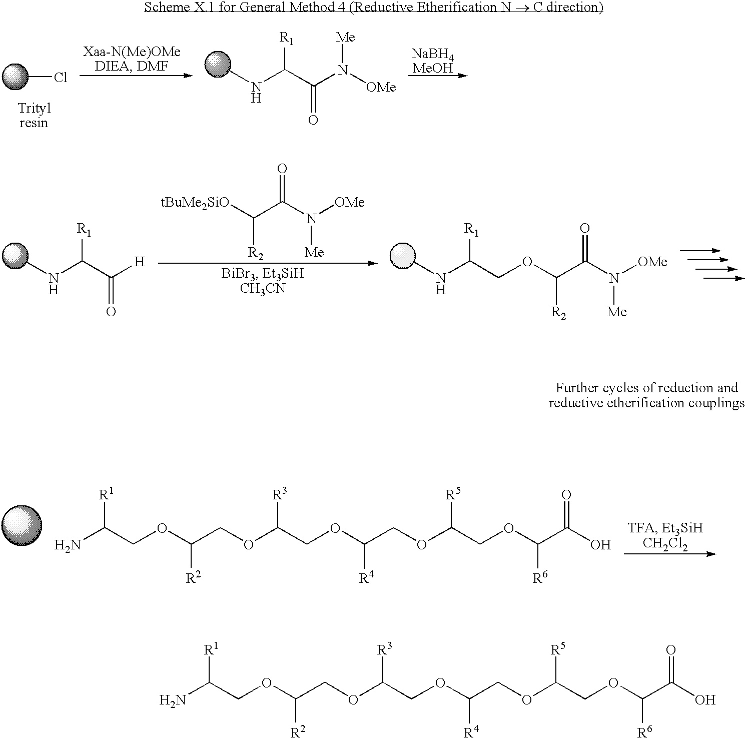

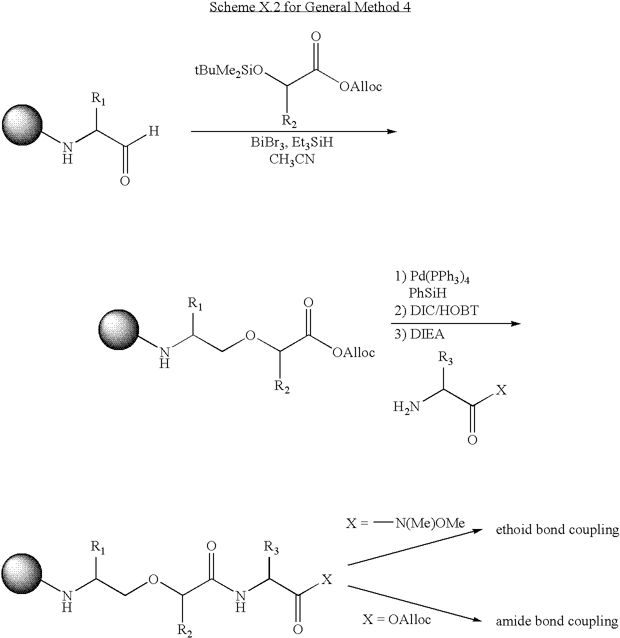

### General Method 5

### Reductive Etherification, C→N Direction, Amino to Hydroxy Conversion on Solid Support

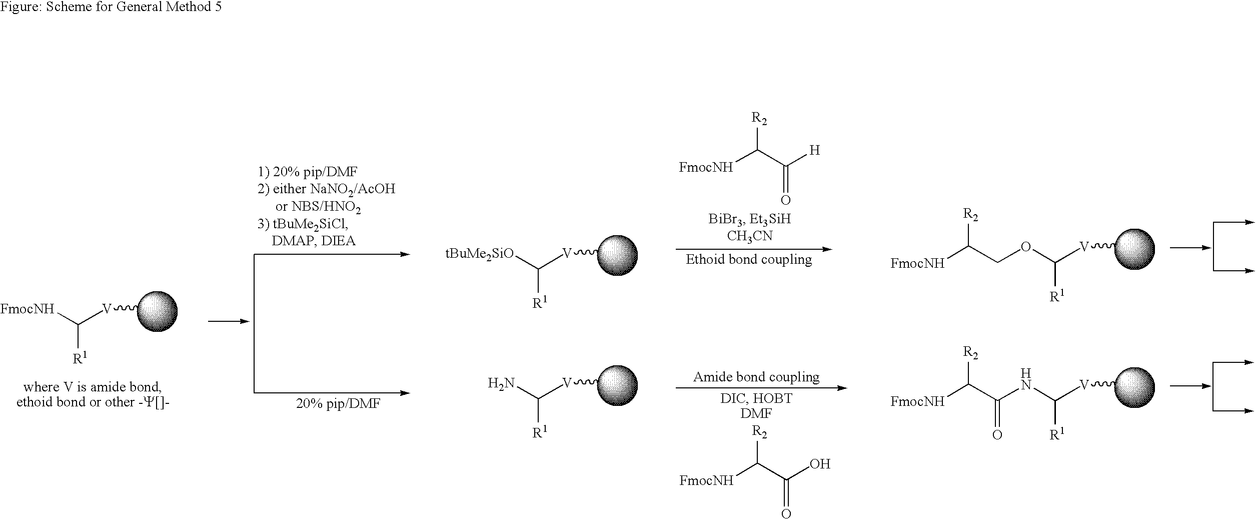

### Materials

Ethoid-peptides are synthesized on SynPhase PA D-series Lanterns derivatized with either a Rink linker for terminal amides or with a hydroxymethyl linker for terminal acids (loading 8 umol/lantern). Fmoc protected amino acids can be used throughout. Side chain protection can be afforded by: Trityl (Trt) for C, H, and N; tert-butyloxycarbonyl (Boc) for K and W; tert-butyl (tBu) ethers or esters for Y, T, S, D and E; 2,2,4,6,7-pentamethyldihydrobenzofuran-5-sulfonyl (Pbf) for R.

### Fmoc Deprotections

Prepare a solution of 20% piperidine in DMF. Immerse the Fmoc-lanterns in Fmoc deprotection solution for 30 minutes at rt. Wash the lanterns in DMF three times for 3 minutes, MeOH two times for 3 minutes, CH₂Cl₂ for 3 minutes and air dried.

### Amide Bond Couplings

General coupling conditions utilize a solution of DMF with reagents at the following ratios and concentrations: Ratios=1× monomer:1.1×HOBt:1×DIC Final conc.=200 mM monomer:220 mM HOBt:200 mM DIC

This activated amino acid solution is prepared from equal volumes of an amino acid stock solution (400 mM Fmoc-Xaa-OH in DMF) and activator solution (440 mM HOBt and 400 mM DIC in DMF). The activator solution should be prepared fresh. Minimum volume of 500 uL for each lantern. Dispense a measured volume of monomer solution, and add an equal volume of activator solution. Allow to mix for 5 minutes at room temperature to activate the monomer. Mix the coupling solution with the amino-lanterns in the suitable vessel and allow to react at room temperature for 1 hour. Wash the lanterns in DMF 3 times for 3 minutes each. The presence of unreacted amino groups can be monitored by the presence of 50 μM bromophenol blue indicator. If necessary, the coupling is repeated using HBTU/HOBT/DIEA instead of DIC/HOBT.

### Conversion of a Solid Supported-Amino Terminal to a Solid Supported-Hydroxy Terminal (Geysen Chemistry Method)

Immerse the amino-lantern in a suspension of 500 mM N-bromosuccinimide in CH₂Cl₂ (with a few drops of DMF (<5%) as needed to dissolve) and shake for 5 minutes, during which time some of the NBS dissolves. The lantern is quickly rinsed in CH₂Cl₂ to remove any excess NBS and then immersed in 200 mM nitrous acid in CH₂Cl₂ (for a 10 mL nitrous acid solution: slowly add 150 uL of TFA to a stirred suspension of 138 mg NaNO₂ in 1 mL DCM. To the green solution add 9 mL CH₂Cl₂).

After 10 minutes at room temperature gas evolution is finished and the lantern washed in CH₂Cl₂ for 3 minutes, MeOH for 3 minutes and CH₂Cl₂ for 3 minutes.

### Conversion of a Solid Supported-Amino Terminal to a Solid Supported-Hydroxy Terminal (Diazo Chemistry Method)

The lantern was submerged in 1.5 mL of a solution of acetic acid/water (20/80). 0.5 mL of a 1 M solution of NaNO₂ was added slowly. The lantern was reacted for 10 min in this solution, and then removed and washed twice with water, twice with methanol and twice with DMF.

When performed on a resin solid support, such as a polystyrene-based resin: The resin is suspended in a solution (10 mL/g of resin) of water/acetonitrile/acetic acid (40/40/20). A 1 M solution of NaNO₂ is added slowly (3 mL/g of resin) and shaken for 10 min. The resin is drained, washed twice with water, then methanol, DMF, methanol, DCM, and finally DMF.

### Silyl-Ether Formation

Immerse the hydroxyl-lantern in a solution of 300 mM DMAP, 600 mM N,N-diisopropylethylamine and 600 mM tert-butyldimethylchlorosilane in CH₂Cl₂. After 1 hour at room temperature the lantern is washed in CH₂Cl₂ for 3 minutes, MeOH for 3 minutes and acetonitrile for 3 minutes.

### Ethoid Bond Formation

Wash the lantern with anhydrous acetonitrile once for 3 min. In a dry atmosphere such as a glovebox purged with dry nitrogen, immerse the lantern in a solution of 500 mM triethylsilane in anhydrous acetonitrile and slowly add dry solid bismuth bromide to give 50 mM (20 mg/mL). Cool the mixture on ice and slowly add a solution of 500 mM Fmoc-α-amino aldehyde in anhydrous acetonitrile. The mixture is shaken for 3 hours at room temperature. Wash the lantern with acetonitrile 3 times for 3 minutes, DMF for 3 minutes, MeOH for 3 minutes, and CH₂Cl₂ for 3 minutes.

### Final Lantern Cleavage and Side Chain Deprotection

Simultaneous side chain deprotection and cleavage is carried out by immersing each lantern (2.5 mL) in a cleavage solution containing 85% TFA/5% anisole/5% thioanisole/2.5% water/2.5% EDT for 2 hours at room temperature. Evaporate most of the TFA under vacuum (or a gentle stream of N₂) and then precipitate the cleaved ethoid-peptide by the addition of cold diethyl ether. Collect the precipitate by centrifugation and decanting of the ether solution and air dry.

### Example 6

### General Method 6

### Reductive Etherification, C→N Direction, 4 Types of Building Blocks

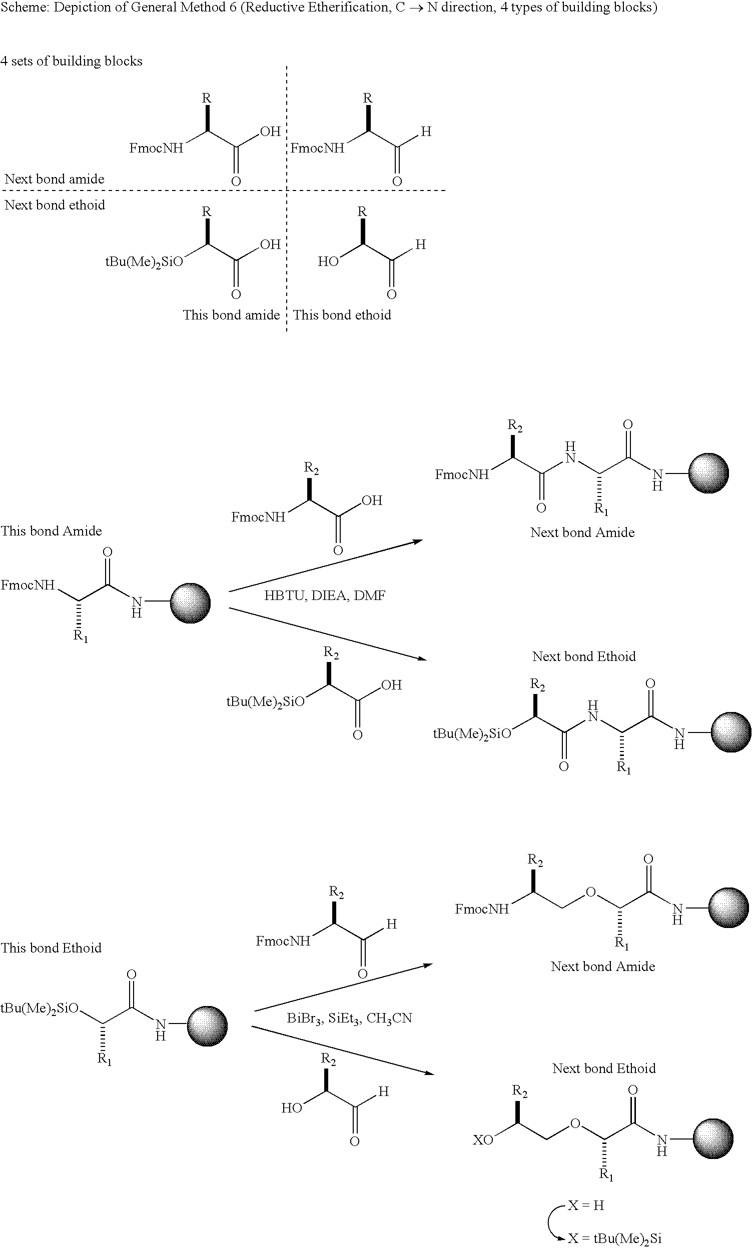

The same general methods as those used for General Method 5 are utilized for General Method 6, as necessary, in combination with the four types of building blocks depicted in Scheme above: N-Fmoc-amino acid, N-Fmoc-amino-aldehyde, hydroxy-aldehyde and silyloxy-acid. A synthetic scheme showing the synthesis of an example polyethoid is shown below.

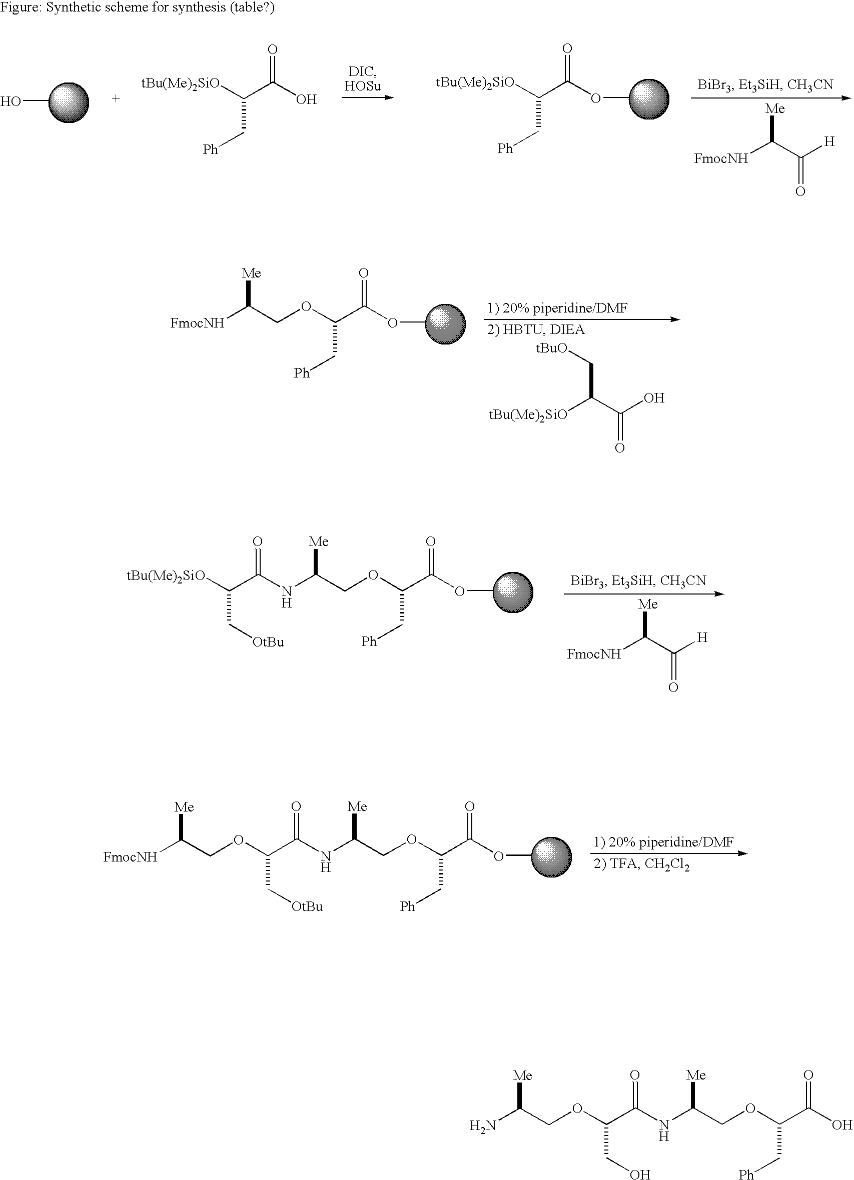

### Example 7

### Retro Inverso

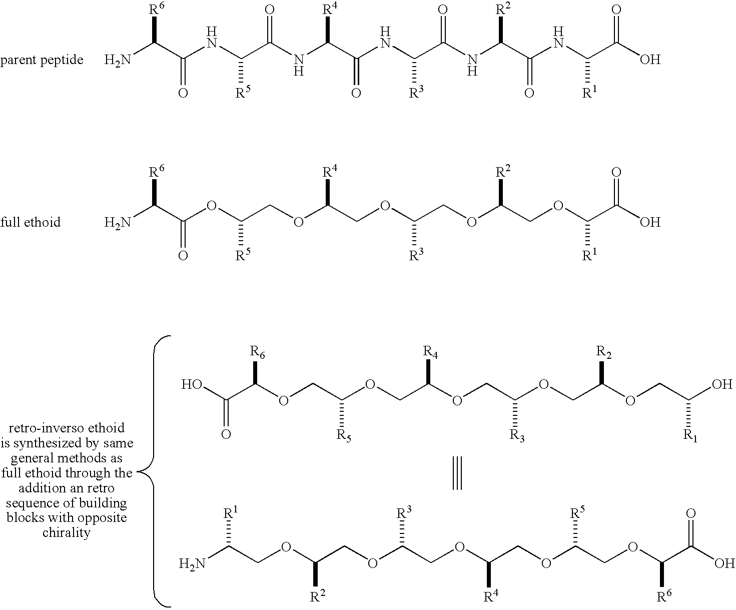

Retro-inverso polyethoids are prepared by the same general methods described above for polyethoids, for example General Method 5. Typically, the order of addition of building blocks is reversed and the chirality of the building blocks are inverted relative to the corresponding polyethoid compound. The terminal groups of retroinverso polyethoids can be manipulated by any suitable method of SPPS or solid phase organic synthesis.

The method described to prepare retroinverso polyethoids can be utilized to prepare compounds that contain retroinverso ethoid bonds, and amide bonds and/or other -ψ[ ]- bonds (or retroinverso versions thereof), by any suitable corresponding methods, that are described above.

### Example 8

### Ethoid Scanning

### Synthesis

The preparation of ethoid compounds is possible with absolute control over the number of ethoid bond replacements and their positions in a sequence. Libraries of ethoid compounds based on a parent sequence, and various systematic replacement strategies, can be prepared. One possible replacement strategy is the ethoid scan, whereby a number of compounds (n−1 compounds, were n is the length of the sequence) are prepared based on a parent sequence. Each compound comprises a single ethoid bond at a different position in the sequence.

One additional strategy is the reverse ethoid scan whereby a number of compounds (n−1 compounds, were n is the length of the sequence) are prepared based on a parent sequence. Each compound comprises a single amide bond at a different position in the sequence.

One preferred synthetic platform to prepare such libraries is a parallel mix-and-split encoded synthetic strategy.

The library of compounds derived from an ethoid scan of the sequence ASAF are shown in Schemes XX and XX. Further figures depict one possible synthesis by General Method 6, utilizing an encoding strategy comprising of Synphase lanterns (Mimotopes, Australia) encoded by the attachment of inert polyethylene cogs to the lantern via a stem.

Several other encoding strategies for solid phase parallel synthesis would be suitable for the synthesis of polyethoids.

The synthesis of a library of LHRH ethoid compounds that comprises an ethoid scan and a reverse ethoid scan of a parent sequence, was performed using General Method 5 and is described in section D, Examples-Synthesis below.

### Assays

Any suitable assay or measurement of a compound property can be applied to a library of ethoid compounds, such as those described below in the General Assays and specific in vitro assays. Relative measurements of properties across a library provides ways to assess the consequences of the inclusion of one or more ethoid bonds at various positions in a parent sequence.

Measurement/assay of various properties across ethoid libraries can demonstrate the consequences of having, an amide bond, an ethoid bond, or other -ψ[ ]-., at a position within a parent sequence, on for example, in vitro activity, peptidase stability or in vivo PK.

Methodology of Ethoid Scanning of Peptide

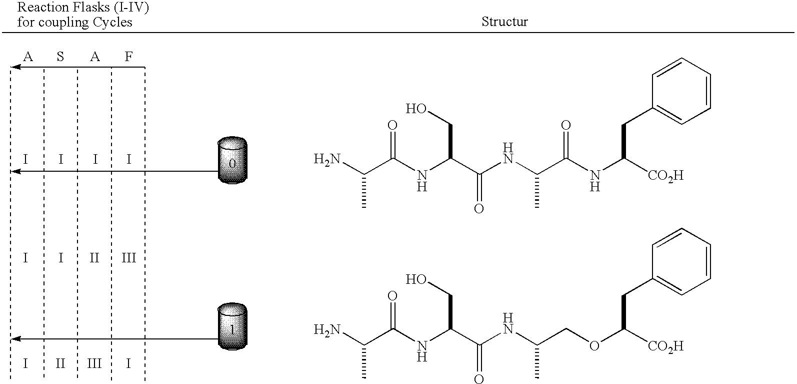

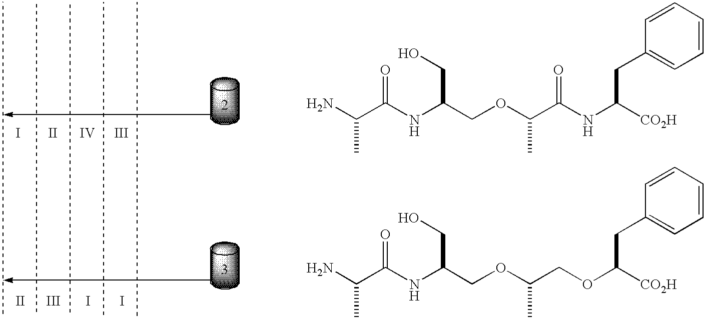

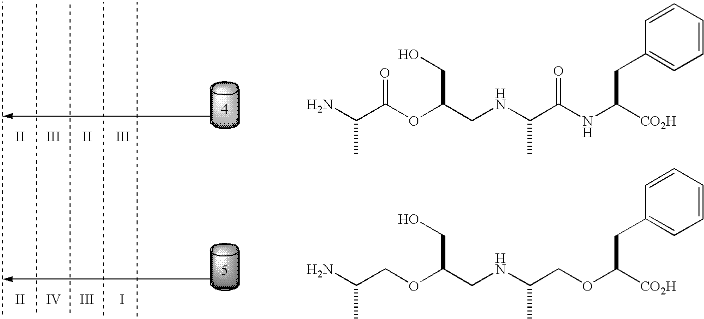

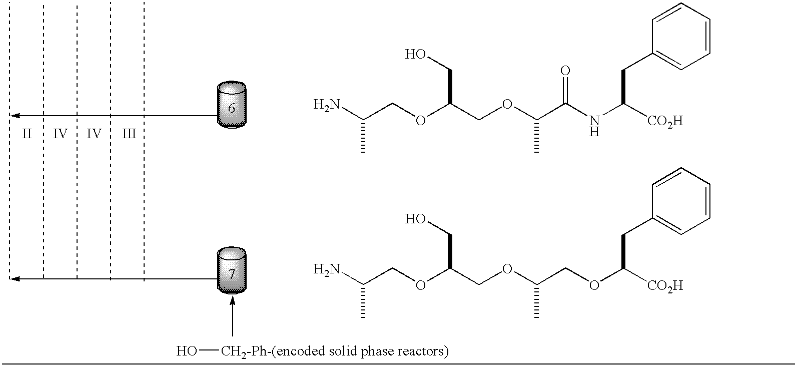

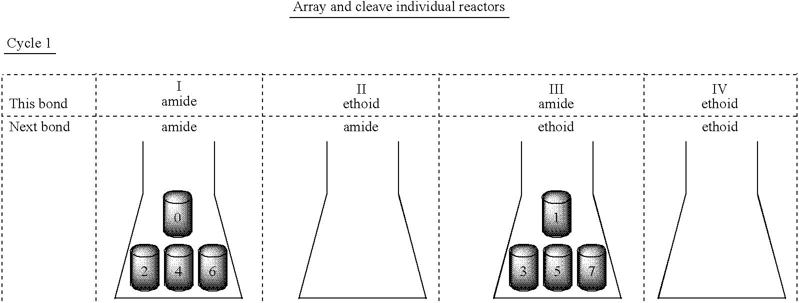

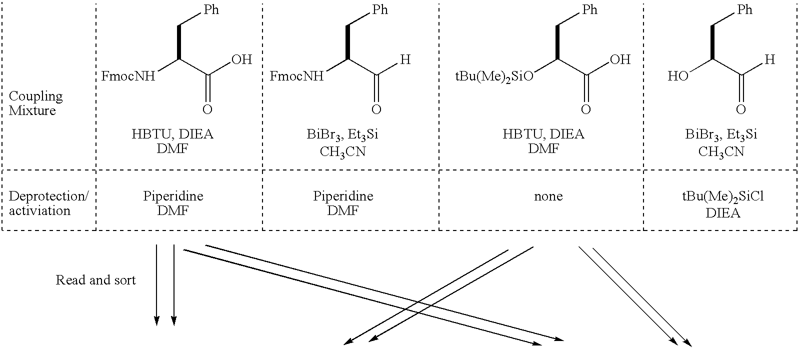

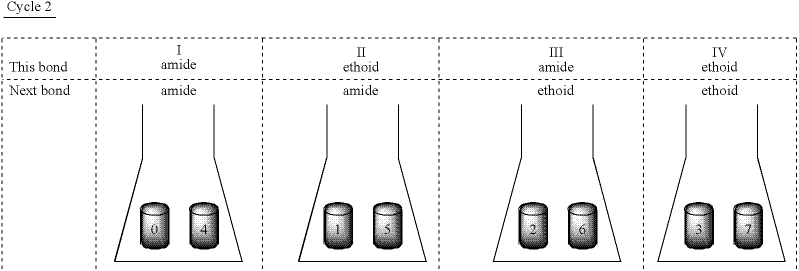

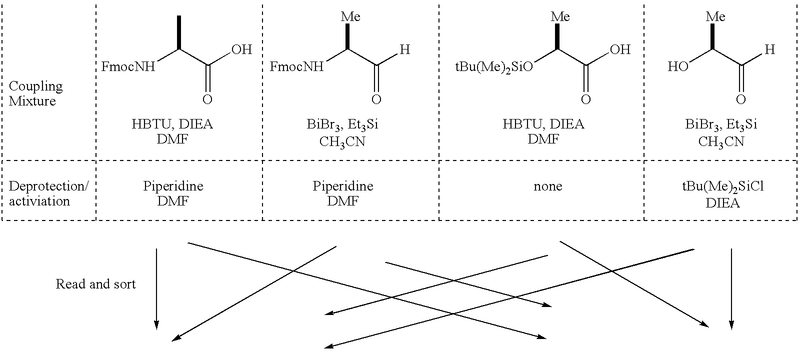

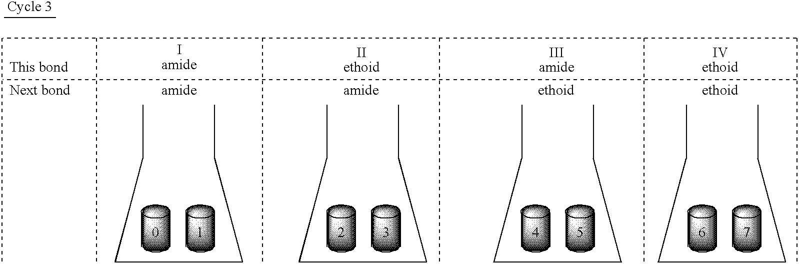

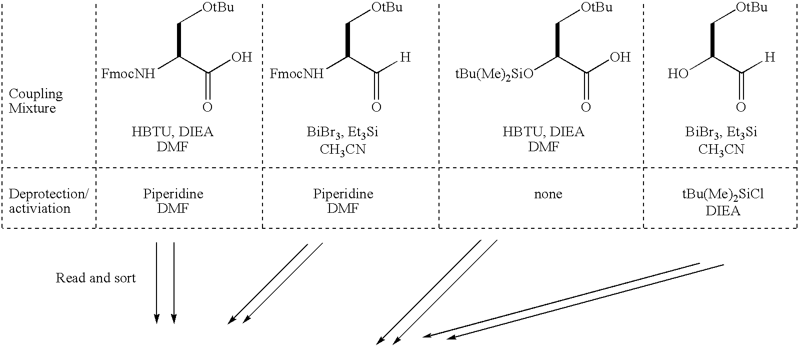

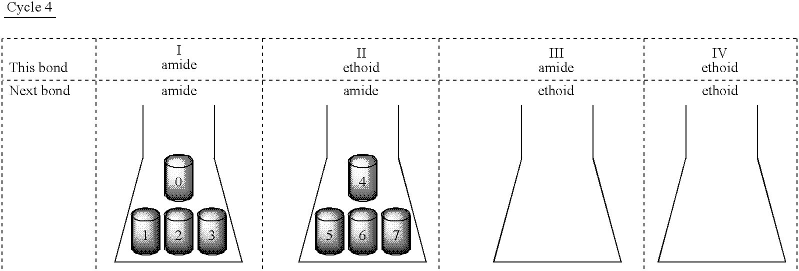

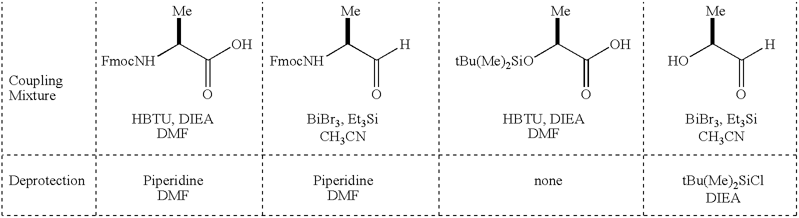

### D. General Assays

### Protease Stability Assays

### DPP4 Enzyme Stability Assay

The compounds (1.5 μmol) are dissolved in 300 μl of 50 mM tris buffer pH 7.5. 1 mU of DDP-4 (Sigma) is added to the solution and incubated at 37° C. At times 0, 1, 4, and 8 hours, 40 μL of the cleavage solution was diluted into 100 μl of 1:1 methanol/acetonitrile (0.1% TFA). The samples were analyzed by HPLC-MS, using a Gilson HPLC with dual wavelengths UV detector (214 and 254 nm). The column was a C₁₈; 4.6×250 mm; 5 μmx 6 A. The solvent system used was A: H₂O/0.1% TFA, B: acetonitrile/0.1% TFA, at 1.0 mL/min, the gradient started at 0% B or going to 100% B in 60 minutes. The mass-spectrometer was an Applied Biosystem MS API 150 EX, and the range scanned was 500-2000 amu.

### Example 9

### GHRH

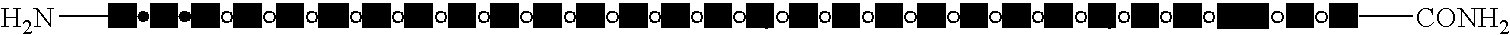

The GHRH(1-29)-NH₂ polyethoid shown above was studied for DPP-4 peptidase resistance by the general method described above, with collection of HPLC-MS data. As shown in the data indicates no degradation of the ethoid compound relative to peptide control which was substantially cleaved to GHRH(3-29)-NH₂ after 8 hours incubation.

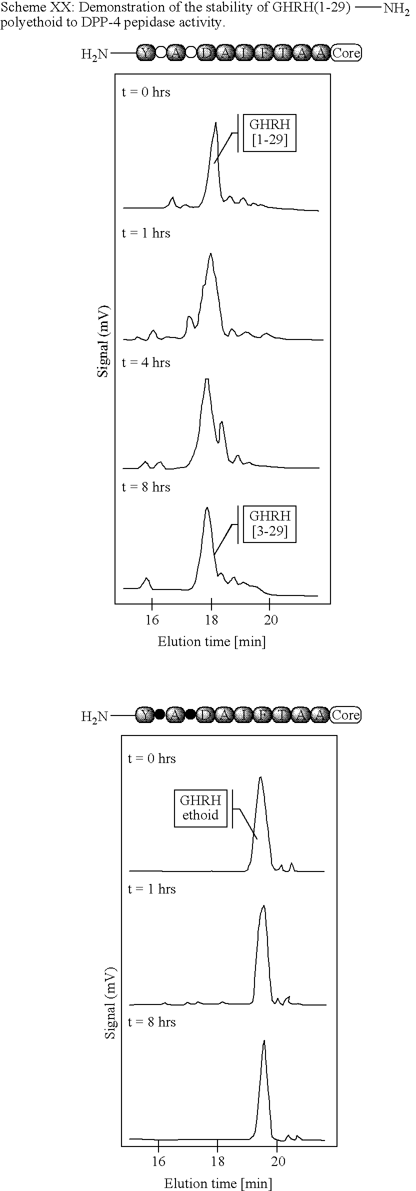

### Trypsin and Subtilisin Enzyme Stability Assay

The assay requires a). 50 mM ammonium bicarbonate solution in deionized water pH 8 b.) Enzyme stock solution at 1 mg/mL in buffer, and c) a peptide or polyethoid compound stock solution at 10 mg/mL in DMSO

Method: 1) In 300 uL of 50 mM ammonium bicarbonate add 3 uL of peptide stock solution. 2) Remove 4 uL sample and add to 200 uL acetonitrile/0.01% AcOH suitable for injection into mass spectrometer for collection of time=0 data. 3) Add 1.5 uL of enzyme stock solution and start timer. 4) Vortex 1 sec and incubate at 25° C. 5) Repeat step 2 at selected time points (suggested 2 min, 10 min and 30 min)

### Example 10

### LHRH

Peptidase stability assays were performed on LHRH compounds according to the general procedure above for subtillisin. Observed ESMS data is shown in Table III below.

TABLE III

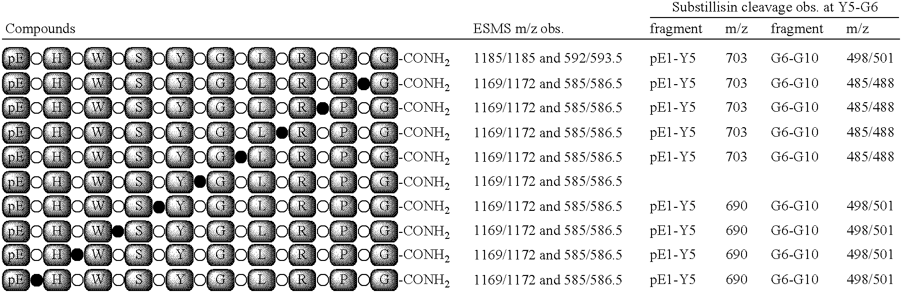

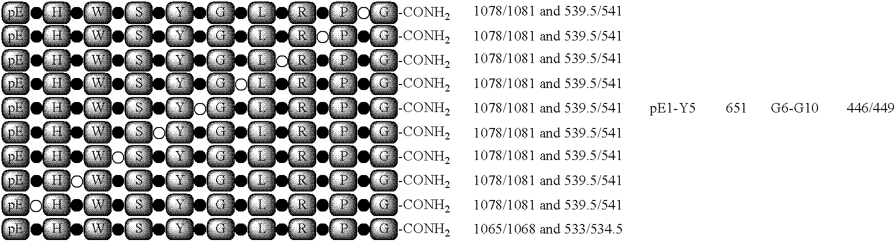

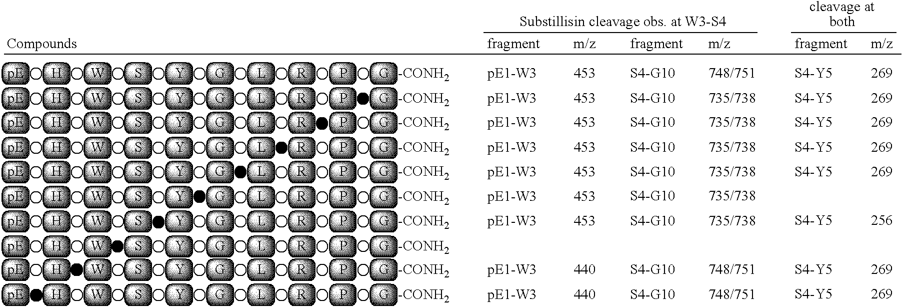

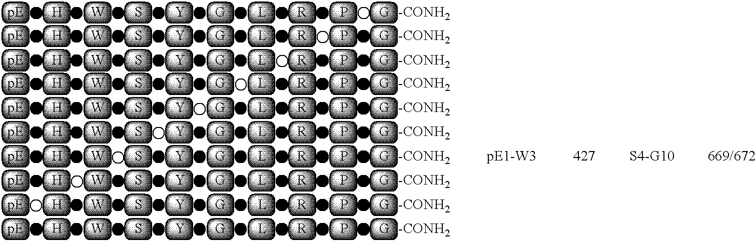

Note:
10Gly⁰:Gly³ isotopic mass peak split

### In Vivo PK

In order to determine the in vivo pharmacokinetic properties of a compound, the compound is administered both intravenously (IV) and orally (PO). Typical doses are 0.5 mg/kg IV and 5 mg/kg PO. Three animals (e.g. rats) are usually used per treatment group. Doubly-jugular vein cannulated male Sprague-Dawley rats are fasted for 12 hours prior to dosing. A predose sample is collected and the animals are dosed with the test compounds by the appropriate route of administration. Plasma samples (300 uL) are collected at the required times, stabilized with an anticoagulant such as K3EDTA, and frozen until LCMSMS analysis. MSMS analyses will typically use positive or negative electrospray or APCI ionization.

Pharmacokinetic properties are generally calculated by fitting the data to compartmental or non-compartmental models. The IV data can be used to calculate the clearance and volume of distribution terms. The IV and PO data together provide bioavailability, and absorption rates.

### E. Specific Examples

### Synthesis

### Example 11

### P3 B-Cell Epitope

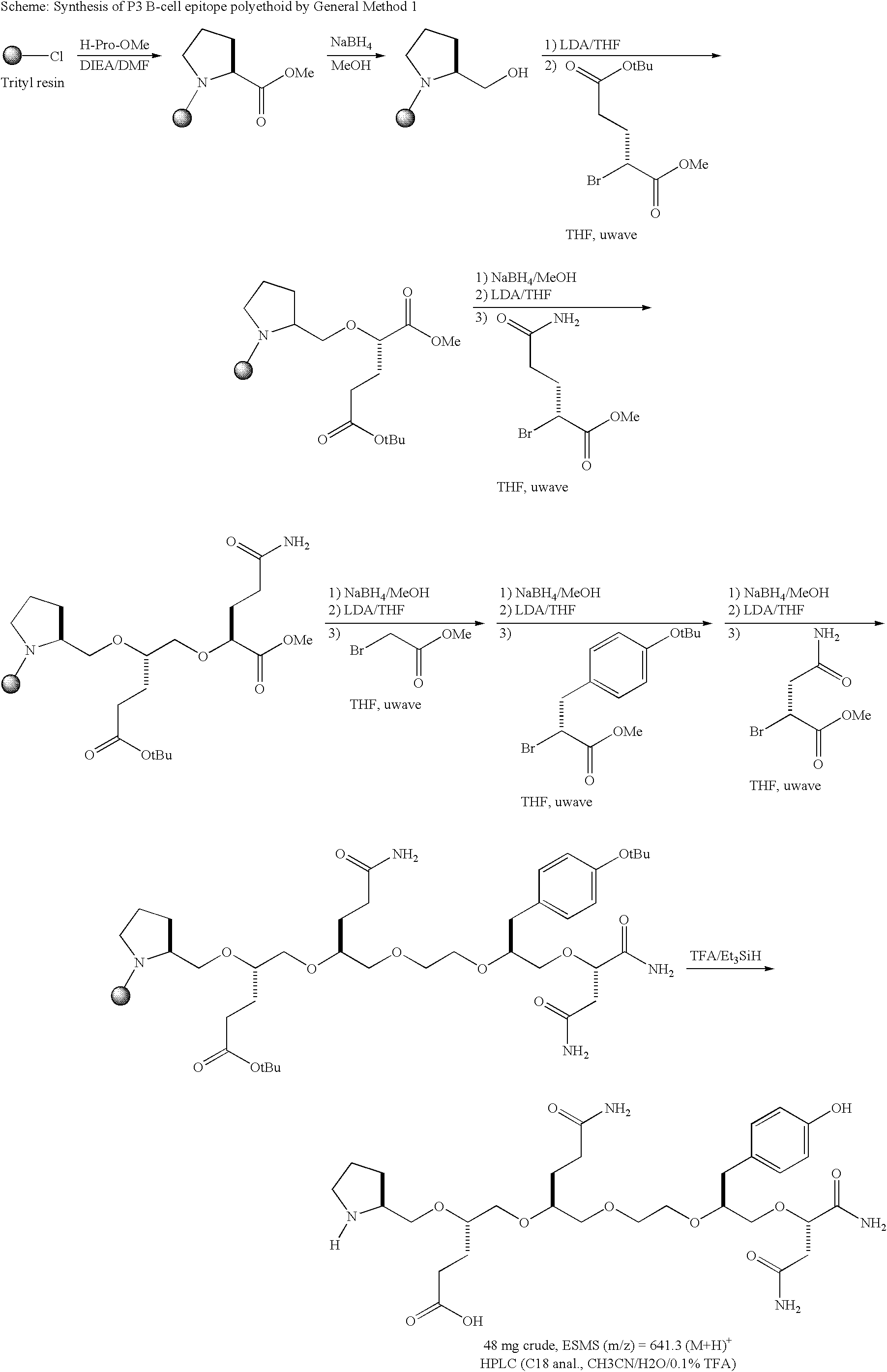

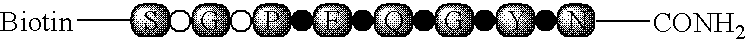

The polyethoid of compound above was prepared by the General Method 1 at the scale of 0.3 g (0.3 mmol) trityl resin, with specific details given below.

### Preparation of Specific Building Blocks

3.0 mmol portions of H-D-Glu(OtBu)-OH, H-D-Gln-OH, H-Gly-OH, H-D-Tyr(OtBu)-OH were each converted to the corresponding α-bromo acids by the general procedure:

(2R)-2-bromo-4-tertbutyloxycarbonyl-butanoic acid, 0.72 g.

(2R)-2-bromo-4-carboxyamide-butanoic acid, 0.514 g (82%).

bromoacetic acid, 0.31 g (76%).

(2R)-2-bromo-3-(4-tertbutyloxyphenyl)propanoic acid, 0.76 g (85%).

4.0 mmol H-D-Asn-NH₂ was also converted to the corresponding α-bromo amide by the same procedure: (2R)-2-bromo-3-carboxyamide-propanamide, 0.597 g (77%).

Each α-bromo acid was converted to the corresponding methyl ester by the general procedure to give the α-bromo esters in quantitative yield. L-proline methyl ester is commercially available.

Repeated cycles of reduction and coupling were performed by the general procedure to sequentially add α-bromo ester monomers derived from D-Glu(OtBu)-OH, D-Gln-OH, Gly-OH, D-Tyr(tBu)-OH. The final monomer added was the α-bromo amide derived from D-Asn-NH₂.

48 mg (25% yield) of crude residue was collected after resin cleavage, HPLC (C₁₈ analytical 5 um column) gradient 10%-100% CH₃CN (0.1% TFA)/H₂O (0.1% TFA) over 10 minutes gave peak retention time of 5.90 minutes, ESMS m/z=641.3 (M+H)⁺. The crude residue was purified by reversed phase C₁₈ HPLC to yield 19 mg (10% yield) of the ethoid H-Proψ[CH₂O] Gluψ[CH₂O]Glnψ[CH₂O]Glyψ[CH₂O]Tyrψ[CH₂O]Asn-NH₂.

Standard solution phase peptide synthesis (DIC/HOBT method) was used to sequentially couple Gly, Ser, and Biotin residues to the N-terminal of the full ethoid.

### P3 Ethoid Scan Synthesis by General Method 2

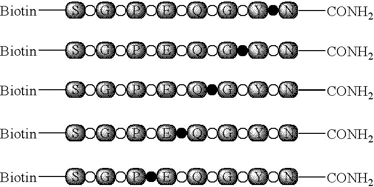

The single ethoid bond containing analogs of P3 B-cell epitope, making up the ethoid scan set, were synthesized according to the General Method 2, where protected ethoid bond-containing fragments (ie., Y*N, G*Y, Q*G, E*Q and P*E, where * denotes an ethoid bond) were prepared in solution, and then coupled into the Biotin-SG-P3 sequence, utilizing standard Fmoc solid phase peptide synthesis methodologies (SPPS).

### Example 12

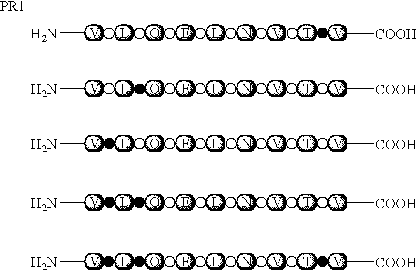

PR1 peptide was prepared by standard Fmoc SPPS methodologies. PR1 ethoid bond-containing compounds shown above were synthesized by General Method 2, preparing the protected ethoid-containing fragments separately (ie., T*V, L*Q, V*L and V*L*Q, where * denotes an ethoid bond), and then coupling into the PR1 sequence using standard Fmoc SPPS methodologies on Rink resin.

PR1 is a T cell epitope derived from the proteinase 3 protein. Proteinase 3 has been shown to be aberrantly expressed in malignant hematologic tissues.

PR1 peptide has also been demonstrated to be a target for active vaccine therapy for the treatment acute myelogenous leukemia (AML) with significant response rates and reversal of disease sustained for many years.

Five PR1 analogs have been prepared. They are shown below.

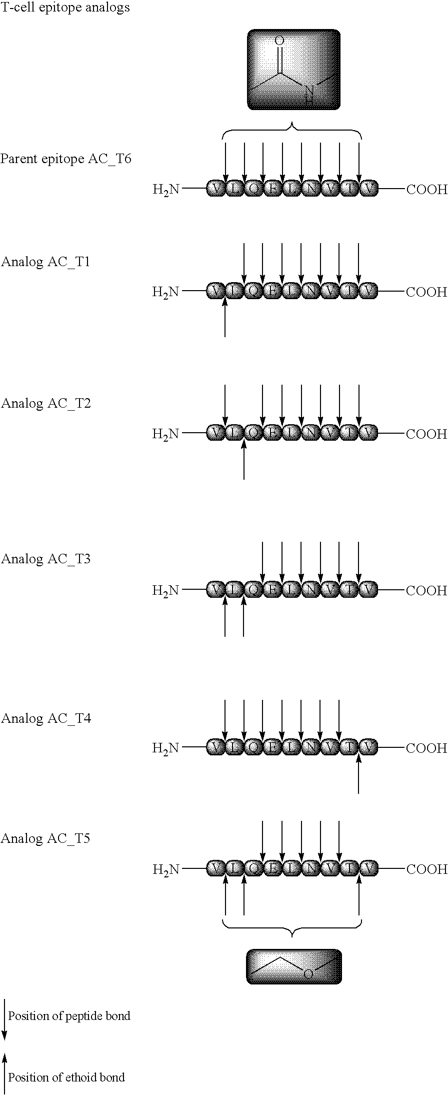

Proteinase-3 peptides for preparation of ethoid-containing analogs include: PR1, VLQELNVTV (SEQ ID NO:2); Protease-3 peptide (1) RFLPDFFTRV (SEQ ID NO:3); Protease-3 peptide (2) VLQELNVTVV (SEQ ID NO:4); Protease-3 peptide (3) NLSASVTSV (SEQ ID NO:5); Protease-3 peptide (4), IIQGIDSFV (SEQ ID NO:6); Protease-3 peptide (5) VLLALLLISGA (SEQ ID NO:7); Protease-3 peptide (6) QLPQQDQPV (SEQ ID NO:8); Protease-3 peptide (7) FLNNYDAENKL (SEQ ID NO:9); Protease-3 peptide (8) VLQELWTV (SEQ ID NO:10); or Protease-3 peptide (9) VLQELNVKV (SEQ ID NO:11); Protease-3 peptide (10) VLQELWKV (SEQ ID NO:12); or Protease-3 peptide (11) VMQELWTV (SEQ ID NO:13).

### Example 13

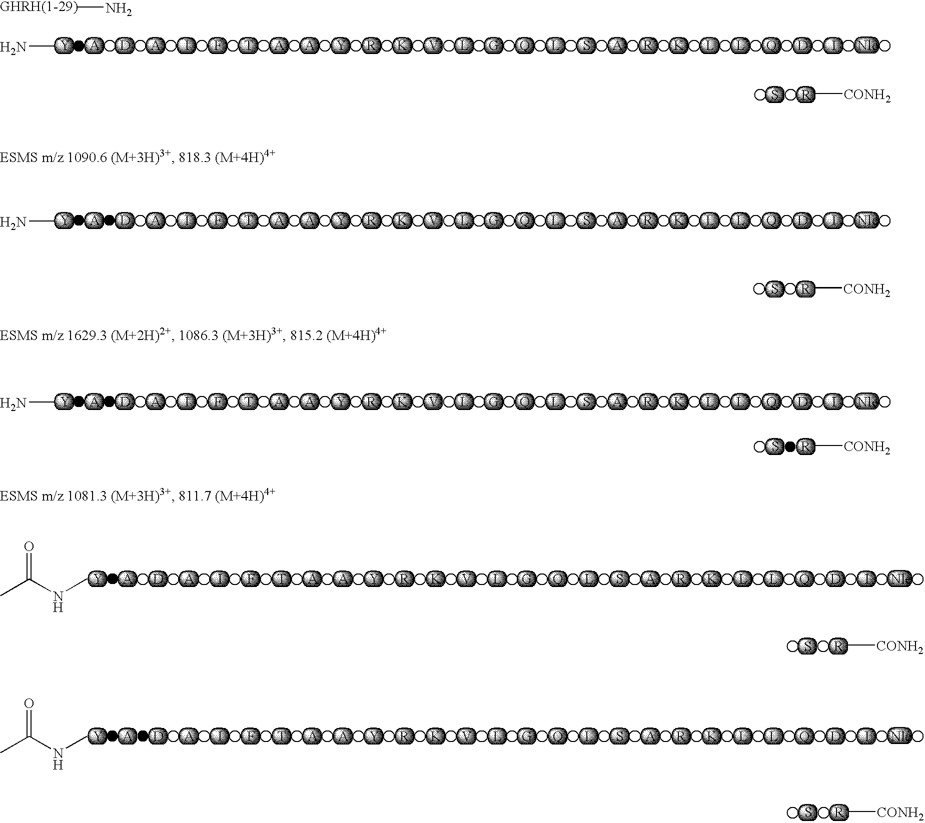

GHRH(1-29)-NH₂ peptide was prepared by standard Fmoc SPPS methodologies, ESMS m/z 1640.9 (M+2H)²⁺, 821.4 (M+4H)⁴⁺. GHRH(1-29)-NH₂ ethoid bond-containing compounds shown above were synthesized by General Method 2, preparing the protected ethoid-containing fragments separately (ie., Y*A, Y*A*D, and S*R, where * denotes an ethoid bond), and then coupling into the GHRH(1-29)-NH₂ sequence using standard Fmoc SPPS methodologies on Rink resin.

### Example 14

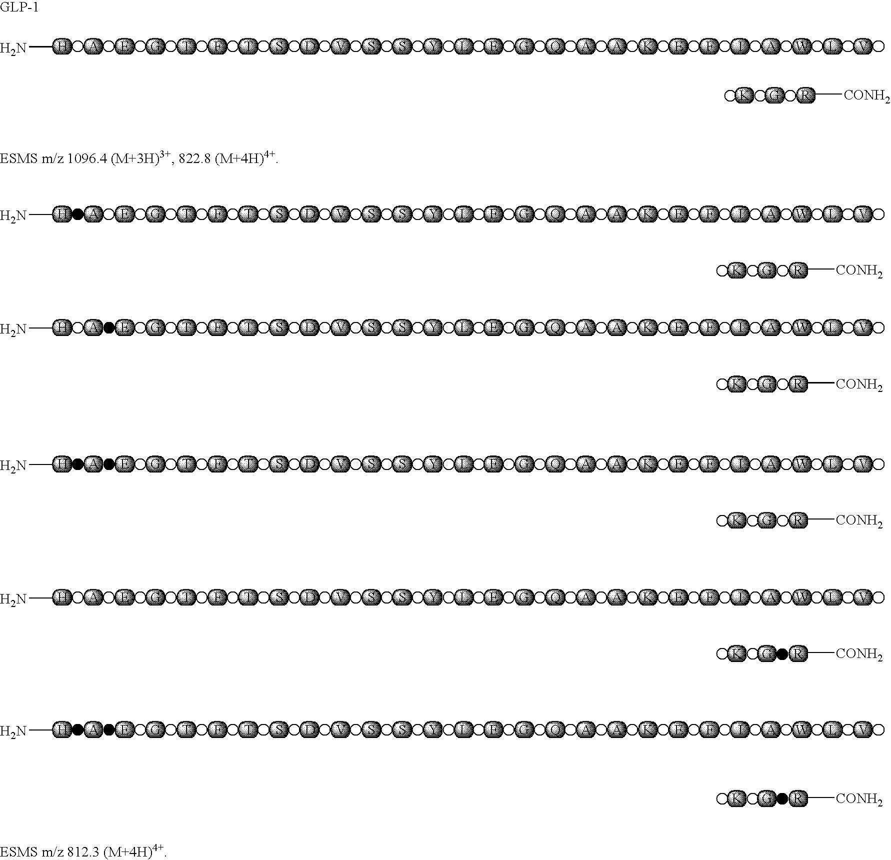

GLP-1(7-36)-NH₂ peptide shown was synthesized by standard Fmoc SPPS methodologies. GLP-1(7-36)-NH₂ ethoids were prepared by the General Method 6. The compounds were analysed by analytical HPLC-MS and purified by preparative HPLC, prior to assay.

### Example 15

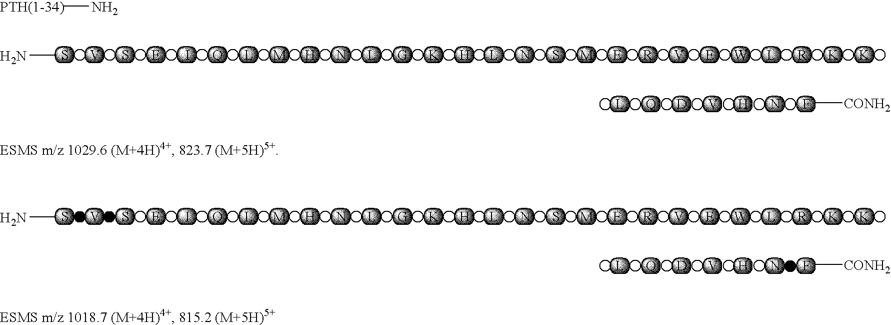

PTH(1-34)-NH₂ peptide shown was synthesized by standard Fmoc SPPS methodologies, PTH(1-34)-NH₂ polyethoid was prepared by the General Method 5. The compounds were analysed by analytical HPLC-MS and purified by preparative HPLC, prior to assay.

### Example 16

### LHRH

### Ethoid Scan and Reverse Scan Library

### Preparation of Fmoc Amino Aldehyde Building Blocks

Fmoc-L-Xaa-NMe(OMe) Weinreb amides were prepared according to the general method above, and converted to Fmoc-α-amino aldehydes which were used without further purification.

Fmoc-Gly-NMe(OMe), yield, 2.82 g, 83%
Fmoc-L-Pro-NMe(OMe), yield 3.61 g
Fmoc-L-Arg(Pbf)-NMe(OMe), yield 5.96 g, 87%
Fmoc-L-Leu-NMe(OMe), yield 3.65 g, 92%
Fmoc-L-Tyr(tBu)-NMe(OMe), yield 4.57 g, 91%
Fmoc-L-Ser(tBu)-NMe(OMe), yield 3.75 g, 88%
Fmoc-L-Trp(Boc)-NMe(OMe), yield 5.12 g, 90%
Fmoc-L-His(Trt)-NMe(OMe), yield 5.68 g, 87%
L-Pyroglutamic Weinreb amide, yield 1.31 g, 76%

### Synthesis of LHRH Library of Compounds by General Method 5

20 SynPhase PA 8 umol Rink Amide Linker derivatised lanterns (Mimotopes, Australia) were uniquely encoded by plugging 1 or 2 colored stems into both ends of the lanterns. The codes are assigned to compounds as shown in table below. Stems were available in 8 different colors (Green=G, Yellow=Y, White=W, Clear=C, Red=R, Maroon=M, Blue=B, Black=N, no stem=0). Compounds were synthesized by manually sorting the lanterns at each cycle in a mix and split strategy. General Method 5 is described above.

### Deprotection:

All 20 lanterns were combined in a flask and immersed in a solution of 20 mL 20% piperidine for Fmoc deprotection.

### Gly10 Coupling and Fmoc Deprotection:

All 20 lanterns were combined in a flask for amide bond coupling to Fmoc-Gly-OH followed by Fmoc deprotection.

### Pro9 Amide Bond Coupling:

Lanterns 1, 3-11 were sorted manually and combined in a flask for amide bond coupling to Fmoc-Pro-OH.

### Pro9 Ethoid Bond Coupling:

Lanterns 2, 12-20 were converted to hydroxyl, activated to silyl ethers for ethoid bond coupling to Fmoc-Pro-H.

### Deprotection:

Lanterns 1-20 were combined for Fmoc deprotection.

### Arg8 Amide Bond Coupling:

Lanterns 1-2, 4-10, 12 were sorted manually and combined in a flask for amide bond coupling to Fmoc-Arg(Pbf)-OH.

### Arg8 Reduced Amide Coupling to H-Pro-Solid Support:

Lanterns 3, 11, 13-20 were sorted manually and combined in a flask. The lanterns were immersed in a solution of 10 mL 0.25M Fmoc-Arg(Pbf)-H in anhydrous THF. Titanium isopropoxide (1 equiv./aldehyde, 1.5 mmol, 448 μl) was added and the reaction shaken overnight. 1.5 ml of a 1M solution of NaBH₄ in MeOH was added dropwise to the reaction and the mixture was stirred at room temperature for 16 hours. The lanterns were washed with MeOH (3 minutes, 3 times), CH₂Cl₂ (3 minutes) and DMF (3 minutes).

### Deprotection:

All 20 lanterns were combined in a flask and immersed in a solution of 20 mL 20% piperidine/DMF for 30 minutes at rt. The lanterns were washed in 20 mL DMF 4 times for 3 minutes each.

Couplings of Monomers Leu7 through pGlu1:

Monomers Leu7, Gly6, Tyr5, Ser4, Trp3, Hist and pGlu1 were sequentially coupled by either amide bond coupling or by ethoid bond coupling according to FIG. 1 and using the procedure described above, with Fmoc deprotections performed between each coupling cycle. The lanterns were manually sorted prior to each coupling as shown below.

### Leu7 Amide Bond Coupling:

Lanterns 1-3, 5-10, 13 were sorted into a flask for amide bond coupling.

### Leu7 Ethoid Bond Coupling:

Lanterns 4, 11-12, 14-20 were sorted into a flask for ethoid bond coupling.

### Fmoc Deprotection:

All lanterns were combined and deprotected.

### Gly6 Amide Bond Coupling:

Lanterns 1-4, 6-10, 14 were sorted into a flask for amide bond coupling.

### Gly6 Ethoid Bond Coupling:

Lanterns 5, 11-13, 15-20 were sorted into a flask for ethoid bond coupling.

### Fmoc Deprotection:

All lanterns were combined and deprotected.

### Tyr5 Amide Bond Coupling:

Lanterns 1-5, 7-10, 15 were sorted into a flask for amide bond coupling.

### Tyr5 Ethoid Bond Coupling:

Lanterns 6, 11-14, 16-20 were sorted into a flask for ethoid bond coupling.

### Fmoc Deprotection:

All lanterns were combined and deprotected.

### Ser4 Amide Bond Coupling:

Lanterns 1-6, 8-10, 16 were sorted into a flask for amide bond coupling.

### Ser4 Ethoid Bond Coupling:

Lanterns 7, 11-15, 17-20 were sorted into a flask for ethoid bond coupling.

### Fmoc Deprotection:

All lanterns were combined and deprotected.

### Trp3 Amide Bond Coupling:

Lanterns 1-7, 9-10, 17 were sorted into a flask for amide bond coupling.

### Trp3 Ethoid Bond Coupling:

Lanterns 8, 11-16, 18-20 were sorted into a flask for ethoid bond coupling.

### Fmoc Deprotection:

All lanterns were combined and deprotected.

### His2 Amide Bond Coupling:

Lanterns 1-8, 10, 18 were sorted into a flask for amide bond coupling.

### His2 Ethoid Bond Coupling:

Lanterns 9, 11-17, 19-20 were sorted into a flask for ethoid bond coupling.

### Fmoc Deprotection:

All lanterns were combined and deprotected.

pGlu Amide Bond Coupling:

Lanterns 1-9, 19 were sorted into a flask for amide bond coupling.

pGlu Ethoid Bond Coupling:

Lanterns 10, 11-18, 20 were sorted into a flask for ethoid bond coupling.

### Final Cleavage and Deprotection:

The lanterns were each immersed separately in 2 ml of a solution of 87.5% TFA/5% anisole/5% thioanisole/2.5% water for 2 hours at room temperature. The lantern was removed and the TFA solution was evaporated under vacuum and the product was precipitated in 3 ml of cold diethyl ether. The solution was decanted and the precipitate was dissolved in water and freeze-dried to yield crude product.

### Analytical HPLC

All the compounds were analyzed by HPLC-MS, using a Gilson HPLC with dual wavelength UV detector (210 and 254 nm). The column used was a C₁₈ reverse phase (4.6×250 mm; 5 μmx 6 A). The solvent gradient and system used was 10% CH₃CN (0.1% TFA)/90% H₂O (0.1% TFA) @ 1 ml/min over 10 minutes, then hold at 100% CH₃CN (0.1% TFA) for 10 minutes. The solvent flow was split coming off the column into an Applied Biosystem API 150 EX mass spectrometer, scanning between 500-2000 amu.

### Preparative HPLC

LHRH compounds were purified by preparative HPLC. The solvent gradient and system used was 10% CH₃CN (0.1% TFA)/90% H₂O (0.1% TFA) @ 4 ml/min over 10 minutes, then hold at 100% CH₃CN (0.1% TFA) for 10 minutes. Fractions were collected and analysed by ESMS.

TABLE IV
Synthetic Summary for LHRH library

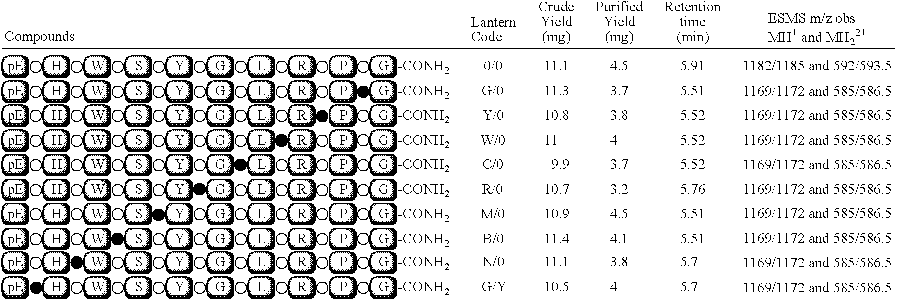

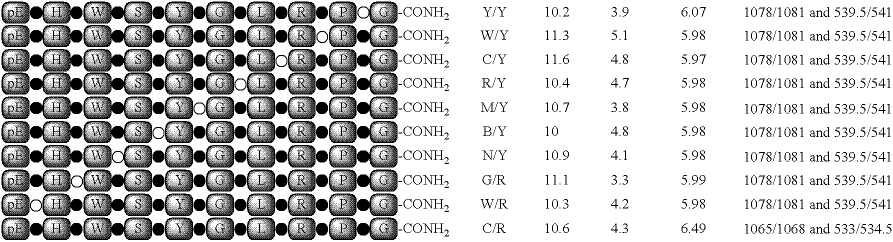

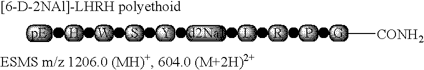

The polyethoid above was prepared by General Method 5. The compound was analysed by analytical HPLC-MS and purified by preparative HPLC, prior to assay.

### Example 17

### Parallel Synthetic Strategy for Polyethoids with Mixed Sequences

The following polyethoid compounds were synthesized by General Method 5, in parallel, utilizing color-encoded lanterns and a mix and split synthetic strategy similar to that described above for the LHRH ethoid scan library. By encoding each SynPhase PA 8 μmol lantern, lanterns can be combined for common steps such as deprotections, amino to silyl ether conversions and washes. Lanterns can be combined for coupling steps if they happen to be coupling the same building block at a particular cycle in the sequence.

All chemical steps and methods were as described above. After cleavage, each compound was analysed by analytical HPLC-MS and purified by preparative HPLC, prior to assay.

The peptide control compounds were all prepared by standard Fmoc SPPS methodologies, also on SynPhase PA 8 μmol lanterns.

TABLE V
Analytical HPLC-MS Data of peptide and polyethoid compounds shown below, prior to purification

| | Peptide | | Polyethoid | |
|---|---|---|---|---|
| | HPLC % purity | ESMS m/z | HPLC % purity | ESMS m/z |
| Substance P | 72 | 1347.9 (MH⁺), 674.7 (MH₂²⁺) | 82 | 1214.8 (MH⁺) |
| Neurokinin A | 84 | 1133.8 (MH⁺) 567.8 (MH₂²⁺) | 97 | 1016.6 (MH⁺) |
| Neurokinin B | 79 | 1210.7 (MH⁺) | 95 | 1093.9 (MH⁺) |
| Bombesin | 69 | 810.9 (MH₂²⁺) | 68 | 1452.0 (MH⁺), 726.6 (MH₂²⁺) |
| CCK-8 | 75 | 1063.7 (M-SO₃H⁺) | 75 | 1052.6 (MH⁺), 527.7 (MH₂²⁺) |
| Somatostatin | 98 | 820 (MH₂²⁺) | 97 | 1466.9 (MH⁺), 734.5 (MH₂²⁺) |
| Leu-enkephalin | 99 | 556.5 (MH⁺) 1111.7 (M₂H)⁺ | 73 | 504.5 (MH⁺), 1007.3 (M₂H⁺) |
| Met-enkephalin | 63 | 574.3 (MH⁺) | 68 | 522.4 (MH⁺), 1043.9 (M₂H)⁺ |

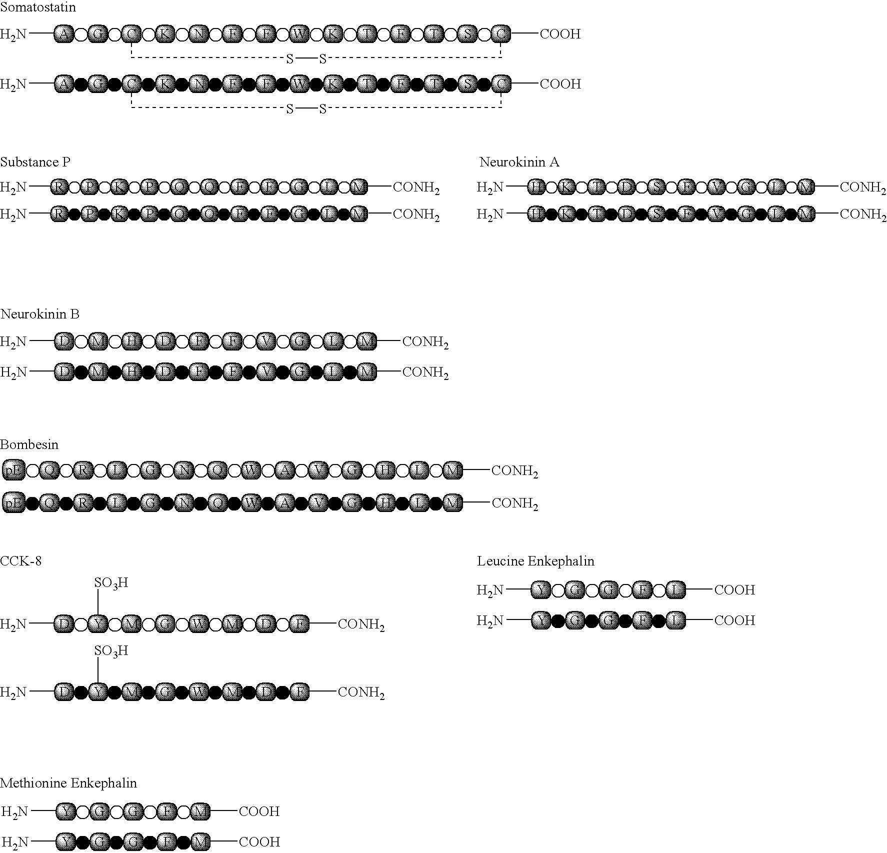

### F. Specific Examples

### Activity Assays

### Example 18

### GHRH

### Assay Details

Functional cell-based assay for hGHRH receptor agonism and antagonism

The assays were performed in Prof. Michael Thorner's lab at the University of Virginia. A cell-based assay for receptor activation by measuring intracellular cAMP levels, was developed using a cell line expressing hGHRH receptor. The receptor was cloned at UVA as described in the following patent (Cloning of a cDNA for human growth hormone releasing hormone receptor. Thorner, M. O.; Gaylinn, B. D.; Harrison, J. K.; Lynch, K. R.; Zysk, J. R. (University of Virginia Patent Foundation, USA). U.S. (1997), 36 pp., Cont.-in-part of U.S. Ser. No. 947,672. CODEN: USXXAM U.S. Pat. No. 5,644,046 A 19970701 patent written in English. Application: US 95-432043 19950501).

Dose response curves for GHRH compounds in two assay experiments are shown in the following figures.

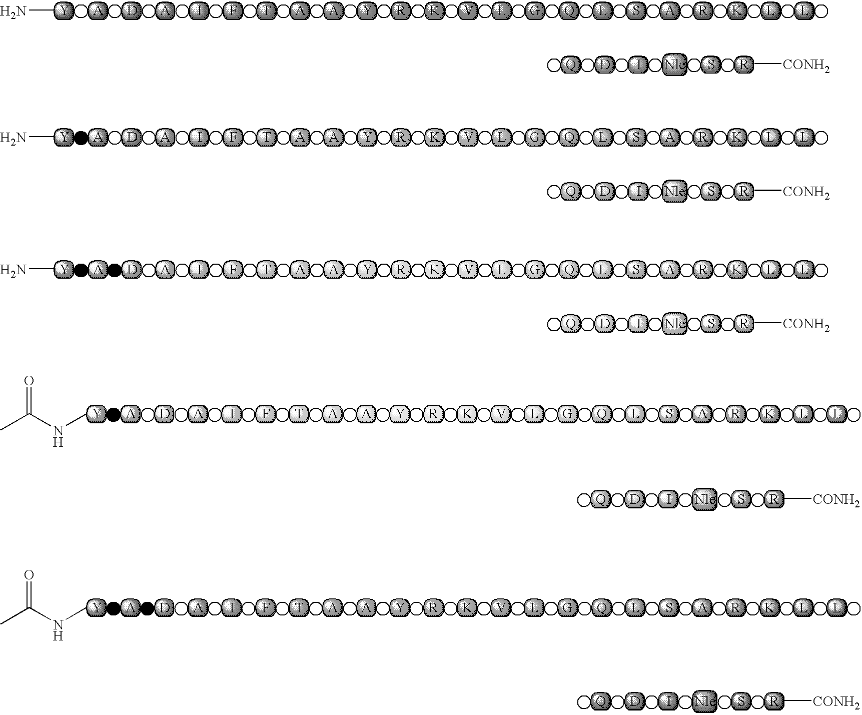

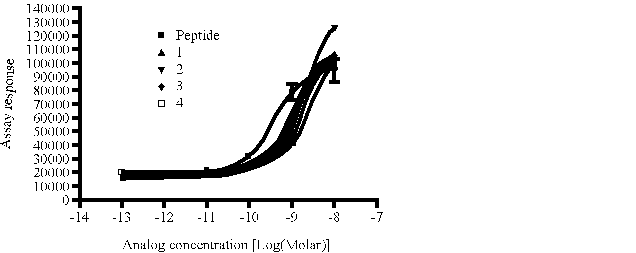

Figure: Dose response for GHRH peptide and GHRH analogs containing ethoid bonds between Y-A (1), Y-A and A-D (2), Y-A (3) and Y-A and A-D (4).

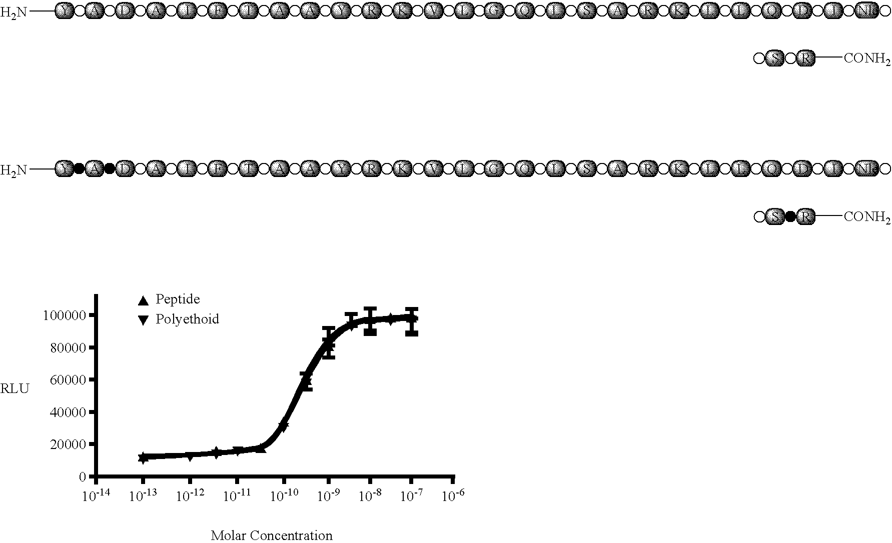

Figure: Dose response for GHRH peptide and polyethoid with structures shown above.

### Example 19

### P3 B-Cell Epitope

### Assay Methods

Assay 1: ELISA screening of immobilized biotinylated compounds: NUNC Maxisorp plates were coated with 5 μg/ml NeutrAvidin in PBS pH 7.4 (4° C. for 16 hours) and then with 5 μg/ml biotinylated compounds in PBS pH 7.4. Unless otherwise noted all incubations were performed at 37° C. for 60 min. The plates were blocked with 1% Blocker BSA in PBS pH 7.4/0.05% Tween20 (PBST). MABs were incubated at 1 in 1000 dilution in blocking buffer. After incubation the plates were washed 3× with PBST and incubated with 0.5 ug/mL goat anti-mouse IgG-HRP conjugate in blocking buffer. The plates were washed 3× with PBS-T and developed with 0.5 mg/mL ABTS (2,2′-azinobis[3-ethylbenzothiazoline-6-sulfonic acid]-diammonium salt) substrate in phosphate-citrate-perborate buffer (Sigma P4922) measuring absorbance at 405 nm after 30 min.

Assay 2: MAB Microarray ELISA assay NUNC Maxisorp plates were spotted (see microarray spotting conditions below) with 0.45 mg/ml goat anti-mouse in 1% glycerol/PBS/0.0005% Tween20 and incubated at RT for 2 hours. Using an identical spacing P3M2, P3M5 and P3M7 MABs were spotted at approx. 0.1 mg/mL in 20% glycerol/PBS/0.0005% Tween20, the plates sealed and incubated at 4° C. for 16 hours. 0.5% casein/PBS blocking buffer was added down side of well and incubated either at 37° C. for 1 hour or stored at 4° C. for later use. The blocking buffer is removed and plate washed (3×PBST). The plates were incubated according to the experiment with biotinylated peptides or ethoids in 1% BSA/PBST incubation buffer. The plates were washed (3×PBST), incubated with 0.5 ug/mL neutravidin-HRP conjugate for 30 min at 37° C., washed again (3×PBST) and chemiluminescent substrate (Supersignal ELISA Femto substrate Pierce #37075) added to each well. Microarrays were immediately imaged with a Kodak Image Station 2000R at best possible resolution (20 μm per pixel). Images were processed using the Kodak 1D software provided (Kodak Scientific Imaging Systems, New Haven, Conn.), by fitting a grid of ROIs (region of interest) set at a fixed size and capturing ROI sum intensity for each spot.

Assay 2: Microarray spotting Microarrays were spotted using a Cartesian xyz robot fitted with a Telechem chipmaker printhead and single CMP10B pin (365 μm diameter spots). Spotting was performed at 60% relative humidity and RT. Standard motion control, pin washing and drying parameters were used (www.arrayit.com). Hexagonal array was set to generate either 37 spot positions (3 rings of spots at 600 μm center to center spacing), 61 positions (4 rings at 600 μm spacing) or 91 positions (5 rings at 480 μm spacing). Five of the vertices were designated as anchor positions. The center position and sixth vertice were designated at negative control positions, leaving 30, 54 or 84 open spot positions for MABs. Higher density arrays are easily accessible by printing smaller diameter spots, ie. 100 μm spots on 200 μm spacing and 12 ring hexagonal array would allow 469 positions in each well of a 96-well plate. Typical microarray imagers have a best resolution of 5-20 μm per pixel.

### Assay Results Summary

A summary of results for the binding of P3 ethoid compounds to three different MABs with specificity for the epitope —PEQGYN-NH₂ by assay method 1 are shown in table VI

TABLE VI

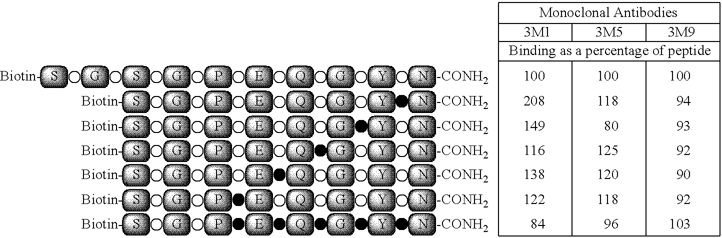

### Example 20

### LHRH

### Assay Method

Functional cell based assay for LHRH/GnRH receptor agonism

Performed by EuroScreen (Belgium).

Materials and Methods

Compounds were received as powders and stored at room-temperature prior to solubilization. Compounds were prepared according to Table VII:

TABLE VII

| | | Compound | | |
|---|---|---|---|---|
| | Solvent | Concentration | | Storing Conditions |
| Master Solution | 100% DMSO | 10 | mM | −20° C. |
| Mother | 30% DMSO | 100 | μM | |

| | | | |
|---|---|---|---|
| Assay Plate | Assay buffer | 1 μM-0.1 nM | Max 4 h at RT |

### Recombinant Cell Line and Assay

AequoScreen™ cell lines expressing the human GnRH recombinant receptor (table 2) were used throughout the study.

| Receptor | Receptor Accession Number | Catalog number |
|---|---|---|
| GnRH | L03380 | FAST-0160A |

AequoScreen™ cells were incubated at room temperature for at least 4 h with coelenterazine h.

For agonist testing, 50 μl of cell suspension were mixed with 50 μl of test or reference agonist in a 96-well plate. The resulting emission of light was recorded using the Hamamatsu Functional Drug Screening System 6000 (FOSS 6000).

Following an incubation of 15 min after the first injection, 100 μl of the resulting cell suspension containing the test compound were mixed with 100 μl of the reference agonist in the 96 well test plates. The resulting emission of light was recorded using the same luminometer as for agonist testing.

To standardize the emission of recorded light (determination of the “100% signal”) across plates and across different experiments, some of the wells contained 100 μM digitonin, a saturating concentration of ATP (20 μM) and a concentration of reference agonist equivalent to the EC₅₀ obtained during test validation. Plates also contained the reference agonist at a concentration equivalent to the EC₁₀₀.

Agonist activities of test compound are expressed as a percentage of the activity of the reference agonist at its EC₁₀₀ concentration. Antagonist activities of test compound are expressed as a percentage of the inhibition of reference agonist activity at its EC₈₀ concentration.

Test Specifications All compounds were tested at the concentrations of 0.1, 1, 10, 100, 1000 nM in duplicate. [D-Trp6] LHRH (Bachem, H4075) was used as the reference agonist and antagonist for the human GnRH receptor.

Quality Control On each day of experimentation and prior to the testing of compounds, reference compounds were tested at several concentrations in duplicate (n=2) to obtain a dose-response curve and an estimated EC₅₀ values.

Reference values thus obtained for the test were compared to historical values obtained from the same receptor and used to validate the experimental session.

A session was considered as valid only if the reference value was found to be within a 0.5 logs interval from the historical value.

Values are indicated in Table 3Table 3

| Receptor | Ref. Compound | EC₅₀ | Historical |
|---|---|---|---|
| GnRH | [D-Trp6] LHRH | 0.14 nM | 0.38 nM |

For replicate determinations, the maximum variability tolerated in the test was of +1-20% around the average of the replicates.

TABLE VIII
Summary Results for LHRH compounds

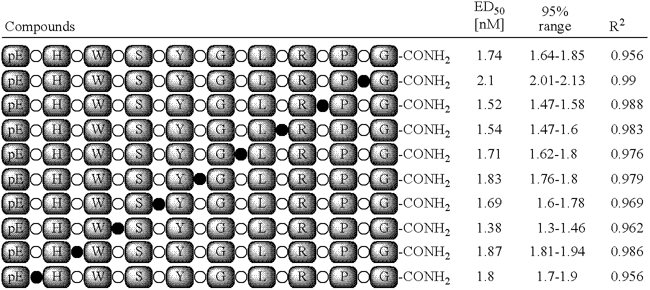

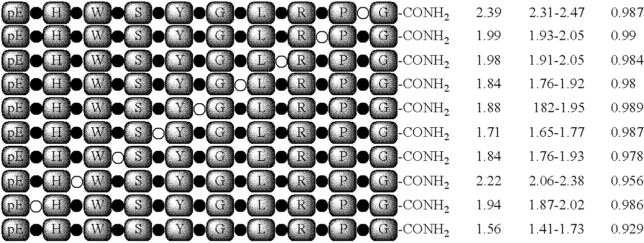

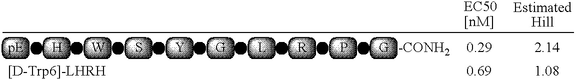

Mean EC₅₀ of 20 compounds 1.83 nM, SD = 0.24 nM
Top Shared 106.3
Bottom Shared −5.1

Table: EC₅₀ values obtained for compounds in LHRH receptor agonist assay.

### Example 21

### GLP-1

Functional cell based GLP-1 receptor assay: Performed by Pharmacelsus (Germany).

List of key instruments UV/VIS spectroscope: Spectramax Plus³⁸⁴ (Molecular Devices); data handling with the standard software SoftmaxPro 3.1.2.

Chemicals 111-CHO-349/18 cells recombinantly expressed the human GLP-1 receptor. Medium (Ham's F12) and supplements (Trypsin-EDTA, Penicillin/Streptomycin, BSA, HEPES) for cell culture were purchased from ccpro (Neustadt, Germany). Fetal calf serum (FCS) was from Gibco-Invitrogen and was used after heat-inactivation at 56° C. for 30 min. Aprotinin (proteinase inhibitor, supplemented for inhibition of unspecific protein digestion during the incubation) was used as Trasylol® from Bayer (Leverkusen, Germany).

Human GLP-1 (7-36) amide was from Bachem (Switzerland). IBMX (3-isobutyl-1-methylxanthine, non-specific inhibitor of cAMP- and cGMP-phosphodiesterases) was from Across Organics (Geel, Belgium). The competitive immunoassay kit used for the quantitative determination of cAMP (Correlate™ EIA Direct Cyclic AMP Enzyme Immunoassay Kit) was from Assay Designs (Ann Arbour, USA).

Cell culture 111-CHO-349/18 cells were grown in Ham's F12 medium supplemented with 1% L-glutamine (200 mM), 1% penicillin/streptomycin (100×) and heat-inactivated FCS (10%) and were maintained in a 5% CO₂ atmosphere at 37° C. The cell culture medium was renewed every 48 h and the cells were split in a 1:20 ratio once a week for maintaining

Receptor activation assay For assays, 111-CHO-349/18 cells (passages 10-15) were harvested with trypsin/EDTA, seeded in 24-well-plates (Corning) at a density of 2×10⁵ cells per well and allowed to attach for 6 h, which resulted in monolayers of 90-95% confluency. Cells were then carefully washed twice with 37° C. warm wash buffer (Ham's F12 medium supplemented with 1% L-glutamine, 1% penicillin/streptomycin, 10% heat-inactivated FCS, 15 mM HEPES, 500 μM IBMX and 0.1% BSA) and were preincubated with this buffer for 1 h.

GLP-1 (Bachem, Switzerland) was dissolved in 0.01 N acetic acid according to the recommendations of the manufacturer. The six test compounds were provided from Allchemie. All compounds were dissolved in 0.01 N acetic acid at a concentration of 0.5 mg/ml and stored aliquoted at −20° C.

For cAMP releasing studies, the test compounds were serially diluted in assay medium (Ham's F12 medium supplemented with 1% L-glutamine, 1% penicillin/streptomycin, 10% heat-inactivated FCS, 15 mM HEPES, 500 μM IBMX, 0.1% BSA and 1% Trasylol). The monolayers were incubated for 30 min with increasing concentrations of test compounds including an untreated control and 1 μM GLP-1 as positive control. Control experiments were performed in duplicate. After the incubation period, the supernatants were removed from the monolayers and the cells were Iyzed with 1% Triton X-100 in 0.1 M HCl.

The lysates were applied for the quantification of released cAMP in the enzyme immunoassay (EIA) according to the manufacturer's instructions. The optical densities (00) of samples were measured at 405 nm (reference wavelength 590 nm).

Data analysis For calculation of results, cAMP levels of the respective samples were calculated from a cAMP standard curve included in the EIA setup. The 00 values were fitted using a logit-Iog approach, for which the logit of each standard (corrected for non specific binding, NSB) was plotted against its log concentration. Logit values were calculated as follows:

The concentration of the samples was calculated from the equation resulting from the standard curve and expressed as fmol cAMP/1000 cells. The cAMP value of each sample was normalized to the maximum cAMP release induced by 1 μM GLP-1, which corresponds to 100% cAMP production.

For each test compound, a dose-response curve was generated by plotting the resulting stimulation of cAMP release (in % of maximum release induced by 1 μM GLP-1) as a function of compound concentration. The data for all GLP-1(7-36)-NH₂ analogs and control is represented in figure below.

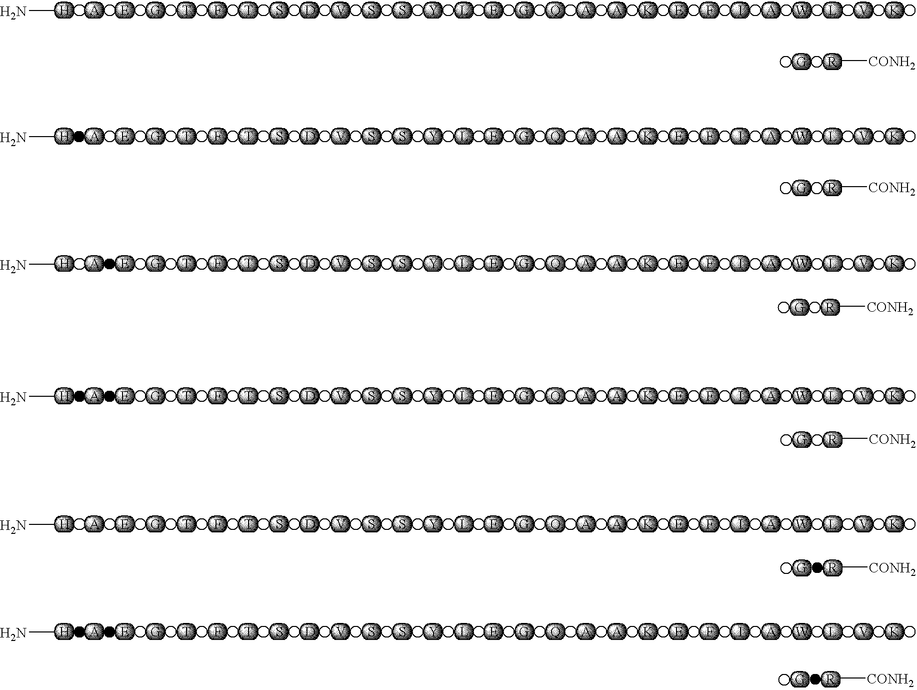

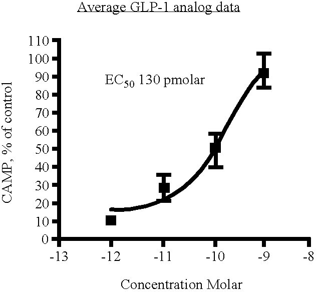

Figure: Mean of the dose response curves for GLP-1(7-36)-NH₂ and ethoid analogs above.

### Example 22

### PTH

Assay Details: Functional cell based assay of PTH receptor 1 agonism. Assays were performed by Multispan (Hayward, Calif.). HEK293T cells transiently-transfected with PTHR1 were seeded in 96-well PDL-coated plate for the Ca++ assay. PTHR1 expression on cell surface was detected by an anti-FLAG antibody conjugate. Agonist experiments were performed by measuring the dose response of intracellular calcium with FlexStation, at 5 concentrations of each compound in duplicate, for semi quantitative estimates of EC50. Full length PTH was assayed as a positive control.

Assay Results are shown in Tables IX.A and IX.B

TABLE IX.A
PTH(1-34)-NH₂ peptide control

| | | | | |
|---|---|---|---|---|
| Sample | Conc. nM | Values | MeanValue | StdDev |

| | | | | |
|---|---|---|---|---|
| 1 | 0 | 3069.226 | 2993 | 108.095 |
| | | 2916.356 | | |
| 2 | 0.1 | 3878.595 | 3685 | 274.225 |
| | | 3490.782 | | |
| 3 | 1 | 4758.973 | 4296 | 654.293 |
| | | 3833.663 | | |
| 4 | 10 | 4379.733 | 3947 | 612.330 |
| | | 3513.767 | | |
| 5 | 100 | 5240.728 | 5562 | 483.158 |
| | | 5924.016 | | |
| 6 | 1000 | 26254.94 | 26306 | 72.148 |
| | | 26356.97 | | |

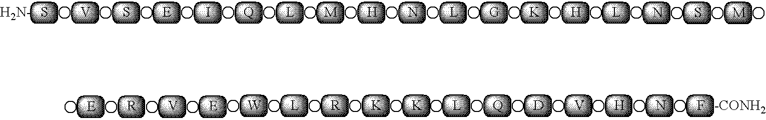

TABLE IX.B
PTH(1-34)-NH₂ polyethoid

| | | | | |
|---|---|---|---|---|
| Sample | Conc. nM | Values | MeanValue | StdDev |

| | | | | |
|---|---|---|---|---|
| 1 | 0 | 3251.209 | 3485 | 330.184 |
| | | 3718.16 | | |
| 2 | 0.1 | 3588.278 | 3723 | 190.408 |
| | | 3857.555 | | |
| 3 | 1 | 5013.285 | 4725 | 408.047 |
| | | 4436.219 | | |
| 4 | 10 | 2882.955 | 3122 | 338.204 |
| | | 3361.248 | | |
| 5 | 100 | 7292.802 | 6587 | 997.533 |
| | | 5882.077 | | |
| [symbol] | [symbol] | [symbol] | [symbol] | [symbol] |

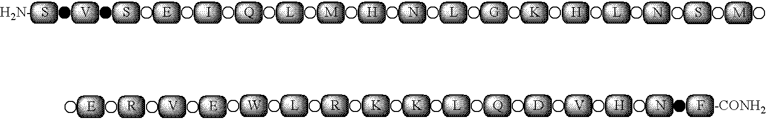

[symbol]

indicates data missing or illegible when filed

### Example 23

### Somatostatin

Assay Details Somatostatin sst1 human receptor cell based competition binding assay. Performed by MDS Pharma Services (Assay #282510)

| source | Human recombinant (CHO-K1 cells) |
|---|---|
| ligand | 0.1 nM [¹²⁵I] Somatostatin-14 |
| non-specific | 1 μM Somatostatin-14 |
| Kd | 0.48 nM |
| Bmax | 5800 fmol/mg Protein |
| specific binding | 93% |

References Patel Y C and Srikant C B. Endocrinology. 135(6):2814, 1994. Liapakis G et al. J Biol Chem. 271(24):20331, 1996.

### Assay Results

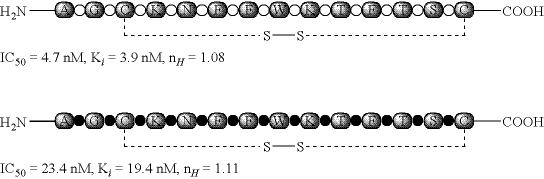

### Example 24

### Substance P

Assay Details Tachykinin NK1 HUMAN receptor cell based competition binding assay. Performed by MDS Pharma Services (Assay #: 255510)

| source | Human recombinant (CHO cells) |
|---|---|
| ligand | 0.25 nM [3H] SR-140333 |
| non-specific | 2 μM L-703,606 |
| Kd | 0.3 nM |
| Bmax | 10000 fmol/mg Protein |
| specific binding | 85% |

References: Patacchini R and Maggi C A. Arch Int Pharmacodyn. 329:161, 1995

Assay Results

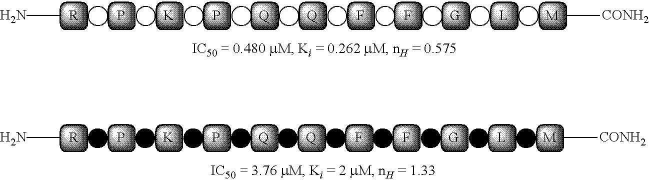

### Example 25

### Neurokinin A

Assay Details Tachykinin NK2 HUMAN receptor cell based competition binding assay. Performed by MDS Pharma Services (Assay #: 255600)

| source | Human recombinant (CHO cells) |
|---|---|
| ligand | 0.5 nM [3H] SR-48968 |
| non-specific | 2 μM MEN-10,376 |
| Kd | 0.25 nM |
| Bmax | 8500 fmol/mg Protein |
| specific binding | 75% |

References: Patacchini R and Maggi C A. Arch Int Pharmacodyn. 329:161, 1995

Assay Results

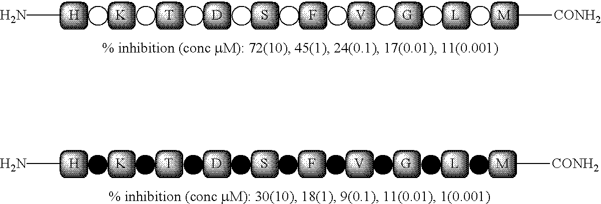

### Example 26

### Neurokinin B

Assay Details Tachykinin NK3 HUMAN receptor cell based competition binding assay. Performed by MDS Pharma Services (Assay #: 255710)

| source | Human recombinant (CHO cells) |
|---|---|
| ligand | 0.06 nM [125I] MePhe7-Neurokinin B |
| non-specific | 50 μM Senktide |
| Kd | 0.28 nM |
| Bmax | 770 fmol/mg Protein |
| specific binding | 88% |

References Krause J E et al. Proc Natl Acad Sci USA. 94: 310, 1997. Sadowski S et al. Neuropeptides. 24(6): 317, 1993.

Assay Results

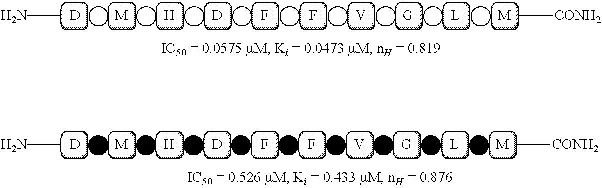

### Example 27

### Bombesin

Assay Details Bombesin BB1 HUMAN receptor cell based competition binding assay. Performed by MDS Pharma Services (Assay #: 211600)

| source | Human recombinant (CHO cells) |
|---|---|
| ligand | 0.05 nM [125I] (Tyr4)-Bombesin |
| non-specific | 1 μM Neuromedin B |
| Kd | 0.045 nM |
| Bmax | 6600 fmol/mg Protein |
| specific binding | 95% |

References Ryan R R et al. J Pharmacol Exp Ther. 290(3): 1202, 1999. Sainz E et al. J Biol Chem. 273(26): 15927, 1998.

Assay Results

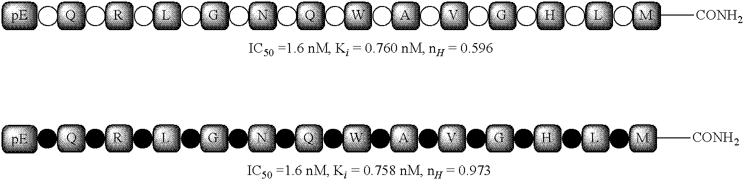

### Example 28

### CCK-8

Assay Details. Cholecystokinin CCK1 (CCKA) Human receptor cell-based competition binding assay, Performed by MDS Pharma Services (Assay #: 218010)

| source | Human recombmant (NIH-3T3 cells) |
|---|---|
| ligand | 0.8 nM [3H] Devazepide (L-364,718) |
| non-specific | 1 μM Devazepide (L-364,718) |
| Kd | 0.2 nM |
| Bmax | 130 fmol/mg Protein |
| specific binding | 80% |

References—Jensen R T et al. Ann NY Acad Sci. 713:88, 1994

### Assay Results

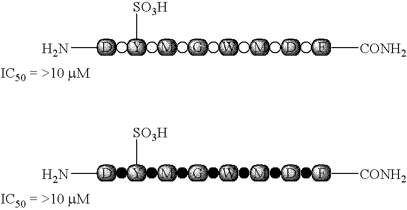

Assay Details Cholecystokinin CCK2 (CCKB) Human receptor cell based competition binding assay. Performed by MDS Pharma Services (Assay #: 218120)

| source | Human FGS-7 Jurkat cells |
|---|---|
| ligand | 0.072 nM [125I] CCK-8 |
| non-specific | 1 μM Sincalide |
| Kd | 0.1 nM |
| Bmax | 750 |
| specific binding | 85% |

References Kaufmann R et al. Neuropeptides. 29:63, 1995. Cuq P et al. Life Sci. 61(5):543, 1997.

Assay Results

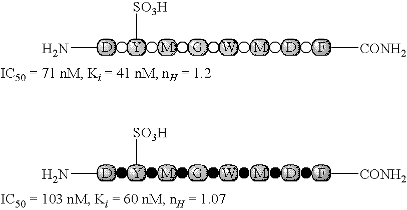

### Example 29

### Leucine Enkephalin and Methionine Enkephalin

Assay Details: Opiate δ (OP1, DOP) human receptor cell based competition binding assay, Performed by MDS Pharma Services (Assay #: 260110)

| source | Human recombinant (CHO cells) |
|---|---|
| ligand | 0.9 nM [3H] Naltrindole |
| non-specific | 10 μM Naloxone |
| Kd | 0.49 nM |
| Bmax | 8600 fmol/mg Protein |
| specific binding | 80% |

References Simonin F et al. Mol Pharmacol. 46:1015, 1994.

Assay Results

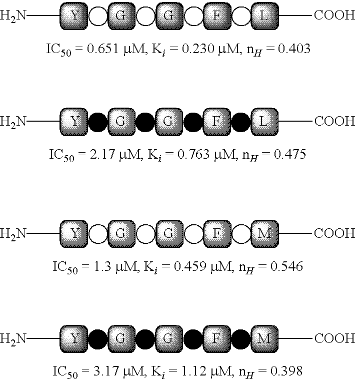

Assay Details Opiate δ (OP2, KOP) human receptor cell based competition binding assay. Performed by MDS Pharma Services (Assay #: 260210)

| source | Human recombinant (HEK-293 cells) |
|---|---|
| ligand | 0.6 nM [3H] Diprenorphine |
| non-specific | 10 μM Naloxone |
| Kd | 0.4 nM |
| Bmax | 1100 fmol/mg Protein |
| specific binding | 90% |

References Maguire P et al. Eur J Pharmacol. 213:219, 1992.+PubMed Simonin F et al. Proc Natl Acad Sci USA. 92(15):7006, 1995.

Assay Results

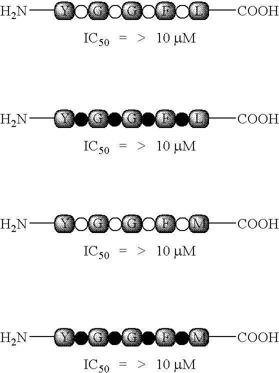

Assay Details Opiate δ (OP3, MOP) human receptor cell based competition binding assay. Performed by MDS Pharma Services (Assay #: 260410)

| source | Human recombinant (CHO-K1 cells) |
|---|---|
| ligand | 0.6 nM [3H] Diprenorphine |
| non-specific | 10 μM Naloxone |
| Kd | 0.41 nM |
| Bmax | 3800 fmol/mg Protein |
| specific binding | 90% |

References Wang J B et al. FEBS Lett. 338:217, 1994.

Assay Results

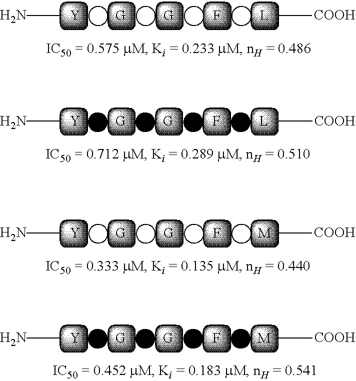

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A compound comprising a polyethoid moiety having a formula 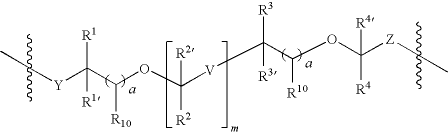
wherein:
m is an integer ≧0;
each a is an independently selected integer=1 or =2;
R¹, each R² and R⁴ are each (i) an independently selected side chain moiety selected from the group consisting of H, C₁-C₁₀ alkyl and substituted C₁-C₁₀ alkyl, which in each case can optionally form one or more ring structures, or (ii) an independently selected side chain moiety having a structure of an amino acid side chain;
R³ is (a) a side chain moiety selected from the group consisting of R_{C}, R_{D}, R_{E}, R_{F}, R_{H}, R_{I}, R_{K}, R_{L}, R_{M}, R_{N}, R_{P}, R_{Q}, R_{R}, R_{T}, R_{U}, R_{V}, R_{W} and R_{Y}, each as delineated in Table I.A, (b) a side chain moiety selected from and having a structure of a non-natural amino acid side chain as delineated in Table I.B.1 or in Table I.C.1 or (c) a protected derivative of the foregoing side chain moieties;
R¹′, each R²′, R³′ and R⁴′ are each independently selected from the group consisting of H, C₁-C₃ alkyl and substituted C₁-C₃ alkyl;
each R¹⁰ is independently selected from the group consisting of H, C₁-C₃ alkyl and substituted C₁-C₃ alkyl;
each V is independently selected from the group consisting of —C(O)NH— and -ψ[ ]-; and
Y and Z are each independently selected from the group consisting of H, hydrocarbyl and substituted hydrocarbyl.

2.-1126. (canceled)
